SEQUENCE LISTING (35 sequences)

SEQ ID NO: 1 - DNA, 35 bp, synthetic construct
  source 1..35: mol_type = other DNA
aaggcctctgtcgaccatggcatcatcacaagtac

SEQ ID NO: 2 - DNA, 37 bp, synthetic construct
  source 1..37: mol_type = other DNA
agaattcgcaagcttattctgacatctgagtggaagt

SEQ ID NO: 3 - DNA, 25 bp, synthetic construct
  source 1..25: mol_type = other DNA
ggatccttatggcatcatcacaagt

SEQ ID NO: 4 - DNA, 22 bp, synthetic construct
  source 1..22: mol_type = other DNA
gcggccgcttcttgtacagctc

SEQ ID NO: 5 - DNA, 38 bp, synthetic construct
  source 1..38: mol_type = other DNA
catttacctctgaagccacgggtttagtgaaccgtcag

SEQ ID NO: 6 - DNA, 28 bp, synthetic construct
  source 1..28: mol_type = other DNA
ttcctcctccaaccgcctgttttaaaac

SEQ ID NO: 7 - DNA, 28 bp, synthetic construct
  source 1..28: mol_type = other DNA
acaggcggttggaggaggaagatctaag

SEQ ID NO: 8 - DNA, 39 bp, synthetic construct
  source 1..39: mol_type = other DNA
tcgagctccaccgcggtggcaacaccccttgtattactg

SEQ ID NO: 9 - DNA, 38 bp, synthetic construct; identical to SEQ ID NO: 5
  source 1..38: mol_type = other DNA

SEQ ID NO: 10 - DNA, 42 bp, synthetic construct
  source 1..42: mol_type = other DNA
tcgagctccaccgcggtggctaagatacattgatgagtttgg

SEQ ID NO: 11 - DNA, 26 bp, synthetic construct
  source 1..26: mol_type = other DNA
atgtccctcgtcatcccagagaagtt

SEQ ID NO: 12 - DNA, 23 bp, synthetic construct
  source 1..23: mol_type = other DNA
attgtccagaccattagcaagga

SEQ ID NO: 13 - DNA, 20 bp, synthetic construct
  source 1..20: mol_type = other DNA
ttcttggtctgcctttgacg

SEQ ID NO: 14 - DNA, 20 bp, synthetic construct
  source 1..20: mol_type = other DNA
gcatggaagtacttggtaag

SEQ ID NO: 15 - DNA, 23 bp, synthetic construct
  source 1..23: mol_type = other DNA
aattacctgactcgactgtatgg

SEQ ID NO: 16 - DNA, 19 bp, synthetic construct
  source 1..19: mol_type = other DNA
ccagtggcgaagaagatca

SEQ ID NO: 17 - DNA, 20 bp, synthetic construct
  source 1..20: mol_type = other DNA
tggcaataaacatcgctttg

SEQ ID NO: 18 - DNA, 20 bp, synthetic construct
  source 1..20: mol_type = other DNA
ttcactggagtcttggagca

SEQ ID NO: 19 - DNA, 22 bp, synthetic construct
  source 1..22: mol_type = other DNA
cgtatgtgctctctcagattgg

SEQ ID NO: 20 - DNA, 23 bp, synthetic construct
  source 1..23: mol_type = other DNA
gtcaatcctgctactgtcgtaaa

SEQ ID NO: 21 - DNA, 20 bp, synthetic construct
  source 1..20: mol_type = other DNA
aggtgaccgatcacccaatg

SEQ ID NO: 22 - DNA, 20 bp, synthetic construct
  source 1..20: mol_type = other DNA
ttaggtttttggcccttctg

SEQ ID NO: 23 - DNA, 26 bp, synthetic construct
  source 1..26: mol_type = other DNA
gagggtcctgatagagaaaaagtgaa

SEQ ID NO: 24 - protein, 9 aa, Danio rerio
PTGTTPQRK

SEQ ID NO: 25 - DNA, 8500 bp, synthetic construct
  source 1..8500: mol_type = other DNA
ccacctaaattgtaagcgttaatattttgttaaaattcgcgttaaatttttgttaaatcagctcattttttaaccaataggccgaaatcggcaaaatcccttataaatcaaaagaatagaccgagatagggttgagtgttgttccagtttggaacaagagtccactattaaagaacgtggactccaacgtcaaagggcgaaaaaccgtctatcagggcgatggcccacgctggctaagaactcatcagcctccccggtccatctacccacgtaccaatgcaccaattggccacaatgacggctactacatggtgccattccttcctctttataggaatggagactacctcctgtccaacaaggctcttggatacgagtacgcctacctgttggacccaggtcattgcacaacaccagaaatgccctctgatctgcaaaagacgtgaatatctgttcagacacccatatccactctgttccacacaggtcagaggtttgtccaggagttcttgacagaggtgtaaaaagtactcaaaaattttactcaagtgaaagtacaagtacttagggaaaattttactcaattaaaagtaaaagtatctggctagaatcttacttgagtaaaagtaaaaaagtactccattaaaattgtacttgagtattaaggaagtaaaagtaaaagcaagaaagaaaactagagattcttgtttaagcttttaatctcaaaaaacattaaatgaaatgcatacaaggttttatcctgctttagaactgtttgtatttaattatcaaactataagacagacaatctaatgccagtacacgctactcaaagttgtaaaacctcagatttaacttcagtagaagctgattctcaaaattgttagtgtcaagcctagctcttttggggctgaaaagcaatcctgcagtgctgaaaagcctctcacaggcagccgatgcgggaagaggtgtattagtcttgatagagaggctgcaaatagcaggaaacgtgagcagagactccctggtgtctgaaacacaggccagatgggccctcgagcaggaaacagctatgaccatgattacgccaagctatcaactttgtatagaaaagttgaacgagaaacgtaaaatgatataaatatcaatatattaaattagattttgcataaaaaacagactacataatactgtaaaacacaacatatccagtcactatggtcgaccgtatagaaaagttggtaacaggatgtgacacgtcgtctgctttaatcagtgtttgagagggcacctctacacactgaatgccaagcaaagaaagtcagaaaatctaaataatagcaaacatacacacacacacatacacaaatatatatatcaactcttcattcagttatttacaaaaaaaactgagaatatctgaactagccagatgaaaaaagagagcgagaaaggagaaaagaaagagagagagagataaaatgaaagagacagacagaaacagacagagggagagggagtcagtctcttgtgacctacatgaaagcaacgtgcctcgctactcctggctaaatgcactctcatattaatattttaaaacgccgtaatgtgacggtgaaaccctcggaggacagatacatcaatttgcaggggcctcggcttcaggaaagagaaacagggataatttgccattaggagaaattgttcattatgggcagcttcttaggaacacataaaccaagtcgcctctctcacgtgagctgcaaatccacacttttgagtgaatggagacgttgcccgagagctttaatgctcctgggtcattagtacaaaaaggtttgttgtgctgttaaatctgctgcatgttgcggtgatcaatcaacatgaaaggggcttcccactaagaccggatcactgtaaataaatcaattaggccgcgagagtcaagcttttatcagaattgttgctacacacacacacacacacacacacacacaatcggcttatagattcagtgctgaacagtccggttcagattcagctcaaaataaataagacatctgagtttgctgatcatcgttcagccagtaaatgacaccagcagcaggccaagtttccatgaagctactgtcttctatcgaacaagcatgcgatatttgccgacttaaaaagctcaagtgctccaaagaaaaaccgaagtgcgccaagtgtctgaagaacaactgggagtgtcgctactctcccaaaaccaaaaggtctccgctgactagggcacatctgacagaagtggaatcaaggctagaaagactggaacagctatttctactgatttttcctcgagaagaccttgacatgattttgaaaatggattctttacaggatataaaagcattgttaacaggattatttgtacaagataatgtgaataaagatgccgtcacagatagattggcttcagtggagactgatatgcctctaacattgagacagcatagaataagtgcgacatcatcatcggaagagagtagtaacaaaggtcaaagacagttgactgtatcgtcgaggtcgaccccgggaattcagatctctcgagccgcccccccgaccgatgtcagcctgggggacgagctccacttagacggcgaggacgtggcgatggcgcatgccgacgcgctagacgatttcgatctggacatgttgggggacggggattccccgggtccgggatttaccccccacgactccgccccctacggcgctctggatatgtagcggatctactagtgcggcccctctcgagcctctagaactatagtgagtcgtattacgtagatccagacatgataagatacattgatgagtttggacaaaccacaactagaatgcagtgaaaaaaatgctttatttgtgaaatttgtgatgctattgctttatttgtaaccattataagctgcaataaacaagttaacaacaacaattgcattcattttatgtttcaggttcagggggaggtgtgggaggttttttaattcgcggccatcaagcttaggcctccaaggcgatccagacatgataagatacattgatgagtttggacaaaccacaactagaatgcagtgaaaaaaatgctttatttgtgaaatttgtgatgctattgctttatttgtaaccattataagctgcaataaacaagttaacaacaacaattgcattcattttatgtttcaggttcagggggaggtgtgggaggttttttaaagcaagtaaaacctctacaaatgtggtatggctgattatccggagtactgtcctccgggctggcggagtactgtcctccggcaaggtcggagtactgtcctccgacactagaggtcggagtactgtcctccgacgcaaggcggagtactgtcctccgacactagaggtcggagtactgtcctccgacactagaggtcggagtactgtcctccgacactagaggtcggagtactgtcctccgacactagaggtcggagtactgtcctccgacactagaggtcggagtactgtcctccgacactagaggtcggagtactgtcctccgacactagaggtcggagtactgtcctccgacactagaggtcggagtactgtcctccgacactagaggtcggagtactgtcctccgtccacttaagctaggtggccagcggtgggatacattgatgagtttggacaaaccacaactagaatgcagtgaaaaaaatgctttatttgtgaaatttgtgatgctattgctttatttctagagggtatataatggatcccatcgcgtctcagcctcactttgagctcctccacacgaattctttggccacaagctgaccctgaagttcatctgcaccaccggcaagctgcccgtgccctggcccaccctcgtgaccaccctgactacggcgtgcagtgcttcagcatggccgtcaatgtgtactccacatctgtgaccagtgaaaatctgagtcgccatgatatgcttgcatgggtcaacgactccctgcacctcaactataccaagatagaacagctttgttcaggggcagcctactgccagttcatggacatgctcttccccggctgtgtgcacttgaggaaagtgaagttccaggccaaactagagcatgaatacatccacaacttcaaggtgctgcaagcagctttcaagaagatgggtgttgacaaaatcattcctgtagagaaattagtgaaaggaaaattccaagataattttgagtttattcagtggtttaagaaattctttgacgcaaactatgatggaaaggattacaaccctctgctggcgcggcagggccaggacgtagcgccacctcctaacccaggtgatcagatcttcaacaaatccaagaaactcattggcacagcagttccacagaggacgtcccccacaggcccaaaaaacatgcagacctctggccggctgagcaatgtggcccccccctgcattctccggaagaatcctccatcagcccgaaatggcggccatgagactgatgcccaaattcttgaactcaaccaacagctggtggacttgaagctgacagtggatgggctggagaaggaacgtgacttctacttcagcaaacttcgtgacatcgagctcatctgccaggagcatgaaagtgaaaacagccctgttatctcaggcatcattggcatcctctatgccacagaggaaggattcgcaccccccatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaagggcggtggaagatctgggaattcaaggcctctcgagcctctagattctgcagccctatagcgccatagtgactggatatgttgtgttttacagtattatgtagtctgttttttatgcaaaatctaatttaatatattgatatttatatcattttacgtttctcgttcaactttattatacatagttgataattcactggccgtcgttttacggtaccatcgatgatgatccagacatgataagatacattgatgagtttggacaaaccacaactagaatgcagtgaaaaaaatgctttatttgtgaaatttgtgatgctattgctttatttgtaaccattataagctgcaataaacaagttaacaacaacaattgcattcattttatgtttcaggttcagggggaggtgtgggaggttttttaaagcaagtaaaacctctacaaatgtggtatggctgattatgatcctctagatcagatctgcgaagatacggccacgggtgctcttgatcctgtggctgattttggactgtgctgctcgcagctgctgatgaatcacatacttcctccattttcttccactgattgactgttataatttccctaatttccaggtcaaggtgctgtgcattgtggtaatagatgtgacatgacgtcacttccaaaggaccaatgaacatgtctgaccaatttcatataatgtgaaaacgattttcataggcagaataaataacatttaaattaaactgggcatcagcgcaattcaattggtttggtaatagcaagggaaaatagaatgaagtgatctccaaaaaataagtactttttgactgtaaataaaattgtaaggagtaaaaagtacttttttttctaaaaaaatgtaattaagtaaaagtaaaagtattgatttttaattgtactcaagtaaagtaaaaatccccaaaaataatacttaagtacagtaatcaagtaaaattactcaagtactttacacctctggttcttgaccccctaccttcagcaagcccagcagatccactagttctagagcggccgccaccgcggtggagctccagcttttgttccctttagtgagggttaattgcgcgcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtg

SEQ ID NO: 26 - DNA, 9673 bp, synthetic construct
  source 1..9673: mol_type = other DNA
cgccattctgcctggggacgtcggagcactcgagagaaaccaggctcagtcgggcacgaccattactccacttgaccaaacgtctgcatatttataaataataatttgcgtattaatatgcagataaagacaattaaaagaagcaacagtagcattaatcattcaaataacagtatacaatcaaacaacattttgtatatatatttaaaaaaacaatacaaggagtaaagacaaaggcaatatcatttagtttttttaatattaacaaaccacaggctgtgtgaaccatcagcaatttcaaaccagagggatctgcttttttgggaacgatgttgcaaatttaatctctagattttttaaatcatggatagcctccctaaagcaaaagctttttaggggctttacttctcctgattgcagcctgtggtataatttgtgattgttaggtgggtcaggttttggggtccagccctttgtgcactttgactattgtcaccttaggctgtcccttaatgctgccacctctgaccccggtgggacacataaataacttctccacccgagttagcatttcaagccagtgcaataaagaaattaaaaataattgatttattaacaactctgttttatctttaaaaaataaaatgttttatatctaatgttcactcaggtacctgatattgtttgctgtgcaataatgtaggttagtcattatattgacaaagaaaattaaatatgatgttgtttatttttaattttacattatagactagattagattttcatttggaatttaattaggagtgcctggagtgcattgagaacgttaaaaaaaaaaaatctgcttactcttatagctttggaaactattttcagattattactccactatagaagcgattcctttttttgaataattaaatatatatatacagtgctcagcataaatgatcatcacagatctctattttaaagtattatttttccataggatgctttacaataatatatgtgtgtgtatacattagaattgtcagttacaaaatcaaaactgcaacaaaataactaaacttaagatctaatgagtacacccaaattaaaccaaacattagtctatgcaaaaattaatatgttaaggaaaaatattaaatacaattttaatattcagtcattcattttcttttttgcttagtccctttattaatctggggtcgccacagtggaattaactgccaatttatccagcatatattttatgcagcggatgctcttccagctgctacccatcactgggaatcacccatacactctcattcacacacatacactacggacaatttagcctacccaattcacctatagcgtatgtctttggacttgtgggggaaaccggaacacacggaggaaacccacgcgaacacagggagatcatgcaaactcagaaacgccaactgatccagccaaggctcgaaccagtgaccttcttgctgtgaggcaatagggctacccattgtgtcactgcaccgtccacaattttaataaacatgaaaaatcccgagaaacataaacaatattaaatgtagttgaaattttgttgtttgaaattgtatttattaaataaaaaaaatgtatgtattattattatttatttatttataaaagatgttaggtggccaaactcttttttttaatggatatagttgtttaataaaacaattttgtttaaataaaacaaaatatattgaatgagccatgttgctgttgttgttttttacaggcttgatgtacaatatgatctttgtgtagttttatagtaataaatcattcatgtcttgtgtattgtacgtcatattatcttaaaagtacgctattaattaaacttgtttatatggtcatcttagacaggactctcattataaaaggatttttgttaattttattagtaccttcctggaaaaataaaagataaataaataaaaatgaataaaagtcttgggcttgttttctgtacgagtaataaaaaagagtaagcaaatattctgcatgacaatattgttcaaagtttaagggctgtattttttataattgttggttattaacatgttttatacaaattactttcaacatggctgaaataataattgttatcactatttgaaacagtcctagaatagattttactatatttacatttattcctctgatgacaaagttgaaattcctctgatgacaaagcaaaactctcaactctagttttattttattgtaataaccatgtcaagggataatgacaatgttttccattatattttcaatatagaataaaattataatttattaatcatttagtagggcagcatggtggcacaatgggtagcacaatcacctcacaccaagaaggttgctagttcgagccccggctgggtcagtttgcatatctgtgtggagaatgttctctctgtgttgccatgggtttcctctgggtgctctagtttccaccacaagtctaaaaacaagtggcatcggtgaattgtgtaggctaaattgtctgtagtgtttgtgtgtgattgagtgtttatggatgtttcccagtgatgggttgcagttggaagggcattctctgcgtaaaactataagttggcggttcattccactgtggtgaccccagattaataaagggattaagccgaaaagaaaatgaatcattaaatgatcatttagttgattggagagcattgcataatgagaatcaaataatgtatagttttttgtaaaatgattgtgtattagaggcacattgtacgtagtgtggaatgtttaggttgtctttctgattgttttcattttgcagaagttcatttgcagtcagtgtagtcaaagcacctcagctcaatttatagcagcagcctgctgtgatttaagtgccacaggagtgaaggctttaacccctgttgtagattgcaaccaatgtttttctaaggtaattagaagcttgtttagctaacgctacccaccccaccccccacaaaaaagttgctctgaatagcagcatctacccccaggagggtgcaagcttctgttttgaagaaatttgctcatggtgttacataggtgtttaaaaatgtatgttttttaatagcatatattttaaaagtgactcattaatgacatcatctgccatctgcctttctcagaactgacaaaggacccttttaaggacccctatacaccacatttttatctatctaatcacaagtgggtgatattagagggatgattcacctaaaaatgaattttccgcattttaatttggggttccaaaccagtttgaattcttttgttctgttgaacacaaaataagatgttttaagaatgttggaaatgttggagacttttatagtaggaaaaacatatactaatatttgcattcatgtttgaaatgtttaaaacatttttcaaaatatcttatttttcaactgttttggaacaagtcaaaagtgagtaaatgatgactgataaacaatttctttaaatacagatgaaaatagcgtctaacacattttgaactagttcactttcaaccacagcaagaggtagtcgaaaacacatttgatcacattttgttgtgggaccgcttgttgttcagtgtgttttcattgttgagttacaaaccggtaagtggcattaaaacatgttttggcatcactgatgatcataaataaattggttaaaaaattaaaaattagatgcttacagtacagtttgtttaaaaacttgcactttttgtagtgtgaaatgacattagtacctgcagtgggctttctttgttctgtatcttctgatgaatctaacataaagacatttaaatattcacaatggagtgggttggttctcatctatctgaaacaacattagtgtgttcaaaacaaatctacaccagtgatctctgatagatacaagctgtcaaccgtagtcctattgtttctcatgcgactgtcacagggccatgctagaggctctttgggtggtgagtagagactcattcttatgcaaacaggctatggatgctacaactgaagagaatttagtgctctaaggtcatcgcagttctataaagcagattccctccaaaaagctagagaaaattatgtgtctggaggcaccatctatcagtgcagtcattcttttattactttttttttttgtccatgcaattttttaatcgcattttgaagtatttaaaccattaagagtgtcaaattatcatgttattatttcaactcataatatctccttttaaagatgattcatttgattgtagttataataattgttttattaatgcaaaaattgcttaaatgtttggcctctgagattttcagttgactcaatatttttggagcctgtttcatgactttatttaaaatataatctgcactcgcaaatattacaattatatcatattttgaaaatcttagatgtacatttgttttgaaaatggtttaaaggaaatttaggtcaaaatgtgtgttttttttaaataataattcagtcttttttttgatataatagggaaaatgacagcaattaaaaggctgttttggaaagacatctaggtctggtgcagacttcgtaacttcttagagttaaatcttgaaaacggagccagaagacgggcaacaccttcctaaaagcagggtggggtgttggacaggtagcttcttggaacctcccctctttgtcgctgtggtatatatagaaatcccattgaattcctatagagaccttaagcccgtaccatttacctctgaagagcttgatttaggtgacactatagaatacaagctacttgttctttttgcaggatcccatcgattcgaattcaaggcctatggccgtcaatgtgtactccacatctgtgaccagtgaaaatctgagtcgccatgatatgcttgcatgggtcaacgactccctgcacctcaactataccaagatagaacagctttgttcaggggcagcctactgccagttcatggacatgctcttccccggctgtgtgcacttgaggaaagtgaagttccaggccaaactagagcatgaatacatccacaacttcaaggtgctgcaagcagctttcaagaagatgggtgttgacaaaatcattcctgtagagaaattagtgaaaggaaaattccaagataattttgagtttattcagtggtttaagaaattctttgacgcaaactatgatggaaaggattacaaccctctgctggcgcggcagggccaggacgtagcgccacctcctaacccaggtgatcagatcttcaacaaatccaagaaactcattggcacagcagttccacagaggacgtcccccacaggcccaaaaaacatgcagacctctggccggctgagcaatgtggcccccccctgcattctccggaagaatcctccatcagcccgaaatggcggccatgagactgatgcccaaattcttgaactcaaccaacagctggtggacttgaagctgacagtggatgggctggagaaggaacgtgacttctacttcagcaaacttcgtgacatcgagctcatctgccaggagcatgaaagtgaaaacagccctgttatctcaggcatcattggcatcctctatgccacagaggaaggattcgcacccccctgagcctctagaactatagtgagtcgtattacgtagatccagacatgataagatacattgatgagtttggacaaaccacaactagaatgcagtgaaaaaaatgctttatttgtgaaatttgtgatgctattgctttatttgtaaccattataagctgcaataaacaagttaacaacaacaattgcattcattttatgtttcaggttcagggggaggtgtgggaggttttttaattcgcggccgcggcgccaatgcattgggcccggtacccagcttttgttccctttagtgagggttaattgcgcgcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctaaattgtaagcgttaatattttgttaaaattcgcgttaaatttttgttaaatcagctcattttttaaccaataggccgaaatcggcaaaatcccttataaatcaaaagaatagaccgagatagggttgagtgttgttccagtttggaacaagagtccactattaaagaacgtggactccaacgtcaaagggcgaaaaaccgtctatcagggcgatggcccactacgtgaaccatcaccctaatcaagttttttggggtcgaggtgccgtaaagcactaaatcggaaccctaaagggagcccccgatttagagcttgacggggaaagccggcgaacgtggcgagaaaggaagggaagaaagcgaaaggagcgggcgctagggcgctggcaagtgtagcggtcacgctgcgcgtaaccaccacacccgccgcgcttaatgcgccgctacagggcgcgtcccattcgccattcaggctgcgcaactgttgggaagggcgatcggtgcgggcctcttcgctattacgccagtcgaccatagccaattcaatatggcgtatatggactcatgccaattcaatatggtggatctggacctgtgccaattcaatatggcgtatatggactcgtgccaattcaatatggtggatctggaccccagccaattcaatatggcggacttggcaccatgccaattcaatatggcggacttggcactgtgccaactggggaggggtctacttggcacggtgccaagtttgaggaggggtcttggccctgtgccaagtccgccatattgaattggcatggtgccaataatggcggccatattggctatatgccaggatcaatatataggcaatatccaatatggccctatgccaatatggctattggccaggttcaatactatgtattggccctatgccatatagtattccatatatgggttttcctattgacgtagatagcccctcccaatgggcggtcccatataccatatatggggcttcctaataccgcccatagccactcccccattgacgtcaatggtctctatatatggtctttcctattgacgtcatatgggcggtcctattgacgtatatggcgcctcccccattgacgtcaattacggtaaatggcccgcctggctcaatgcccattgacgtcaataggaccacccaccattgacgtcaatgggatggctcattgcccattcatatccgttctcacgccccctattgacgtcaatgacggtaaatggcccacttggcagtacatcaatatctattaatagtaacttggcaagtacattactattggaaggacgccagggtacattggcagtactcccattgacgtcaatggcggtaaatggcccgcgatggctgccaagtacatccccattgacgtcaatggggaggggcaatgacgcaaatgggcgttccattgacgtaaatgggcggtaggcgtgcctaatgggaggtctatataagcaatgctcgtttagggaac

SEQ ID NO: 27 - DNA, 11961 bp, synthetic construct
  source 1..11961: mol_type = other DNA
ccacctaaattgtaagcgttaatattttgttaaaattcgcgttaaatttttgttaaatcagctcattttttaaccaataggccgaaatcggcaaaatcccttataaatcaaaagaatagaccgagatagggttgagtgttgttccagtttggaacaagagtccactattaaagaacgtggactccaacgtcaaagggcgaaaaaccgtctatcagggcgatggcccactacgtgaaccatcaccctaatcaagttttttggggtcgaggtgccgtaaagcactaaatcggaaccctaaagggagcccccgatttagagcttgacggggaaagccagaggtgtaaaaagtactcaaaaattttactcaagtgaaagtacaagtacttagggaaaattttactcaattaaaagtaaaagtatctggctagaatcttacttgagtaaaagtaaaaaagtactccattaaaattgtacttgagtattaaggaagtaaaagtaaaagcaagaaagaaaactagagattcttgtttaagcttttaatctcaaaaaacattaaatgaaatgcatacaaggttttatcctgctttagaactgtttgtatttaattatcaaactataagacagacaatctaatgccagtacacgctactcaaagttgtaaaacctcagatttaacttcagtagaagctgattctcaaaattgttagtgtcaagcctagctcttttggggctgaaaagcaatcctgcagtgctgaaaagcctctcacaggcagccgatgcgggaagaggtgtattagtcttgatagagaggctgcaaatagcaggaaacgtgagcagagactccctggtgtctgaaacacaggccagatcgcgttgtaaaacgacggccagtgagcgcgcgtaatacgactcactatagggcgaattgggtacgatccaccggtcgctcgagagaaaccaggctcagtcgggcacgaccattactccacttgaccaaacgtctgcatatttataaataataatttgcgtattaatatgcagataaagacaattaaaagaagcaacagtagcattaatcattcaaataacagtatacaatcaaacaacattttgtatatatatttaaaaaaacaatacaaggagtaaagacaaaggcaatatcatttagtttttttaatattaacaaaccacaggctgtgtgaaccatcagcaatttcaaaccagagggatctgcttttttgggaacgatgttgcaaatttaatctctagattttttaaatcatggatagcctccctaaagcaaaagctttttaggggctttacttctcctgattgcagcctgtggtataatttgtgattgttaggtgggtcaggttttggggtccagccctttgtgcactttgactattgtcaccttaggctgtcccttaatgctgccacctctgaccccggtgggacacataaataacttctccacccgagttagcatttcaagccagtgcaataaagaaattaaaaataattgatttattaacaactctgttttatctttaaaaaataaaatgttttatatctaatgttcactcaggtacctgatattgtttgctgtgcaataatgtaggttagtcattatattgacaaagaaaattaaatatgatgttgtttatttttaattttacattatagactagattagattttcatttggaatttaattaggagtgcctggagtgcattgagaacgttaaaaaaaaaaaatctgcttactcttatagctttggaaactattttcagattattactccactatagaagcgattcctttttttgaataattaaatatatatatacagtgctcagcataaatgatcatcacagatctctattttaaagtattatttttccataggatgctttacaataatatatgtgtgtgtatacattagaattgtcagttacaaaatcaaaactgcaacaaaataactaaacttaagatctaatgagtacacccaaattaaaccaaacattagtctatgcaaaaattaatatgttaaggaaaaatattaaatacaattttaatattcagtcattcattttcttttttgcttagtccctttattaatctggggtcgccacagtggaattaactgccaatttatccagcatatattttatgcagcggatgctcttccagctgctacccatcactgggaatcacccatacactctcattcacacacatacactacggacaatttagcctacccaattcacctatagcgtatgtctttggacttgtgggggaaaccggaacacacggaggaaacccacgcgaacacagggagatcatgcaaactcagaaacgccaactgatccagccaaggctcgaaccagtgaccttcttgctgtgaggcaatagggctacccattgtgtcactgcaccgtccacaattttaataaacatgaaaaatcccgagaaacataaacaatattaaatgtagttgaaattttgttgtttgaaattgtatttattaaataaaaaaaatgtatgtattattattatttatttatttataaaagatgttaggtggccaaactcttttttttaatggatatagttgtttaataaaacaattttgtttaaataaaacaaaatatattgaatgagccatgttgctgttgttgttttttacaggcttgatgtacaatatgatctttgtgtagttttatagtaataaatcattcatgtcttgtgtattgtacgtcatattatcttaaaagtacgctattaattaaacttgtttatatggtcatcttagacaggactctcattataaaaggatttttgttaattttattagtaccttcctggaaaaataaaagataaataaataaaaatgaataaaagtcttgggcttgttttctgtacgagtaataaaaaagagtaagcaaatattctgcatgacaatattgttcaaagtttaagggctgtattttttataattgttggttattaacatgttttatacaaattactttcaacatggctgaaataataattgttatcactatttgaaacagtcctagaatagattttactatatttacatttattcctctgatgacaaagttgaaattcctctgatgacaaagcaaaactctcaactctagttttattttattgtaataaccatgtcaagggataatgacaatgttttccattatattttcaatatagaataaaattataatttattaatcatttagtagggcagcatggtggcacaatgggtagcacaatcacctcacaccaagaaggttgctagttcgagccccggctgggtcagtttgcatatctgtgtggagaatgttctctctgtgttgccatgggtttcctctgggtgctctagtttccaccacaagtctaaaaacaagtggcatcggtgaattgtgtaggctaaattgtctgtagtgtttgtgtgtgattgagtgtttatggatgtttcccagtgatgggttgcagttggaagggcattctctgcgtaaaactataagttggcggttcattccactgtggtgaccccagattaataaagggattaagccgaaaagaaaatgaatcattaaatgatcatttagttgattggagagcattgcataatgagaatcaaataatgtatagttttttgtaaaatgattgtgtattagaggcacattgtacgtagtgtggaatgtttaggttgtctttctgattgttttcattttgcagaagttcatttgcagtcagtgtagtcaaagcacctcagctcaatttatagcagcagcctgctgtgatttaagtgccacaggagtgaaggctttaacccctgttgtagattgcaaccaatgtttttctaaggtaattagaagcttgtttagctaacgctacccaccccaccccccacaaaaaagttgctctgaatagcagcatctacccccaggagggtgcaagcttctgttttgaagaaatttgctcatggtgttacataggtgtttaaaaatgtatgttttttaatagcatatattttaaaagtgactcattaatgacatcatctgccatctgcctttctcagaactgacaaaggacccttttaaggacccctatacaccacatttttatctatctaatcacaagtgggtgatattagagggatgattcacctaaaaatgaattttccgcattttaatttggggttccaaaccagtttgaattcttttgttctgttgaacacaaaataagatgttttaagaatgttggaaatgttggagacttttatagtaggaaaaacatatactaatatttgcattcatgtttgaaatgtttaaaacatttttcaaaatatcttatttttcaactgttttggaacaagtcaaaagtgagtaaatgatgactgataaacaatttctttaaatacagatgaaaatagcgtctaacacattttgaactagttcactttcaaccacagcaagaggtagtcgaaaacacatttgatcacattttgttgtgggaccgcttgttgttcagtgtgttttcattgttgagttacaaaccggtaagtggcattaaaacatgttttggcatcactgatgatcataaataaattggttaaaaaattaaaaattagatgcttacagtacagtttgtttaaaaacttgcactttttgtagtgtgaaatgacattagtacctgcagtgggctttctttgttctgtatcttctgatgaatctaacataaagacatttaaatattcacaatggagtgggttggttctcatctatctgaaacaacattagtgtgttcaaaacaaatctacaccagtgatctctgatagatacaagctgtcaaccgtagtcctattgtttctcatgcgactgtcacagggccatgctagaggctctttgggtggtgagtagagactcattcttatgcaaacaggctatggatgctacaactgaagagaatttagtgctctaaggtcatcgcagttctataaagcagattccctccaaaaagctagagaaaattatgtgtctggaggcaccatctatcagtgcagtcattcttttattactttttttttttgtccatgcaattttttaatcgcattttgaagtatttaaaccattaagagtgtcaaattatcatgttattatttcaactcataatatctccttttaaagatgattcatttgattgtagttataataattgttttattaatgcaaaaattgcttaaatgtttggcctctgagattttcagttgactcaatatttttggagcctgtttcatgactttatttaaaatataatctgcactcgcaaatattacaattatatcatattttgaaaatcttagatgtacatttgttttgaaaatggtttaaaggaaatttaggtcaaaatgtgtgttttttttaaataataattcagtcttttttttgatataatagggaaaatgacagcaattaaaaggctgttttggaaagacatctaggtctggtgcagacttcgtaacttcttagagttaaatcttgaaaacggagccagaagacgggcaacaccttcctaaaagcagggtggggtgttggacaggtagcttcttggaacctcccctctttgtcgctgtggtatatatagaaatcccattgaattcctatagagaccttaagcccgtaccatttacctctgaagccacggatcccatggtgatggcatcatcacaagtaccagcagccaaaaaagatgagaagggcagaaacatacaagtggttgtacgatgcagaccctttaacacagtggagcgtaaatctggctctcacactgttgttgaatgtgaccagaaccggaaagaggtgattatgcgtactggaggtgccacagacaaagcagcaagaaaaacatacacttttgatatggtttttggcccttctgccaaacaaattgaagtttataggagtgtggtttgccccatattagatgaagttatcatgggctataactgcacaatctttgcatatgggcaaaccggaacaggcaaaaccttcacaatggaaggtgaccgatcacccaatgaggagtttacttgggaagaggaccctctggctggaataattcccaggactcttcatcagatctttgaaaaactgtctaataatggcacagagttctcagtgaaggtgtctctgctggaaatttataacgaggaactgtttgacctgctcagccctgctcctgatgtcacagagagattacagctctttgatgatcccagaaataaaaggggtgtgaccattaaaggtctggaggaaatcaccgtacacaataagaatgaggtgtatcagatcctggagagaggagcagccaagaggaagactgcctccacactcatgaatgcctattccagtcgatcccactctgtgttctctgtcactattcacatgaaggaaatcacactggatggggaagagctagttaagattggtaaactcaacttggtggatcttgcaggtagtgagaacatcggacgatctggtgctgtggataagcgtgcacgtgaagctggcaacatcaaccagtctctgctaactctgggtcgtgtaatcaaggctttggtggagagagggcctcacgtgccctacagagagtctaaactaacccgcatactgcaggactcgctaggaggacgcaccaaaacctctatcatcgccactgtgtctcctgcctccatcaatctggaggaaactctgagcactttagactatgctaacagagctaagagcatcatgaataagcctgaggtcaatcaaaagcttaccaagagaaccctcatcaaggaatacacagaggaaattgagagactgaagagagatttggctgccacccgtgacaaacatggagtgtacctttccgttgataactatgagactttgaatggtaaaattgtgtctcaggaagagcagattacagagtacactgagcggatagctgctatggaggaggagctcaaaaagattatagacctgttcacagatagtaagcagaaattggaacagtgcaccgaggacctgcaagacaaaaaccagagactggaggaggctcacaaagacctatcggagaccaggcaccgcctcaatcaggaggaattcatcagtacacagcttcaaaccaatgagagtcacctctataacactgctgaccagttgttaagcactgctgaggccagcacacaggatgtcggtggtctccatgctaagctacaaaggaagaaggatgtggagcttcataacagtaaggttcaggagagcttctcccagtgcatggagaactgctacaacagcatgcagacctcactgaaggagcagagccaaaaacatgctgctatgattgattattaccgctcctctgtgggtgagctgctgaacaccaatggcaaggtgtttaaggagactttgggtgctgtgtgcgagtcttacagcagtattaaaggagctgtgggagaaggtgtagagcggtgtaaggagcaagtgttaaatcaggagaaactctctcaggatgctcaaaacagcatcctggaaattctggatgaacacaaacagcatcttgaggaggttctggttgcccaggcggtgccaggtatcaggtctgtcatgtccatgaatgacaatttgaagcaaacccttcacaaataccacaatctggctgagcagatgcagggtgtgaaagcagatatgatgacgttttttgatgcgtacactgaatcactggccagtatgcgagagtgcgctttgcagggttttaacacactgcgtgccgaacatgacaaactcaaacaacaaatcagccaagctggaaacagccatcaagtgcgtgtggccgagctggttcagtgtttgcagaaccaaatgaatctgctggcggtggacactcagaatgactttgagggtctttcacaagcagcatctgcccagattcctccactggaaacattacagagctccatagagagtaaatgcacagtggctgaggagcaggctgtgtctgttcgtgctcgactgggttcctctgttcatggagtgatttcagagatgaatagtgtgactaaggagggagagagggcgctggaggagtgtgctggttattgtggacacctgcagacatcgctggactcgctggctgagtcaggactcaagtggtgcgatgaagctaaaggcttgactgagagtaaagcccaggagcaactcaaactcatcagacagacagacacggctgtgcaagacctgctgaagtctgtggaggaaaagggtgagaaagctgttcaggactgtgaagccagattgggtcaaatgcagcaggaaatggaagcaaccctgggtcgtgtggaaatgcagaccagcaaagatgaagcgacattgcaggagcacagagagaccctgtcctccatcaacactcaggcactggacaccgtacacaacttcatcagctcagtgctacggcaggatttaccaacaggaacgacccctcagcgcaaagagtacatgtacccacgtgtgctgagcaggccgaggagtagagaggagctggaagaggagttcagagctcagcaagagcagcttcagtctgaactgaagccgtgtgagatcgtaatggaggtggaggaggagaaacctgtcgaccaggactctctggaggatgacgtcagtgtttctagtgatggaaacaacactgaacagtcgtgctctgatgagaacctgatatgttatgagaatggaaggattccttttttcaagaagaaaagcaagaaggaaaatggcagtaaatcactgaaccgttcaaaggtggagaatgacagcatgtcaacaccaccccgctctaaacttccactcagatgtcagagcaagggcgccgagctgttcaccggcatcgtgcccatcctgatcgagctgaatggcgatgtgaatggccacaagttcagcgtgagcggcgagggcgagggcgatgccacctacggcaagctgaccctgaagttcatctgcaccaccggcaagctgcctgtgccctggcccaccctggtgaccaccctgagctacggcgtgcagtgcttctcacgctaccccgatcacatgaagcagcacgacttcttcaagagcgccatgcctgagggctacatccaggagcgcaccatcttcttcgaggatgacggcaactacaagtcgcgcgccgaggtgaagttcgagggcgataccctggtgaatcgcatcgagctgaccggcaccgatttcaaggaggatggcaacatcctgggcaataagatggagtacaactacaacgcccacaatgtgtacatcatgaccgacaaggccaagaatggcatcaaggtgaacttcaagatccgccacaacatcgaggatggcagcgtgcagctggccgaccactaccagcagaatacccccatcggcgatggccctgtgctgctgcccgataaccactacctgtccacccagagcgccctgtccaaggaccccaacgagaagcgcgatcacatgatctacttcggcttcgtgaccgccgccgccatcacccacggcatggatgagctgtacaagtgagcggccgccaccgcggtggagctcgaattaattcatcgatgatgatccagacatgataagatacattgatgagtttggacaaaccacaactagaatgcagtgaaaaaaatgctttatttgtgaaatttgtgatgctattgctttatttgtaaccattataagctgcaataaacaagttaacaacaacaattgcattcattttatgtttcaggttcagggggaggtgtgggaggttttttaaagcaagtaaaacctctacaaatgtggtatggctgattatgatcctctagatcaggccgccaccgcggtggagctccagcttttgttccctttagtgagggttaattgcgcgcttggcgtaatcatggtcatagctgtttcctgtgtggcgaagatacggccacgggtgctcttgatcctgtggctgattttggactgtgctgctcgcagctgctgatgaatcacatacttcctccattttcttccactgattgactgttataatttccctaatttccaggtcaaggtgctgtgcattgtggtaatagatgtgacatgacgtcacttccaaaggaccaatgaacatgtctgaccaatttcatataatgtgaaaacgattttcataggcagaataaataacatttaaattaaactgggcatcagcgcaattcaattggtttggtaatagcaagggaaaatagaatgaagtgatctccaaaaaataagtactttttgactgtaaataaaattgtaaggagtaaaaagtacttttttttctaaaaaaatgtaattaagtaaaagtaaaagtattgatttttaattgtactcaagtaaagtaaaaatccccaaaaataatacttaagtacagtaatcaagtaaaattactcaagtactttacacctctgggggaggttccctttagtgagggttaattgcgcgcttggcgtaatcatggtcatagctgtttcctgtgtgaaattgttatccgctcacaattccacacaacatacgagccggaagcataaagtgtaaagcctggtgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtg

SEQ ID NO: 28 - DNA, 10693 bp, synthetic construct
  source 1..10693: mol_type = other DNA
ccacctaaattgtaagcgttaatattttgttaaaattcgcgttaaatttttgttaaatcagctcattttttaaccaataggccgaaatcggcaaaatcccttataaatcaaaagaatagaccgagatagggttgagtgttgttccagtttggaacaagagtccactattaaagaacgtggactccaacgtcaaagggcgaaaaaccgtctatcagggcgatggcccactacgtgaaccatcaccctaatcaagttttttggggtcgaggtgccgtaaagcactaaatcggaaccctaaagggagcccccgatttagagcttgacggggaaagccggcgaacgtggcgagaaaggaagggaagaaagcgaaaggagcgggcgctagggcgctggcaagtgtagcggtcacgctgcgcgtaaccaccacacccgccgcgcttaatgcgccgctacagggcgcgtcccattcgccattcaggctgcgcaactgttgggaagggcgatcggtgcgggcctcttcgctattacgccagctggcgaaagggggatgtgctgcaaggcgattaagttgggtaacgccagggttttcccagtcacgacgttgtaaaacgacggccagtgagcgcgcgtaatacgactcactatagggcgaattgggtacctcgagtcgagcagggggatcatctaatcaagcacaaataaggggcgtgtaacacaaaagccagcgaccctttccaatgcaaatcaaacttgcaattctttgccgtttttatcatttaagtgtcggcttaaggtccactatcagatgtaaacagccttatctaacaaaggtatcattacattctgaaattctcaggcatgcaagctagcttatgacgcactagggagtgccacccttcctttcgccctaacttcgtgataactcgcgcgtttcactcaacagctgcatccgccctagtgctactgggagttgtagtatacaagacgcttacaggctgaatgttctgtcaagaccccgcctctagcactttgggaattctggacttgatgatgtcatggttaatccccgcccagtagaggcggctatataaagggtggttaaggcccggttcgctctcttcctcaccgggtctgcggcgagttctagctgaagcttcctgcaggtcgaccgatcctgagaacttcagggtgagtttggggacccttgattgttctttctttttcgctattgtaaaattcatgttatatggagggggcaaagttttcagggtgttgtttagaatgggaagatgtcccttgtatcaccatggaccctcatgataattttgtttctttcactttctactctgttgacaaccattgtctcctcttattttcttttcattttctgtaactttttcgttaaactttagcttgcatttgtaacgaatttttaaattcacttttgtttatttgtcagattgtaagtactttctctaatcacttttttttcaaggcaatcagggtatattatattgtacttcagcacagttttagagaacaattgttataattaaatgataaggtagaatatttctgcatataaattctggctggcgtggaaatattcttattggtagaaacaactacaccctggtcatcatcctgcctttctctttatggttacaatgatatacactgtttgagatgaggataaaatactctgagtccaaaccgggcccctctgctaaccatgttcatgccttcttctctttcctacagctcctgggcaacgtgctggttgttgtgctgtctcatcattttggcaaagaattcctcgacggatccaccggtcgccaccatggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaagtaaagcggccgccaccgcggtggagctcgaattaattcatcgatgatgatccagacatgataagatacattgatgagtttggacaaaccacaactagaatgcagtgaaaaaaatgctttatttgtgaaatttgtgatgctattgctttatttgtaaccattataagctgcaataaacaagttaacaacaacaattgcattcattttatgtttcaggttcagggggaggtgtgggaggttttttaaagcaagtaaaacctctacaaatgtggtatggctgattatgatcctctagatcagatctgtcccgatctttctcttctgtgctgtcaatttagagtagtttttgaggtaatttgggtgcattctctgtaaaaacaacaaataaatatttatattgtatgcaataaatgtaacaattacacatacactctctgattacagcattaaagggttagttcacccaaaaatgaaaagatgtcattaatgactcaccctcatgtcgttccaagcccgtaacacctccgttcatcttcggaacacagtttaagatattttagatttagtccgagagctttctgtccctccactgaaaatgtatgtacggtatactgtccatgtccagaaaggtaataaaaacatcaaagtagtccatgtgacatcagtgggttaggtagaattatttgaagcatcgaaaatacattttggtccaaaaataacaaaacctacgactttattcagcattgtagcgtcactgcgaagtcgtgaacgcggattgacaacagacccggaagagaatacaatgccgaataaagtcgtaggttttgttatttttggaccaaaatgtattcgatgcttcaaaaaattctaactaacccactgatgtcacatggactactttgatgtttttattacctttctggacatggacagtataccgtacatacattctcaatggaggcacagaaagctctcggactaaatctaaaatatcttaaactgtgttctgaagatgaacggaggtcttacgggcttggaacgacatgagggcgagtcattaatgacattattttcatttttgggtgaactaaccctttaataaaactgacatttgcagccaattgcacactttataatgttaaatacttgttgattttagaaatacagtagcatttagtgacaatcaaacattaattttatttctgattatattgacaaaaacacaggactgtaaggctgacatgacagggtgaattgatttcattacattagttttgatggtaacacttttacaataaggttcgttagttaactacattagttaacatgaactaataatgaactgcacgtatacatgttaatttcaacatttactaatactttatatcttgttaacattagttaatgcactgtgaactaacatgaacaaacaatgaacatctgtatttctattaactaacgttaacaaagattaacagatacagtaacaaatgtattgctcatggttatttaatgttagttaatacatgaactaatcaacctcattgtaaagtgttaccgttttgacaatacaacatgtttggaaaaaaatttctcgcttggttttcttgtgttttgcatgaattcaaacacaatgagagcacggaatgataatgattattcaaagctgcaataggcgccaaatttcccgtggacctaacgttaccaattacaaatgcattttagcattttattacgtgagccttatattgtcaatcgcatgcattagtgaaaataggcctactacacatataaaaggcgcgccatcaacgtcaaaacatgcacacccaaaaaaaaaaaaaaaaaaaaaaaaaaaaactatcagtaaaacaaaagatccaccggtcgccaccatggtgatggagatggcaagccagcagggcgagacgatgtccggaccgctgcacattggtttgattcccacagttggaccgtacctgctaccgcatattatccctatgctgcaccagacctttccaaagctggaaatgtatctgcatgaagcacagacccaccagttactggcgcaactggacagcggcaaactcgattgcgtgatcctcgcgctggtgaaagagagcgaagcattcattgaagtgccgttgtttgatgagccaatgttgctggctatctatgaagatcacccgtgggcgaaccgcgaatgcgtaccgatggccgatctggcaggggaaaaactgctgatgctggaagatggtcactgtttgcgcgatcaggcaatgtccgccggctacaacagcgacaacgtctatatcatggccgacaagcagaagaacggcatcaaggccaacttcaagatccgccacaacgtcgaggacggcagcgtgcagctcgccgaccactaccagcagaacacccccatcggcgacggccccgtgctgctgcccgacaaccactacctgagcttccagtccgtcctgagcaaagaccccaacgagaagcgcgatcacatggtcctgctggagttcgtgaccgccgccgggatcactctcggcatggacgagctgtacaacgtggatggcggtagcggtggcaccggcagcaagggcgaggagctgttcaccggggtggtgcccatcctggtcgagctggacggcgacgtaaacggccacaagttcagcgtgtccggcgagggcgagggcgatgccacctacggcaagctgaccctgaagctgatctgcaccaccggcaagctgcccgtgccctggcccaccctcgtgaccaccctcggctacggcctgaagtgcttcgcccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcggcttcaaggaggacggcaacatcctggggcacaagctggagtacaacggcaccggtttctgttttgaagccggggcggatgaagatacacacttccgcgcgaccagcctggaaactctgcgcaacatggtggcggcaggtagcgggatcactttactgccagcgctggctgtgccgccggagcgcaaacgcgatggggttgtttatctgccgtgcattaagccggaaccacgccgcactattggcctggtttatcgtcctggctcaccgctgcgcagccgctatgagcagctggcagaggccatccgcgcaagaatggatggccatttcgataaagttttaaaacaggcggtttaatgagcggccgccaccgcggtggagctcgaattaattcatcgatgatgatccagacatgataagatacattgatgagtttggacaaaccacaactagaatgcagtgaaaaaaatgctttatttgtgaaatttgtgatgctattgctttatttgtaaccattataagctgcaataaacaagttaacaacaacaattgcattcattttatgtttcaggttcagggggaggtgtgggaggttttttaaagcaagtaaaacctctacaaatgtggtatggctgattatgatcctctagatcagatctgtcccgatctttctcttctgtgctgtcaatttagagtagtttttgaggtaatttgggtgcattctctgtaaaaacaacaaataaatatttatattgtatgcaataaatgtaacaattacacatacactctctgattacagcattaaagggttagttcacccaaaaatgaaaagatgtcattaatgactcaccctcatgtcgttccaagcccgtaacacctccgttcatcttcggaacacagtttaagatattttagatttagtccgagagctttctgtccctccactgaaaatgtatgtacggtatactgtccatgtccagaaaggtaataaaaacatcaaagtagtccatgtgacatcagtgggttaggtagaattatttgaagcatcgaaaatacattttggtccaaaaataacaaaacctacgactttattcagcattgtagcgtcactgcgaagtcgtgaacgcggattgacaacagacccggaagagaatacaatgccgaataaagtcgtaggttttgttatttttggaccaaaatgtattcgatgcttcaaaaaattctaactaacccactgatgtcacatggactactttgatgtttttattacctttctggacatggacagtataccgtacatacattctcaatggaggcacagaaagctctcggactaaatctaaaatatcttaaactgtgttctgaagatgaacggaggtcttacgggcttggaacgacatgagggcgagtcattaatgacattattttcatttttgggtgaactaaccctttaataaaactgacatttgcagccaattgcacactttataatgttaaatacttgttgattttagaaatacagtagcatttagtgacaatcaaacattaattttatttctgattatattgacaaaaacacaggactgtaaggctgacatgacagggtgaattgatttcattacattagttttgatggtaacacttttacaataaggttcgttagttaactacattagttaacatgaactaataatgaactgcacgtatacatgttaatttcaacatttactaatactttatatcttgttaacattagttaatgcactgtgaactaacatgaacaaacaatgaacatctgtatttctattaactaacgttaacaaagattaacagatacagtaacaaatgtattgctcatggttatttaatgttagttaatacatgaactaatcaacctcattgtaaagtgttaccgttttgacaatacaacatgtttggaaaaaaatttctcgcttggttttcttgtgttttgcatgaattcaaacacaatgagagcacggaatgataatgattattcaaagctgcaataggcgccaaatttcccgtggacctaacgttaccaattacaaatgcattttagcattttattacgtgagccttatattgtcaatcgcatgcattagtgaaaataggcctactacacatataaaaggcgcgccatcaacgtcaaaacatgcacacccaaaaaaaaaaaaaaaaaaaaaaaaaaaaactatcagtaaaacaaaatacttaatgtacttacctcaatatgcttccttaggtttgatggcgaacttttgaaggccgatatttctttattaagcgggagacagaggacacatttcatcttgaatgaatctttatttacaccacttaaagaaaagaattcgcgaagatacggccacgggtgctcttgatcctgtggctgattttggactgtgctgctcgcagctgctgatgaatcacatacttcctccattttcttccactgattgactgttataatttccctaatttccaggtcaaggtgctgtgcattgtggtaatagatgtgacatgacgtcacttccaaaggaccaatgaacatgtctgaccaatttcatataatgtgaaaacgattttcataggcagaataaataacatttaaattaaactgggcatcagcgcaattcaattggtttggtaatagcaagggaaaatagaatgaagtgatctccaaaaaataagtactttttgactgtaaataaaattgtaaggagtaaaaagtacttttttttctaaaaaaatgtaattaagtaaaagtaaaagtattgatttttaattgtactcaagtaaagtaaaaatccccaaaaataatacttaagtacagtaatcaagtaaaattactcaagtactttacacctctggttcttgaccccctaccttcagcaagcccagcagatccactagttctagagcggccgccaccgcggtggagctccagcttttgttccctttagtgagggttaattgcgcgcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtg

SEQ ID NO: 29 - DNA, 9974 bp, synthetic construct
  source 1..9974: mol_type = other DNA
gccaccgcggtggagctcgaattaattcatcgatgatgatccagacatgataagatacattgatgagtttggacaaaccacaactagaatgcagtgaaaaaaatgctttatttgtgaaatttgtgatgctattgctttatttgtaaccattataagctgcaataaacaagttaacaacaacaattgcattcattttatgtttcaggttcagggggaggtgtgggaggttttttaaagcaagtaaaacctctacaaatgtggtatggctgattatgatcctctagatcaggccgccaccgcggtggagctccagcttttgttccctttagtgagggttaattgcgcgcttggcgtaatcatggtcatagctgtttcctgtgtggcgaagatacggccacgggtgctcttgatcctgtggctgattttggactgtgctgctcgcagctgctgatgaatcacatacttcctccattttcttccactgattgactgttataatttccctaatttccaggtcaaggtgctgtgcattgtggtaatagatgtgacatgacgtcacttccaaaggaccaatgaacatgtctgaccaatttcatataatgtgaaaacgattttcataggcagaataaataacatttaaattaaactgggcatcagcgcaattcaattggtttggtaatagcaagggaaaatagaatgaagtgatctccaaaaaataagtactttttgactgtaaataaaattgtaaggagtaaaaagtacttttttttctaaaaaaatgtaattaagtaaaagtaaaagtattgatttttaattgtactcaagtaaagtaaaaatccccaaaaataatacttaagtacagtaatcaagtaaaattactcaagtactttacacctctgggggaggttccctttagtgagggttaattgcgcgcttggcgtaatcatggtcatagctgtttcctgtgtgaaattgttatccgctcacaattccacacaacatacgagccggaagcataaagtgtaaagcctggtgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctaaattgtaagcgttaatattttgttaaaattcgcgttaaatttttgttaaatcagctcattttttaaccaataggccgaaatcggcaaaatcccttataaatcaaaagaatagaccgagatagggttgagtgttgttccagtttggaacaagagtccactattaaagaacgtggactccaacgtcaaagggcgaaaaaccgtctatcagggcgatggcccactacgtgaaccatcaccctaatcaagttttttggggtcgaggtgccgtaaagcactaaatcggaaccctaaagggagcccccgatttagagcttgacggggaaagccagaggtgtaaaaagtactcaaaaattttactcaagtgaaagtacaagtacttagggaaaattttactcaattaaaagtaaaagtatctggctagaatcttacttgagtaaaagtaaaaaagtactccattaaaattgtacttgagtattaaggaagtaaaagtaaaagcaagaaagaaaactagagattcttgtttaagcttttaatctcaaaaaacattaaatgaaatgcatacaaggttttatcctgctttagaactgtttgtatttaattatcaaactataagacagacaatctaatgccagtacacgctactcaaagttgtaaaacctcagatttaacttcagtagaagctgattctcaaaattgttagtgtcaagcctagctcttttggggctgaaaagcaatcctgcagtgctgaaaagcctctcacaggcagccgatgcgggaagaggtgtattagtcttgatagagaggctgcaaatagcaggaaacgtgagcagagactccctggtgtctgaaacacaggccagatcgcgttgtaaaacgacggccagtgagcgcgcgtaatacgactcactatagggcgaattgggtacgatccaccggtcggccggccgattaattaacgagctcgagagaaaccaggctcagtcgggcacgaccattactccacttgaccaaacgtctgcatatttataaataataatttgcgtattaatatgcagataaagacaattaaaagaagcaacagtagcattaatcattcaaataacagtatacaatcaaacaacattttgtatatatatttaaaaaaacaatacaaggagtaaagacaaaggcaatatcatttagtttttttaatattaacaaaccacaggctgtgtgaaccatcagcaatttcaaaccagagggatctgcttttttgggaacgatgttgcaaatttaatctctagattttttaaatcatggatagcctccctaaagcaaaagctttttaggggctttacttctcctgattgcagcctgtggtataatttgtgattgttaggtgggtcaggttttggggtccagccctttgtgcactttgactattgtcaccttaggctgtcccttaatgctgccacctctgaccccggtgggacacataaataacttctccacccgagttagcatttcaagccagtgcaataaagaaattaaaaataattgatttattaacaactctgttttatctttaaaaaataaaatgttttatatctaatgttcactcaggtacctgatattgtttgctgtgcaataatgtaggttagtcattatattgacaaagaaaattaaatatgatgttgtttatttttaattttacattatagactagattagattttcatttggaatttaattaggagtgcctggagtgcattgagaacgttaaaaaaaaaaaatctgcttactcttatagctttggaaactattttcagattattactccactatagaagcgattcctttttttgaataattaaatatatatatacagtgctcagcataaatgatcatcacagatctctattttaaagtattatttttccataggatgctttacaataatatatgtgtgtgtatacattagaattgtcagttacaaaatcaaaactgcaacaaaataactaaacttaagatctaatgagtacacccaaattaaaccaaacattagtctatgcaaaaattaatatgttaaggaaaaatattaaatacaattttaatattcagtcattcattttcttttttgcttagtccctttattaatctggggtcgccacagtggaattaactgccaatttatccagcatatattttatgcagcggatgctcttccagctgctacccatcactgggaatcacccatacactctcattcacacacatacactacggacaatttagcctacccaattcacctatagcgtatgtctttggacttgtgggggaaaccggaacacacggaggaaacccacgcgaacacagggagatcatgcaaactcagaaacgccaactgatccagccaaggctcgaaccagtgaccttcttgctgtgaggcaatagggctacccattgtgtcactgcaccgtccacaattttaataaacatgaaaaatcccgagaaacataaacaatattaaatgtagttgaaattttgttgtttgaaattgtatttattaaataaaaaaaatgtatgtattattattatttatttatttataaaagatgttaggtggccaaactcttttttttaatggatatagttgtttaataaaacaattttgtttaaataaaacaaaatatattgaatgagccatgttgctgttgttgttttttacaggcttgatgtacaatatgatctttgtgtagttttatagtaataaatcattcatgtcttgtgtattgtacgtcatattatcttaaaagtacgctattaattaaacttgtttatatggtcatcttagacaggactctcattataaaaggatttttgttaattttattagtaccttcctggaaaaataaaagataaataaataaaaatgaataaaagtcttgggcttgttttctgtacgagtaataaaaaagagtaagcaaatattctgcatgacaatattgttcaaagtttaagggctgtattttttataattgttggttattaacatgttttatacaaattactttcaacatggctgaaataataattgttatcactatttgaaacagtcctagaatagattttactatatttacatttattcctctgatgacaaagttgaaattcctctgatgacaaagcaaaactctcaactctagttttattttattgtaataaccatgtcaagggataatgacaatgttttccattatattttcaatatagaataaaattataatttattaatcatttagtagggcagcatggtggcacaatgggtagcacaatcacctcacaccaagaaggttgctagttcgagccccggctgggtcagtttgcatatctgtgtggagaatgttctctctgtgttgccatgggtttcctctgggtgctctagtttccaccacaagtctaaaaacaagtggcatcggtgaattgtgtaggctaaattgtctgtagtgtttgtgtgtgattgagtgtttatggatgtttcccagtgatgggttgcagttggaagggcattctctgcgtaaaactataagttggcggttcattccactgtggtgaccccagattaataaagggattaagccgaaaagaaaatgaatcattaaatgatcatttagttgattggagagcattgcataatgagaatcaaataatgtatagttttttgtaaaatgattgtgtattagaggcacattgtacgtagtgtggaatgtttaggttgtctttctgattgttttcattttgcagaagttcatttgcagtcagtgtagtcaaagcacctcagctcaatttatagcagcagcctgctgtgatttaagtgccacaggagtgaaggctttaacccctgttgtagattgcaaccaatgtttttctaaggtaattagaagcttgtttagctaacgctacccaccccaccccccacaaaaaagttgctctgaatagcagcatctacccccaggagggtgcaagcttctgttttgaagaaatttgctcatggtgttacataggtgtttaaaaatgtatgttttttaatagcatatattttaaaagtgactcattaatgacatcatctgccatctgcctttctcagaactgacaaaggacccttttaaggacccctatacaccacatttttatctatctaatcacaagtgggtgatattagagggatgattcacctaaaaatgaattttccgcattttaatttggggttccaaaccagtttgaattcttttgttctgttgaacacaaaataagatgttttaagaatgttggaaatgttggagacttttatagtaggaaaaacatatactaatatttgcattcatgtttgaaatgtttaaaacatttttcaaaatatcttatttttcaactgttttggaacaagtcaaaagtgagtaaatgatgactgataaacaatttctttaaatacagatgaaaatagcgtctaacacattttgaactagttcactttcaaccacagcaagaggtagtcgaaaacacatttgatcacattttgttgtgggaccgcttgttgttcagtgtgttttcattgttgagttacaaaccggtaagtggcattaaaacatgttttggcatcactgatgatcataaataaattggttaaaaaattaaaaattagatgcttacagtacagtttgtttaaaaacttgcactttttgtagtgtgaaatgacattagtacctgcagtgggctttctttgttctgtatcttctgatgaatctaacataaagacatttaaatattcacaatggagtgggttggttctcatctatctgaaacaacattagtgtgttcaaaacaaatctacaccagtgatctctgatagatacaagctgtcaaccgtagtcctattgtttctcatgcgactgtcacagggccatgctagaggctctttgggtggtgagtagagactcattcttatgcaaacaggctatggatgctacaactgaagagaatttagtgctctaaggtcatcgcagttctataaagcagattccctccaaaaagctagagaaaattatgtgtctggaggcaccatctatcagtgcagtcattcttttattactttttttttttgtccatgcaattttttaatcgcattttgaagtatttaaaccattaagagtgtcaaattatcatgttattatttcaactcataatatctccttttaaagatgattcatttgattgtagttataataattgttttattaatgcaaaaattgcttaaatgtttggcctctgagattttcagttgactcaatatttttggagcctgtttcatgactttatttaaaatataatctgcactcgcaaatattacaattatatcatattttgaaaatcttagatgtacatttgttttgaaaatggtttaaaggaaatttaggtcaaaatgtgtgttttttttaaataataattcagtcttttttttgatataatagggaaaatgacagcaattaaaaggctgttttggaaagacatctaggtctggtgcagacttcgtaacttcttagagttaaatcttgaaaacggagccagaagacgggcaacaccttcctaaaagcagggtggggtgttggacaggtagcttcttggaacctcccctctttgtcgctgtggtatatatagaaatcccattgaattcctatagagaccttaagcccgtaccatttacctctgaagccacgggtttagtgaaccgtcagatccgctagcatgtccgtcctgacgccgctgctgctgcggggcttgacaggctcggcccggcggctcccagtgccgcgcgccaagatccattcgttgggggatctgtccgtcctgacgccgctgctgctgcggggcttgacaggctcggcccggcggctcccagtgccgcgcgccaagatccattcgttgggggatccaccggtcgccaccgagatggcaagccagcagggcgagacgatgtccggaccgctgcacattggtttgattcccacagttggaccgtacctgctaccgcatattatccctatgctgcaccagacctttccaaagctggaaatgtatctgcatgaagcacagacccaccagttactggcgcaactggacagcggcaaactcgattgcgtgatcctcgcgctggtgaaagagagcgaagcattcattgaagtgccgttgtttgatgagccaatgttgctggctatctatgaagatcacccgtgggcgaaccgcgaatgcgtaccgatggccgatctggcaggggaaaaactgctgatgctggaagatggtcactgtttgcgcgatcaggcaatgtccgccggctacaacagcgacaacgtctatatcatggccgacaagcagaagaacggcatcaaggccaacttcaagatccgccacaacgtcgaggacggcagcgtgcagctcgccgaccactaccagcagaacacccccatcggcgacggccccgtgctgctgcccgacaaccactacctgagcttccagtccgtcctgagcaaagaccccaacgagaagcgcgatcacatggtcctgctggagttcgtgaccgccgccgggatcactctcggcatggacgagctgtacaacgtggatggcggtagcggtggcaccggcagcaagggcgaggagctgttcaccggggtggtgcccatcctggtcgagctggacggcgacgtaaacggccacaagttcagcgtgtccggcgagggcgagggcgatgccacctacggcaagctgaccctgaagctgatctgcaccaccggcaagctgcccgtgccctggcccaccctcgtgaccaccctcggctacggcctgaagtgcttcgcccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcggcttcaaggaggacggcaacatcctggggcacaagctggagtacaacggcaccggtttctgttttgaagccggggcggatgaagatacacacttccgcgcgaccagcctggaaactctgcgcaacatggtggcggcaggtagcgggatcactttactgccagcgctggctgtgccgccggagcgcaaacgcgatggggttgtttatctgccgtgcattaagccggaaccacgccgcactattggcctggtttatcgtcctggctcaccgctgcgcagccgctatgagcagctggcagaggccatccgcgcaagaatggatggccatttcgataaagttttaaaacaggcggtttaaagcggccgcgactctagatcataatcagccataccacatttgtagaggttttacttgctttaaaaaacctcccacacctccccctgaacctgaaacataaaatgaatgcaattgttgttgttaacttgtttattgcagcttataatggttacaaataaagcaatagcatcacaaatttcacaaataaagcatttttttcactgcattctagttgtggtttgtccaaactcatcaatgtatctta

SEQ ID NO: 30 - DNA, 11235 bp, synthetic construct
  source 1..11235: mol_type = other DNA
ctgacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgcttacaatttgccattcgccattcaggctgcgcaactgttgggaagggcgatcggtgcgggcctcttcgctattacgccagctggcgaaagggggatgtgctgcaaggcgattaagttgggtaacgccagggttttcccagtcacgacgttgtaaaacgacggccagtgaattgtaatacgactcactatagggcgaccctgcagaggtgtaaaaagtactcaaaaattttactcaagtgaaagtacaagtacttagggaaaattttactcaattaaaagtaaaagtatctggctagaatcttacttgagtaaaagtaaaaaagtactccattaaaattgtacttgagtattaaggaagtaaaagtaaaagcaagaaagaaaactagagattcttgtttaagcttttaatctcaaaaaacattaaatgaaatgcatacaaggttttatcctgctttagaactgtttgtatttaattatcaaactataagacagacaatctaatgccagtacacgctactcaaagttgtaaaacctcagatttaacttcagtagaagctgattctcaaaattgttagtgtcaagcctagctcttttggggctgaaaagcaatcctgcagtgctgaaaagcctctcacaggcagccgatgcgggaagaggtgtattagtcttgatagagaggctgcaaatagcaggaaacgtgagcagagactccctggtgtctgaaacacaggccagatggccccgcggccgggccctcggccggccgattaattaacgagctcgagagaaaccaggctcagtcgggcacgaccattactccacttgaccaaacgtctgcatatttataaataataatttgcgtattaatatgcagataaagacaattaaaagaagcaacagtagcattaatcattcaaataacagtatacaatcaaacaacattttgtatatatatttaaaaaaacaatacaaggagtaaagacaaaggcaatatcatttagtttttttaatattaacaaaccacaggctgtgtgaaccatcagcaatttcaaaccagagggatctgcttttttgggaacgatgttgcaaatttaatctctagattttttaaatcatggatagcctccctaaagcaaaagctttttaggggctttacttctcctgattgcagcctgtggtataatttgtgattgttaggtgggtcaggttttggggtccagccctttgtgcactttgactattgtcaccttaggctgtcccttaatgctgccacctctgaccccggtgggacacataaataacttctccacccgagttagcatttcaagccagtgcaataaagaaattaaaaataattgatttattaacaactctgttttatctttaaaaaataaaatgttttatatctaatgttcactcaggtacctgatattgtttgctgtgcaataatgtaggttagtcattatattgacaaagaaaattaaatatgatgttgtttatttttaattttacattatagactagattagattttcatttggaatttaattaggagtgcctggagtgcattgagaacgttaaaaaaaaaaaatctgcttactcttatagctttggaaactattttcagattattactccactatagaagcgattcctttttttgaataattaaatatatatatacagtgctcagcataaatgatcatcacagatctctattttaaagtattatttttccataggatgctttacaataatatatgtgtgtgtatacattagaattgtcagttacaaaatcaaaactgcaacaaaataactaaacttaagatctaatgagtacacccaaattaaaccaaacattagtctatgcaaaaattaatatgttaaggaaaaatattaaatacaattttaatattcagtcattcattttcttttttgcttagtccctttattaatctggggtcgccacagtggaattaactgccaatttatccagcatatattttatgcagcggatgctcttccagctgctacccatcactgggaatcacccatacactctcattcacacacatacactacggacaatttagcctacccaattcacctatagcgtatgtctttggacttgtgggggaaaccggaacacacggaggaaacccacgcgaacacagggagatcatgcaaactcagaaacgccaactgatccagccaaggctcgaaccagtgaccttcttgctgtgaggcaatagggctacccattgtgtcactgcaccgtccacaattttaataaacatgaaaaatcccgagaaacataaacaatattaaatgtagttgaaattttgttgtttgaaattgtatttattaaataaaaaaaatgtatgtattattattatttatttatttataaaagatgttaggtggccaaactcttttttttaatggatatagttgtttaataaaacaattttgtttaaataaaacaaaatatattgaatgagccatgttgctgttgttgttttttacaggcttgatgtacaatatgatctttgtgtagttttatagtaataaatcattcatgtcttgtgtattgtacgtcatattatcttaaaagtacgctattaattaaacttgtttatatggtcatcttagacaggactctcattataaaaggatttttgttaattttattagtaccttcctggaaaaataaaagataaataaataaaaatgaataaaagtcttgggcttgttttctgtacgagtaataaaaaagagtaagcaaatattctgcatgacaatattgttcaaagtttaagggctgtattttttataattgttggttattaacatgttttatacaaattactttcaacatggctgaaataataattgttatcactatttgaaacagtcctagaatagattttactatatttacatttattcctctgatgacaaagttgaaattcctctgatgacaaagcaaaactctcaactctagttttattttattgtaataaccatgtcaagggataatgacaatgttttccattatattttcaatatagaataaaattataatttattaatcatttagtagggcagcatggtggcacaatgggtagcacaatcacctcacaccaagaaggttgctagttcgagccccggctgggtcagtttgcatatctgtgtggagaatgttctctctgtgttgccatgggtttcctctgggtgctctagtttccaccacaagtctaaaaacaagtggcatcggtgaattgtgtaggctaaattgtctgtagtgtttgtgtgtgattgagtgtttatggatgtttcccagtgatgggttgcagttggaagggcattctctgcgtaaaactataagttggcggttcattccactgtggtgaccccagattaataaagggattaagccgaaaagaaaatgaatcattaaatgatcatttagttgattggagagcattgcataatgagaatcaaataatgtatagttttttgtaaaatgattgtgtattagaggcacattgtacgtagtgtggaatgtttaggttgtctttctgattgttttcattttgcagaagttcatttgcagtcagtgtagtcaaagcacctcagctcaatttatagcagcagcctgctgtgatttaagtgccacaggagtgaaggctttaacccctgttgtagattgcaaccaatgtttttctaaggtaattagaagcttgtttagctaacgctacccaccccaccccccacaaaaaagttgctctgaatagcagcatctacccccaggagggtgcaagcttctgttttgaagaaatttgctcatggtgttacataggtgtttaaaaatgtatgttttttaatagcatatattttaaaagtgactcattaatgacatcatctgccatctgcctttctcagaactgacaaaggacccttttaaggacccctatacaccacatttttatctatctaatcacaagtgggtgatattagagggatgattcacctaaaaatgaattttccgcattttaatttggggttccaaaccagtttgaattcttttgttctgttgaacacaaaataagatgttttaagaatgttggaaatgttggagacttttatagtaggaaaaacatatactaatatttgcattcatgtttgaaatgtttaaaacatttttcaaaatatcttatttttcaactgttttggaacaagtcaaaagtgagtaaatgatgactgataaacaatttctttaaatacagatgaaaatagcgtctaacacattttgaactagttcactttcaaccacagcaagaggtagtcgaaaacacatttgatcacattttgttgtgggaccgcttgttgttcagtgtgttttcattgttgagttacaaaccggtaagtggcattaaaacatgttttggcatcactgatgatcataaataaattggttaaaaaattaaaaattagatgcttacagtacagtttgtttaaaaacttgcactttttgtagtgtgaaatgacattagtacctgcagtgggctttctttgttctgtatcttctgatgaatctaacataaagacatttaaatattcacaatggagtgggttggttctcatctatctgaaacaacattagtgtgttcaaaacaaatctacaccagtgatctctgatagatacaagctgtcaaccgtagtcctattgtttctcatgcgactgtcacagggccatgctagaggctctttgggtggtgagtagagactcattcttatgcaaacaggctatggatgctacaactgaagagaatttagtgctctaaggtcatcgcagttctataaagcagattccctccaaaaagctagagaaaattatgtgtctggaggcaccatctatcagtgcagtcattcttttattactttttttttttgtccatgcaattttttaatcgcattttgaagtatttaaaccattaagagtgtcaaattatcatgttattatttcaactcataatatctccttttaaagatgattcatttgattgtagttataataattgttttattaatgcaaaaattgcttaaatgtttggcctctgagattttcagttgactcaatatttttggagcctgtttcatgactttatttaaaatataatctgcactcgcaaatattacaattatatcatattttgaaaatcttagatgtacatttgttttgaaaatggtttaaaggaaatttaggtcaaaatgtgtgttttttttaaataataattcagtcttttttttgatataatagggaaaatgacagcaattaaaaggctgttttggaaagacatctaggtctggtgcagacttcgtaacttcttagagttaaatcttgaaaacggagccagaagacgggcaacaccttcctaaaagcagggtggggtgttggacaggtagcttcttggaacctcccctctttgtcgctgtggtatatatagaaatcccattgaattcctatagagaccttaagcccgtaccatttacctctgaagcctaggatgaagctactgtcttctatcgaacaagcatgcgatatttgccgacttaaaaagctcaagtgctccaaagaaaaaccgaagtgcgccaagtgtctgaagaacaactgggagtgtcgctactctcccaaaaccaaaaggtctccgctgactagggcacatctgacagaagtggaatcaaggctagaaagactggaacagctatttctactgatttttcctcgagaagaccttgacatgattttgaaaatggattctttacaggatataaaagcattgttaacaggattatttgtacaagataatgtgaataaagatgccgtcacagatagattggcttcagtggagactgatatgcctctaacattgagacagcatagaataagtgcgacatcatcatcggaagagagtagtaacaaaggtcaaagacagttgactgtatcgtcgaggtcgaccccgggaattcagatctctcgagccgcccccccgaccgatgtcagcctgggggacgagctccacttagacggcgaggacgtggcgatggcgcatgccgacgcgctagacgatttcgatctggacatgttgggggacggggattccccgggtccgggatttaccccccacgactccgccccctacggcgctctggatatggcccccccgaccgatgtcagcctgggggacgagctccacttagacggcgaggacgtggcgatggcgcatgccgacgcgctagacgatttcgatctggacatgttgggggacggggattccccgggtccgggatttaccccccacgactccgccccctacggcgctctggatatggccgacttcgagtttgagcagatgtttaccgatgcccttggaattgacgagtacggtgggggcggccggccgcgactctagatcataatcagccataccacatttgtagaggttttacttgctttaaaaaacctcccacacctccccctgaacctgaaacataaaatgaatgcaattgttgttgttaacttgtttattgcagcttataatggttacaaataaagcaatagcatcacaaatttcacaaataaagcatttttttcactgcattctagttgtggtttgtccaaactcatcaatgtatggcggccgcagctcggcgcccttgctcaccatggtggcgaccggtcttgtacagctcatccatgccgtgggtgatggcggcggcggtcacgaagccgaagtagatcatgtgatcgcgcttctcgttggggtccttggacagggcgctctgggtggacaggtagtggttatcgggcagcagcacagggccatcgccgatgggggtattctgctggtagtggtcggccagctgcacgctgccatcctcgatgttgtggcggatcttgaagttcaccttgatgccattcttggccttgtcggtcatgatgtacacattgtgggcgttgtagttgtactccatcttattgcccaggatgttgccatcctccttgaaatcggtgccggtcagctcgatgcgattcaccagggtatcgccctcgaacttcacctcggcgcgcgacttgtagttgccgtcatcctcgaagaagatggtgcgctcctggatgtagccctcaggcatggcgctcttgaagaagtcgtgctgcttcatgtgatcggggtagcgtgagaagcactgcacgccgtagctcagggtggtcaccagggtgggccagggcacaggcagcttgccggtggtgcagatgaacttcagggtcagcttgccgtaggtggcatcgccctcgccctcgccgctcacgctgaacttgtggccattcacatcgccattcagctcgatcaggatgggcacgatgccggtgaacagctcggcgcccttgctcaccatggatcccgtctagagggtatataatggatcccatcgcgtctcagcctcactttgagctcctccacacgaattctttggcccggagtactgtcctccgggctggcggagtactgtcctccggcaaggtcggagtactgtcctccgacactagaggtcggagtactgtcctccgacgcaaggcggagtactgtcctccgtctagagggtatataatggatcccatcgcgtctcagcctcactttgagctcctccacacgaattccctcgacctcgaagaccttaagttcgaattaggcgcgccaggtgatcatcatgaatccgctcattgatatctttttttttcctttcttgtacaaagtgggggatccagacatgataagatacattgatgagtttggacaaaccacaactagaatgcagtgaaaaaaatgctttatttgtgaaatttgtgatgctattgctttatttgtaaccattataagctgcaataaacaagttaacaacaacaattgcattcattttatgtttcaggttcagggggaggtgtgggaggttttttccaactttattatacaggtaccaaggccttcgatttttcgcgattttttgcgaagatacggccacgggtgctcttgatcctgtggctgattttggactgtgctgctcgcagctgctgatgaatcacatacttcctccattttcttccactgattgactgttataatttccctaatttccaggtcaaggtgctgtgcattgtggtaatagatgtgacatgacgtcacttccaaaggaccaatgaacatgtctgaccaatttcatataatgtgaaaacgattttcataggcagaataaataacatttaaattaaactgggcatcagcgcaattcaattggtttggtaatagcaagggaaaatagaatgaagtgatctccaaaaaataagtactttttgactgtaaataaaattgtaaggagtaaaaagtacttttttttctaaaaaaatgtaattaagtaaaagtaaaagtattgatttttaattgtactcaagtaaagtaaaaatccccaaaaataatacttaagtacagtaatcaagtaaaattactcaagtactttacacctctgggggaggttccctttagtgagggttaattgcgcgcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccac

SEQ ID NO: 31 - DNA, 10033 bp, synthetic construct
  source 1..10033: mol_type = other DNA
ccacctaaattgtaagcgttaatattttgttaaaattcgcgttaaatttttgttaaatcagctcattttttaaccaataggccgaaatcggcaaaatcccttataaatcaaaagaatagaccgagatagggttgagtgttgttccagtttggaacaagagtccactattaaagaacgtggactccaacgtcaaagggcgaaaaaccgtctatcagggcgatggcccacgctggctaagaactcatcagcctccccggtccatctacccacgtaccaatgcaccaattggccacaatgacggctactacatggtgccattccttcctctttataggaatggagactacctcctgtccaacaaggctcttggatacgagtacgcctacctgttggacccaggtcattgcacaacaccagaaatgccctctgatctgcaaaagacgtgaatatctgttcagacacccatatccactctgttccacacaggtcagaggtttgtccaggagttcttgacagaggtgtaaaaagtactcaaaaattttactcaagtgaaagtacaagtacttagggaaaattttactcaattaaaagtaaaagtatctggctagaatcttacttgagtaaaagtaaaaaagtactccattaaaattgtacttgagtattaaggaagtaaaagtaaaagcaagaaagaaaactagagattcttgtttaagcttttaatctcaaaaaacattaaatgaaatgcatacaaggttttatcctgctttagaactgtttgtatttaattatcaaactataagacagacaatctaatgccagtacacgctactcaaagttgtaaaacctcagatttaacttcagtagaagctgattctcaaaattgttagtgtcaagcctagctcttttggggctgaaaagcaatcctgcagtgctgaaaagcctctcacaggcagccgatgcgggaagaggtgtattagtcttgatagagaggctgcaaatagcaggaaacgtgagcagagactccctggtgtctgaaacacaggccagatgggccctcgagcaggaaacagctatgaccatgattacgccaagctatcaactttgtatagaaaagttgaacgagaaacgtaaaatgatataaatatcaatatattaaattagattttgcataaaaaacagactacataatactgtaaaacacaacatatccagtcactatggtcgaccagagagggagagagggatagggacaagagtggcccatcaatacagtgaccaaacaccggagctcaacacacacccctaactctaatcgcaagcatctcgatttcgggaagtcctgcaacacctcgaaccctcggccactttcccaccttcacagataaaaacacgggcactaagggccttctgtccggttttaaaagtggacctaacaccgccttactttcttgacatgggagacatgggggatccaccgaaaaaaaagcgtctaatctcgttgtgtgtcggctgtgggaatcaaatccatgaccagtatattctgcgcgtgtcgccggatctggagtggcacgcggcgtgtttgaaatgtgcagaatgtaaccagtatctggacgagtcctgtacatgttttgtgcgagacgggaaaacttactgtaaacgggactacatcaggttatacgggatcaaatgtgcaaaatgcaacatcggtttcagcaagaatgactttgtgatgagagcacgttcgaaggtttatcatattgagtgttttagatgtgtggcgtgtagtcggcagctcatcccaggagatgagttcgctctgcgggaagacgggctcttctgcagggccgaccatgacgtggtggagcgggcaacaatgggtgctggtgacccattaagcccattacatccggcgagacctttacaaatggcagcagagcccatttcggcacgtcagcctgcgcttcgacctcatgtgcacaagcaacctgagaaaacaacccgcgtccggacagtcctcaacgaaaaacagctccataccttgaggacttgttacaatgccaaccctcgacccgacgccctcatgaaagagcagctcgttgagatgacgggtcttagtccgagagtcatcagggtttggtttcaaaacaagcgctgcaaggataaaaagaggagcatactgatgaagcaactccagcagcagcaacccaacgacaaaacgaacatccaggggatgacaggtactccaatggtggcgaccagtccagagagacacgacggtggtttgcaggcaaaccaagtggaggtgcagagttaccaaccgccttggaaagtcctaagtgacttcgcactgcagagtgacatcgaccagcctgctttccaacaactggttaatttttctgaaggtggtccaggctcaaactccacagggagcgaggtcgcgtcaatgtcctcgcaactgccagatacaccaaacagcatggtagcgagtcctatagaggcctaggcgagtggattatctcctgcatctgaagtttgcgaaaggacgtgtccgggaaccaatctcagagagaggctcgtgaagtttcttccatccctatttcatttagggccgcttctgaataaaagcccatcctccgactgcacgaggcctctatatcgaaacaacctttggttgtatttatacactgtgatgacaatcgtgggatttaaacacactctgaccgggcaagtccaacgagtgttgcgaaaagaccatttaaagttgcagactccagcgaaagtggaccctgccggcgcacatattcacataccgaacctgacatgatcattgcagtgctccgcggccgagtggacctggtcggctacacttttgccaaatacccctccacacttttcccacttttatgacaaatcaatggacctctacggatataagacgcaagagaacttagtctaatatattcattcatatatagtctacttctgtgcgcaacgtaagtagcctataatgaaaccatgactgtttattctgtttctataaaagatcgagacggactatacttgctcacaaataagggtatggcagccgaggtctactgcgattacagttttgttatatggtctcttttgaagcccaagtactaaggcattgcaacaaggtatacctctattttgccacaagctgtgtgggaatttcatgtgttcgtgtccgtccaagaagttttttttctttcccaaagatgtgtataggtttaatttaagttaatatgactgactagttctggaggttttgtttcgttcctgtattacaaaatattttattatttattgtcaaaagacatgccacttttgtgtacttttacttgctgaagaagtaaaaaaggaggaaaagaatattgtcgactccgaatgatctatcataaattgacgtgtcctgtcgtttgatcaaagactgatgtaagaacaacgccatatgtagagttatcttcaggaaaatcttgatcaataaaaatctgacgtgtactgtaagtccatgaagctactgtcttctatcgaacaagcatgcgatatttgccgacttaaaaagctcaagtgctccaaagaaaaaccgaagtgcgccaagtgtctgaagaacaactgggagtgtcgctactctcccaaaaccaaaaggtctccgctgactagggcacatctgacagaagtggaatcaaggctagaaagactggaacagctatttctactgatttttcctcgagaagaccttgacatgattttgaaaatggattctttacaggatataaaagcattgttaacaggattatttgtacaagataatgtgaataaagatgccgtcacagatagattggcttcagtggagactgatatgcctctaacattgagacagcatagaataagtgcgacatcatcatcggaagagagtagtaacaaaggtcaaagacagttgactgtatcgtcgaggtcgaccccgggaattcagatctctcgagccgcccccccgaccgatgtcagcctgggggacgagctccacttagacggcgaggacgtggcgatggcgcatgccgacgcgctagacgatttcgatctggacatgttgggggacggggattccccgggtccgggatttaccccccacgactccgccccctacggcgctctggatatgtagcggatctactagtgcggcccctctcgagcctctagaactatagtgagtcgtattacgtagatccagacatgataagatacattgatgagtttggacaaaccacaactagaatgcagtgaaaaaaatgctttatttgtgaaatttgtgatgctattgctttatttgtaaccattataagctgcaataaacaagttaacaacaacaattgcattcattttatgtttcaggttcagggggaggtgtgggaggttttttaattcgcggccatcaagcttaggcctccaaggcgatccagacatgataagatacattgatgagtttggacaaaccacaactagaatgcagtgaaaaaaatgctttatttgtgaaatttgtgatgctattgctttatttgtaaccattataagctgcaataaacaagttaacaacaacaattgcattcattttatgtttcaggttcagggggaggtgtgggaggttttttaaagcaagtaaaacctctacaaatgtggtatggctgattatccggagtactgtcctccgggctggcggagtactgtcctccggcaaggtcggagtactgtcctccgacactagaggtcggagtactgtcctccgacgcaaggcggagtactgtcctccgacactagaggtcggagtactgtcctccgacactagaggtcggagtactgtcctccgacactagaggtcggagtactgtcctccgacactagaggtcggagtactgtcctccgacactagaggtcggagtactgtcctccgacactagaggtcggagtactgtcctccgacactagaggtcggagtactgtcctccgacactagaggtcggagtactgtcctccgacactagaggtcggagtactgtcctccgtccacttaagctaggtggccagcggtgggatacattgatgagtttggacaaaccacaactagaatgcagtgaaaaaaatgctttatttgtgaaatttgtgatgctattgctttatttctagagggtatataatggatcccatcgcgtctcagcctcactttgagctcctccacacgaattctttggccacaagctgaccctgaagttcatctgcaccaccggcaagctgcccgtgccctggcccaccctcgtgaccaccctgactacggcgtgcagtgcttcagcaatatactcgagttatggtgagcaagggcgaggaggtcatcaaagagttcatgcgcttcaaggtgcgcatggagggctccatgaacggccacgagttcgagatcgagggcgagggcgagggccgcccctacgagggcacccagaccgccaagctgaaggtgaccaagggcggccccctgcccttcgcctgggacatcctgtccccccagttcatgtacggctccaaggcgtacgtgaagcaccccgccgacatccccgattacaagaagctgtccttccccgagggcttcaagtgggagcgcgtgatgaacttcgaggacggcggtctggtgaccgtgacccaggactcctccctgcaggacggcacgctgatctacaaggtgaagatgcgcggcaccaacttcccccccgacggccccgtaatgcagaagaagaccatgggctgggaggcctccaccgagcgcctgtacccccgcgacggcgtgctgaagggcgagatccaccaggccctgaagctgaaggacggcggccactacctggtggagttcaagaccatctacatggccaagaagcccgtgcaactgcccggctactactacgtggacaccaagctggacatcacctcccacaacgaggactacaccatcgtggaacagtacgagcgctccgagggccgccaccacctgttcctggggcatggcaccggcagcaccggcagcggcagctccggcaccgcctcctccgaggacaacaacatggccgtcatcaaagagttcatgcgcttcaaggtgcgcatggagggctccatgaacggccacgagttcgagatcgagggcgagggcgagggccgcccctacgagggcacccagaccgccaagctgaaggtgaccaagggcggccccctgcccttcgcctgggacatcctgtccccccagttcatgtacggctccaaggcgtacgtgaagcaccccgccgacatccccgattacaagaagctgtccttccccgagggcttcaagtgggagcgcgtgatgaacttcgaggacggcggtctggtgaccgtgacccaggactcctccctgcaggacggcacgctgatctacaaggtgaagatgcgcggcaccaacttcccccccgacggccccgtaatgcagaagaagaccatgggctgggaggcctccaccgagcgcctgtacccccgcgacggcgtgctgaagggcgagatccaccaggccctgaagctgaaggacggcggccactacctggtggagttcaagaccatctacatggccaagaagcccgtgcaactgcccggctactactacgtggacaccaagctggacatcacctcccacaacgaggactacaccatcgtggaacagtacgagcgctccgagggccgccaccacctgttcctgtacggcatggacgagctgtacaagtttatgcatattacgcgccatagtgactggatatgttgtgttttacagtattatgtagtctgttttttatgcaaaatctaatttaatatattgatatttatatcattttacgtttctcgttcaactttattatacatagttgataattcactggccgtcgttttacggtaccatcgatgatgatccagacatgataagatacattgatgagtttggacaaaccacaactagaatgcagtgaaaaaaatgctttatttgtgaaatttgtgatgctattgctttatttgtaaccattataagctgcaataaacaagttaacaacaacaattgcattcattttatgtttcaggttcagggggaggtgtgggaggttttttaaagcaagtaaaacctctacaaatgtggtatggctgattatgatcctctagatcagatctgcgaagatacggccacgggtgctcttgatcctgtggctgattttggactgtgctgctcgcagctgctgatgaatcacatacttcctccattttcttccactgattgactgttataatttccctaatttccaggtcaaggtgctgtgcattgtggtaatagatgtgacatgacgtcacttccaaaggaccaatgaacatgtctgaccaatttcatataatgtgaaaacgattttcataggcagaataaataacatttaaattaaactgggcatcagcgcaattcaattggtttggtaatagcaagggaaaatagaatgaagtgatctccaaaaaataagtactttttgactgtaaataaaattgtaaggagtaaaaagtacttttttttctaaaaaaatgtaattaagtaaaagtaaaagtattgatttttaattgtactcaagtaaagtaaaaatccccaaaaataatacttaagtacagtaatcaagtaaaattactcaagtactttacacctctggttcttgaccccctaccttcagcaagcccagcagatccactagttctagagcggccgccaccgcggtggagctccagcttttgttccctttagtgagggttaattgcgcgcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtg

SEQ ID NO: 32 - DNA, 167 bp, synthetic construct
  source 1..167: mol_type = other DNA
gtttgattaaagatatgggcaaaccggaacaggcaaaaccttcacaatggaaggtgaccgatcacccaaggaggatttttgggaagaggaaggattagctccgctcttaaacagccgaaangacaacccccctatttttttttttttttcctcttcttatttcctcc

SEQ ID NO: 33 - DNA, 170 bp, synthetic construct
  source 1..170: mol_type = other DNA
gtttgattaaagatatgggcaaaccggaacaggcaaaaccttcacaatggaaggtgaccgatcacccaatgaggagtttacttgggaagaggtatgatttgcgcagttctgaaactgtctaaatggcaacccaattattttttgtttcatttcctattctgaattcctcc

SEQ ID NO: 34 - DNA, 167 bp, synthetic construct
  source 1..167: mol_type = other DNA
gtttgattaaagatatgggcaaaccggaacaggcaaaaccttcacaatggaaggtgaccgatcacccaaggaggatttttgnngagaggagggattatttccgcacttaaaaagctgaaangacgaccccccaattttttttttttttttttcctcttcttacctcc

SEQ ID NO: 35 - DNA, 167 bp, synthetic construct
  source 1..167: mol_type = other DNA
gtttgattaaagatatgggcaaaccggaacaggcaaaaccttcacaatggaaggtgaccgatcacccgaggaggttttttgggaagaggaaggaatagctccgctcttnnacagccgaaaaaacaacccccctatttttttttttttttcctcttcttatttcctcc

(19) United States
(12) Patent Application Publication
(10) Pub. No.: US 2026/0345584 A1
(43) Pub. Date: Nov. 5, 2026

(54) METHODS AND PHARMACEUTICAL COMPOSITIONS FOR TREATING CHEMOTHERAPY-INDUCED PERIPHERAL NEUROPATHY WITH EG5 INHIBITORS

(71) Applicant: UNIVERSITY OF MIAMI, Miami, FL (US)

(72) Inventor: Sandra RIEGER, Miami, FL (US)

(21) Appl. No.: 19/485,501

(22) PCT Filed: May 20, 2024
(86) PCT No.: PCT/US2024/030257

§ 371 Date: Nov. 18, 2025

Related U.S. Application Data
(60) Provisional application No. 63/503,154, filed on May 18, 2023.

(51) Int. Cl. A61K 31/4741; A61P 25/02

(52) U.S. Cl. CPC A61K 31/4741; A61P 25/02

(57) ABSTRACT

This disclosure provides methods and pharmaceutical compositions for preventing or treating chemotherapy-induced peripheral neuropathy (CIPN) in a subject undergoing a chemotherapeutic treatment.

### CROSS-REFERENCE TO RELATED APPLICATIONS

This application claims priority to U.S. provisional application No. 63/503,154, filed May 18, 2023, the disclosure of which is expressly incorporated by reference herein.

### STATEMENT REGARDING FEDERALLY SPONSORED RESEARCH

This invention was made with government support under 1R01CA215973-05 awarded by the National Institutes of Health. The government has certain rights in the invention.

### REFERENCE TO AN ELECTRONIC SEQUENCE LISTING

The instant application contains a Sequence Listing that has been submitted electronically and is hereby incorporated by reference in its entirety. The Sequence Listing was created on May 17, 2024, is named “23-0726-WO_SequenceListing.xml”, and is 104,608 bytes in size.

### BACKGROUND OF THE DISCLOSURE

A wide variety of chemotherapeutic agents, including paclitaxel (Brand name Taxol), cause sensory-dominant peripheral neuropathy (Argyriou et al., 2010, J BU ON 15: 435-446; Beijers et al., 2012, Neth J Med 70: 18-25; Argyriou et al., 2012, Crit Rev Oncol Hematol 82: 51-77; Gornstein et al., 2014, Neuropharmacology 76 Pt A: 175-183; Boyette-Davis et al., 2015, Pain Manag 5: 285-296; Flatters et al., 2017, Br J Anaesth 119: 737-749; Argyriou et al., 2017, Nat Rev Neurol 13: 492-504; Argyriou et al., 2019, Journal of neurology, neurosurgery, and psychiatry 90:1361-1369). Studies have shown that the earliest signs of degeneration are detected in an unmyelinated sensory neuron population that innervates the epidermis (Bennett et al., 2011, Eur J Neurosci 33: 1667-1676). Symptoms range from pain and tingling to temperature sensitivity and numbness originating in the hands and feet and progressing proximally (Staff et al., 2020, Experimental neurology 324: 11312). One in every two chemotherapy patients suffers from chemotherapy-induced peripheral neuropathy (CIPN), and one in every three patients requires a dose reduction or discontinuation of this life-saving treatment (Seretny et al., 2014, Pain 155: 2461-2470), therefore decreasing the chance of survival. There are currently no treatments to prevent or reverse CIPN, mostly due to the lack of understanding about the molecular mechanisms of CIPN.

Recent research on the effects of chemotherapeutic drug-induced peripheral neuropathy has focused particularly on paclitaxel-induced damage of healthy cells. Putative mechanisms for paclitaxel neurotoxicity have been suggested based on rodent in vitro and in vivo studies (Staff et al., 2020, Id.), including reduced local mRNA translation due to decreased axonal microtubule transport and changes in mitochondrial functions (Pease-Raissi et al., 2017, Neuron 96: 373-386 e376; Bobylev et al., 2015, Neurobiol Dis 82: 321-33), leading to the deregulation of intracellular calcium dynamics and neuropeptide release in sensory neurons (Li et al., 2015, J Neurosci 35: 13487-1350). Also the induction of inflammatory cascades in rat dorsal root ganglion neurons following paclitaxel treatment involving chemokines, such as CXCL1/8/MCP-1/CCL-2 and their cognate receptors has been described (Brandolini et al., 2017, Oncotarget 8: 23188-23201). Studies in zebrafish further demonstrate that paclitaxel damages epidermal keratinocytes prior to promoting cutaneous sensory axon degeneration (Lisse et al., 2016, Proc. Natl. Acad. Sci. USA 113: E2189-2198). It was also demonstrated that paclitaxel treatment stimulated the formation of reactive oxygen species (ROS) in epidermal keratinocytes, which induced Matrix-Metalloproteinase 13 (MMP-13, collagenase-3) expression, resulting in extracellular matrix (ECM) degradation and axon degeneration (Lisse et al., 2016, Id; Cirrincione et al., 2020, Sci Rep 10: 3970). In addition, pharmacological inhibition of MMP-13 in zebrafish, rats, and mice rescued paclitaxel neurotoxicity and restored epidermal integrity. Studies in Drosophila and mice subsequently demonstrated that overexpression of the collagen-binding β-integrin 1 (ITGB1) in sensory neurons rescued paclitaxel neurotoxicity (Shin et al., 2021, Proc. Natl. Acad. Sci. USA 118: e2006050118). This is consistent with epidermal collagen degradation and neuronal downregulation of integrin receptors in a ROS-dependent manner resulting from paclitaxel induced MMP13 expression. This in turn promoted axonal detachment from the ECM and axon degeneration. However, the mechanisms upstream of MMP13 that are responsible for triggering CIPN remain unknown. There is thus a need in the art for a greater understanding of CIPN and methods and pharmaceutical compositions for treating this disorder.

### SUMMARY

The disclosure provides compositions associated with inhibition of paclitaxel induced ROS formation in epidermal keratinocytes upstream of MMP-13. Methods for using these compounds are also disclosed.

Disclosed herein in a first aspect is a method of preventing or treating chemotherapy-induced peripheral neuropathy (CIPN) in a subject undergoing a chemotherapeutic treatment, the method comprising: administering to the subject a therapeutic agent capable of inhibiting kinesin-5 (Eg5) function or expression.

In one embodiment of the method the therapeutic agent is an Eg5 inhibitor. In some embodiments, the Eg5 inhibitor is monastrol, ispinesib, (+)-S-trityle-L-cysteine (TriC), BRD9876, filanesib (ARRY-520), litronesib (LY2523355), AZD4877, SB-743921, MK0731, 4SC-205, ARQ 621, or EMD534085. In one embodiment, the Eg5 inhibitor is EMD534085. In some embodiments, the therapeutic agent is administered before, during, and after administration of the chemotherapeutic treatment.

In a further embodiment of the method the therapeutic agent inhibits expression of kif11 gene encoding EG5. More particularly, the therapeutic agent is a CRISPR-Cas9 complex comprising a single guide RNA specific for kif11 gene.

In another embodiment the chemotherapeutic treatment is paclitaxel, docetaxel, cabazitaxel, TPI-287, and albumin paclitaxel, or a combination thereof. The therapeutic agent can be delivered via intravenous administration or topical administration.

In a further embodiment, the therapeutic agent is capable of preventing or reducing degeneration of neurons innervating epidermal keratinocytes.

In another embodiment the chemotherapeutic treatment includes a chemotherapeutic agent capable of stabilizing microtubules in keratinocytes.

In a second aspect is a pharmaceutical composition comprising a therapeutic agent capable of inhibiting Eg5 function or expression, a chemotherapeutic agent capable of stabilizing microtubule in keratinocytes, and a pharmaceutically acceptable carrier. The pharmaceutical composition can contain the therapeutic agent which is an Eg5 inhibitor.

In another embodiment the pharmaceutical composition contains an Eg5 inhibitor that is monastrol, ispinesib, (+)-S-trityle-L-cysteine (TriC), BRD9876, filanesib (ARRY-520), litronesib (LY2523355), AZD4877, SB-743921, MK0731, 4SC-205, ARQ 621, or EMD534085. In another embodiment of the pharmaceutical composition the therapeutic agent inhibits expression of kif11 gene encoding Eg5. The therapeutic agent in certain embodiments is a CRISPR-Cas9 complex comprising a single guide RNA specific for kif11 gene.

In other embodiments of the pharmaceutical compositions disclosed herein the chemotherapeutic agent is paclitaxel, docetaxel, cabazitaxel, TPI-287, or albumin paclitaxel, or a combination thereof, wherein the pharmaceutical composition is delivered via intravenous administration or topical administration to a patient to prevent CIPN.

In a further embodiment the pharmaceutical composition is capable of preventing or reducing degeneration of neurons innervating epidermal keratinocytes.

In a further embodiment the pharmaceutical composition is used to prevent chemotherapy-induced peripheral neuropathy (CIPN) by administering a therapeutically effecting amount thereof to a patient undergoing chemotherapeutic treatment with an agent that promotes degeneration of neurons innervating the skin.

These and other features, objects, and advantages of this invention will become better understood from the description that follows. In the description, reference was made to the accompanying drawings, which form a part hereof and in which there was shown by way of illustration, not limitation, embodiments of the invention. The description of preferred embodiments was not intended to limit the invention to cover all modifications, equivalents, and alternatives. Reference should therefore be made to the claims recited herein for interpreting the scope of the invention.

### BRIEF DESCRIPTION OF THE DRAWINGS

The disclosure will be better understood and features, aspects, and advantages other than those set forth above will become apparent when consideration was given to the following detailed description thereof. Such detailed description refers to the following drawings.

FIG. 1A through FIG. 1 are images and analysis of detyrosination and fasciculation of microtubules following paclitaxel treatment. FIG. 1A is a transmission electron microscopy image of a larval zebrafish caudal fin at 6 days post fertilization (dpf) showing an infolded epidermis and a single keratinocyte wrapping around the distal caudal fin edge (green). Mesenchymal cells are located medially between the infolded epidermis. FIG. 1B is a schematic of the caudal fin shown in FIG. 1A depicting the cell types that are present. Two contralateral sensory neurons (red and blue) are shown that innervate the epidermis and arborize between the periderm and basal keratinocyte layer. FIG. 1C is a schematic of the experimental design for anti-detyrosinated tubulin antibody (GluTub) staining of detyrosinated (stabilized) microtubules (dMTs) shown in FIG. 1D-1F. FIG. 1D is the normalized intensity ratio of GluTub stained microtubules in the caudal fin (see region of interest, ROT, arrowhead; left panel) which demonstrated increased detyrosination following 96 hr, but not 3 hr, of paclitaxel treatment compared with vehicle (0.05% DMSO) controls (right panel). GluTub staining was normalized to Hoechst33342 nuclear stain within each fin. FIG. 1E is enhanced GluTub labelling in linear mesenchymal cells at 2dpf following 3 hr vehicle treatment (left column), which was absent in mesenchymal cells in the presence of paclitaxel (right column) but instead cutaneous sensory axons (dotted linear structures) were labelled. Diffuse dMT staining was present in the caudal fin following 96 hr vehicle treatment, whereas 96 hr paclitaxel treatment showed fasciculated dMTs (dfMTs) in keratinocytes (thin arrows). FIG. 1F is GluTub staining in a 2dpf zebrafish embryo. Mesenchymal cells (thin arrows) were strongly labeled in both 3 hr vehicle (0.05% DMSO; top row left image) and paclitaxel treated fish (top row right image). Axonal dMTs were evident following paclitaxel treatment (thick arrows) but rarely present in vehicle controls at 3 hours. Keratinocyte plasma membranes were visualized with tp63:GFP-CAAX (green) demonstrating mesenchymal cell microtubules span multiple keratinocyte cell diameters. FIG. 1G demonstrates that at 6dpf, dMTs were present in mesenchymal cells and axons of vehicle-treated fish. Following 96 hr paclitaxel treatment (right panel), dMTs were fasciculated in individual keratinocytes in the caudal fin (thin arrow—top arrow of the right image) and most prominently at the fin edge (oval dotted line). Cutaneous axons (thick arrows in left panel—control, and bottom left of the right panel—paclitaxel) displayed a weak punctate detyrosination pattern in the control animal, which was more prominent following paclitaxel treatment. FIG. 1H are polar plots depicting orientation and shape of dMTs in the caudal fin (180°=proximal; 0°=distal fin) (n=4 animals/plot). FIG. 1I demonstrates increased percent animals with dfMTs after 48 and 96 hr paclitaxel but not vehicle treatment (n≥15 animals/group). FIG. 1J demonstrates increased caudal fin keratinocytes per 100 μm² with dfMTs following 22 μM paclitaxel treatment, while few keratinocytes harbor dfMTs when animals were treated with 100 nM paclitaxel (n≥17 animals/group). Quantification of the fasciculation width of dfMTs in FIG. 1K demonstrates an increase in animals treated with 100 nM paclitaxel for 96 hr compared with 22 μM paclitaxel treatment (n=5 animals/group). FIG. 1L demonstrates a single injection of 20 mg/kg paclitaxel into mice at 5 weeks caused dfMT and rosette formation 48 hr after the injection, assessed with alpha-tubulin staining. dfMT formation is evident in the suprabasal epidermis.

FIG. 2A through FIG. 2I demonstrate mechanotransduction activation of Nox1 upstream of MMP-13 and cutaneous axon degeneration. FIG. 2A shows that keratinocyte mitochondria showed subtle changes in the reactive oxygen species (H₂O₂) following paclitaxel treatment. Mitochondria in basal keratinocytes were labelled with tp63:HyPer-mito to detect H₂O₂ dependent oxidation of HyPer with ratiometric imaging. Unoxidized hydrogen peroxide sensor (HyPer) was detected when HyPer is excited at 420 nm. Oxidized HyPer was detected when excited at 505 nm. Quantification of oxidized:unoxidized HyPer ratios showed that oxidation was not different when comparing 3 hr and 48 hr vehicle versus 22 μM paclitaxel treatment. FIG. 2B is a summary of HyPer-mito ratio at 3 hr and 48 hour of vehicle versus paclitaxel treatment. FIG. 2C are quantitative PCR (qPCR) results showing increased Nox1 expression in 4dpf zebrafish following 48 hr paclitaxel treatment (i.e. axon degeneration onset) (n=3 biological replicates with 10 animals per replicate). FIG. 2D demonstrates that Nox1 was distributed in the cytoplasm and nucleus of caudal fin keratinocytes when fish were treated with vehicle (0.05% DMSO) and translocated to the plasma membrane and clusters in the nucleus following prolonged paclitaxel treatment. Membrane:cytoplasmic ratio of Nox1 staining (left, lower panel) increased upon continued paclitaxel treatment (n≥6 animals/group), similar to the nuclear:cytoplasmic ratio (n≥9 animals/group) (right, lower panel). FIG. 2E showing Tg(tp63:GFP-CAAX) transgenic animals with fluorescently labelled basal keratinocyte plasma membranes used to measure keratinocyte length and width by comparing medial and edge (dash line) keratinocytes. Quantifications were performed before and after zebrafish stretch on the stretcher, which revealed that fin edge keratinocytes increased in length but not in width whereas no effect was seen for medial keratinocytes. FIG. 2F demonstrates immunofluorescence staining for alpha tubulin, showing microtubule stretching along the fin edge in wildtype (left 2 panels) and homozygous cyba−/− fish (right 2 panels) treated for 48 hours with vehicle. Microtubule stretching at the fin edge was less evident following paclitaxel treatment (white arrows—top panels vehicle treatment, versus yellow arrows—bottom panels paclitaxel treatment). FIG. 2G shows ratiometric HyPer imaging and summary oxidation data from 5 min pre-stretch to 90 min post-stretch in wildtype and cyba−/− mutants treated with vehicle or paclitaxel illustrated by the top-most panel. Paclitaxel treatment resulted in faster activation of H₂O₂ production in wildtype animals (right upper graph) compared with vehicle-treated controls (left upper graph), whereas HyPer oxidation was largely reduced or absent in vehicle (left lower graph) and paclitaxel-treated cyba−/− mutants (right lower graph), respectively (n=5 animals/group). FIG. 2H shows that prolonged paclitaxel, but not vehicle, treatment reduced the number of axon branches in the caudal fin of wildtype but not cyba−/− fish (n=6-9 animals/group). Stretch was associated with axon degeneration in wildtype fish treated with paclitaxel, but not cyba−/− fish. FIG. 2I is qPCR data showing enhanced mmp13a (MMP13 homolog) expression in wildtype fish treated with paclitaxel, but not in cyba−/− mutants (n=10-20 animals/group in 3 biological replicates).

FIG. 3A through FIG. 3M show that paclitaxel treatment induces cell cycle genes in the skin of mice and patients with CIPN. FIG. 3A shows a treatment scheme for mouse RNAseq analysis (n=4 animals per group). FIG. 3B is a heatmap showing up (red) and down (blue) regulated genes. FIG. 3C is gProfiler analysis at peak neuropathy day 7 (D7) revealing Gene Ontology (GO) terms for extracellular matrix and collagen-related processes, and cell cycle regulation. FIG. 3D is STRING interactome analysis of genes in FIG. 3C identifying a major cluster for cell cycle genes (magenta, blue), and proteins involved in disulfide bond formation (yellow). FIG. 3E is validation of mouse RNAseq data confirming the upregulation of spindle checkpoint regulators (Plk1m Cdc20, Ndc80, Bub1, Dlgap5) following paclitaxel treatment relative to vehicle controls, with the highest expression increase at D7 during peak neuropathy. FIG. 3F shows that STRING interactome analysis predicted the interactions of these genes with Kif11 and other mitotic genes. FIG. 3G are normalized gene counts for upregulated Kif genes following paclitaxel treatment (shown are days 4, 7, 11, and 23 of treatment). FIG. 3H are normalized gene counts for Nox genes and their regulators detected in the skin (shown are days 4, 7, 11, and 23 of treatment). FIG. 3I through FIG. 3K show RNAseq using human lower leg skin from 3 CIPN patients (CIPN5, 31, 35 weeks) and 3 control subjects (CTRL1-3). FIG. 3I is a heatmap and PCA plot revealing the separation of CIPN patients from the controls. FIG. 3J shows relative gene expression of upregulated KIF genes (left to right: KIF11, KIF12, KIF13B, KIF17, KIF1C, KIF26A). FIG. 3L shows that NOX1 was most strongly induced and showed great variability together with NOX4 and CYBB. FIG. 3K shows STRING network analysis which revealed KIF11 as a central hub for co-upregulated KIF genes. FIG. 3M is iDEP.96 analysis of the 1200 most variable differentially expressed genes showing three distinct clusters. The first cluster (A, blue) harbours genes that are downregulated in the CIPN patients. Cluster B (yellow) harbours genes that are upregulated in the patient diagnosed with CIPN 5 weeks prior. Cluster C (purple) harbours genes that are upregulated in the patients diagnosed with CIPN 35 and 31 weeks prior).

FIG. 4A through FIG. 4J demonstrate that paclitaxel induces Eg5 expression and Eg5-dependent dfMT and nuclear X-ROS formation. FIG. 4A is Eg5 immunofluorescence staining showing punctate staining in proximal caudal fin keratinocytes of vehicle treated animals. Staining along the fin edge (white dashed line) is absent. Paclitaxel treatment promoted Eg5 activation in the proximal fin (white arrows) and in fin edge keratinocytes leading to aster-like formations (white dashed line, thick white arrow—bottom arrow of bottom right panel). FIG. 4B shows that EMD534085 co-administration with 100 nM and 22 μM paclitaxel rescued microtubule fasciculation but not detyrosination. NM=lateral line neuromast. kif11 CRISPR knockout prevented detyrosination and fasciculation of MTs. FIG. 4C shows percentage of animals with dfMTs/keratinocyte was reduced when paclitaxel is administered in combination with EMD534085 or in kif11 CRISPR knockout fish (n≥23 animals/group). FIG. 4D shows that EMD534085 treatment or kif11 CRISPR knockout in presence of paclitaxel reduced the number of keratinocytes with dfMTs per animal compared with paclitaxel alone (n≥21 animals per group). FIG. 4E shows zebrafish kif11 CRISPR knockout without Eg5 induction in caudal fin following paclitaxel treatment. Vehicle (top) and paclitaxel (bottom) treatment for 96 hours did not induce Eg5 expression detected with an Eg5 specific antibody using immunofluorescence staining. FIG. 4F shows normalized intensity ratios of GluTub suggest that kif11 CRISPR knockout rescued microtubule detyrosination induced by paclitaxel. FIG. 4G shows that paclitaxel induced total and phospho-Eg5 in mouse suprabasal keratinocytes. Mice were i.p. injected once with either 0.9% NaCl (vehicle) or 30 mg/kg paclitaxel, followed by fixation 48 hours post injection. Alpha-tubulin antibody staining was used as counterstain. DAPI was used to depict nuclei. Top panel: Weak Eg5 expression in the vehicle control epidermis (left) but strong induction of Eg5 following paclitaxel treatment (right). Lower panel: Eg5 phosphorylation was absent in the skin following vehicle injection (left). Paclitaxel injection induced Eg5 phosphorylation in basal and suprabasal keratinocytes. Ep: Epidermis; hpi: hours post injection. FIG. 4H and FIG. 4I show that Nox1 nuclear but not plasma membrane translocation induced by 96 hr paclitaxel treatment was rescued with EMD534085 co-administration. FIG. 4J shows that nuclear HyPer oxidation induced by paclitaxel treatment was rescued with EMD534085 co-administration.

FIG. 5A through FIG. 5D show detyrosinated fasciculated microtubules associate with nuclei. FIG. 5A shows that dfMTs detected with anti-GluTub staining associate with a keratinocyte nucleus labelled with Hoechst33342 (left image). 3D reconstruction of the same image using Imaris (right image). FIG. 5B shows that 3D-rendered dfMTs pinch off nuclear content (left and right, thin arrow) and perforated the nucleus (right, thick arrows). FIG. 5C shows nuclear volume of keratinocytes following 96 hours of 22 μM paclitaxel treatment. The nuclear volume of keratinocytes with dfMTs appearance was significantly larger than the nuclear volume of keratinocytes with wildtype appearance (n≥5 animals/group). FIG. 5D shows decreased nuclear sphericity of keratinocytes with dfMTs appearance (n≥5 animals/group). DFL-MT: detyrosinated fasciculated looped microtubule.

FIG. 6A through FIG. 6F demonstrate that dfMTs modulate mitosis but not apoptosis. FIG. 6A shows keratinocyte divisions at 3 and 6dpf (96 hr treatment) assessed by time-lapse imaging in caudal fins of Tg(h2a:h2a-GFP) fish (n=28 at 3dpf; n=12 at 6dpf). FIG. 6B is percent animals without and ≥1 mitotic divisions/12 hr (n≥9 animals/group). FIG. 6C shows that EMD534085 combination with 22 μM paclitaxel increased cell divisions/animal compared with paclitaxel treatment (n≥8 animals/group). FIG. 6D shows that 96 hr treatment with EMD534085 and 22 μM paclitaxel increased cell death (n≥8 animals/group). FIG. 6E is time-lapse imaging of keratinocyte cell death in caudal fin. Caudal fin keratinocyte nuclei were visualized in a Tg(h2a:h2a-GFP) zebrafish caudal fin at 6dpf. The fish was treated with paclitaxel+EMD534085 for 96 hr. The intact keratinocyte nucleus (top, arrow) condenses within 15 minutes (middle, arrow) and subsequently fragments (bottom, arrow), indicative of cell death. Right panels show higher magnification images. FIG. 6F is a model for Eg5-dependent dfMT formation and downstream events: paclitaxel activated Eg5 as part of the cell cycle checkpoint, however, independent of mitosis in interphase keratinocytes leading to crosslinking and fasciculation of long-term stabilized, detyrosinated microtubules. dfMTs constrained the nucleus and promoted Nox1 nuclear accumulation and X-ROS formation upstream of mmp13 induction.

FIG. 7A through FIG. 7H demonstrate that paclitaxel modulated microtubule growth dynamics in keratinocytes independent of Eg5. Microtubule growth dynamics were captured and quantified in caudal fin keratinocytes expressing tp63:EB3-GFP. FIG. 7A and FIG. 7B show keratinocytes with uniform EB3-GFP localization along the microtubule lattice. Paclitaxel treatment increased the number of keratinocytes with uniform EB3-GFP labelling (n=5 animals). FIG. 7C shows increased straightness of microtubule growth tracks following 48 and 96 hr paclitaxel treatment (1=straight, 0=curved). FIG. 7D are kymographs of growing microtubules generated with MTrackJ shows more rapid depolymerization in the presence of paclitaxel, consistent with quantifications in FIG. 7F. FIG. 7E shows that decreased comet velocity following 3 hr paclitaxel treatment was not rescued by co-administration of EMD534085 (n≥8 animals/group). FIG. 7F shows that decreased track length upon 3 hr paclitaxel treatment was not rescued by co-administration of EMD534085 (n≥8 animals/group). FIG. 7G shows that comet duration was not changed for any of the treatments (n≥8 animals/group). FIG. 7H shows microtubule average growth distance in μm over 50 seconds in epidermal keratinocytes in the caudal fin of 6dpf larval zebrafish treated for 96 hr either with vehicle, EMD534085, 22 μm paclitaxel, or 22 μM paclitaxel in combination with EMD534085.

FIG. 8A through FIG. 8H show microtubule behavior in genetically stabilized microtubules. FIG. 8A shows microtubule network in CMV-Tau-Bfp2 expressing keratinocytes following 96 hr vehicle treatment. Polar plot shows tracings to outline microtubule conformations (n=5 animals/plot). FIG. 8B shows that 96 hr paclitaxel treatment induced linearization of Tau-Bfp2-stabilized microtubules. The polar plot validates increased linearity (n≥4 animals/plot). FIG. 8C demonstrates that microtubule track straightness in keratinocytes expressing Tau-Bfp2 with and without 96 hr paclitaxel treatment showed enhanced linearization in the presence of paclitaxel: Straightness (1=straight, 0=curved) (n≥5 animals/group). FIG. 8D is 3D reconstruction of EB3-GFP plus-ends in a keratinocyte following 96 hr paclitaxel treatment. FIG. 8E shows more than 80% of EB3-GFP plus-ends colocalized with Tau-Bfp2 regardless of low or high Tau expression. Paclitaxel treatment for 96 hr further increased co-localization (n≥5 animals/group). FIG. 8F shows that paclitaxel treatment for 96 hr increased EB3-GFP velocity in the presence of Tau-Bfp2. FIG. 8G and FIG. 8H demonstrates that colocalized EB3-GFP and Tau-Bfp2 microtubules (arrow) showed overlapping growth tracks when traced along curvatures.

FIG. 9A through FIG. 9I show that Eg5 inhibition rescued and keratinocyte-specific overexpression induced paclitaxel neurotoxicity. FIG. 9A shows degeneration of cutaneous axons detected with anti-acetylated tubulin antibody staining following 96 hr treatment with 22 μM paclitaxel, but not when treated with 100 nM paclitaxel or 22 μM paclitaxel+EMD534085 (n≥11 animals/group). FIG. 9B is quantification showing an increased axon branch number for paclitaxel+EMD534085, whereas kif11 CRISPR knockout rescues axon branch number to wildtype levels (n=6 animals/group). FIG. 9C shows that paclitaxel induced axonal detyrosination and keratinocytes acetylation. Increased axonal dMT formation (thin white arrows) and keratinocyte-specific microtubule acetylation at lysine position 40 (K40) (thick arrows) were produced following 22 μM paclitaxel treatment for 96 hours with and without EMD534085. FIG. 9D is quantification of normalized acetylation intensity showing an increase in keratinocyte microtubule acetylation after 96 hr paclitaxel treatment with and without EMD534085. FIG. 9E shows increased axonal dMTs along axon segments in distal caudal fin seen with 22 μM paclitaxel compared to EMD534085 (n=5-7 animals/group). FIG. 9F top panel: Co-expression of is11:Gal4VP16_14×UAS-tdTomato (magenta) in axons and tp63:kif11-AcGFP (blue) in few keratinocytes that were not in contact with cutaneous branches did not promote axon degeneration. Bottom panel: Axon degeneration was prominent when tp63:kif11-AcGFP expressing basal keratinocytes were abundant and established contact with cutaneous branches. FIG. 9G is magnification of FIG. 9F showing nuclear Eg5 and weaker cytoplasmic localization in 6dpf zebrafish expressing tp63:kif11-AcGFP. FIG. 9H is quantification of axon degeneration showing that low presence of keratinocytes expressing kif11-AcGFP was insufficient to induce axon degeneration. FIG. 9I is a model showing paclitaxel-dependent Eg5 induction leading to dfMT formation and Nox1-dependent mmp13 expression in keratinocytes, which promoted ECM degradation and axon degeneration.

FIG. 10 is a depiction of the forward primer used for sequencing a single zebrafish and yielded deletions in transiently injected embryos in experiments involving CRISPR.

FIG. 11A through FIG. 11C show that EMD534085 reduced detyrosinated microtubules (dMT) caused by paclitaxel treatment. FIG. 11A shows the treatment plan for 12 week-old mice receiving intraperitoneal injections of vehicle (0.5% DMSO/1×PBS), paclitaxel (24 mg/kg; one injection), or paclitaxel plus EG5 inhibitor, EMD534085 (20 mg/kg; 3 injections: 1 before, during, and after paclitaxel). The mice were then sacrificed 24 hours after the last injection and the skin of the hind paw pad was collected. FIG. 11B show images of collected skin tissues stained with anti-GluTub (to detect detyrosinated microtubules) and DAPI to detect nuclei. The arrows in the circle point to brittle skin in the stratum spinosum. The other arrows point to cells that have increased detyrosination. FIG. 11C is the quantification of number of cells with dMT per 250 μm² area with each treatment.

### DETAILED DESCRIPTION OF THE DISCLOSURE

Provided herein are compositions and methods for the treatment of chemotherapy-induced peripheral neuropathy (CIPN).

It is to be understood that the particular aspects of the specification are described herein are not limited to specific embodiments presented and can vary. It also will be understood that the terminology used herein is for the purpose of describing particular aspects only and, unless specifically defined herein, is not intended to be limiting. Moreover, particular embodiments disclosed herein can be combined with other embodiments disclosed herein, as would be recognized by a skilled person, without limitation

### DEFINITIONS

Throughout this specification, unless the context specifically indicates otherwise, the terms “comprise” and “include” and variations thereof (e.g., “comprises,” “comprising,” “includes,” and “including”) are understood to indicate the inclusion of a stated component, feature, element, or step or group of components, features, elements or steps but not the exclusion of any other component, feature, element, or step or group of components, features, elements, or steps. Any of the terms “comprising”, “consisting essentially of”, and “consisting of” can be replaced with either of the other two terms, while retaining their ordinary meanings

As used herein, the singular forms “a,” “an,” and “the” include plural referents unless the context clearly indicates otherwise.

Unless otherwise indicated or otherwise evident from the context and understanding of one of ordinary skill in the art, values herein that are expressed as ranges can assume any specific value or sub-range within the stated ranges in different embodiments of the disclosure, to the tenth of the unit of the lower limit of the range, unless the context clearly dictates otherwise.

As used herein and in the drawings, ranges and amounts can be expressed as “about” a particular value or range. About also includes the exact amount. For example, “about 5%” means “about 5%” and also “5%.” The term “about” can also refer to ±10% of a given value or range of values. Therefore, about 5% also means 4.5%-5.5%, for example.

As used herein, the terms “or” and “and/or” are utilized to describe multiple components in combination or exclusive of one another. For example, “x, y, and/or z” can refer to “x” alone, “y” alone, “z” alone, “x, y, and z,” “(x and y) or z,” “x or (y and z),” or “x or y or z.”

“Subject” or “patient” as used herein are used interchangeably and refer to a warm-blooded animal such as a mammal, preferably a human, which is afflicted with, or has the potential to be afflicted with cancer as described herein. The subject can be a human patient that was at risk for, or suffering from, peripheral neuropathy induced by chemotherapy treatment.

“Contacting” as used herein includes the physical contact of at least one substance to another substance.

“Express” or “expression” as used herein refers to transcription and translation of a nucleic acid coding sequence resulting in production of the encoded polypeptide.

### Compositions

“Pharmaceutical composition” as used herein refers to a composition that includes one or more therapeutic agents, such as an Eg5 inhibitor, a pharmaceutically acceptable carrier, a solvent, an adjuvant, and/or a diluent, or any combination thereof. The exact nature of the carrier, solvent, adjuvant, or diluent will depend upon the desired use of the composition (e.g., route of administration), and can range from being suitable or acceptable for veterinary uses to being suitable or acceptable for human use.

The terms “chemotherapy” and “chemotherapeutic agents,” as used herein refer to chemicals/drugs that are used to treat cancer. In a preferred embodiment the therapeutic agent is used to treat CIPN. The “therapeutic agents,” described herein or compositions thereof, will generally be used in an amount effective to achieve the intended result, for example, to treat cancer or to treat CIPN. Some chemotherapeutic agents described herein are capable of stabilizing the cellular microtubule. One skill in the art will readily recognize that chemotherapy agents can be in multiple forms including, but not limited to, capsules, tablets, injectables, or patches.

“Chemotherapy-induced peripheral neuropathy” (CIPN) is a common side effect wherein the chemotherapy causes damages or degeneration of peripheral neurons, especially sensory neurons. CIPN symptoms include tingling, pain, decreased sensation, increased sensitivity (to touch, temperature, pressure and pain), and muscle weakness.

“Stabilized microtubule” as used herein refers to a shift in the equilibrium of tubulin polymer that made up the microtubule from the soluble to the polymerized form. A preferred mechanism of microtubule stabilization occurs post-translationally, by detyrosination (dMT)—a process to remove the terminal tyrosine on α-tubulin by tubulin carboxypeptidase (Hallak et al., 1977, FEBS letters 73: 147-150), thereby exposing a glutamate residue (Nieuwenhuis et al., 2019, Trends Cell Biol 29: 80-92). Chemotherapeutic agents that are microtubule stabilizers include paclitaxel, docetaxel, cabazitaxel, TPI-287, and albumin paclitaxel. The structures and commercial sources/publications of these compounds are provided below.

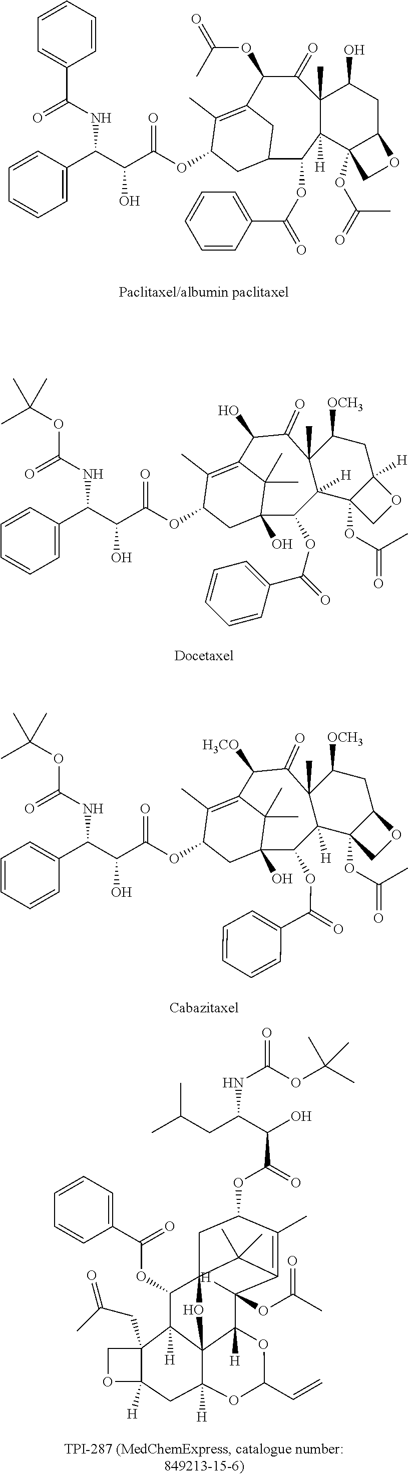

As used herein “inhibitors” refer to biologically active compounds that reduce a protein's function or expression.

As used herein “Eg5” refers to kinesin-5 protein, which can be expressed in mammals, zebrafish, or other animals. For the purpose of this application, both Eg5 and EG5 mean kinesin-5 protein.

As described herein, Eg5 inhibitors are capable of reducing detyrosination of microtubules in keratinocytes and degeneration of axons innervating the keratinocytes caused by chemotherapeutic drugs such as paclitaxel treatment. In some embodiments, such Eg5 inhibitors include, but are not limited to, monastrol, ispinesib, (+)-S-trityle-L-cysteine (TriC), BRD9876, filanesib (ARRY-520), litronesib (LY2523355), AZD4877, SB-743921, MK0731, 4SC-205, ARQ 621, and EMD534085. The structures and commercial sources/publications of these compounds are provided below. In some embodiments, therapeutic effects produced by Eg5 inhibitors can be achieved by reducing Eg5 gene expression, inter alia, by genome-editing tools such as CRISPR-Cas9, TALE nucleases, zinc-finger nucleases, and the like that target Eg5 specifically.

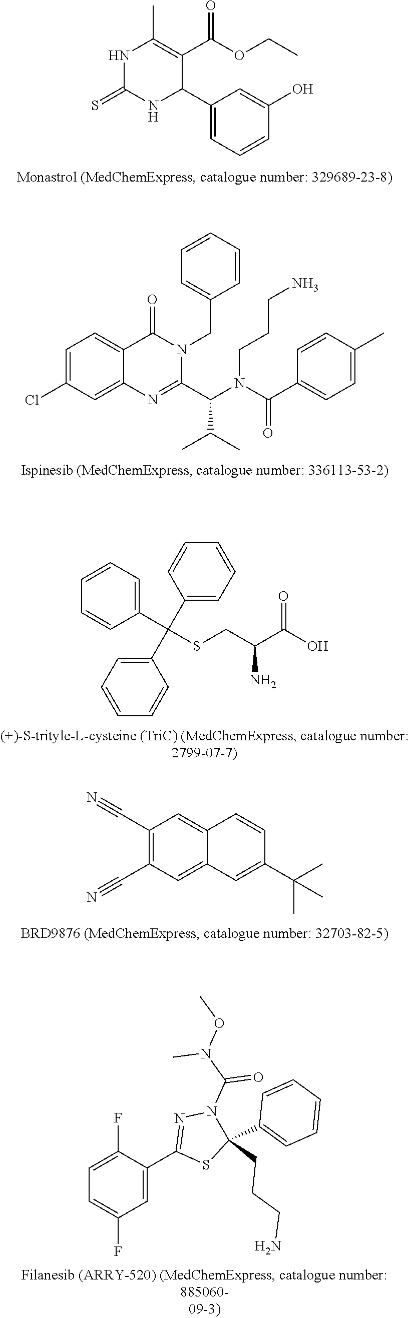 [SMILES: CCOC(=O)C1=C(C)NC(=S)NC1c1cccc(O)c1 ; Cc1ccc(C(=O)N(CCCN)[C@@H](c2nc3cc(Cl)ccc3c(=O)n2Cc2ccccc2)C(C)C)cc1 ; N[C@@H](CSC(c1ccccc1)(c1ccccc1)c1ccccc1)C(=O)O ; CC(C)(C)c1ccc2cc(C#N)c(C#N)cc2c1 ; CON(C)C(=O)N1N=C(c2cc(F)ccc2F)S[C@@]1(CCCN)c1ccccc1]

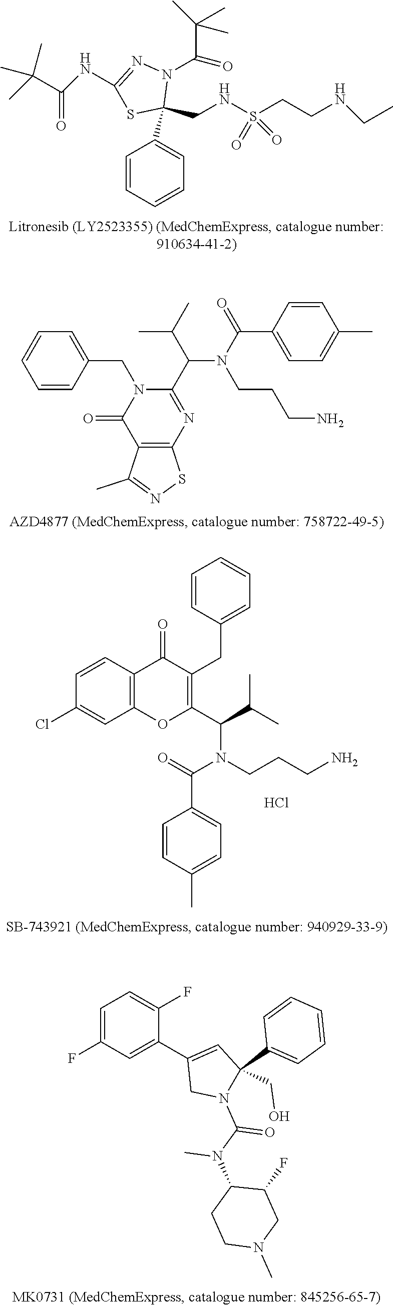 [SMILES: CCNCCS(=O)(=O)NC[C@@]1(c2ccccc2)SC(NC(=O)C(C)(C)C)=NN1C(=O)C(C)(C)C ; Cc1ccc(C(=O)N(CCCN)C(c2nc3snc(C)c3c(=O)n2Cc2ccccc2)C(C)C)cc1 ; Cc1ccc(C(=O)N(CCCN)[C@@H](c2oc3cc(Cl)ccc3c(=O)c2Cc2ccccc2)C(C)C)cc1 ; Cl ; CN1CC[C@H](N(C)C(=O)N2CC(c3cc(F)ccc3F)=C[C@@]2(CO)c2ccccc2)[C@H](F)C1]

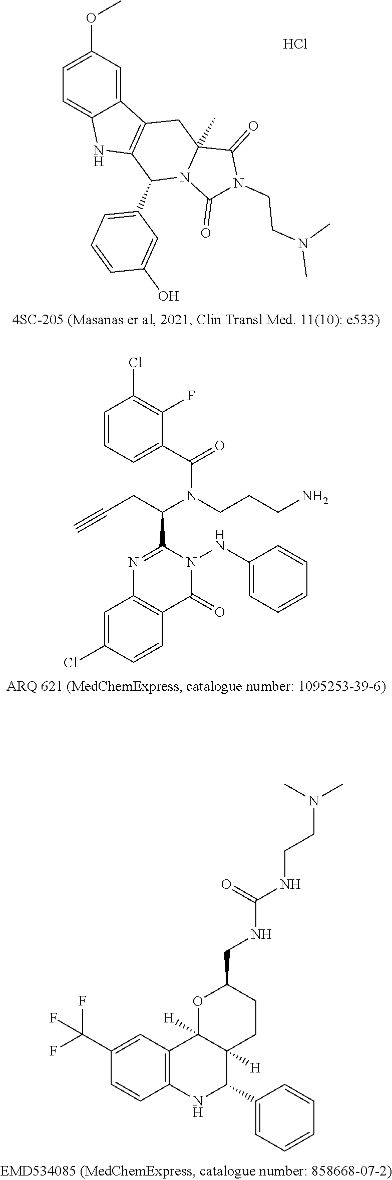

Examples of compositions appropriate for such therapeutic applications include preparations for parenteral, subcutaneous, transdermal, intradermal, intramuscular, intravenous (e.g., injectable), intrajoint, intratendon, intraligament, intrasynovial, extrasynovial, or intratracheal administration, such as sterile suspensions, emulsions, and aerosols. In some cases, pharmaceutical compositions appropriate for therapeutic applications can be in admixture with one or more pharmaceutically acceptable excipients, diluents, or carriers such as sterile water, physiological saline, glucose, or the like. For example, the compounds described herein can be administered to a subject as a pharmaceutical composition comprising a carrier solution. In some cases, pharmaceutical compositions are lyophilized. In other cases, pharmaceutical compositions as provided herein contain auxiliary substances such as wetting or emulsifying agents, pH buffering agents, gelling or viscosity enhancing additives, preservatives, flavoring agents, colors, and the like, depending upon the route of administration and the preparation desired. The pharmaceutical compositions can be formulated according to conventional pharmaceutical practice (see, e.g., Remington: The Science and Practice of Pharmacy, 20th edition, 2000, ed. A. R. Gennaro, Lippincott Williams & Wilkins, Philadelphia, and Encyclopedia of Pharmaceutical Technology, eds. J. Swarbrick and J. C. Boylan, 1988-1999, Marcel Dekker, New York).

A variety of dosage schedules are contemplated by this disclosure. For example, a subject can be dosed monthly, every other week, weekly, daily, or multiple times per day. Dosage amounts and dosing frequency can vary based on the dosage form and/or route of administration, and the age, weight, sex, and/or severity of the subject's disease.

“Therapeutic benefit” refers to the rebuilding and/or remodeling of damaged tissue thereby eradicating or ameliorating one or more of the symptoms associated with the injury or surgical recovery such that a subject being treated with the therapeutic agent reports an improvement in feeling or condition, notwithstanding that the subject can still have incomplete healing or injury resolution.

### Pharmaceutical Formulations and Methods of Treatment

In exemplary embodiments, an Eg5 inhibitor is provided to a subject as part of a pharmaceutical composition comprising an inhibitor, a chemotherapeutic agent, and a pharmaceutically acceptable carrier, wherein the chemotherapeutic agent is a microtubule stabilizer. The pharmaceutical composition can be delivered to the subject locally or systemically via topical administration, intravascular administration, such as intravenous, intramuscular, or intra-arterial administration, intraperitoneal administration, and the like.

In some embodiments, the Eg5 inhibitor is administered before, during, and/or after the administration of the chemotherapeutic agent. The Eg5 inhibitor can be included with the chemotherapeutic agent in one injection to be given to the patients; or two injections—one including Eg5 inhibitor and one including the chemotherapeutic agent can be given.

As used herein, “treatment” refers to the clinical intervention made in response to a disease, disorder, or physiological condition of the subject or to which a subject can be susceptible. The aim of treatment includes the alleviation or prevention of symptoms, slowing or stopping the progression or worsening of a disease, disorder, or condition and/or the remission of the disease, disorder, or condition.

The terms “prevent” and “preventing” refer to prophylactic or preventive measures intended to inhibit undesirable physiological changes or detrimental progression of a condition.

The terms “effective amount” or “therapeutically effective amount” refer to an amount sufficient to effect beneficial or desirable biological and/or clinical results. In other words, a “therapeutically effective” amount is an amount that will provide some alleviation, mitigation, or decrease in at least one clinical symptom in the subject.

Unless otherwise defined, all technical terms used herein have the same meaning as commonly understood by a person of ordinary skill in the art to which this disclosure belongs.

For the purposes of promoting an understanding of the principles of the disclosure, reference will now be made to embodiments and specific language will be used to describe the same. It will nevertheless be understood that no limitation of the scope of the disclosure was thereby intended, such alteration and further modifications of the disclosure as illustrated herein, being contemplated as would normally occur to one skilled in the art to which the disclosure relates.

Various exemplary embodiments of compositions and methods according to this invention are now described in the following non-limiting Examples. The Examples are offered for illustrative purposes only and are not intended to limit the scope of this invention in any way. Indeed, various modifications of the invention in addition to those shown and described herein will become apparent to those skilled in the art from the foregoing description and the following examples and fall within the scope of the appended claims.

### EXAMPLES

### Materials and Methods

### Zebrafish Husbandry and Transgenic Lines

Wildtype strains of Zebrafish (Nacre/mitfa), Tuebingen and AB strains were purchased from the Zebrafish International Resource Center (ZIRC) and embryos raised and bred according to NIH guidelines. Tuebingen wildtype fish were used for CRISPR studies. Nacre fish were used for membrane labelling of keratinocytes in tp63:GFP-CAAX. All remaining analyses were performed in AB fish. Animals were handled in strict accordance with good animal practices as approved by the appropriate IACUC committees (MDI Biological Laboratory IACUC number #A13-20; University of Miami's #A-3224-01 and AALAC accreditation site: 001069). Zebrafish eggs were collected in a strainer and rinsed with deionized water, and then transferred into Petri dishes with Embryo medium (Instant Ocean salt water+methylene blue, Zebrafish book protocol (Westerfield, 2007, University of Oregon Press, Eugene 5^{th} edition, incorporated by reference herein in its entirety, incorporated by reference herein in its entirety) or Ringers solution. Following overnight incubation at 28.5 °C, embryos were cleaned and fresh Ringer's solution with phenol-thio-urea was added for further incubation. Embryos were kept in a 14:10 hr light/dark cycle.

Transgenic lines: Tg(h2a:h2a-GFP, Cat. No. ZL1087) transgenic fish were obtained from the Zebrafish International Resource Center (ZIRC). tp63:GFP-CAAX fish were previously published (Lisse et al., 2016, Proc. Natl. Acad. Sci. USA 113: E2189-2198).

### Plasmids

CREST3:EB3-GFP: pCS2_CMV:EB3-GFP was a gift from the Koester Lab (University of Braunschweig, Germany) (pCS2_CMV:EB3-GFP sequence is from PMID: 12684451, for CREST3-EB3-GFP see SEQ ID NO: 25). The CMV promoter was removed from pCS2 by digestion with HindIII, followed by Klenow fragment incubation to create blunt-ends, purification and subsequent digestion done with SalI. The CREST3 promoter in Tol2_CREST3:Gal4VP16_14×UAS-GFP (gift from Alvaro Sagasti, UCLA; and see Rieger et al., 2011, PLoS Biol. 9: e1000621) was digested with EcoRV (created blunt-end site) and Sal. The gel-purified fragment was ligated into the pCS2 EB3-GFP plasmid overnight at 16 °C and transformed into Top10 cells for purification and injection

A construct identified as tp63:EB3-GFP (SEQ ID NO: 26) was generated by removing the CMV promoter as above. The tp63 promoter was removed by digestion of pBSK-JC_T2_tp63_Gal4VP16_GFP-5×UAS-MCS (SEQ ID NO: 30) with ApaI and AvrII, followed by Klenow treatment to generate blunt ends. Following gel purification, the tp63 promoter fragment was ligated into pCS2-EB3-GFP, as above. Both constructs were sequenced and further verified by in vivo imaging.

A construct identified as tp63:kif11-AcGFP (SEQ ID NO: 27) was generated by PCR amplification of zebrafish kif11 from cDNA (Horizon Discovery, Clone ID: 3815942) for ligation into the pAcGFP-N1 (Takara, Cat. No. 632501) fusion vector. The following primers were used: Fwd 5′-AAGGCCTCTGTCGACCATGGCATCATCACAAGTAC-3′ (SEQ ID NO: 1) and rev 5′-AGAATTCGCAAGCTTATTCTGACATCTGAGTGGAAGT-3′ (SEQ ID NO: 2) and Q5 P Polymerase (NEBNext® High-Fidelity 2×PCR Master Mix). This was followed by PCR purification (QIAGEN PCR Purification kit) and ligation of the amplicon into pAcGFP-N1. The plasmid was transformed into One-Shot Top10 cells (ThermoScientific, Cat. No. C404010) and plasmids were purified from colony minipreps using a MiniPrep kit (QIAGEN). The insert (kif11-AcGFP) was subsequently amplified from using the following primers: Fwd 5′-GGATCCTTATGGCATCATCACAAGT-3′ (SEQ ID NO: 3) and Rev 5′-GCGGCCGCTTCTTGTACAGCTC-3′ (SEQ ID NO: 4) that added BamHI/NotI restriction sites. KXIG:tp63:AcGFP (Lisse et al., 2016, Proc Natl Acad Sci USA 113(15): E2189-98) was digested with BamHI/NotI to remove AcGFP with gel extraction (QIAGEN) and BamHI_kif11-AcGFP_NotI was inserted via ligation using T4 DNA ligase (Promega, Cat. No M180A) at room temperature for 2 hours. The ligation reaction was transformed into One-Shot Top10 cells and colonies were screened via PCR for positive inserts. Plasmid DNA from positive colonies were isolated and purified using the QIAGEN MiniPrep kit and verified via sequencing. Positive plasmids were injected into zebrafish and verified for fluorescence, which was only visible if kif11 was cloned in-frame with AcGFP.

A construct identified as isl1:Gal4VP16_14×UAS-tdTomato (SEQ ID NO: 31) was provided to the laboratory. mTagBFP2-MAPTau-C-10 was provided by Michael Davidson (Addgene plasmid #55311; PMID: 22174863, RRID: Addgene_55311).

A construct identified as tp63:HyPer-CAAX: KXIG:tp63:AcGFP (Lisse et al., 2016, Proc Natl Acad Sci USA 113(15): E2189-98) was digested with BamHI/NotI (NEB) to excise AcGFP. Hyper was amplified from pHyPer-dMito (Evrogen) without the stop codon using the following primers: Fwd: 5′-3′ catttacctctgaagccacgggtttagtgaaccgtcag (SEQ ID NO: 5) and Rev: 5′-ttcctcctccAACCGCCTGTTTTAAAAC-3′ (SEQ ID NO: 6). The CAAX motif was amplified using the primers 5′-acaggcggttGGAGGAGGAAGATCTAAG-3′ (SEQ ID NO: 7) and 5′-tcgagctccaccgcggtggcAACACCCCTTGTATTACTG-3′ (SEQ ID NO: 8) from the pME-EGFP-CAAX vector (gift from Chi-Bin Chien). Both amplicons were purified using the PCR Purification kit (QIAGEN) and ligated via HiFi Assembly using the 2×HiFi Master mix (NEB, Cat. No. M0541) at 50 °C for 20 minutes. The assembly reaction was transformed into NEB 5-alpha competent cells provided with the HiFi Assembly kit (NEB, Cat. No. E5520) and colonies grown and purified via the Plasmid Miniprep kit (Qiagen) and subsequently sequenced.

A construct identified as tp63:HyPer-mito: KXIG:tp63:HyPer-mito was cloned by digesting tp63:AcGFP with BamHI/NotI (NEB) to excise AcGFP (tp63:Hyper-mito sequence is SEQ ID NO: 29). Hyper-mito, a hydrogen peroxide sensor specific to the mitochondria was amplified from pHyPer-dMito (Evrogen) using the following primers Fwd: 5′-catttacctctgaagccacgggtttagtgaaccgtcag-3′ (SEQ ID NO: 9) and Rev: 5′-tcgagctccaccgcggtggctaagatacattgatgagtttgg-3′ (SEQ ID NO: 10). The amplicon was ligated into the vector containing the tp63 promoter using the Hi-Fi Assembly Master mix (NEB) at 50 °C for 20 minutes. The assembly reaction was transformed into NEB 5-alpha competent cells and purified using the plasmid miniprep kit by Qiagen, and subsequently sequenced.

### Pharmacological Agents

Paclitaxel was purchased from Sigma-Aldrich (Cat No. T7402) and upon arrival stored as powder at 4 °C. A stock solution was prepared in 100% fresh DMSO to make 5.9 mM paclitaxel. The stock solution was divided into 20-30 μl aliquots and stored at −20 °C until use (maximal 6 months). Immediately prior to use, paclitaxel was diluted in Ringer's solution and added to dechorionated larval zebrafish. The larval fish were placed individually or in small groups into wells of a 12-well plate containing the treatment solutions. The control group was 0.05% DMSO. Plates were incubated at 28.5 °C and protected from light. Treatment lengths are indicated in the Drawings, Description of the Drawings, and Examples where appropriate. Paclitaxel and DMSO solutions were exchanged every 48 hr if longer incubations were used. EMD534085, Eg5 inhibitor (MedChemExpress, Cat. No. HY-15000) was diluted in 100% DMSO to make a 10 mM stock solution and stored at −20 °C. Immediately prior to use, the inhibitor was diluted to 25 μM and added at 4dpf, following two days of either vehicle or paclitaxel incubation. For Nox1 staining experiments, the inhibitor was added together with paclitaxel at 2dpf. The solutions were exchanged every 48 hr.

### Immunofluorescence/Zebrafish Immunofluorescence Staining

Larval fish were transferred into 20m glass vials and fixed in 4% paraformaldehyde (PFA)/1× Phosphate-buffered saline (PBS) for 1.5 hr at room temperature, with gentle rocking. Following a 5-minute incubation in 1×PBS+0.1% Tween-20 (PBST), zebrafish were permeabilized in 1×PBS+0.1% Triton X-100 for 10-30 min. Larval fish were transferred into 2 ml reaction tubes and incubated in blocking buffer (1×PBST+5% BSA) for 30 min at room temperature, with rocking. Larval fish were further incubated overnight in antibody solution (Millipore Sigma, Cat. No. AB3201, 1:300 rabbit anti-GluTub; Sigma, Cat No. T6793, 1:500 mouse anti-acetylated tubulin) in blocking buffer at 4 °C, on a rotator. The next morning, larval fish were transferred into 6-well plates and washed 4×15 min in 1×PBST at room temperature, with rocking. This was followed by incubation in secondary antibody (goat anti-rabbit Cy5, Abcam, Cat. No. ab97077; goat anti-mouse Cy3, Abcam, Cat. No. ab97035) and 1:10,000 Hoechst 33342 (ThermoFisher Scientific, Cat. No. 62249, 20 mM solution) in blocking buffer for 1 hr at room temperature, with rocking. Fish were covered to avoid bleaching. Fish were washed for 4×15 min in 1×PBST at room temperature, with rocking, and immediately mounted for imaging on glass bottom petri dishes (Spectrum Laboratory Products, Cat. No. 750-10403-UE) using 1% agarose (Thermo Fisher Scientific, Cat. No. 16520050). A 1:200 dilution was used for mouse alpha-tubulin antibody (Proteintech, Cat. No. 66031-1) and a 1:1,000 dilution of donkey anti-mouse IgG Alexa488 (ThermoFisher Scientific, Cat. No. A-21202) for secondary detection. For Nox1 immunostaining, a 1:300 dilution of primary antibody (Anti-NOX1 Rabbit Polyclonal Antibody, Avantar, Cat. No. 102164-796) and a 1:1,000 dilution of secondary goat anti-rabbit Cy2 (Abcam, Cat. No. ab6940) antibody was used. Eg5 antibody (Abcam, Cat. No. 61199) was used at a 1:100 and secondary goat anti-rabbit Cy2 (Abcam, Cat. No. ab6940) at 1:1,000 dilution.

### Quantitative PCR

Zebrafish larvae (AB/Nacre) were treated in pools of ~20-30 larvae per treatment per biological replicate (BR); each BR consisted of embryos from distinct parents. Larvae were collected into 1.5 ml reaction tubes post-treatment and RNA extraction was performed using the rNeasy Plus Micro kit (Qiagen, Cat. No. 74034). cDNA was synthesized from ~300 ng total RNA using the Superscript IV VILO kit (Thermo Fisher, Cat. No. 11756050). Primers in target genes were designed wherein primers annealed in two exons separated by one or several large introns that could not be amplified with the selected PCR settings, to avoid genomic DNA contamination. Quantitative PCR was performed with the Applied Biosystems QuantStudio 3 using PowerUp SYBR Green (ThermoFisher, Cat No. A25741). Amplification signals for expressed target genes were normalized to zebrafish 18S rRNA signals. qPCR conditions were used as follows: 95 °C for 10 minutes, 40 cycles of 95 °C for 15 seconds, 50 °C for 30 seconds, 72 °C for 30 seconds. Each biological replicate was run in quadruplicate. Data were presented as relative expression compared to control using the delta-vdelta Ct (2^{\hat{ }-ΔΔCt}) method.

TABLE 1
Primer sequence for qPCR:

| | | | Target | SEQ ID |
|---|---|---|---|---|
| Gene | Primer | Sequence (5′ → 3′) | Exon | NO: |
| 18S | forward | ATGTCCCTCGTCATCCCAGAGAAGTT | 2 | 11 |
| 18S | reverse | ATTGTCCAGACCATTAGCAAGGA | 5 | 12 |
| duox | forward | TTCTTGGTCTGCCTTTGACG | 20 | 13 |
| duox | reverse | GCATGGAAGTACTTGGTAAG | 21 | 14 |
| mmp13a | forward | AATTACCTGACTCGACTGTATGG | 2 | 15 |
| mmp13a | reverse | CCAGTGGCGAAGAAGATCA | 3 | 16 |
| nox1 | forward | TGGCAATAAACATCGCTTTG | 2 | 17 |
| nox1 | reverse | TTCACTGGAGTCTTGGAGCA | 5 | 18 |
| nox2 | forward | CGTATGTGCTCTCTCAGATTGG | 5 | 19 |
| nox2 | reverse | GTCAATCCTGCTACTGTCGTAAA | 6 | 20 |

kif11 CRISPR Knockout

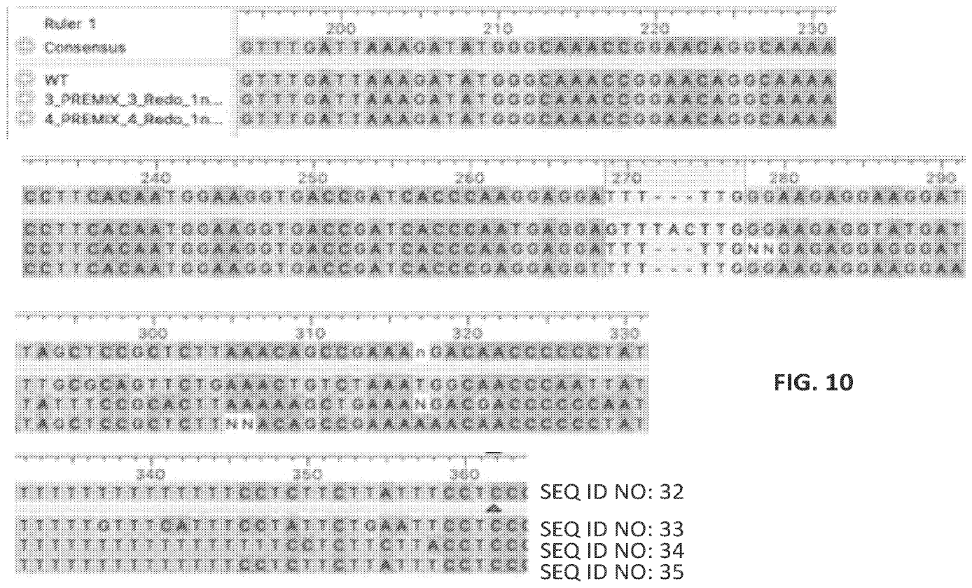

CRISPR oligos targeting kif11 were designed using the IDT CRISPR design tool (https://www.idtdna.com/site/order/designtool/index/CRISPR_CUSTOM). The oligo (5′-AGGTGACCGATCACCCAATG (SEQ ID NO: 21) was designed to anneal within exon 5 of 23 exons total in zebrafish kif11 with expected mutations ~ position 270 bp in the sequenced region using the following primers for PCR amplification: Fwd 5′-TTAGGTTTTTGGCCCTTCTG-3′ (SEQ ID NO: 22) and Rev 5′-GAGGGTCCTGATAGAGAAAAAGTGAA-3′ (SEQ ID NO: 23). The forward primer was used for sequencing and yielded deletions in transiently injected embryos, as shown in example below in which single zebrafish were sequenced (FIG. 10).

The CRISPR oligo was injected using the Alt-R system (IDT, https://www.idtdna.com/pages/technology/crispr/crispr-genome-editing/Alt-R-systems/cas9), which has provided highly reliable and efficient zebrafish knockout results as used herein. The IDT-recommended protocol (below) was used for CRISPR oligo preparations. Fertilized eggs were injected ~15 minutes post fertilization with CRISPR oligos to ensure maximal efficiency.

### Zebrafish Embryo Microinjection Protocol (IDT)

Ribonucleoprotein delivery using the Alt-R™ CRISPR-Cas9 System. Alt-R crRNA and tracrRNA were suspended in Nuclease-Free IDTE Buffer to final concentrations of 100 μM each. A gRNA (3 μM gRNA) solution was prepared using the components in Table 2.

TABLE 2
gRNA Solution Components

| Component | Amount |
|---|---|
| 100 μM Alt-R ™ CRISPR-Cas9 crRNA | 3 μL |
| 100 μM Alt-R ™ CRISPR-Cas9 tracrRNA | 3 μL |
| Nuclease-Free Duplex Buffer (IDT) | 94 μL |
| Final volume | 100 μL |

Samples were heated at 95 °C for 5 min, allowed to cool to room temperature (15-25 °C). Final concentrations of crRNA and tracrRNA were 36 ng/μL and 67 ng/μL, respectively. Cas9 protein was diluted to a to a 0.5 μg/μL working concentration in the buffer as shown in Table 3.

TABLE 3
Cas9 working concentration buffer components

| Component | Amount |
|---|---|
| 10 μg/μL Cas9 protein | 0.5 μL |
| Cas9 working buffer (20 mM HEPES; 150 | 9.5 μL |
| mM KCI, pH 7.5) | |
| Final volume | 10 μL |

RNA complexes were assembled by combining 3 μL of gRNA with 3 μL of diluted Cas9 protein, followed by incubation at 37 °C for 10 min, and then allowed to cool to room temperature. Embryos were collected at the 1-cell stage and 3 nL of RNP complex injected. Un-injected control embryos were also used in experiments. Injected fish were monitored for toxicity at 8 hours, 1 day, 2 days, and 4 days post injection. Four days post-injection genomic DNA was collected using the NaOH method. Target specific PCR was performed and PCR products analyzed using 2% agarose gels.

### Imaging

Confocal immunofluorescence imaging of zebrafish was performed with a 20× air objective and zooms between 1×-3.8×. For microtubule detection, 1 μm sections in varying stack sizes were recorded to capture the full diameter of the caudal fin at either 512×512; 860×860; or 1024×1024 pixel resolution. Scanning was performed at variable scan speed to ensure high quality images.

### Live Imaging of Zebrafish:

Fish were anesthetized in 2-phenoxyethanol (1:1000) in Ringer's solution prior to imaging and mounted in 1.2% agarose. EB3-GFP and Tau-Bfp2 were imaged with a scan speed kept at the maximum level in single slice mode with is intervals for a total of 60s to capture microtubule dynamics. h2a:H2A-GFP time-lapse recordings were produced (Zeiss LSM880 AiryScan confocal microscope) with 1 μm sections recorded every 10 min for 12 hr using 800×800 pixels. All other live fish were imaged using 1024×1024 pixels.

### Image Processing:

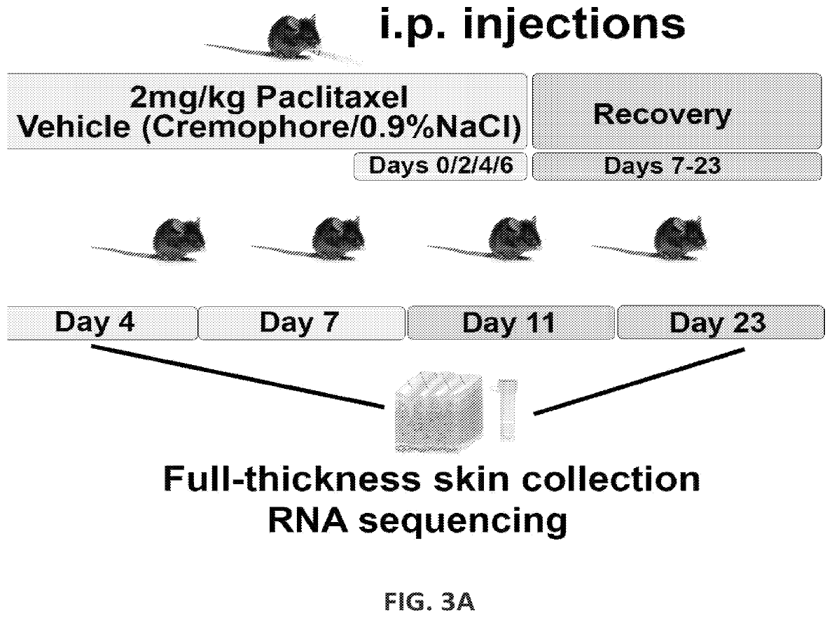

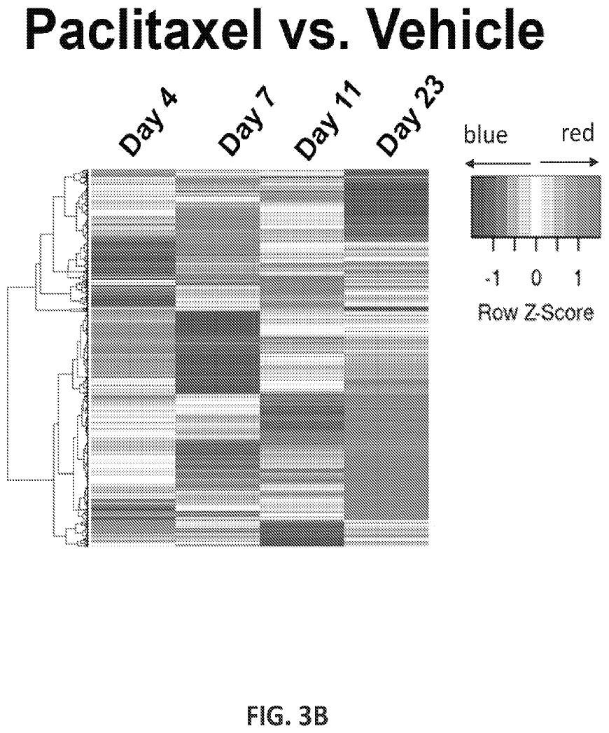

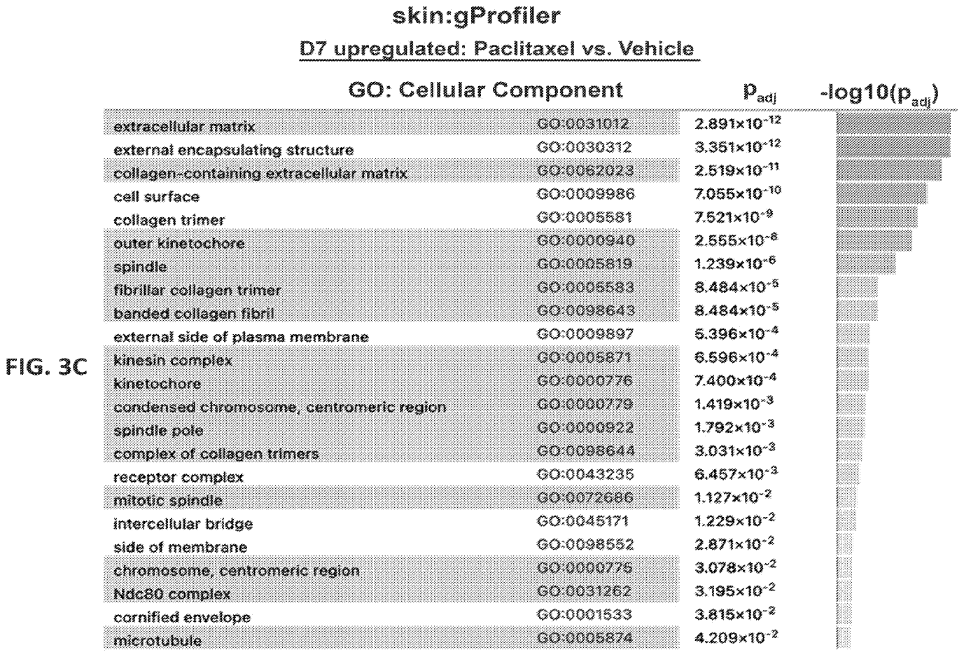

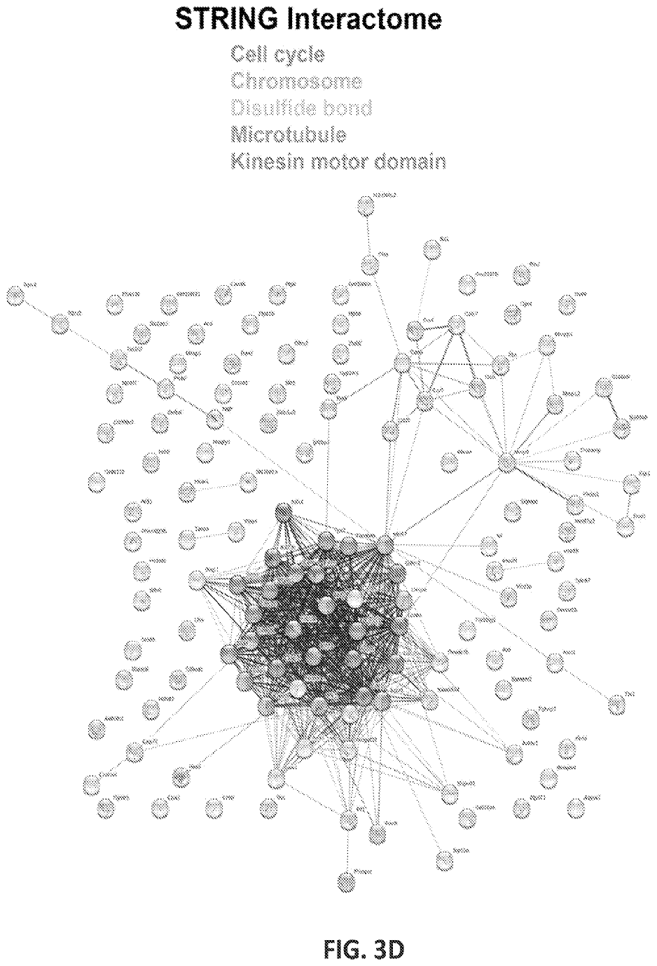

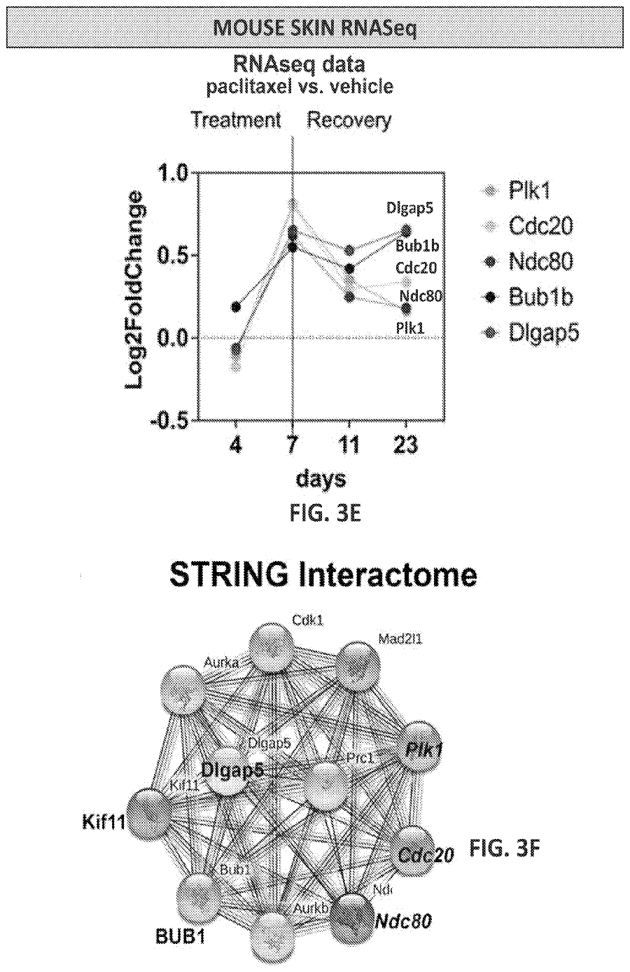

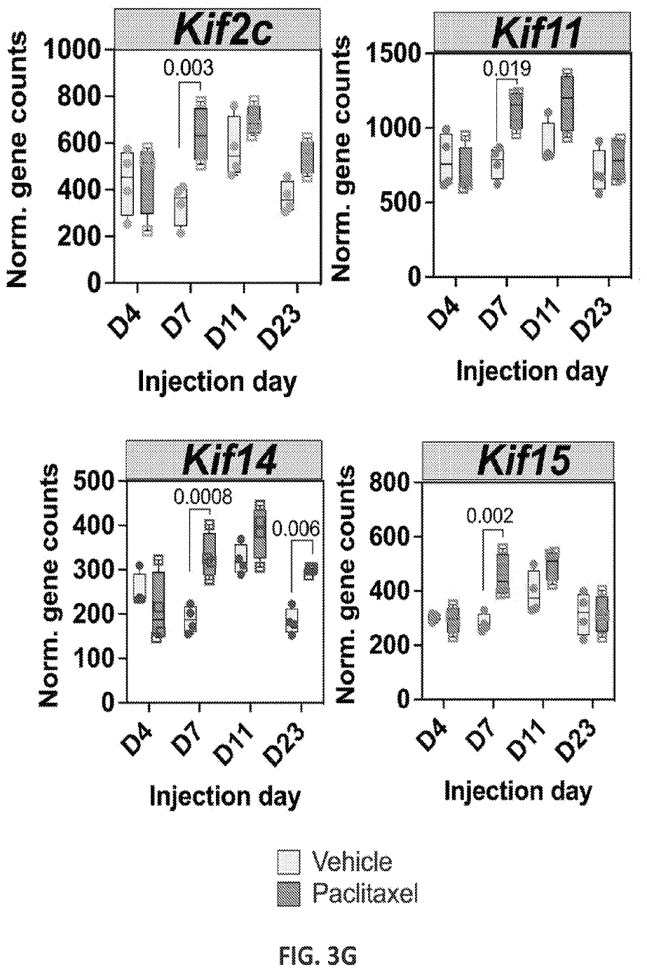

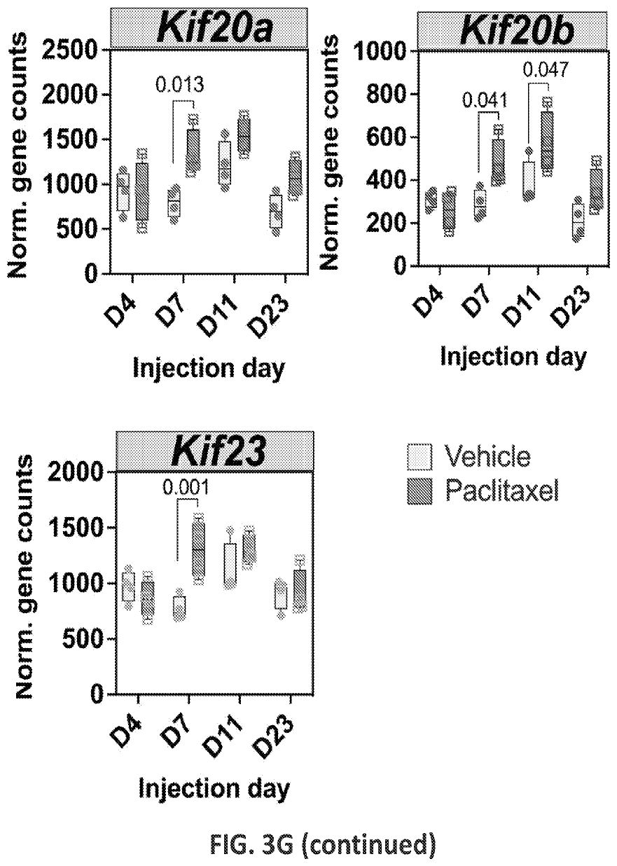

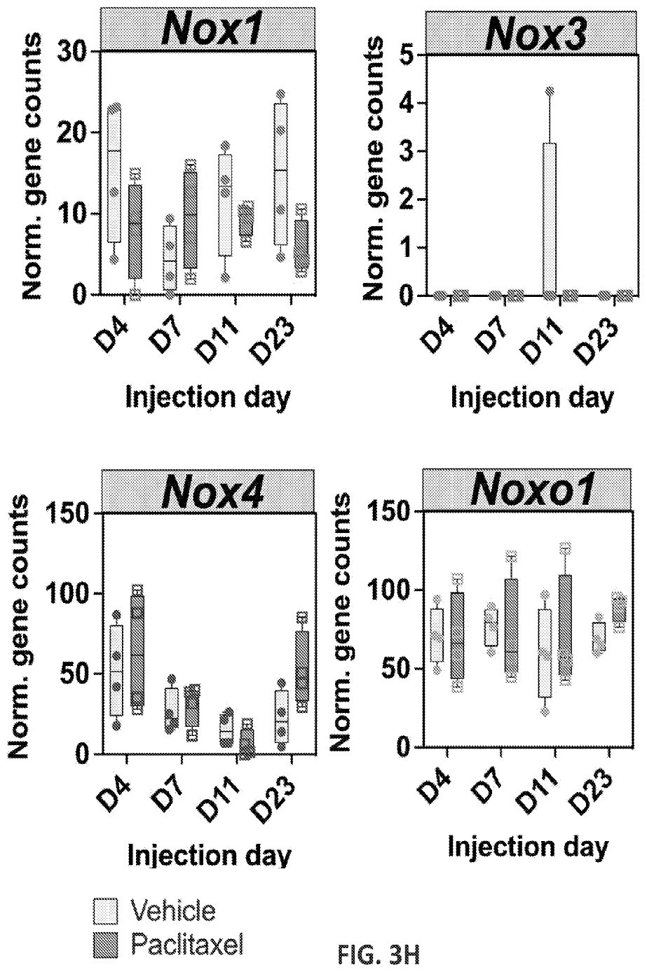

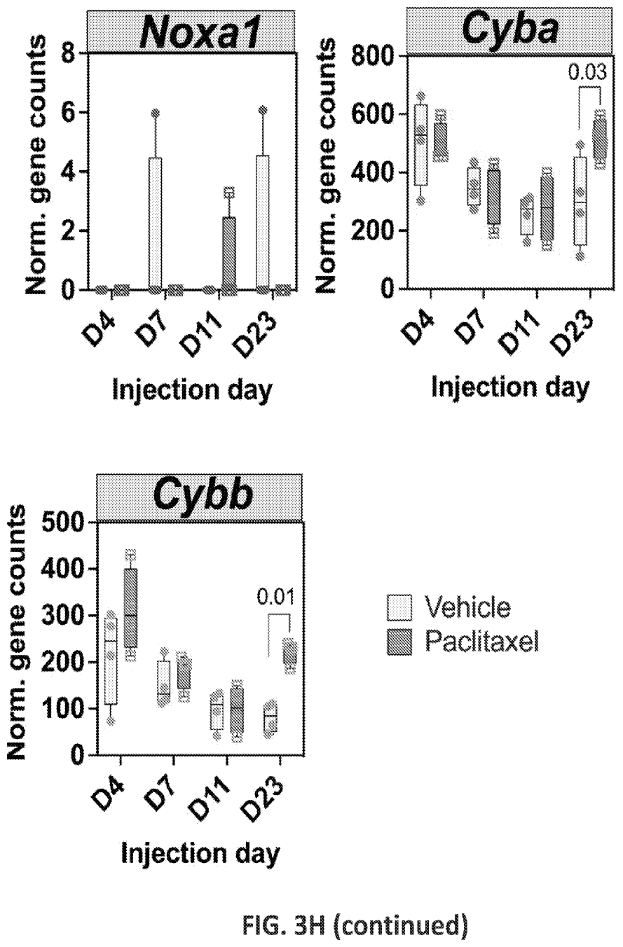

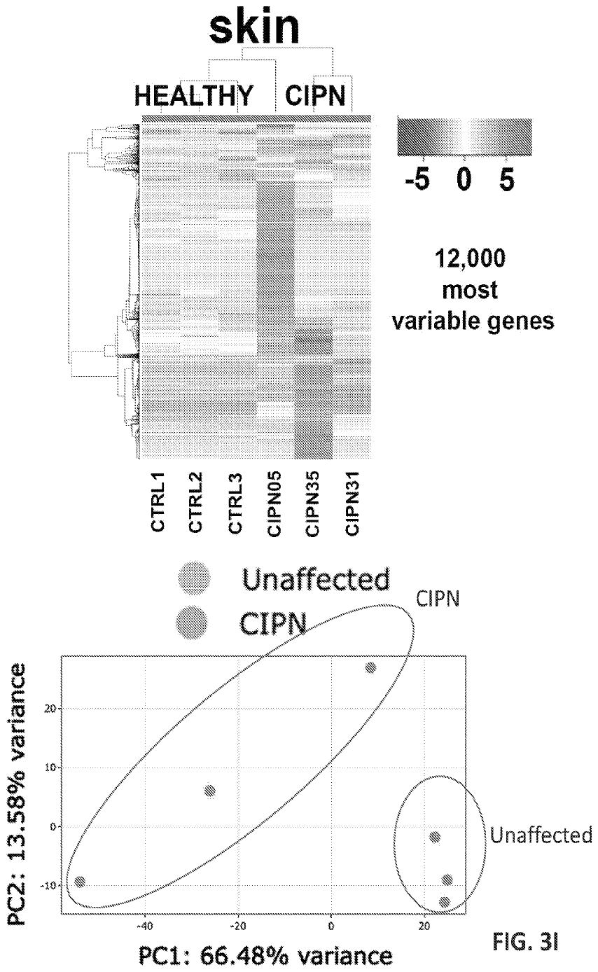

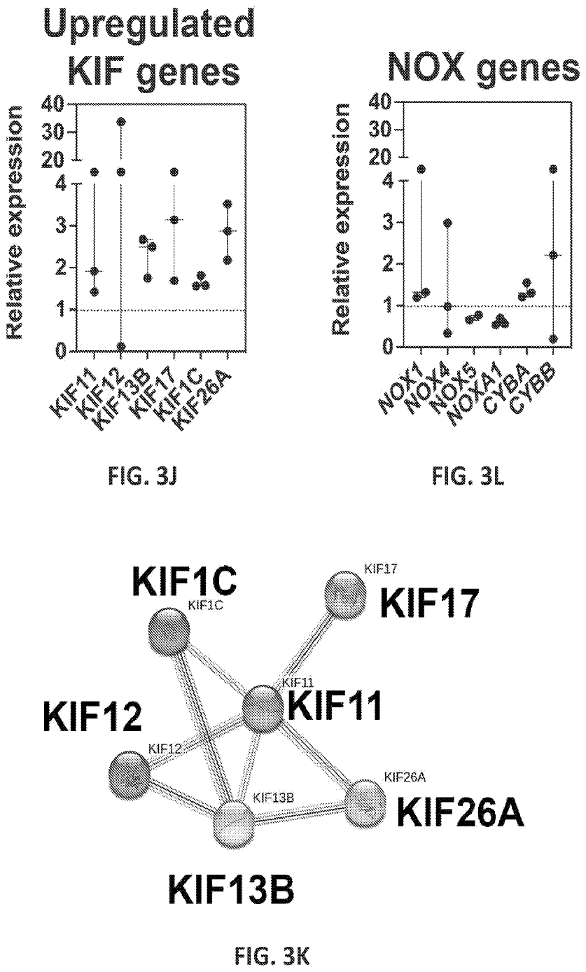

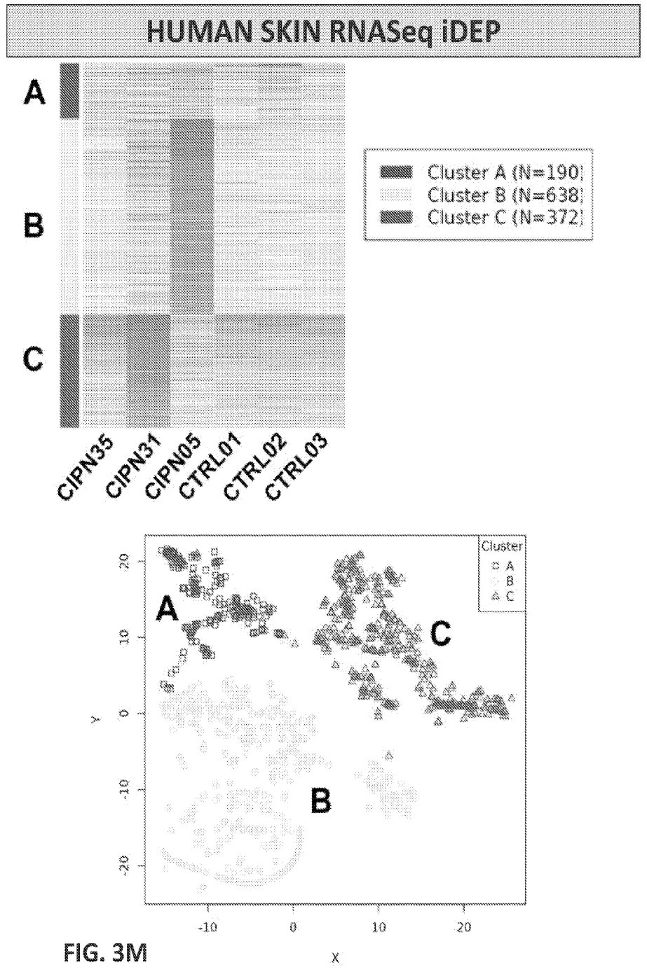

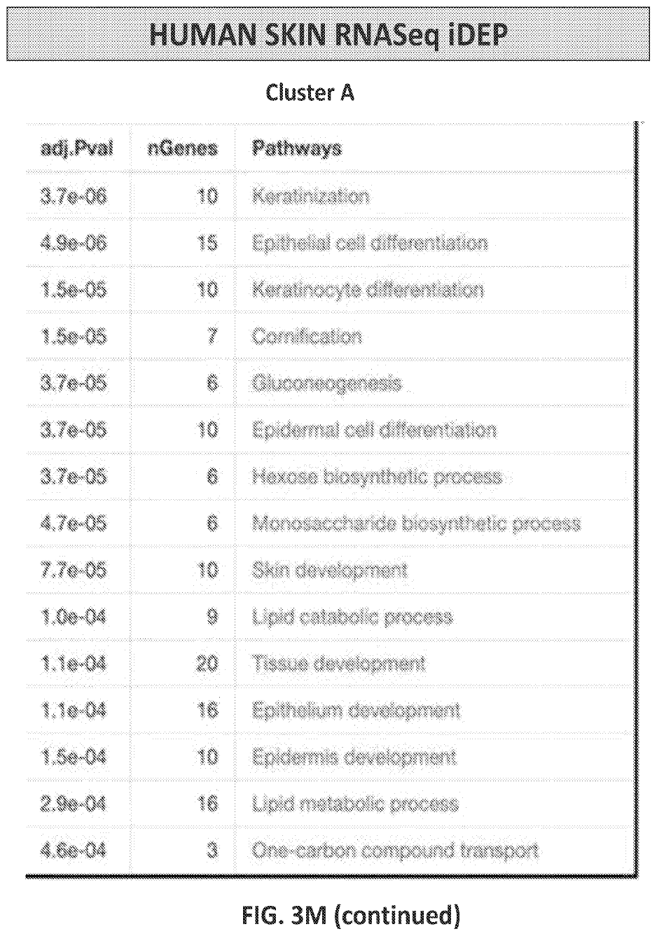

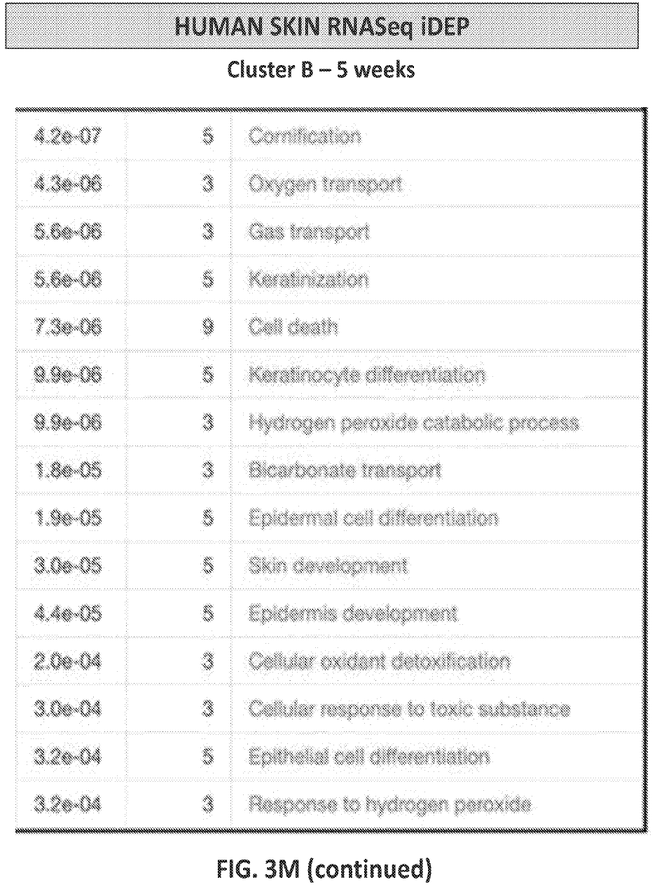

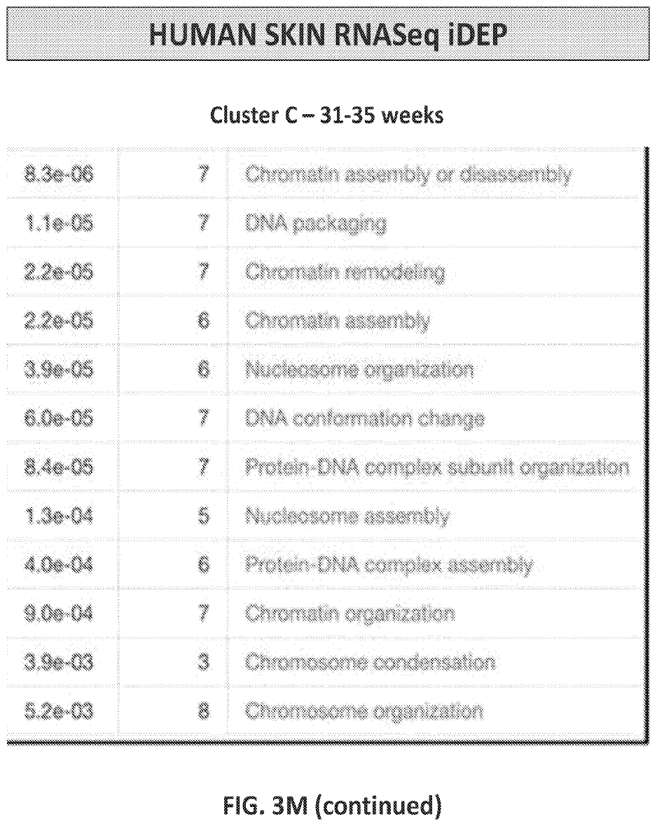

Images were processed and analyzed in Imaris 9.5.1 (Bitplane, Switzerland) or Fiji. Projected stacks were saved as .tif files using the Snap tool, followed by processing in Photoshop to assemble the figures. 3D reconstructions were performed in Imaris using the semi-automated Surface details were set either automatically or manually adjusted depending on the final render quality. Threshold detection and voxel number settings were automatically set. The rendered images were adjusted through repeated re-adjustments of each setting, until a suitable fit was observed. The settings were kept the same for groups that were compared. Graphs were generated and statistics performed in Prism 9 (GraphPad) software. Schematics were prepared in Adobe Illustrator. HyPer mitochondria were ratiometrically displayed using the Image calculator function in ZEN Black whereby the 420 nm unoxidized and 505 nm oxidized channels were added into the division calculator and the signal was amplified by a factor of 150.

### Quantifications

Microtubule analyses was performed in Imaris 9.5.1 (Bitplane) by measuring the straightness of filaments (microtubules) using the “Filaments—Dendrite Straightness” tool, which was defined as the ratio between filament length and radial distance between two branch points (h). The value was always smaller than 1 since the Dendrite Straightness of 1 defines straight objects.

Fluorescence intensities were measured using the Imaris MATLAB plugin or Fiji. A line was typically placed over the region of interest and the fluorescence automatically quantified. The values were normalized to nuclear (Hoechst33342) fluorescence intensities within the same fish and region to obtain an intensity ratio.

Cell counting in the caudal fin was performed in three 100 μm² boxes in the dorsal, medial and ventral caudal fin (~100 μm from the edge).

Nuclear sphericity and volume were calculated in Imaris following 3D rendering of round keratinocyte nuclei using the Surface tool. First, background subtractions were performed, followed by automatic threshold detection. The threshold was adjusted if necessary to fit the nucleus. Thresholds were kept constant for comparisons.

Cell divisions and cell death were manually counted in 12 hr movie recordings. The caudal fin was divided into 6 quadrants and individual cells in each quadrant were manually followed over 12 hr at least 3 times sequentially to capture cell divisions and dying cell behaviors.

dfMT thicknesses (defined as the mean width of a given dfMT) was manually measured at high magnification using the Slice mode in Imaris and the Line tool for measuring the distance between two points. The innermost and outermost microtubule cable was defined as perimeter for the width measurements. Measurements were taken in 3 dfMT positions that covered the minimum, medium, and maximum width within a given dfMT to obtain an overall range distribution.

EB3-GFP tracking was done in Fiji using the MTrackJ plugin to trace microtubules over time and create Kymographs, measure the velocity, track length, and comet duration. Tracking was also performed using the Spots and Surface tools in Imaris. Settings were initially automatically detected to fit the fluorescence and subsequently modified manually for improved fit. Settings were maintained between treatment groups.

Axon branch number quantifications were performed by drawing a 50 μm line across the dorsal, medial, and ventral caudal fin edge region at a distance of 100 μm parallel to the fin edge (which was determined to be the most reliable region to quantify distal branch numbers). Axons traversing this line were counted and averaged per treatment group. Axonal fluorescence measurements were taken using the Imaris Spots function by manually placing spots along the axon and using the fluorescence intensity profile measurements MatLab tool.

HyPer fluorescence was measured in the oxidized (505 nm) and unoxidized (420 nm) channels by using the region of interest (ROI) function in ZEN Black (Zeiss) whereby ROIs were selected inside highly magnified mitochondria (HyPer-mito), cytoplasm (HyPer-cyto) or the plasma membrane (HyPer-CAAX). The data was exported, and ratios determined in Excel. H₂O₂ measurements in zebrafish transiently injected with tp63:Hyper were manually performed by placing spot objects in Imaris 9.5.1 onto individual cells such that the spots extended to the lateral edges. Fluorescence intensities were measured inside the spots and 505 nm/420 nm ratios were subsequently calculated.

### Membrane/Cytoplasmic/Nuclear Ratio Measurements for Nox1 Staining

Measurements were determined by measuring fluorescence intensity in Fiji using single slice and single channel modes. First, a line using the line tool was placed along the plasma membrane that was clearly distinguishable from the cytoplasm due to increased fluorescence compared with the cytoplasm in all treatment groups. The measurement tool was subsequently selected to measure the mean fluorescence intensity. Three lines per caudal fin keratinocyte were averaged for 5 cells per animal and at least 3 animals per treatment group. To determine the mean cytoplasmic fluorescence intensity, nuclei were first overlaid with Nox1 fluorescence to ensure the exclusion of nuclear measurements. These images were then used to draw lines within the cytoplasm as above. For nuclear measurements, the line was placed inside a nucleus such that it spanned the longest extent of the nucleus. The fluorescence data was exported to Excel and the ratios calculated.

### Statistical Analyses

Student's t-test was used to compare groups. A one-way ANOVA was used to compare more than two groups and a single variable. A two-way ANOVA was used for comparisons of multiple groups and multiple variables. Significance was set at p<0.05, p<0.01, p<0.001, p<0.0001.

### In Vivo Experiments

### Animal Care and Treatment:

C57BL/6 mouse, 6 weeks old (15-22 g), were IP injected with 20 mg/kg paclitaxel (Athenex). The mice in each cage were randomly allocated to different treatment groups. Food and water were available ad libitum and experiments were performed during the light cycle (7:00 am to 7:00 pm). Animals were euthanized via CO₂ asphyxiation, followed by cervical dislocation. Animals that showed behavioral disturbances unrelated to chemotherapy-induced pain were excluded from further behavioral testing. The animal's husbandry conformed with established NIH and Institutional Animal Care and Use Committee (IACUC) approved protocols.

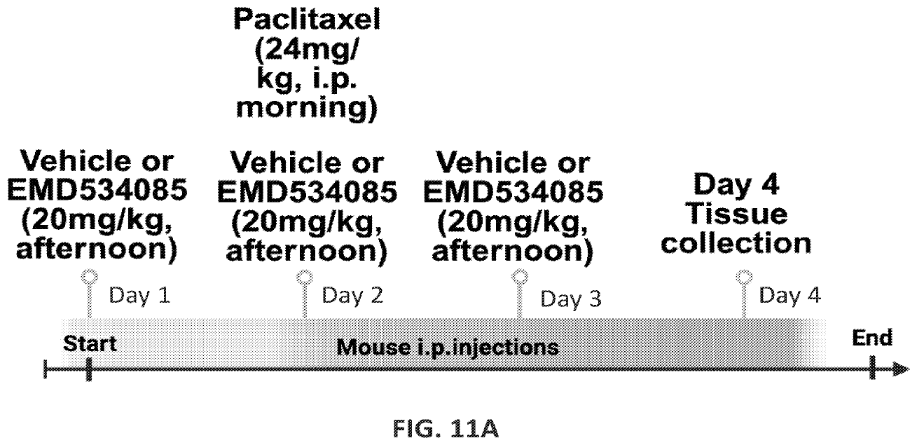

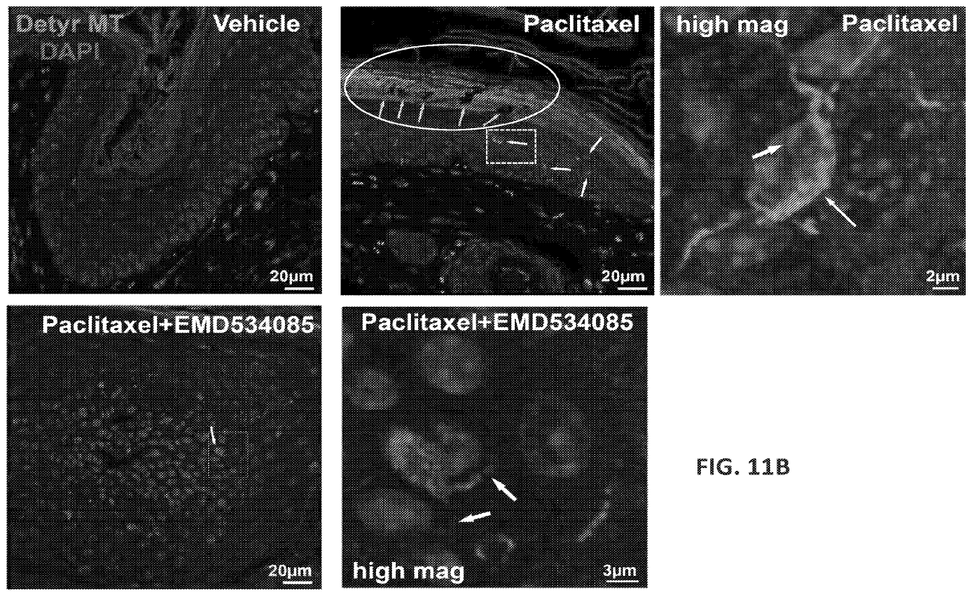

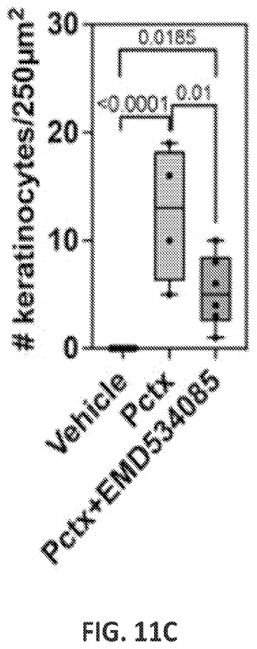

For producing the experimental results illustrated in FIG. 11A-11C, C57BL6/J mice at 12 weeks old were IP injected with vehicle (0.5% DMSO/1×PBS), paclitaxel (24 mg/kg; one injection), and the EG5 inhibitor, EMD534085 (20 mg/kg; 3 injections: 1 before, during, and after paclitaxel). The mice were then sacrificed 24 hours after the last injection (EMD534085 or vehicle) or 48 hours after paclitaxel injection, and the skin of the hind paw pad was collected for histology studies.

### Tissue Staining:

Paws and back skin were collected at predetermined time points, placed in 10% formalin, and stored overnight at 4 °C. Samples were embedded in paraffin and sectioned at 7 μm, deparaffinized, washed with PBS, and incubated at room temperature for 5 min in 0.1% Triton X-100 in PBS and then blocked for 30 min (5% BSA). Sections were incubated with primary antibody diluted in blocking solution, overnight at 4 °C in a humidity chamber. Following PBS washes, sections were incubated for 1 h at room temperature with a secondary antibody. Tissues were mounted and assessed for staining. Antibodies used were against detyrosinated tubulin Millipore AB3201, p-KIF11 Invitrogen PA5-38647, alpha-tubulin Proteintech 66031-1-Ig, mouse Alexa Fluor 488 Invitrogen A21202, and rabbit Alexa flour 546 Invitrogen A10040. DAPI was used for nuclei counterstaining.

### Imaging:

Immunofluorescence staining was performed with a widefield microscopy using a Plan-Apochromatix 100× objective lens (oil immersion, numerical aperture [NA]1.4), 20× and 10× objective lens on inverted Zeiss Axio Observer Z1 using AxioVision 4.8 software. Images were acquired using a monochrome Zeiss Axio Cam MRm CCD camera. Images were processed with the NIH Image J software.

### Mouse RNAseq Analysis:

The RNAseq data set was published in Cirrincione et al. (2022, Data 7: 72). Following differential gene expression analysis (FDR<0.01) comparing paclitaxel-treated to vehicle control animals (n=4), identified genes were queried in gProfiler and grouped by their molecular function. Using normalized gene counts, Kif genes were analyzed for their expression and plotted over time. Significance values comparing paclitaxel and vehicle controls were established using Prism 9 (GraphPad). Kif11 was queried in STRING to identify associated networks and interacting proteins compared to the RNAseq data set and differentially expressed genes were graphed in Prism 9.

### Human RNAseq

Three female breast cancer survivors were recruited who received standard adjuvant paclitaxel treatment. Paclitaxel was administered in these patients over the course of 12 weeks by infusions of 80 mg/m² each. A retrospective review of the patient's medical records was conducted to confirm the date of CIPN diagnosis (5 weeks for XMMP003, 31 weeks for XMMP002, and 35 weeks for XMMP0001 prior to the performed skin biopsies) and record paclitaxel administration to the patient. Neurological history, examination, and QLQ-CIPN20 quality of life questionnaire, which has been validated to detect the presence of CIPN (Postma et al., 2005, Eur J Cancer 41: 1135-1139), was completed in all subjects. Consent was given to obtain two skin punch biopsies of approximately 4 mm diameter. These were collected 10 cm proximal to the lateral malleolus on the distal leg as described previously (Engelstad et al., 2012, Neurology 79: 2187-2193). The biopsies were carried out under anesthesia following local injection of 2% lidocaine with epinephrine, using a sterile technique. In addition, three healthy volunteers of similar age and same gender (female) were recruited as controls. Volunteers serving as controls had no history of neuropathy, diabetes, or familial neuropathies. One skin biopsy was used for RNA sequencing (Azenta/Genewiz) whereas the other biopsy was used in other experiments as set forth herein. RNA isolation and quality control were performed, and paired-end Illumina sequencing (HiSeq 2×150 bp) was conducted. Sequence reads were trimmed to remove possible adapter sequences and nucleotides with poor quality using Trimmomatic v.0.36. The trimmed reads were mapped to the Homo sapiens GRCh38 reference genome available on ENSEMBL using the STAR aligner v.2.5.2b. The STAR aligner was a splice aligner that detects splice junctions and incorporates them to help align the entire read sequences. BAM files were generated through this step. Unique gene hit counts were calculated by using featureCounts from the Subread package v.1.5.2. The hit counts were summarized and reported using the gene_id feature in the annotation file. Only unique reads that fell within exon regions were counted. If a strand-specific library preparation was performed, the reads were strand-specifically counted. After extraction of gene hit counts, the gene hit counts table was used for downstream differential expression analysis. Using DESeq2, a comparison of gene expression between the customer-defined groups of samples was performed. The Wald test was used to generate p-values and log 2 fold changes. Genes with an adjusted p-value <0.05 and absolute log 2 fold change >1 were called as differentially expressed genes for each comparison. Below are the results of the number of differentially expressed genes for all comparisons provided. A gene ontology analysis was performed on the statistically set of genes by implementing the software GeneSCF v.1.1-p2. The goa_human GO list was used to cluster the set of genes based on their biological processes and determine their statistical significance. A list of genes clustered based on their gene ontologies was generated.

Differentially expressed candidate genes for control subjects and CIPN patients were subsequently graphed either as normalized gene counts or relative expression using GraphPad Prism 9. Upregulated KIF genes were queried in STRING to identify networks. The data was uploaded to the Gene Omnibus Database with the GEO Accession number GSE228633.

### Example 1: Paclitaxel Treatment Promoted Microtubule Detyrosination and Fasciculation in Epidermal Keratinocytes

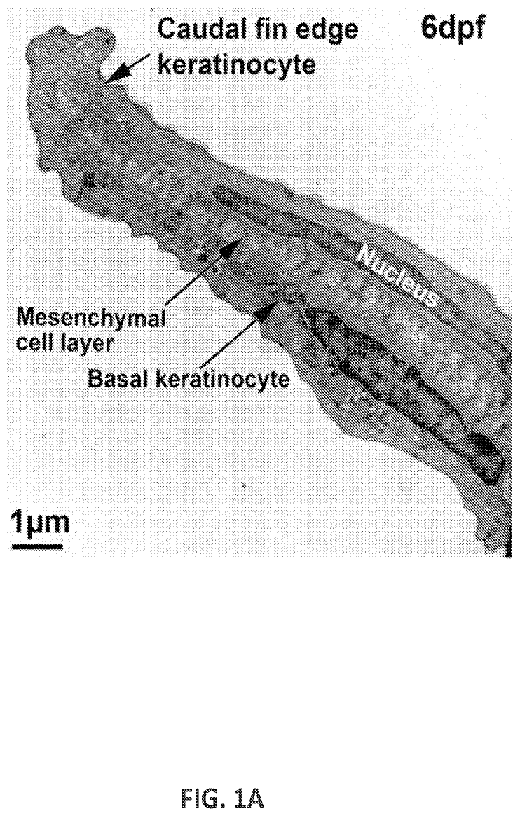

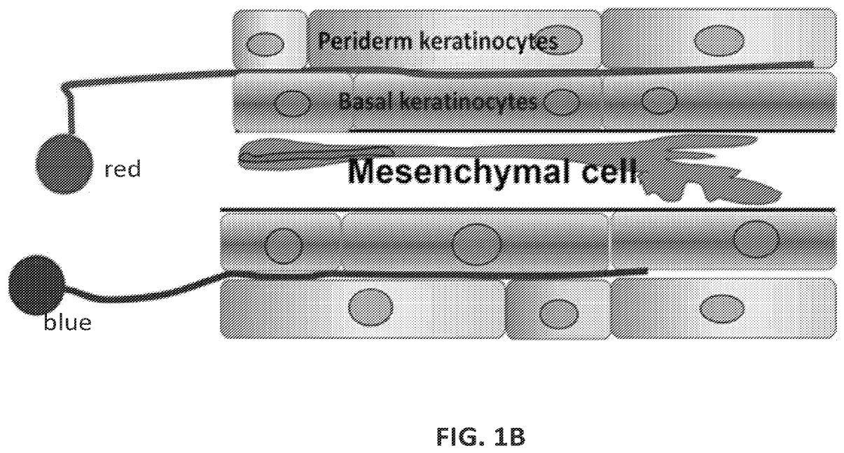

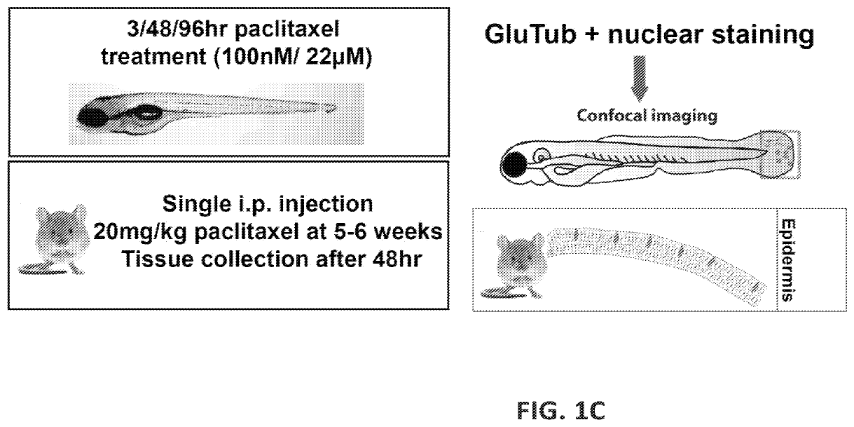

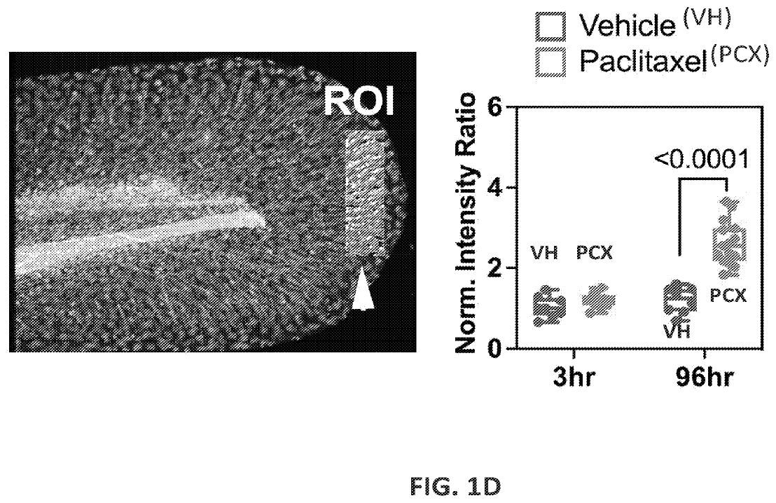

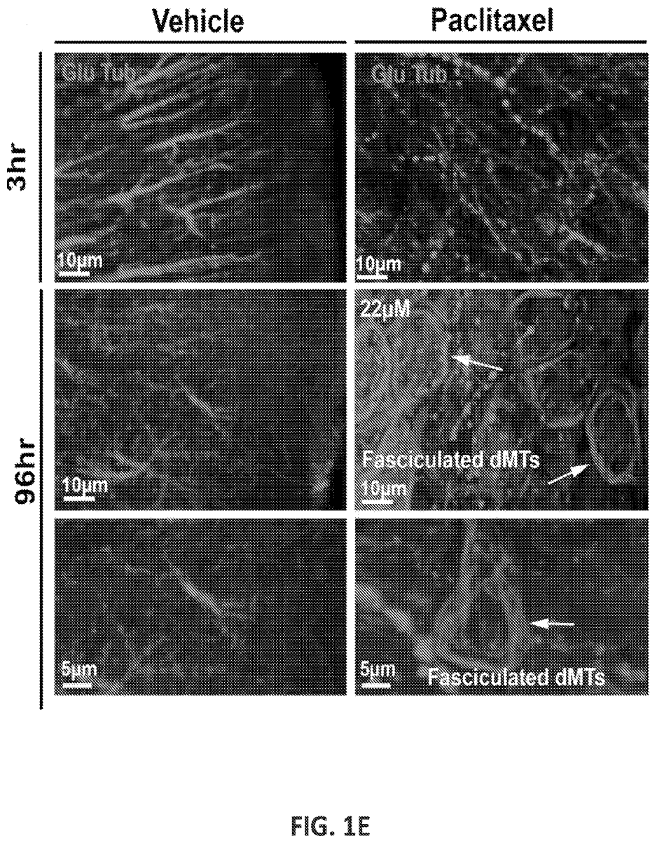

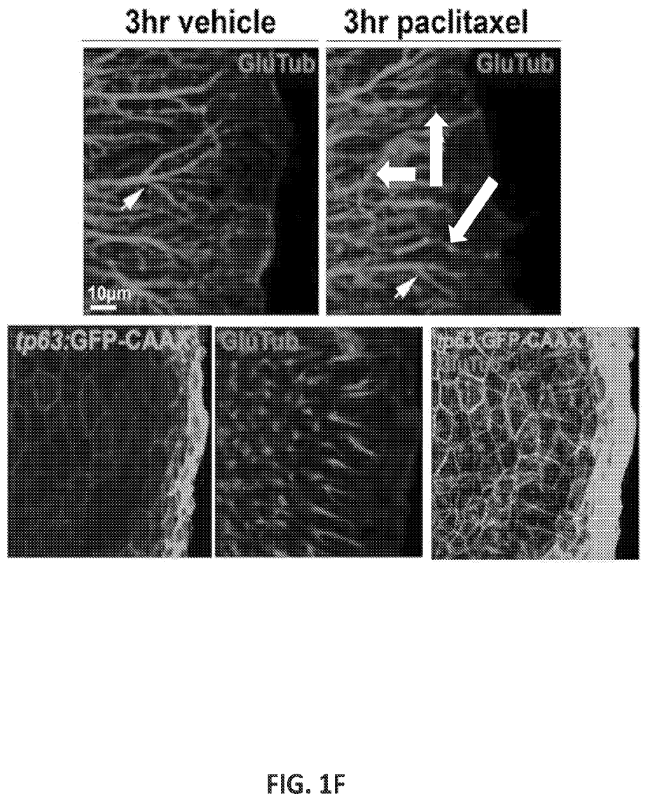

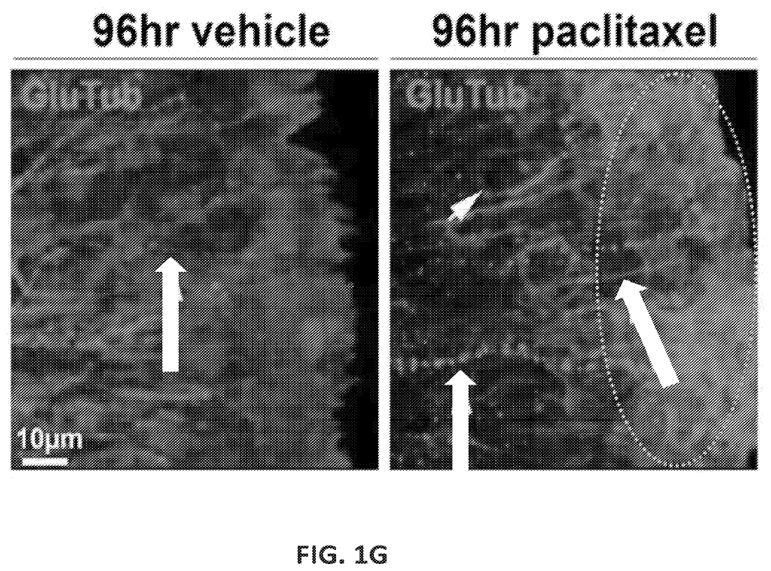

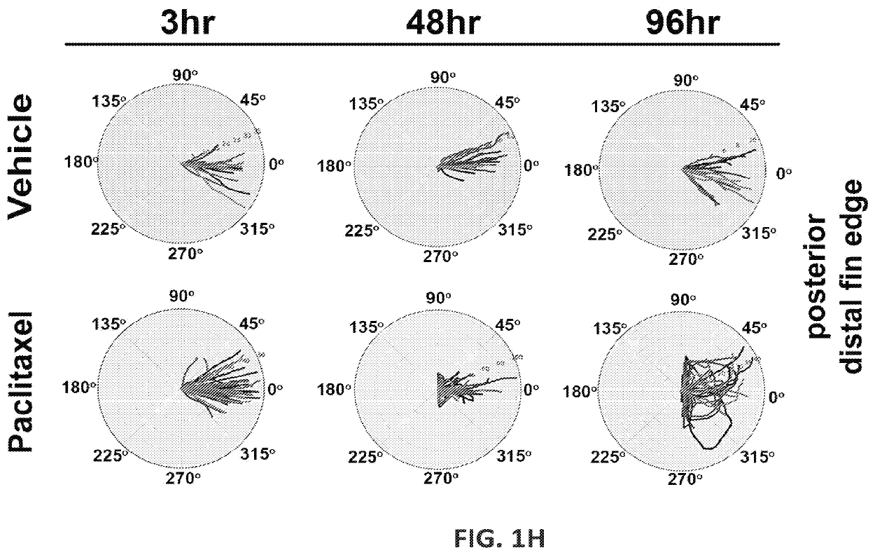

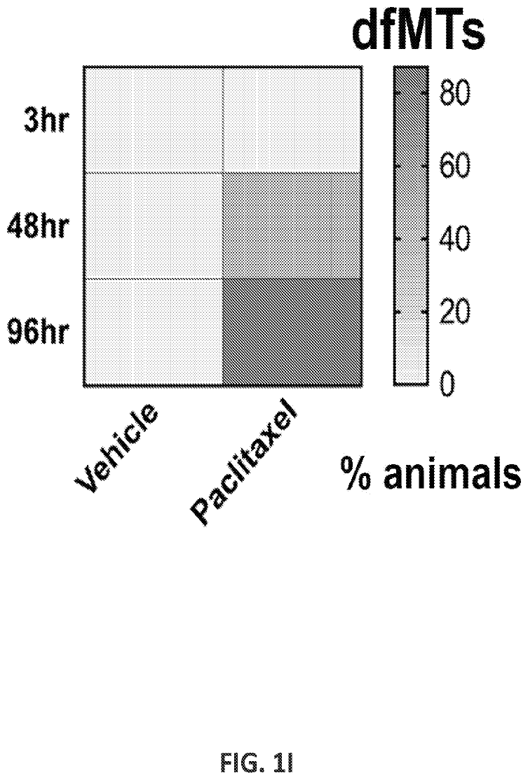

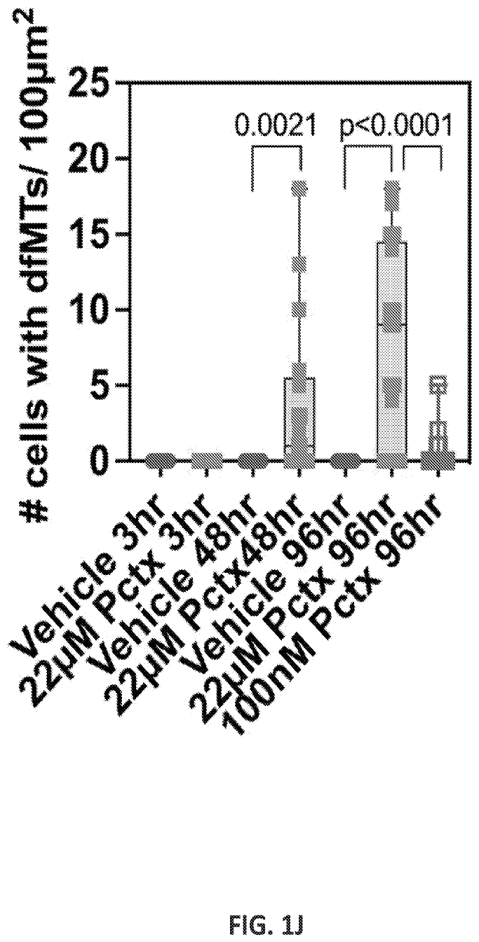

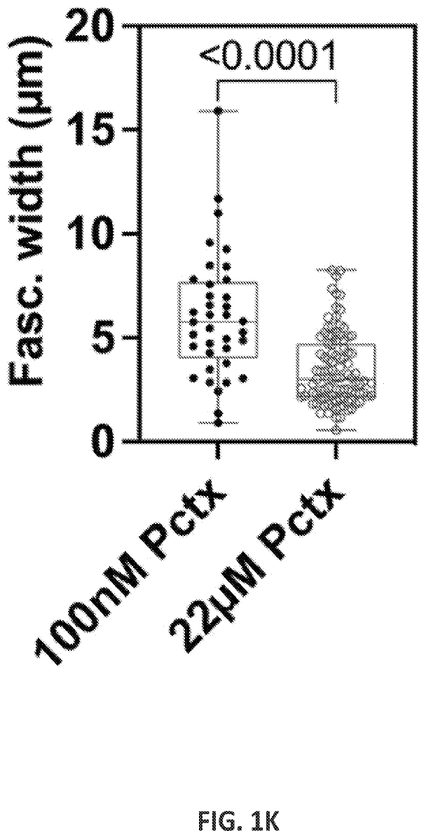

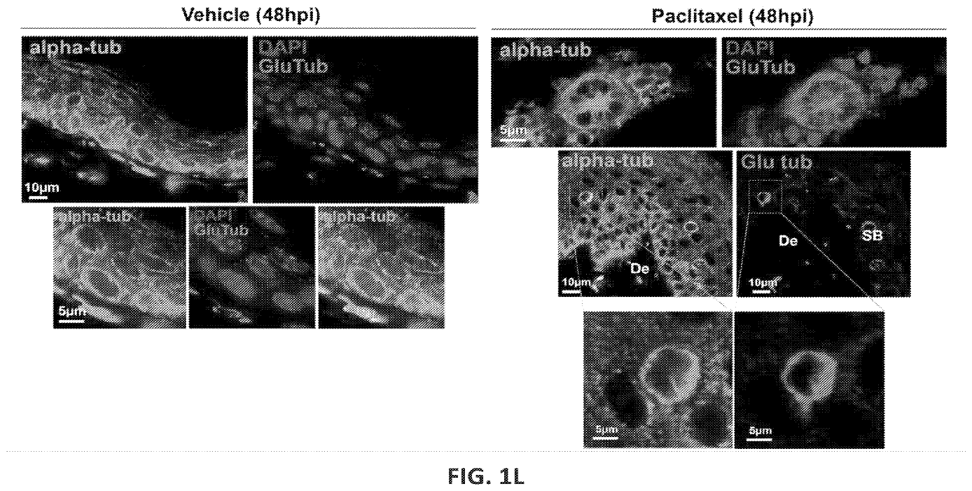

It was first determined whether paclitaxel treatment of larval zebrafish stimulated microtubule stabilization. Stabilized microtubules undergo a modification whereby the terminal tyrosine residue was removed, leading to detyrosination (dMT) (Roll-Mecak et al., 2020, Developmental cell 54: 7-20), which was associated with particularly long-term stabilized microtubule populations (Khawaja et al., 1988, The Journal of cell biology 106: 141-149). Exposure of the penultimate glutamate residue can be detected using a GluTub antibody. Previous findings showed that cutaneous axon degeneration was most prevalent in the distal caudal fin of zebrafish, which consists of two layers of epidermal keratinocytes that are infolded. Each of the two layers are innervated by unmyelinated axons of somatosensory neurons and mesenchymal cells are medially located between the infolded epidermis (as shown in FIG. 1A and FIG. 1B). This distal fin axon degeneration model was used in all subsequent studies. Caudal fin keratinocytes in vehicle (0.05% DMSO)-treated animals did not show stable microtubule populations following treatment for 3 and 96 hours (as shown in FIG. 1C-E). However, dMTs were visible in mesenchymal cells at 2 days post fertilization and could be distinguished by their elongate shape spanning multiple keratinocytes (as shown in FIG. 1A, FIG. 1B, FIG. 1E, and FIG. 1F). Paclitaxel (22 μM) treatment promoted microtubule detyrosination in keratinocytes following long-term (96 hr) but not short-term (3 hr) treatment (normalized fluorescence intensity ratio: 3 hr (2dpf), vehicle: 1.039±0.093 vs. paclitaxel: 1.197±0.053; 96 hr (6dpf), vehicle: 1.203±0.115 vs. paclitaxel: 2.621±0.14) (as shown in FIG. 1D). Intriguingly, dMTs became fasciculated in a subset of keratinocytes and looped around the cell periphery, most prominently in the distal caudal fin in which axon degeneration commences (Lisse et al., 2016, Proceedings of the National Academy of Sciences of the United States of America 113: E2189-2198) (as shown in FIG. 1E and FIG. 1G). Microtubule detyrosination, fasciculation, and looping (abbreviated as dfMT) was evident after 48 hr paclitaxel treatment in ~45% of analyzed animals, and in ~90% after 96 hours, but never in vehicle controls, or after 3 hr paclitaxel treatment (as shown in FIG. 1H and FIG. 1I). dfMT presence ranged from 0 to ~20 keratinocytes/100 μm²/animal but on average, these were detected in 3.64±1.28 keratinocytes/100 μm² following 48 hr paclitaxel treatment, and in 7.7±1.6 keratinocytes/100 μm² following 96 hr paclitaxel treatment (as shown in FIG. 1J). A low dose of paclitaxel (100 nM) also promoted dfMT formation, but the overall number of affected keratinocytes per animal was lower compared with high dose experiments (100 nM paclitaxel: 0.4±0.21 dfMT-positive keratinocytes/100 μm²) (as shown in FIG. 1J), suggesting that the extent of dfMT formation was dose-dependent. Interestingly, despite the low number of keratinocytes harboring dfMTs with 100 nM paclitaxel treatment, the fascicle width of dMTs was enhanced (100 nM paclitaxel: 6.0±0.49 μm vs. 22 μM paclitaxel: 3.4±0.17 μm) (as shown in FIG. 1K). To determine if this phenotype was inherent to zebrafish or conserved in mammals, dfMT formation in the mouse epidermis was analyzed. A single injection of vehicle or 20 mg/kg paclitaxel into mice (5-6 weeks) resulted in dfMT formation in a subset of suprabasal keratinocytes. These occasionally formed large cell clusters with rosette-like dfMTs connecting them (as shown in FIG. 1L). In contrast, vehicle-injected control animals never showed dfMT formation although some degree of detyrosination was present. Together, these findings demonstrated that paclitaxel treatment promoted a dose-dependent dfMT formation in subpopulations of epidermal keratinocytes,

### Example 2: Paclitaxel Promoted X-ROS Formation Via Altered Microtubule Mechanotransduction

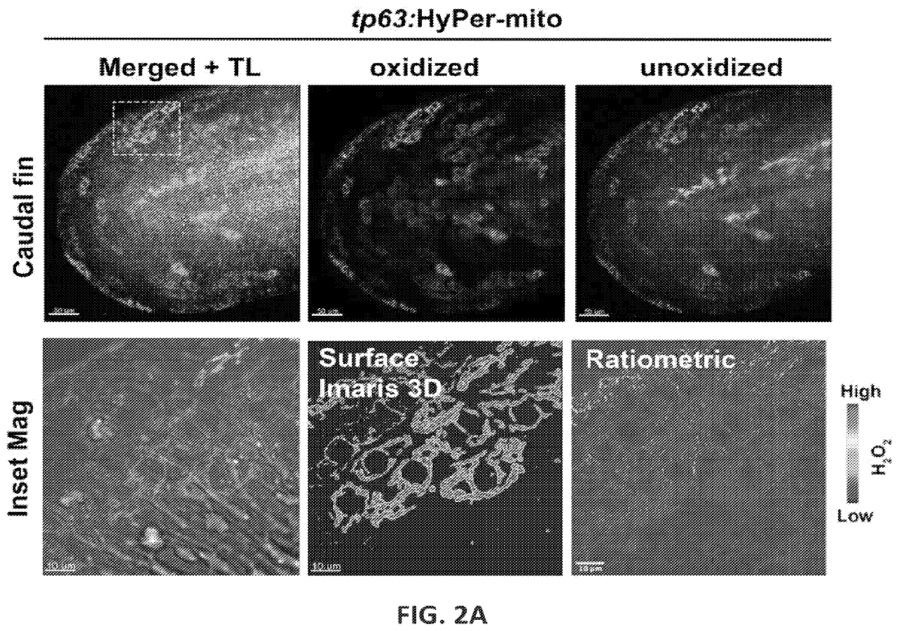

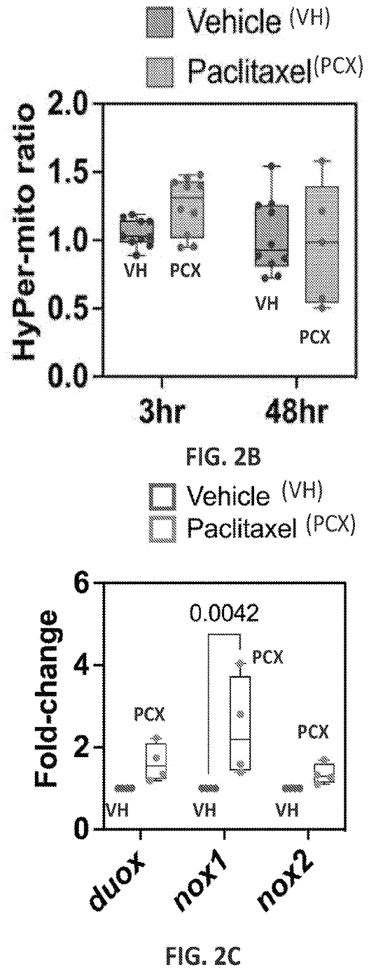

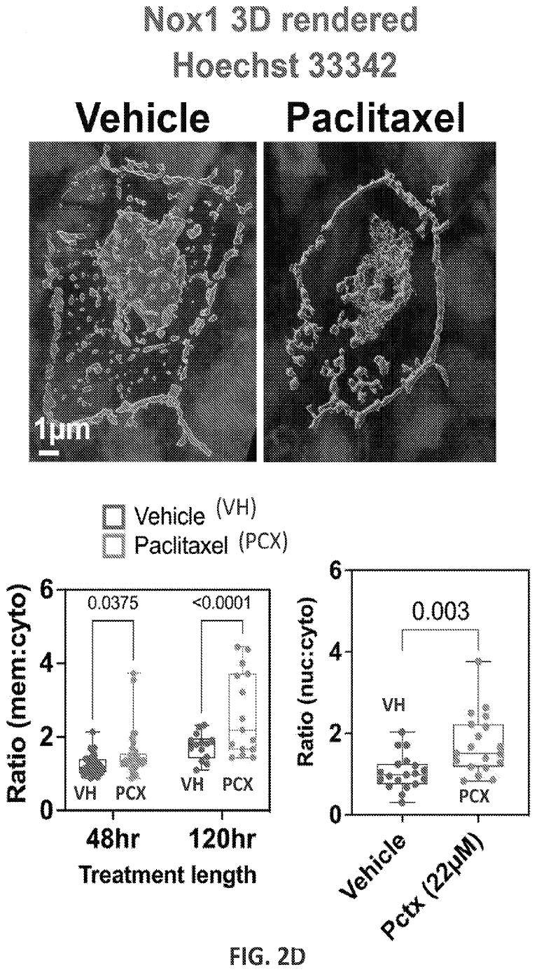

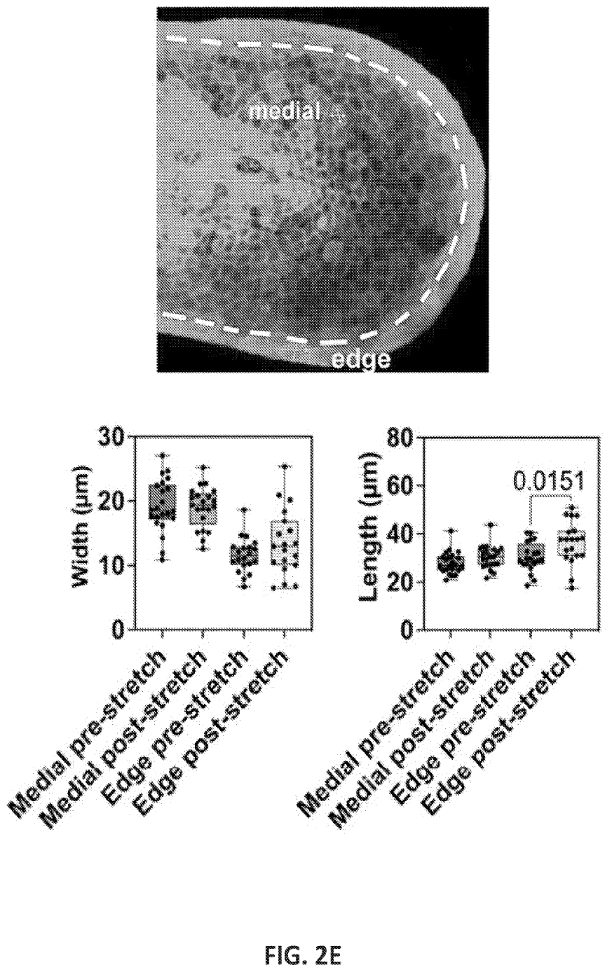

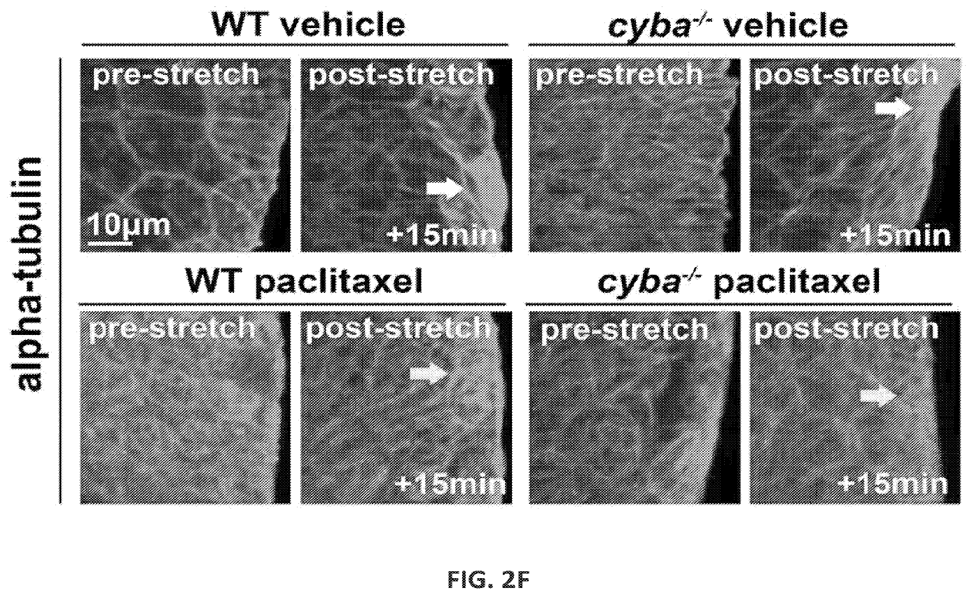

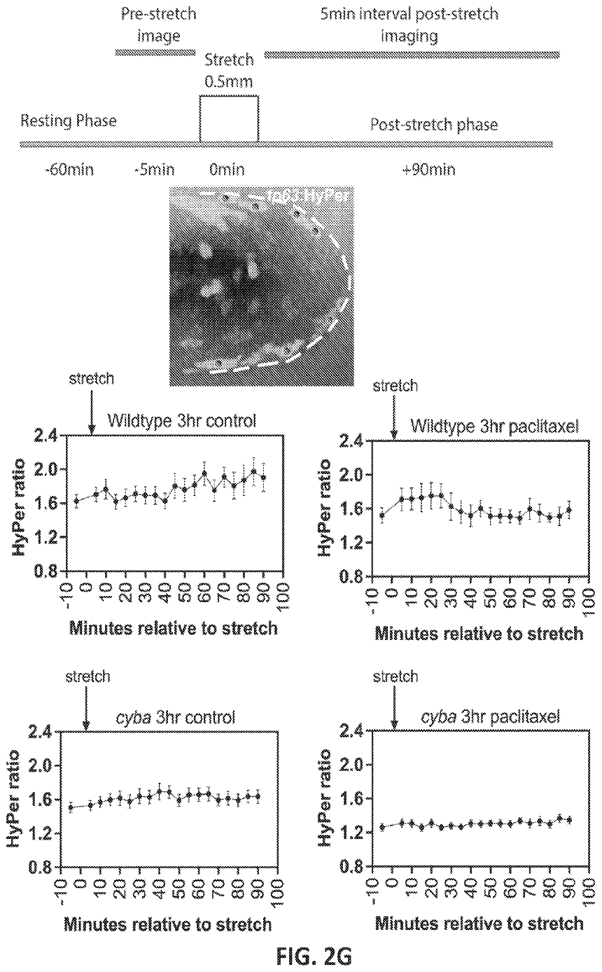

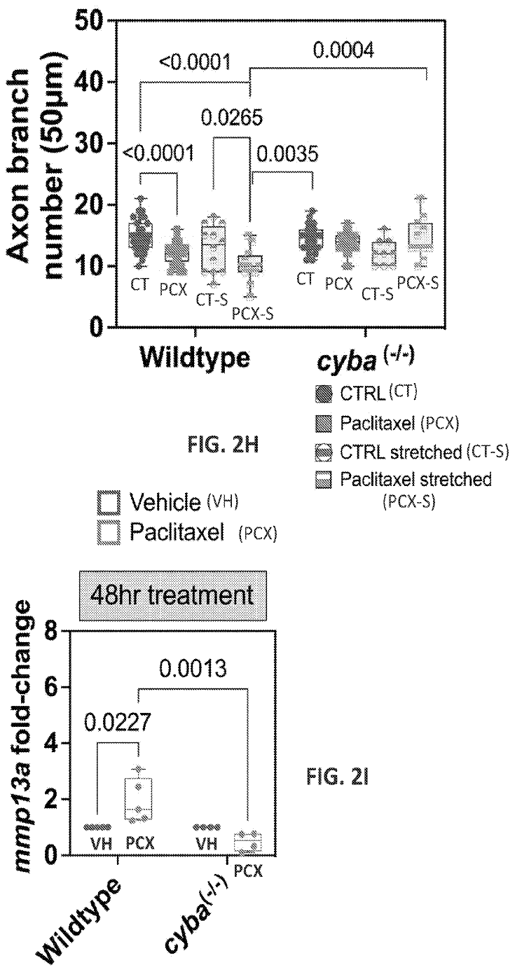

It was then determined whether keratinocyte dfMT formation contributed to ROS production. ROS can be derived from two major sources within cells, mitochondria and membrane-bound NADPH oxidases. Previously detected morphological changes in keratinocyte mitochondria following paclitaxel treatment (Cirrincione et al., 2020, Sci Rep 10, 3970) suggested that keratinocyte-specific mitochondrial reactive oxygen species (mitoROS) might be a source, supported by mitochondrial diseases having been linked to peripheral neuropathy (Cassereau et al., 2014, Revue neurologique 170: 366-374; Flatters et al., 2015, Biol Transl Sci. 131:119-146; Pareyson et al., 2013, Lancet Neurol. 12: 1011-1024). To assess mitoROS levels in keratinocytes, tp63:HyPer-mito, a mitochondria-targeted genetic sensor for the ROS, hydrogen peroxide (H₂O₂) (Bilan et al., 2018, Antioxid Redox Signal 29: 569-584; Bilan et al., 2013, ACS Chem Biol 8: 535-542) was expressed in zebrafish. Treatment with paclitaxel for 3 hr and 48 hr, which was when cytoplasmic ROS were abundant (Lisse et al., 2016, Proceedings of the National Academy of Sciences of the United States of America 113: E2189-2198, Cirrincione et al., 2022, Data 7: 72) did not increase mitochondrial HyPer oxidation (3 hr vehicle: 1.054±0.031 vs. paclitaxel: 1.252±0.066; 48 hr vehicle: 1.029±0.086 vs. paclitaxel: 0.972±0.2) (as shown in FIG. 2A and FIG. 2B). These results suggested that paclitaxel was not contributing to ROS formation in mitochondria and that NADPH oxidases were the likely source of ROS in keratinocytes, consistent with previous findings that 3 hr and 48 hr paclitaxel treatment induces cytoplasmic H₂O₂ production (Lisse et al., 2016, Proceedings of the National Academy of Sciences of the United States of America 113: E2189-2198; Cirrincione et al., 2020, Sci Rep 10, 3970).

NADPH oxidases were therefore an alternative source for ROS. The NADPH oxidase family consists of Nox1-5 and Duox1/2, with Duox2 being species-dependent (Donkó et al., 2005, Lond B Biol Sci 360: 2301-2308) and Nox1-4 being activated by the subunit p22phox (Bedard et al., 2007, Physiological reviews 87, 245-313; Ushio-Fukai et al., 1996, The Journal of biological chemistry 271: 23317-23321). Quantitative PCR (qPCR) was used initially in zebrafish to analyze expression of the two known epithelium-specific NADPH oxidases, nox1 and duox, in addition to nox2, which was shown to be activated by mechanotransduction mechanisms in cardiomyocytes (Prosser et al., 2011, Science 333: 1440-1445). QPCR on whole larval zebrafish showed only nox1 being upregulated following 48 hr paclitaxel treatment (fold-change from vehicle control: duox: 1.63±0.23, nox1: 2.5±0.61, nox2: 1.35±0.12) (as shown in FIG. 2C). Immunofluorescence staining and 3D reconstruction of Nox1, and nuclear staining using Hoechst33342, further showed a time-dependent recruitment of Nox1 to the plasma membrane and nucleus in epidermal keratinocytes following paclitaxel, but not vehicle, treatment (membrane:cytoplasmic ratio, 48 hr vehicle: 1.23±0.04 vs. paclitaxel: 1.5±0.09; 120 hr vehicle: 1.74±0.09 vs. paclitaxel: 2.63±0.29) (as shown in FIG. 2D). These findings indicate that paclitaxel modulates Nox1 activity by promoting its subcellular translocation, which could be mediated by the altered mechanotransduction of dfMTs, consistent with findings in muscle cells.

Paclitaxel induced Nox-dependent ROS formation was studied by altering microtubule tension, exerted on a zebrafish caudal fin while simultaneously permitting ROS formation to be monitored using time-lapse imaging. The experiments were conducted on the basis that stretch in the presence and absence of paclitaxel would modify microtubule dynamics and this should impact ROS production in wildtype fish, whereas Nox-deficient cyba−/− mutants do not produce ROS in response to stretch. The extent of stretch that was induced in wildtype transgenic Tg(tp63:GFP-CAAX) (Lisse et al., 2016, Proceedings of the National Academy of Sciences of the United States of America 113: E2189-2198) fish in which keratinocyte plasma membranes are fluorescently labelled was characterized. Comparison of vehicle-treated pre- and 15 min post-stretch keratinocytes showed an increase in keratinocyte length, but not width, at the fin edge (keratinocyte length, medial fin: pre-stretch: 27.99±0.97 μm vs. post-stretch: 30.37±1.05 μm; fin edge: pre-stretch: 30.35±1.36 μm vs. post-stretch: 36.46±2.04 μm; keratinocyte width, medial fin: pre-stretch: 19.19±0.86 μm vs. post-stretch: 19.01±0.69 μm, fin edge: pre-stretch: 11.62±0.59 μm vs. post-stretch: 13.52±1.16 μm) as shown in (FIG. 2E). Subsequent fixation of stretched wildtype and cyba−/− mutant fish followed by immunofluorescence staining for alpha-tubulin confirmed stretching of microtubules in fin edge keratinocytes of vehicle-treated, but not in paclitaxel-treated, animals (FIG. 2F), confirming altered mechanical characteristics of stabilized microtubules.

To further investigate whether paclitaxel in combination with stretch alters ROS formation, HyPer imaging was performed. Dual channel imaging of oxidized and unoxidized HyPer in 5 min intervals for 90 minutes pre- and post-stretch showed that HyPer oxidation in vehicle control fish was relatively sluggish, with a maximum increase at ~80 min post-stretch (HyPer ratio (505/420) pre- vs. post-stretch (max): 1.62 vs. 1.97) (as shown in FIG. 2G). Paclitaxel-treated animals (3 hr treatment), in contrast, showed rapid HyPer oxidation that peaked between 10-20 min (HyPer ratio pre- vs. post-stretch (max): 1.49 vs. 1.75) and thereafter declined. This result indicates that altered mechanical characteristics of dfMTs contribute to ROS formation in keratinocytes following paclitaxel treatment. As predicted, HyPer oxidation was Nox-dependent since stretching of cyba−/− mutants with and without paclitaxel treatment resulted in a 10-fold decrease in HyPer oxidation in the vehicle controls, and the absence of a response to stretch in the paclitaxel group (cyba−/−, vehicle: HyPer ratio pre- vs. post-stretch (max): 1.50 vs. 1.69 vs. paclitaxel: 1.25 vs. 1.36). These results further indicated that dfMTs promoted NOX1-dependent ROS (X-ROS) formation in keratinocytes following paclitaxel treatment. The remaining HyPer oxidation in vehicle-treated cyba−/− mutants suggested that changes in MT tension can also stimulate other P22phox-independent NADPH oxidases, such as Duox, or stimulate mitochondrial ROS production.

To further determine whether microtubule stretch enhanced paclitaxel-induced axon degeneration in a Nox-dependent manner, the axon branch number in wildtype and cyba−/− fish treated with vehicle and paclitaxel either stretched or unstretched was compared. Stretching was performed after 96 hr treatment, followed by 2 hr post-stretch fixation, and staining for detection of acetylated tubulin in axons. Axon branch quantifications showed a decrease in axon branches in the caudal fin of wildtype paclitaxel-treated but not vehicle control fish, and no change was found within the cyba−/− group. While stretching slightly enhanced degeneration within wildtype animals, it was rescued in paclitaxel-treated and stretched cyba−/− mutants (as shown in FIG. 2H). To determine whether Nox activation occurred upstream of MMP-13, mmp13 expression was analyzed by qPCR in whole larval wildtype and cyba−/− mutant fish in the absence and presence of paclitaxel. Wildtype but not cyba−/− fish treated for 48 hr with paclitaxel to stimulate axon degeneration displayed increased mmp13 expression (as shown in FIG. 2I). Together these data supported a model by which paclitaxel-induced microtubule stabilization induced X-ROS formation and MMP-13 expression upstream of cutaneous sensory axon degeneration.

### Example 3: Paclitaxel Treatment Stimulated Cell Cycle Gene Expression in Skin

Molecular mechanisms underlying paclitaxel-induced microtubule fasciculation were then investigated. Because previously identified CIPN dependence on MMP-13 was conserved (Lisse et al., 2016, Proc. Natl. Acad. Sci. U.S. 113: E2189-2198; Cirrincione et al., 2020, Sci Rep 10, 3970), a mouse RNAseq dataset in which mice received four intraperitoneal injections of either vehicle or 2 mg/kg paclitaxel every other day four times (Cirrincione et al., 2022, Data 7: 72) was analyzed. In this experiment, skin was harvested on days 4, 7, 11, and 23 for comparisons of gene expression profiles (shown in FIG. 3A). Peripheral neuropathy was correlated with these times points using von Frey behavioral testing, which determined that peak neuropathy was present at day 7 (D7). Peripheral neuropathy was further determined by gene co-expression profiles (log 2 fold-change: ≥0.65) (shown in FIG. 3C). This identified several biological categories during peak neuropathy with the largest clusters being implicated in extracellular matrix organization (e.g. “ECM”, “collagen-containing ECM”, “collagen trimer”) and cell cycle regulation (e.g. “outer kinetochore”, “kinesin complex”, condensed chromosome, centromeric region”, “mitotic spindle”, “microtubules”), consistent with our findings in zebrafish. Interactome analysis using STRING (Szklarczyk et al., 2021, Nucl. Acids Res 49: D605-D612) to predict protein association networks further identified major clusters involved in cell cycle regulation, which contained various kinesins, microtubule and nucleus regulators, as well as one cluster annotated as “disulfide bond”, consistent with a role for oxidative signal activation (Leonard et al., 2011, Current opinion in chemical biology 15: 88-102) (shown in FIG. 3D). Given that paclitaxel is a known cell cycle checkpoint regulator (Jia et al., 2016, Nature communications 7: 10818; Leonard et al., 2011, Current opinion in chemical biology 15: 88-102), expression of the known checkpoint regulators, Plk1, Cdc20, Ndc80, Bub1c, Dlgap5 was further analyzed and showed a co-upregulation from D7 onward (shown in FIG. 3E). Additional genes in the data set that were upregulated during peak neuropathy and formed a network included the mitosis regulating genes, including Cdk1, Aurka, Aurkb, and Kif11 (shown in FIG. 3F). Kif11, which encodes Eg5 (also known as Kinesin-5), was especially informative because 1) members of the Kinesin family are implicated in microtubule regulation and cell cycle progression (Hirokawa et al., 2009, Nat Rev Mol Cell Biol 10: 682-696), 2) the implication of Eg5 in the regulation of cytokinesis via its microtubule crosslinking activity (Leary et al., 2019, Curr Biol 29: 3825-3837.e3823), consistent with the microtubule fasciculation phenotype, and 3) Eg5 inhibition in mice with the small molecule inhibitor, monastrol, has been shown to alleviate CIPN caused by bortezomib (Bobylev et al., 2017, Neurotox Res 32: 555-562). Besides Kif11, other upregulated Kif genes were found during peak neuropathy: Kif2c, Kif14, Kif15, Kif20a, Kif20b and K23 (shown in FIG. 3G). Expression profiles of several Nox family genes were further analyzed and showed that Cyba and Cybb, the latter being specific to immune cells, were upregulated at D23, whereas other genes, such as Nox1 and Nox4 were slightly but not increased at varying days (shown in FIG. 3H).

To determine the extent to which these findings are conserved in humans, RNAseq was conducted on three paclitaxel-treated breast cancer patients with CIPN and three age/sex-matched healthy controls. Upon enrolment, each participant completed a questionnaire and physical examination, followed by a full-thickness skin punch biopsy. The patients were diagnosed with CIPN 35 weeks (CIPN35), 31 weeks (CIPN31), and 5 weeks (CIPN05) prior to the biopsy. After RNA extraction and processing, Illumina RNAseq analysis was performed, followed by heatmap and PCA plot generation using iDEP (Ge et al., 2018, BMC Bioinformatics 19: 534). Differences in gene expression between the three CIPN patients and healthy controls were evident from these two analyses (shown in FIG. 3I). Further enrichment analysis showed that the patients with their first CIPN diagnosis 31 and 35 weeks prior to the skin biopsy shared distinct gene clusters that separated from the patient with the most recent CIPN diagnosis (5 weeks prior). Although KIF11 was upregulated in all three patients compared with controls, the expression differences were not due to a strong expression increase in one of the CIPN patients (CIPN31). Differential gene expression analysis further identified several other upregulated KIF genes (shown in FIG. 3J). STRING interactome analysis of upregulated KIF genes revealed that KIF11 serves as a central hub for these co-upregulated KIF genes (shown in FIG. 3K). Expression analysis of NADPH oxidases identified NOX1 and CYBB as the most highly upregulated genes in the paclitaxel-treated patient skin compared with healthy controls (shown in FIG. 3L). Using the top 1,200 most variable differentially expressed genes, three major clusters were further identified with iDEP (shown in FIG. 3M). The first cluster showed pathways downregulated in the CIPN patients, which included processes, such as “epithelium/epidermis development”, “keratinocyte differentiation”, “gluconeogenesis”, and “lipid catabolic processes”. The second cluster (B) harbored genes upregulated in the 5-week CIPN patient skin, and these were involved in processes like “hydrogen peroxide catabolic process”, “cell death”, and “response to hydrogen peroxide”. Cluster C contained genes upregulated in the 31/35-week CIPN patients, with functions in “chromatin remodeling/assembly/disassembly”, “chromosome condensation”, “chromosome organization”, and “DNA conformation change”. These results establish conserved skin-specific gene expression profiles for mice and humans whereby paclitaxel promoted expression of genes involved in cell cycle and oxidative stress regulation.

### Example 4: Kif11/Eg5 Promoted dfMT and Nuclear X-ROS Formation in Zebrafish

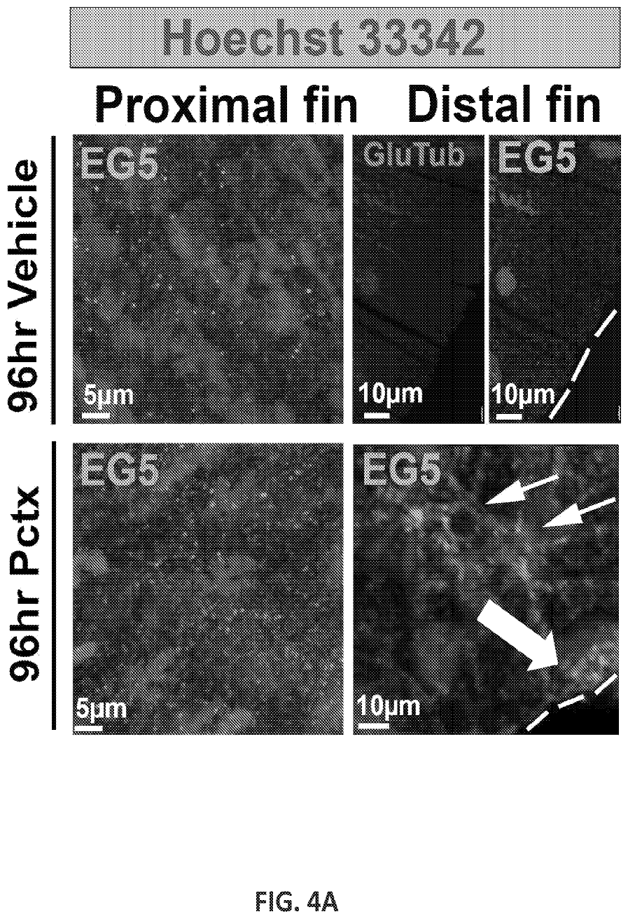

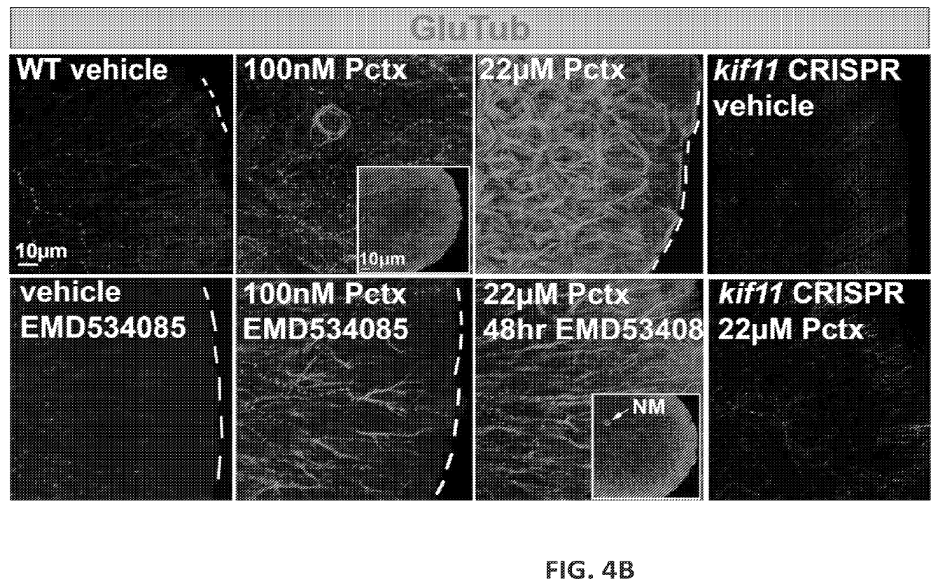

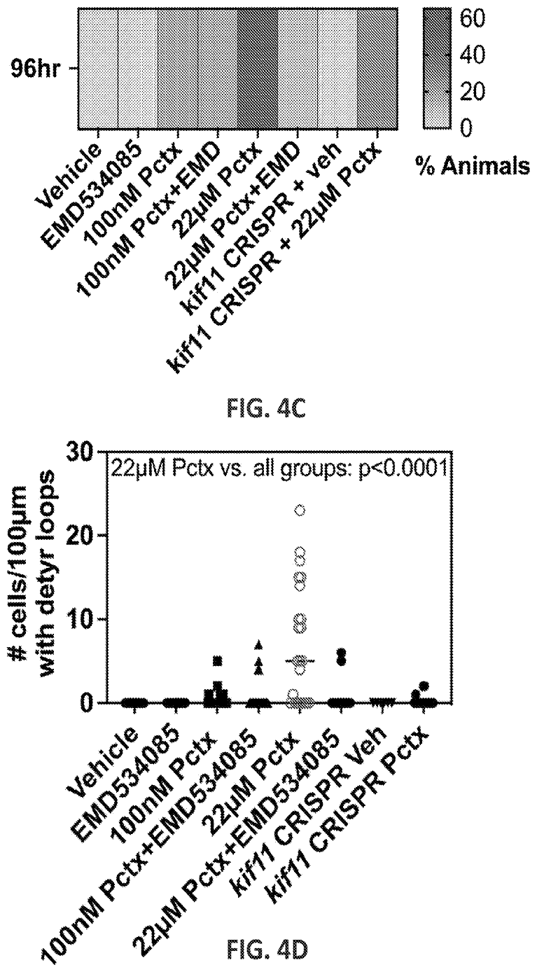

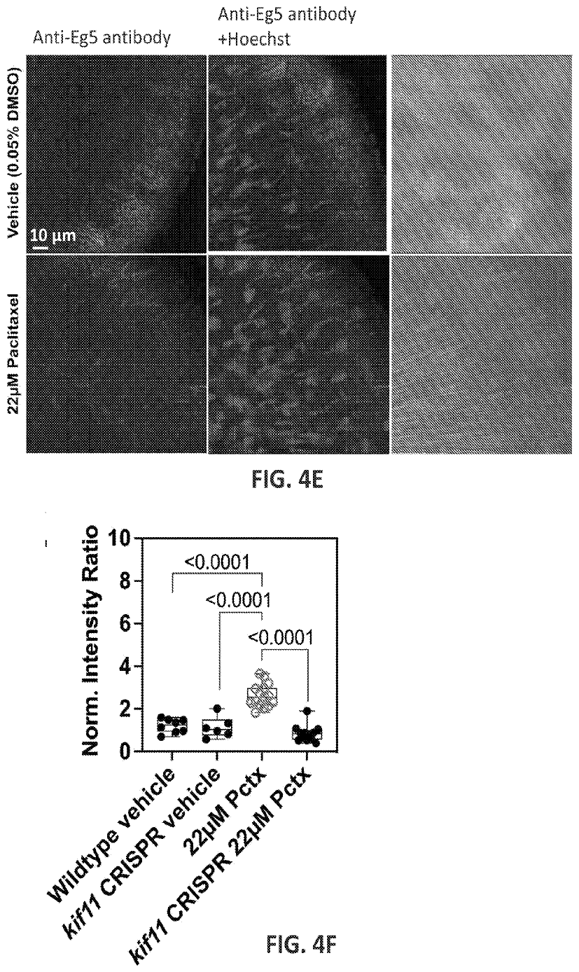

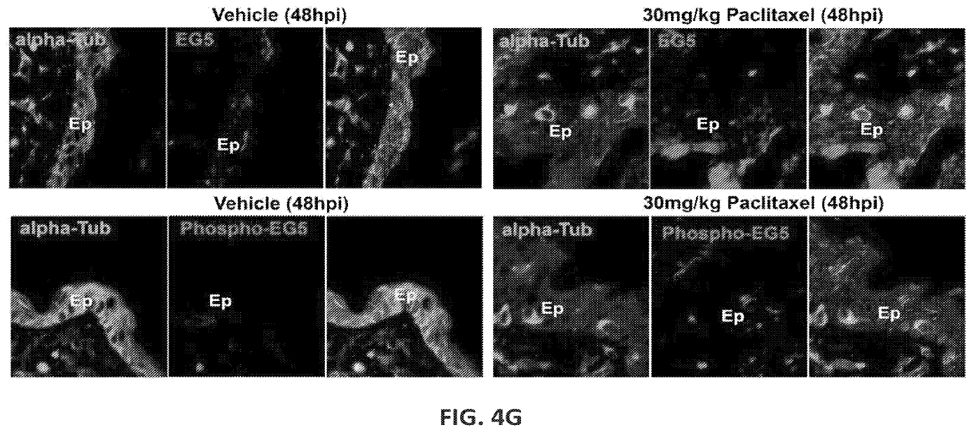

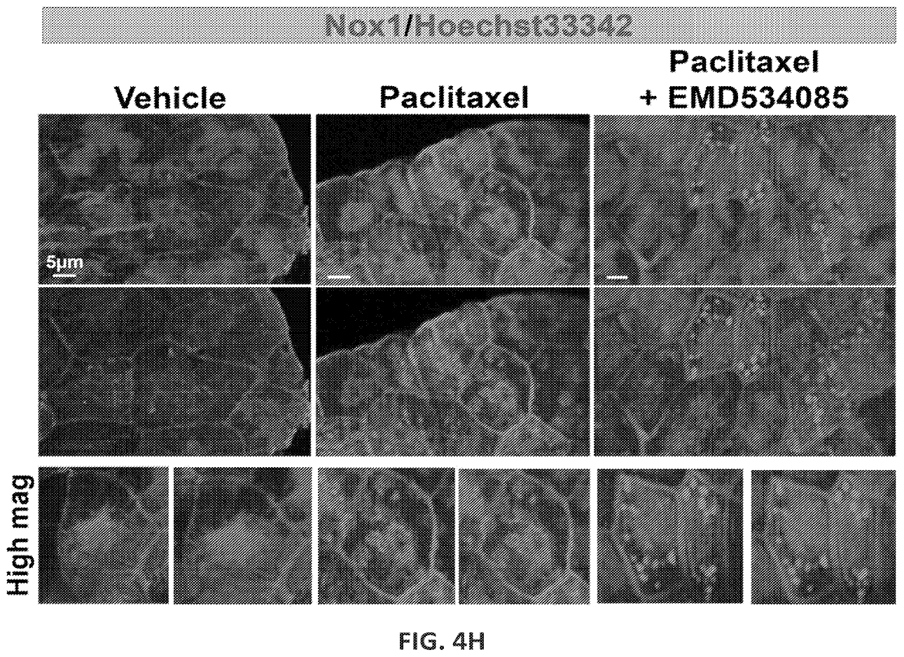

Because Eg5 has been linked to bortezomib-induced peripheral neuropathy, subsequent analyses were focused on this cell cycle regulator, using primarily zebrafish due to their in vivo imaging capabilities. First, whether paclitaxel induced Eg5 expression in caudal fin keratinocytes was determined. Following 96 hr vehicle treatment, Eg5 immunofluorescence staining with an antibody targeting the conserved phosphorylation site, Thr927 (PTGTTPQRK—SEQ ID NO: 24), revealed a uniform punctate Eg5 localization in the caudal fin except around the fin edge (shown in FIG. 4A). Treatment with 22 μM paclitaxel for 96 hours promoted Eg5 puncta formation and Eg5 location to structures reminiscent of microtubule asters, such as formed during early mitosis (Meaders et al., 2020, Cell Rep 33: 108213), which was most prominent at the fin edge. Because of the microtubule crosslinking activity of Eg5 during spindle formation and elongation (Goulet et al., 2013, Int Rev Cell Mol Biol 304: 419-466; Mann et al., 2019, Trends Cell Biol 29: 66-79), which resembles the observed Eg5 expression pattern in keratinocytes, whether Eg5 played a role in paclitaxel-dependent dfMT formation was determined. Indeed, combination of the Eg5 inhibitor, EMD534085, with 22 μM paclitaxel largely prevented dfMTs formation in the majority of animals (vehicle: 0%, EMD534085: 0%, 100 nM paclitaxel: 20%, 100 nM paclitaxel+EMD534085: 16%, 22 μM paclitaxel: 65.22%, 22 μM paclitaxel+EMD534085: 9.5%) (shown in FIG. 4B and FIG. 4C). Similarly, the number of dfMT-harboring keratinocytes per animal was reduced with EMD534085/paclitaxel co-administration (96 hr vehicle: 0±0 vs. EMD534085: 0±0 vs. 100 nM paclitaxel: 0.4±0.21 vs. 100 nM paclitaxel+EMD534085: 0.8±0.38 vs. 22 μM paclitaxel: 6.957±1.49 vs. 22 μM paclitaxel+EMD534085: 0.52±0.36 cells/animal) (shown in FIG. 4D). Since 25 μM EMD534085 was administered after 2d of vehicle and paclitaxel treatment, the few keratinocytes harboring dfMTs in the presence of 22 μM paclitaxel and EMD534085 likely formed prior to inhibitor administration, consistent with the finding that dfMTs were already present in some animals after 48 hr paclitaxel treatment (shown in FIG. 1I and FIG. 1J).

To validate the specificity of Eg5, kif11 was transiently deleted by injecting CRISPR oligos into 1-cell stage embryos. These were directed to exon 5 (~90aa of 1072aa), which eliminated Eg5 protein expression (shown in FIG. 4E) (percent animals with dfMTs: CRISPR+vehicle: 0%; CRISPR+22 μM paclitaxel: 25%; number of keratinocytes per animal with dfMTs: CRISPR+vehicle: 0±0; CRISPR+22 μM paclitaxel: 0.29±0.16) (shown in FIG. 4B-D). Interestingly, transient kif11 CRISPR knockout also reduced microtubule detyrosination when comparing 96 hr vehicle and paclitaxel treated animals, suggesting a role for Eg5 in detyrosination, which was not modified when animals were treated with EMD534085 (normalized detyrosination fluorescence intensity: vehicle: 1.2±0.11 vs. CRISPR+vehicle: 1.12±0.2; 22 μM paclitaxel: 2.62±0.14 vs. CRISPR+paclitaxel: 0.85±0.11) (shown in FIG. 4F). Importantly, increased total and phospho-Eg5 levels were also detected in basal and suprabasal epidermal keratinocytes of mice following a single injection of either vehicle or 20 mg/kg paclitaxel (shown in FIG. 4G). These findings established a conserved role for paclitaxel in epidermal Eg5 induction.

To determine whether Eg5 acted upstream of X-ROS and sensory axon degeneration, the subcellular localization of Nox1 in the presence and absence of EMD534085 was first analyzed. Immunofluorescence staining showed that EMD534085 co-administration with paclitaxel led to more condensed cytoplasmic vesicles compared with paclitaxel alone. Although EMD534085 did not affect paclitaxel-induced Nox1 plasma membrane translocation (Membrane:cytoplasmic ratio: vehicle: 1.78±0.09 vs. EMD534085: 1.7±0.1 vs. paclitaxel: 2.35±0.17 vs. paclitaxel+EMD534085: 2.072±0.12), Nox1 was however largely absent from the nucleus in EMD534085-treated animals (Nucleus:cytoplasmic ratio: vehicle: 1.04±0.09 vs. EMD534085: 1.36±0.08 vs. paclitaxel: 1.73±0.1.6 vs. paclitaxel+EMD534085: 1.35±0.08) (shown in FIG. 4H and FIG. 4I). This indicated that Eg5 induced Nox1 nuclear, but not plasma membrane, translocation in a paclitaxel-dependent manner.

Further quantification of compartmentalized HyPer oxidation near the plasma membrane and the nucleus (co-labelled with Hoechst33342) showed increased oxidation within the nucleus upon paclitaxel treatment, but surprisingly not at the plasma membrane. Nuclear HyPer oxidation was reduced upon co-administration of EMD534085 without effects on plasma membrane oxidation (shown in FIG. 4J). These findings supported a role for Eg5 in nuclear X-ROS production as a result of microtubule fasciculation and increased mechanical tension.

### Example 5: Detyrosinated Fasciculated Microtubules Associated with Nuclei

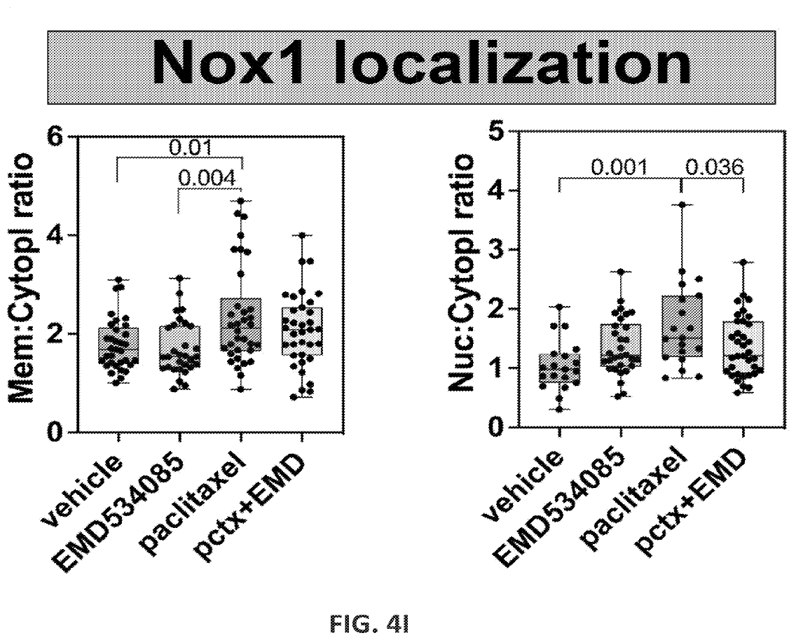

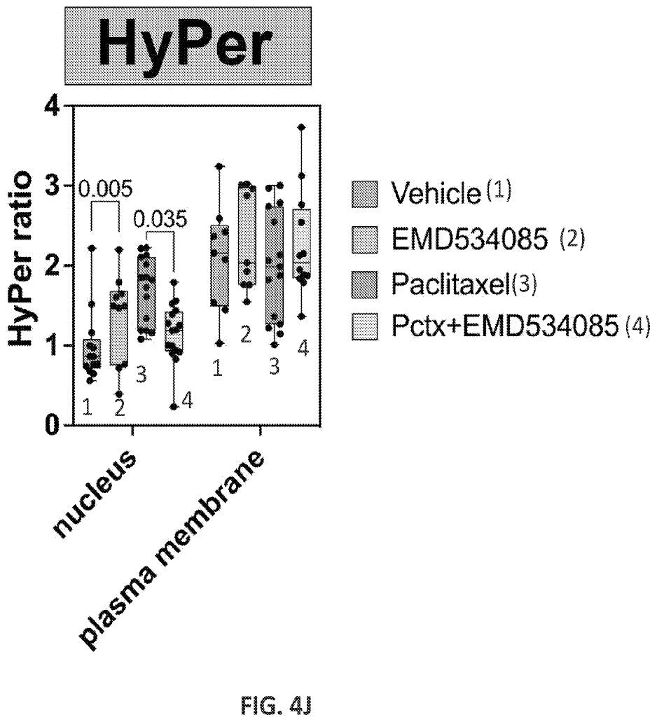

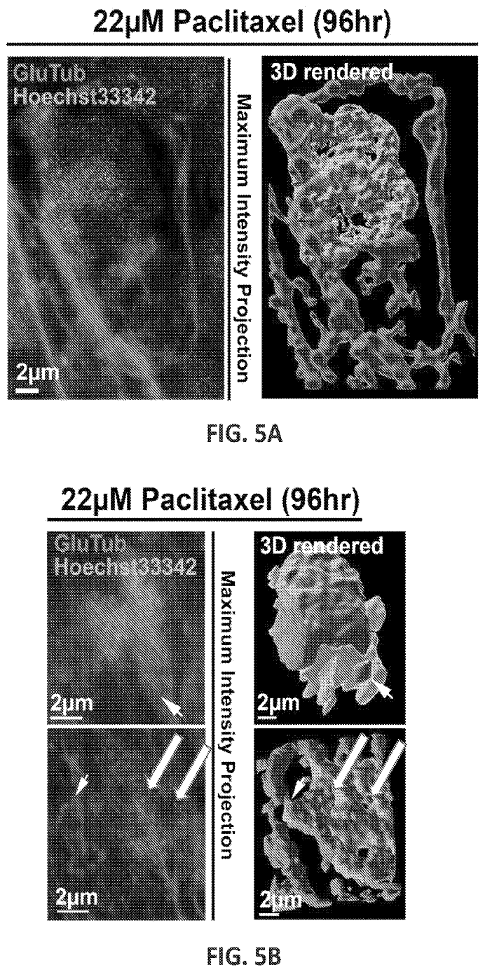

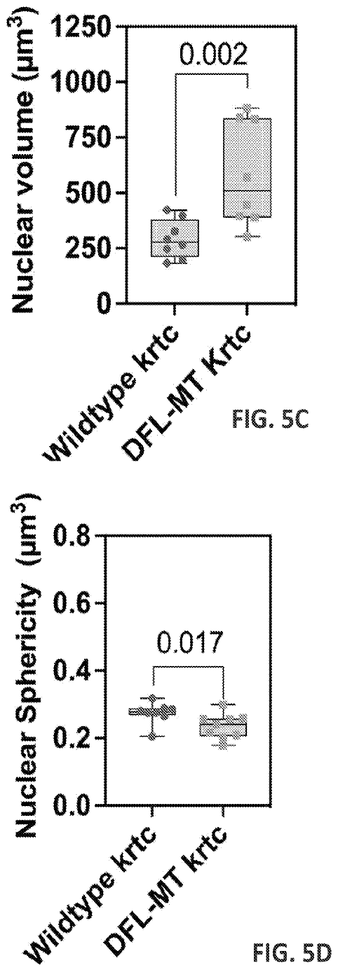

It was expected that dfMTs should be in close proximity to the nucleus in order to activate Nox1 by mechanotransduction. To further confirm this expectation, high resolution imaging and 3D rendering of keratinocyte dfMTs and nuclei were used. These experiments demonstrated that 96 hr paclitaxel treatment led to a partial association between dfMTs and nuclei (shown in FIG. 5A) whereby the nuclear content was sometimes pinched off or nuclei were perforated by dfMTs (shown in FIG. 5B). Moreover, dfMTs were typically associated with enlarged nuclei (vehicle nuclear volume: 291.3±30.62 μm3 vs. paclitaxel: 582±83.18 μm3) (shown in FIG. 5C) that were less spherical (1=spherical vs. 0=nonspherical: vehicle: 0.27±0.01 vs. paclitaxel: 0.23±0.01) (shown in FIG. 5D). These results further suggested that dfMTs formed a physical barrier that impairs nuclear function.

### Example 6: EMD534085 Promoted Keratinocyte Mitosis and Cell Death in the Presence of Paclitaxel

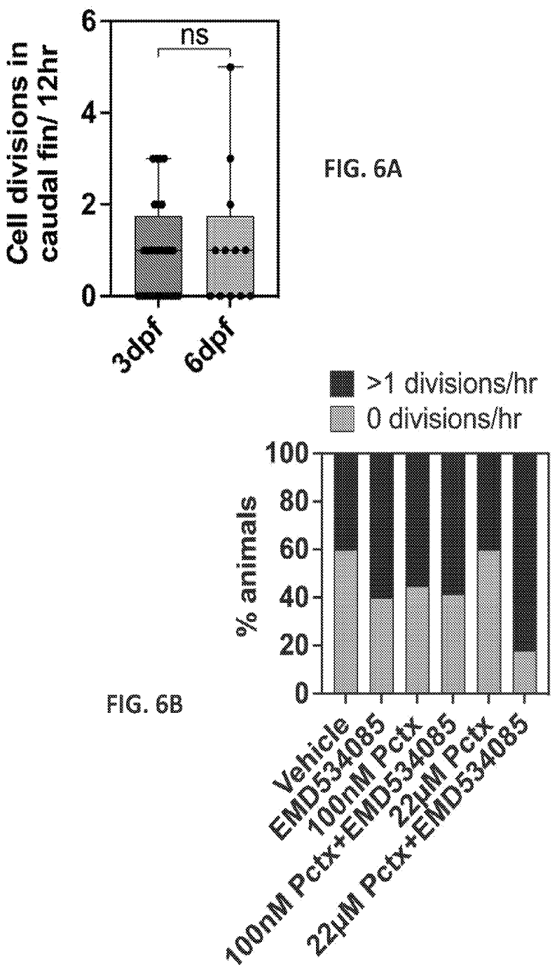

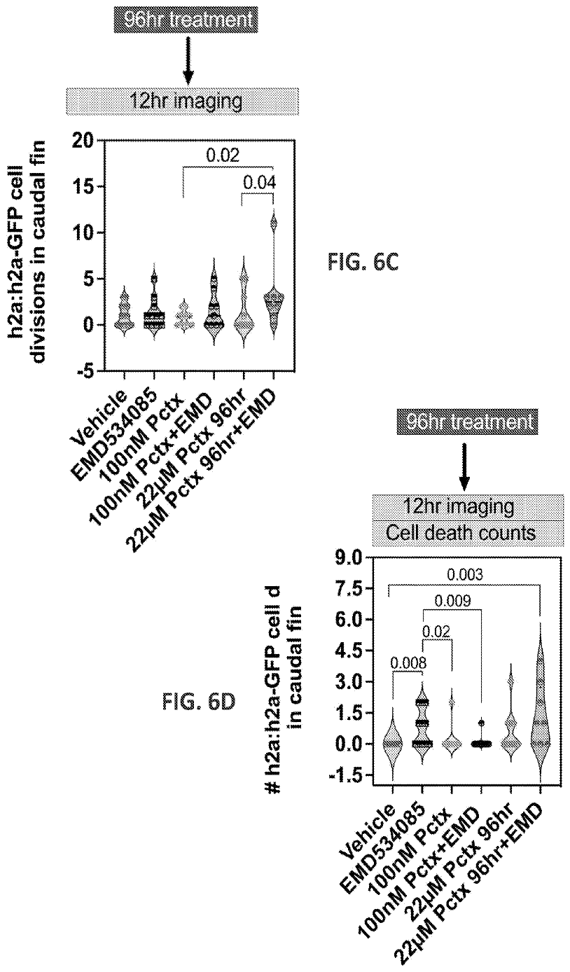

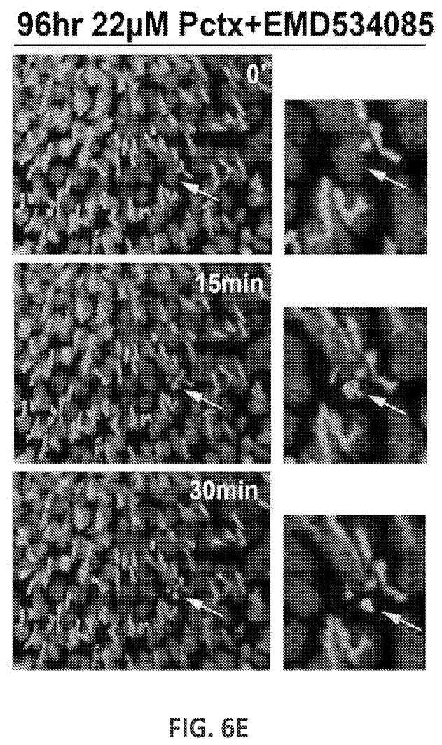

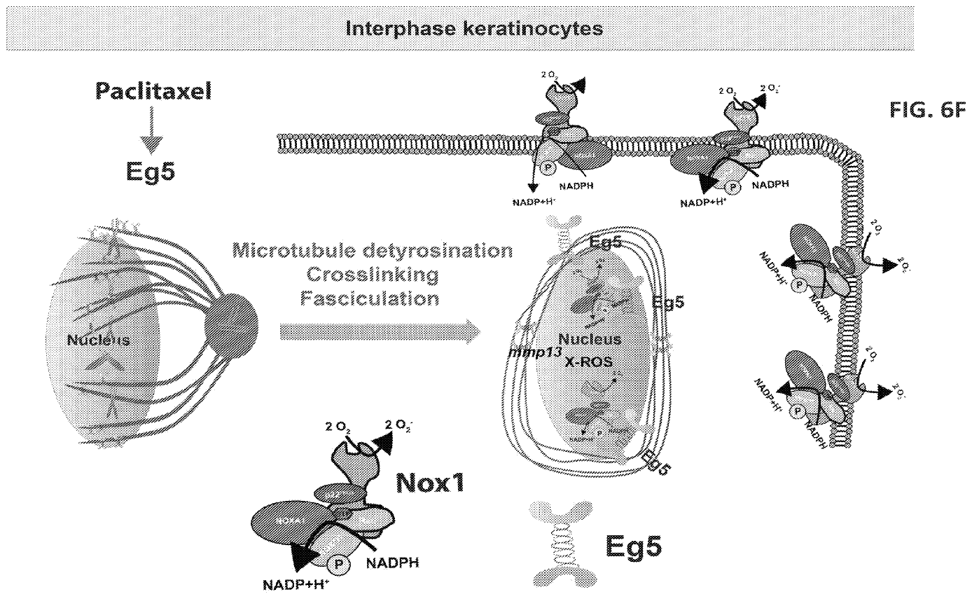

Because paclitaxel induced Eg5 expression in keratinocytes, and in cancer cells has a major role in preventing mitosis while promoting apoptosis (Jordan et al., 1993, Proceedings of the National Academy of Sciences of the United States of America 90: 9552-9556; Zasadil et al., 2019, Sci Transl Med 6: 229ra243; Zhao et al., 2022, Apoptosis 27: 647-667), whether Eg5 inhibition in combination with paclitaxel influenced keratinocyte mitosis and apoptosis was investigated. If dfMTs form a physical barrier, increased mitosis should be seen when EMD534085 was co-administered with paclitaxel. Consistent with this hypothesis, in vitro studies using paclitaxel sensitive and resistant ovarian cancer cells showed that Eg5 inhibition with HR22C16-A1 antagonized the effects of paclitaxel on mitotic inhibition (Marcus et al., 2018, Mol Med Rep 17: 8289-8299). To examine the effects of EMD534085 on mitosis, cell divisions in keratinocytes expressing nuclear h2a-h2a:GFP were quantified using 12 hr time-lapse imaging. Surprisingly, cell divisions in caudal fin keratinocytes at 3dpf (embryonic to larval transition) and 6dpf (late larval stage) were rare. The few divisions present were mostly restricted to the distal notochord region, with an average of 0.96±0.20/12 hr divisions at 3dpf and 1.16±0.44/12 hr at 6dpf in this region (shown in FIG. 6A). At least 1 cell division/12 hr was present in 40-60% of animals either treated with vehicle, 100 nM and 22 μM paclitaxel, as well as upon treatment with EMD534085 and 100 nM paclitaxel/EMD534085. Combination treatment with 22 μM paclitaxel and EMD534085 increased the percentage of animals with at least 1 cell division to 88% (shown in FIG. 6B). Mitotic divisions per animal were also increased with EMD534085 and 22 μM paclitaxel in combination, possibly due to reduced fasciculation leading to less impaired nuclei to divide (96 hr, vehicle: 1.07±0.3 vs. 100 nM paclitaxel: 0.72±0.423 vs. 22 μM paclitaxel: 1.4±0.67 vs. EMD534085: 1.27±0.46/12 hr vs. 100 nM paclitaxel+EM534085: 1.55±0.62/12 hr vs. 22 μM paclitaxel+EMD534085: 3.125±1.18) (shown in FIG. 6C).

Since apoptosis has been found to be the primary mechanism of cell death upon paclitaxel treatment (Zasadil et al., 2019, Sci Transl Med 6: 229ra243; Zhao et al., 2022, Apoptosis 27: 647-667; Marcus et al., 2018, Mol Med Rep 17: 8289-8299; Ren et al., 2018, Mol Med Rep 17: 8289-8299; Jordan et al., 1996, Cancer research 56: 816-825), the effects of 96 hr paclitaxel/EMD534085 combination treatment on cell death by imaging h2a:h2a-GFP fish were further explored with in vivo time-lapse imaging for 12 hours. Overall, there was either none or very little cell death in vehicle and 100 nM paclitaxel-treated animals, whereas 22 μM paclitaxel increased the number of dying cells, however, this was not significant due to great variability (vehicle: 0±0 vs. 100 nM paclitaxel: 0.18±0.18 vs. 22 μM paclitaxel: 0.6±0.3 dying cells/caudal fin). EMD534085 alone increased cell death slightly above 22 μM paclitaxel treatment, and surprisingly reduced cell death when co-administered with 100 nM paclitaxel (EMD534085: 0.81±0.26 vs. 100 nM paclitaxel+EMD534085: 0.07±0.07). Nevertheless, EMD534085 combination with 22 μM paclitaxel significantly increased cell death compared with vehicle (22 μM paclitaxel+EMD534085: 1.37±0.53) but not with paclitaxel (shown in FIG. 6D and FIG. 6E). Thus, while EMD534085 in combination with a paclitaxel slightly enhanced mitosis, its effects on cell death were negligible. These findings indicate that keratinocyte dfMTs were formed independently of mitosis but impact mitotic behavior, while effects on apoptosis in the presence of paclitaxel are not enhanced (shown in FIG. 6F).

### Example 7: Eg5 Did not Influence Microtubule Growth Behavior

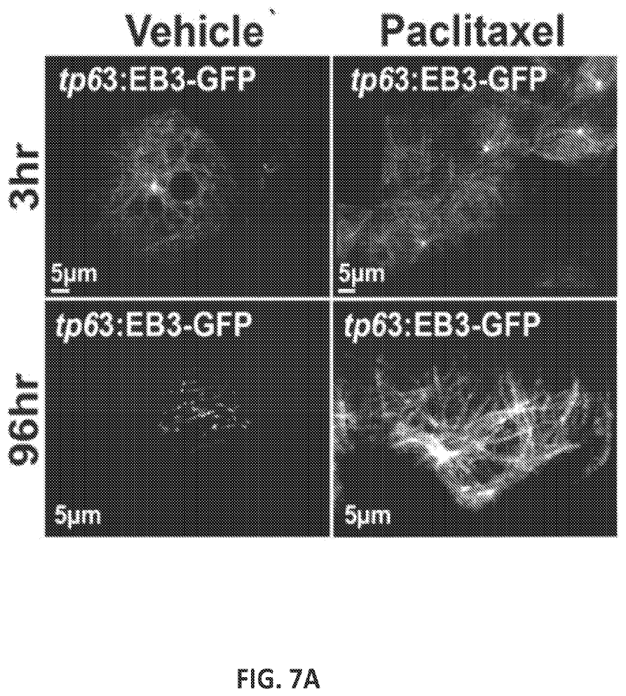

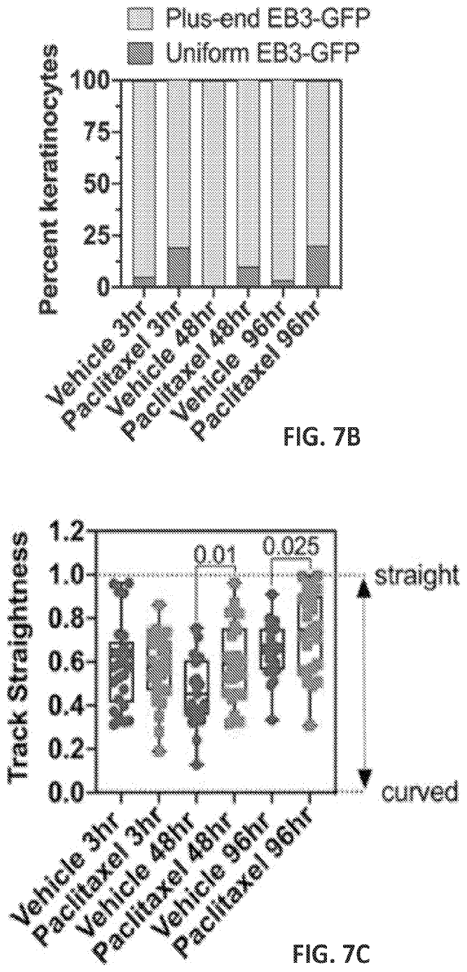

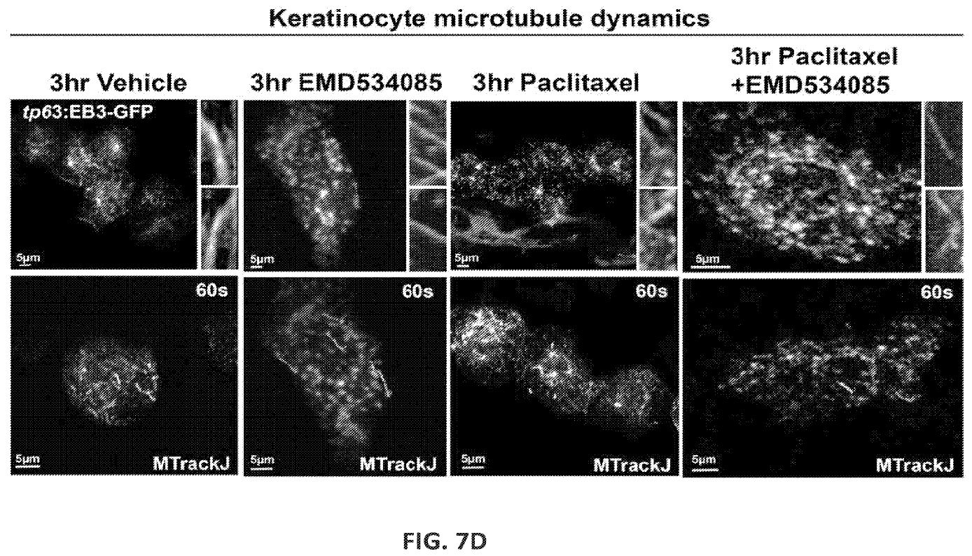

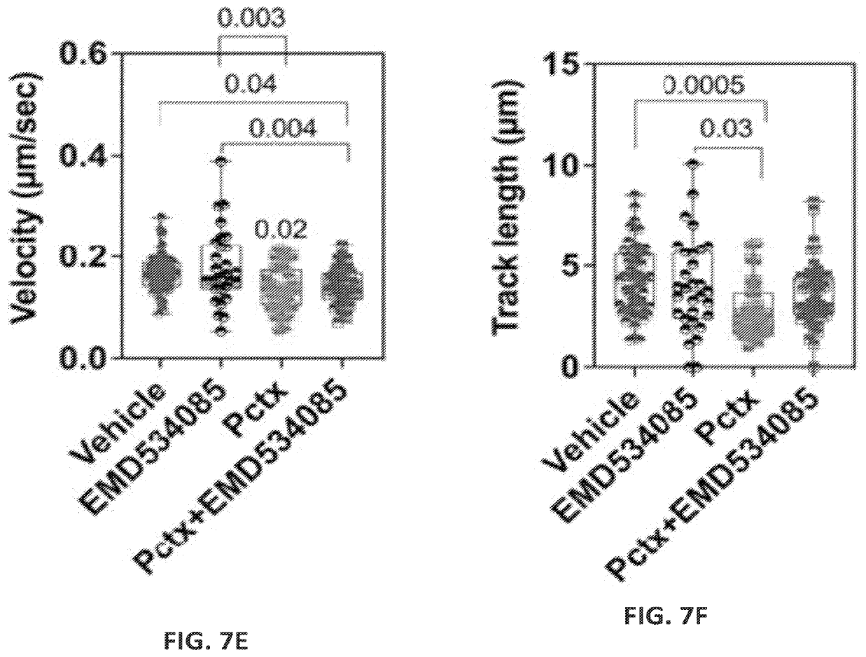

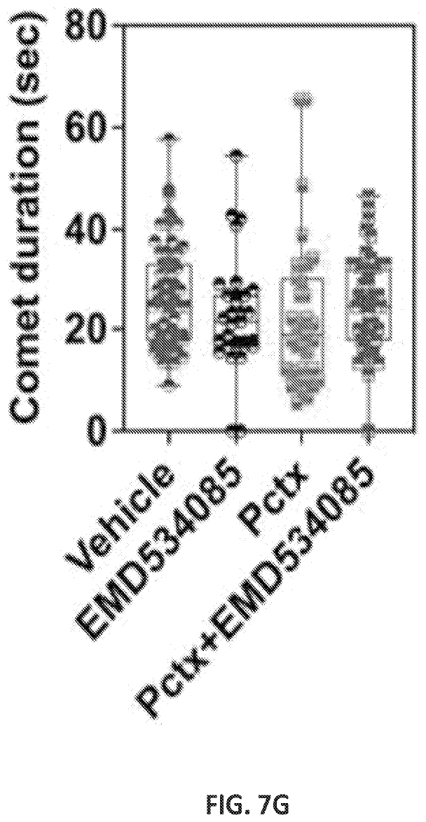

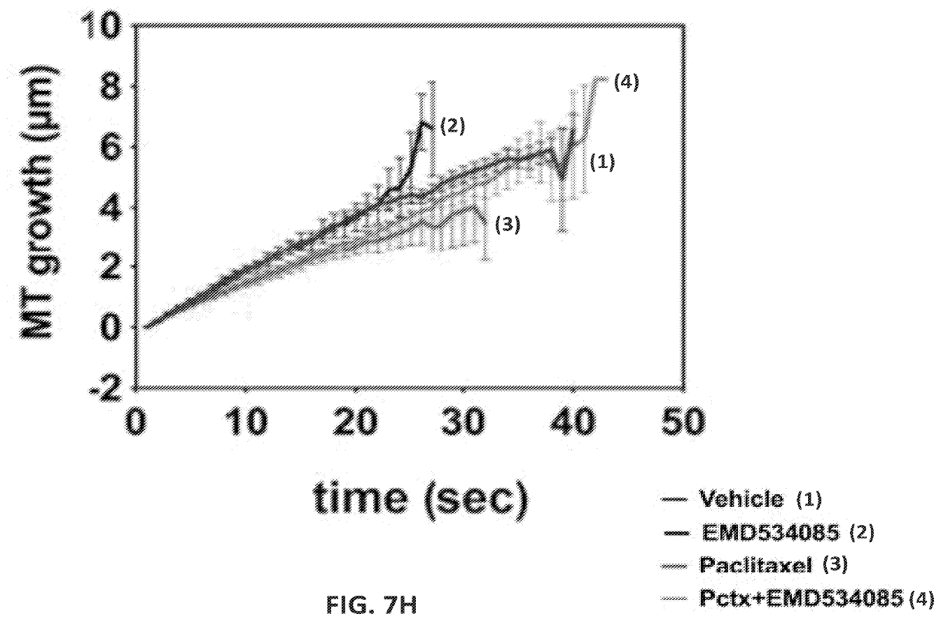

These findings set forth above suggested that Eg5 acts on stable microtubules. To validate this, microtubule growth dynamics were characterized in basal keratinocytes expressing the microtubule plus-end binding protein, EB3-GFP (courtesy of R. Koester) (Distel et al., 2010, The Journal of cell biology 191: 875-890). Time-lapse recordings (1 fr/sec for 60 sec) showed that, as expected, EB3-GFP mostly localized to microtubule plus-ends during growth. However, a small proportion of keratinocytes also harbored uniformly labelled EB3-positive microtubules, consistent with previous observations in murine axons (Kleele et al., 2015, Proceedings of the National Academy of Sciences of the United States of America 112: 7501-7506). Paclitaxel treatment for 3 hr resulted in an increased number of keratinocytes with uniform EB3 labelling (3 hr vehicle: 5.12% vs. paclitaxel: 19.29%; 48 hr vehicle: 0% vs. paclitaxel: 10.09%; 96 hr vehicle: 3.44% vs. 22 μM paclitaxel: 20.13%) (shown in FIG. 7A and FIG. 7B), indicative of microtubule stabilization. Intriguingly, however, microtubules with uniform EB3-GFP binding appeared linear and continuous treatment for 48 hr and 96 hr with paclitaxel further enhanced the linearity (shown in FIG. 7C). This behavior therefore contrasts the curved conformation of stable dfMTs. Further analysis of microtubule growth dynamics showed that 3 hr paclitaxel treatment decreased the growth velocity and track length of microtubules, whereas comet duration per se was not affected (shown in FIG. 7D-7H). Combination treatment of EMD534085 and 22 μM paclitaxel did not alter the effects induced by paclitaxel alone, suggesting that Eg5 does not influence paclitaxel-dependent microtubule growth dynamics.

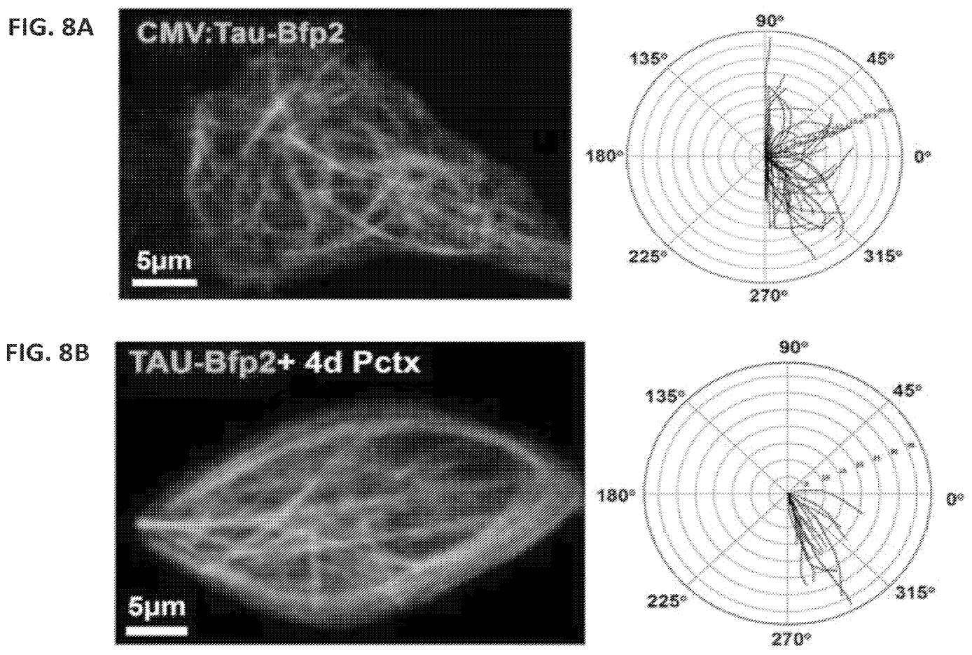

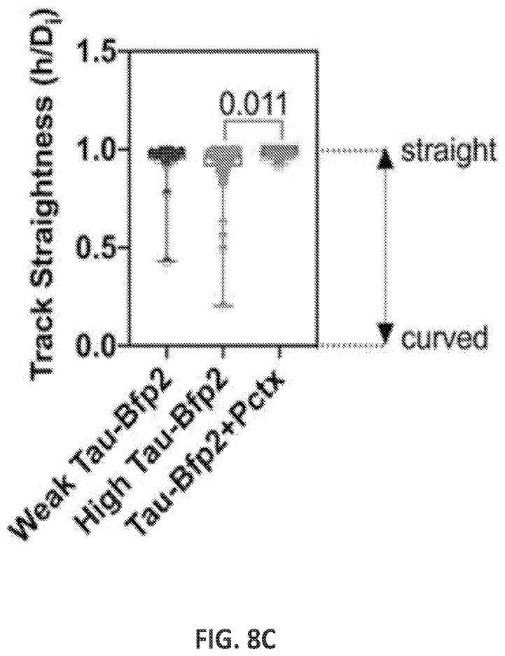

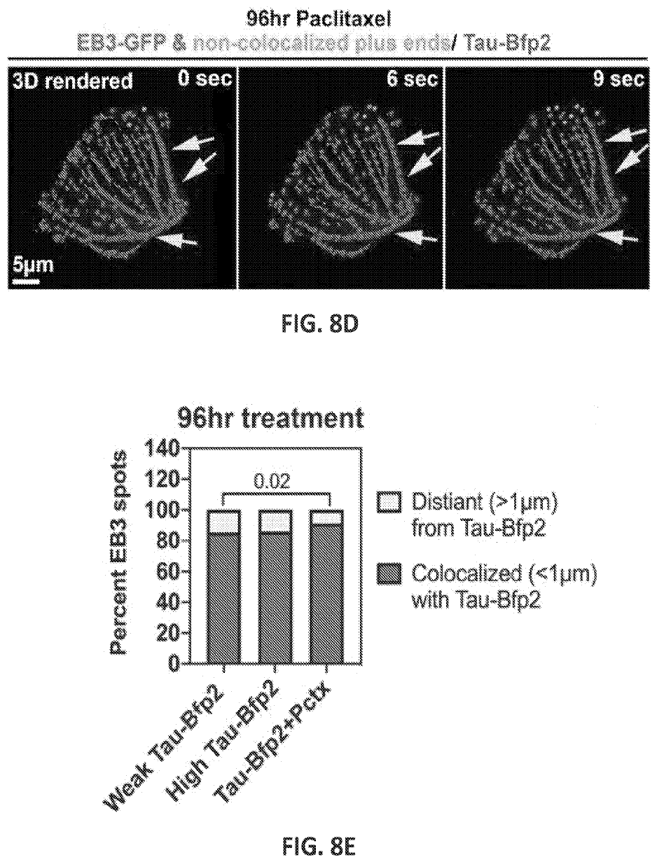

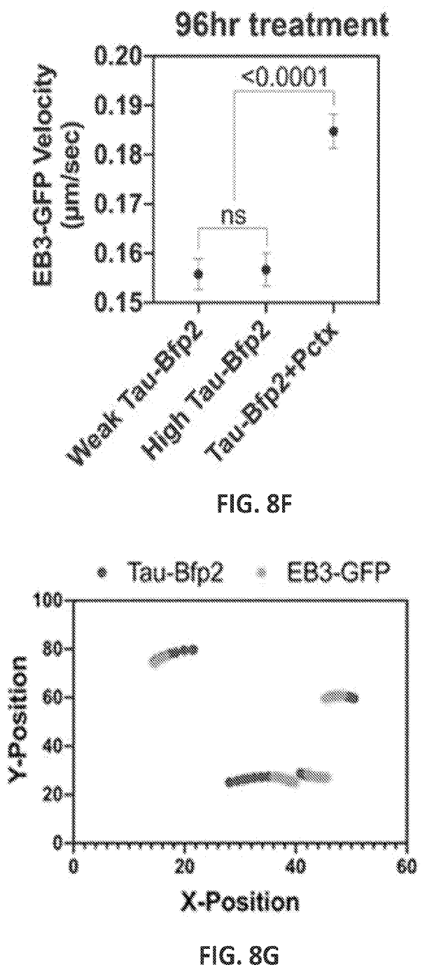

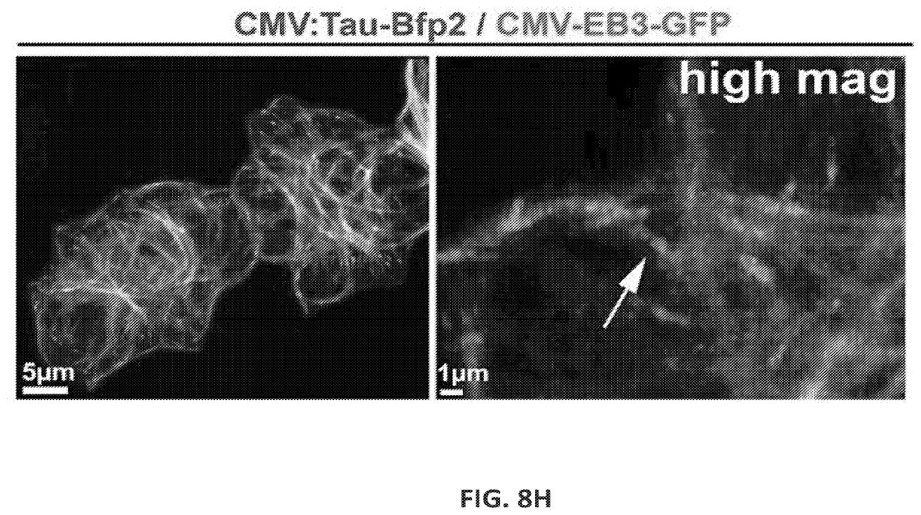

To further determine whether microtubule stabilization per se leads to curved microtubules, or whether this was a specific property pertaining to paclitaxel, microtubules in keratinocytes of zebrafish injected with CMV:Tau-Bfp2 were analyzed. This microtubule-associated protein has been shown to induce microtubule stabilization by binding to the interface between α and β-tubulin heterodimers (Kadavath et al., 2015, Proceedings of the National Academy of Sciences of the United States of America 112: 7501-7506; Ross et al., 2004, Proceedings of the National Academy of Sciences of the United States of America 101: 12910-12915). In experiments disclosed herein it was found that Tau-Bfp2 induced fasciculation and mild curving of microtubules (shown in FIG. 8A), which differed from the curved dfMT conformation induced by paclitaxel. Intriguingly, treatment of zebrafish expressing Tau-Bfp2 in keratinocytes for 96 hr with paclitaxel promoted linearization of microtubules rather than circularization (1=straight, 0=curved: 96 hr weak Tau-Bfp2 expression: 0.94±0.01, strong Tau-Bfp2 labelling: 0.93±0.01, strong Tau-Bfp2+paclitaxel: 0.98±0.002) (shown in FIG. 8B and FIG. 8C), consistent with previous findings (Kadavath et al., 2015, Proceedings of the National Academy of Sciences of the United States of America 112: 7501-7506). Tau therefore seems to prevent paclitaxel-induced dfMT formation. Together these data suggested that dfMTs formed as a consequence of paclitaxel dependent Eg5 activation rather than microtubule stabilization per se.

Interestingly, co-expression of EB3-GFP with Tau-Bfp2 in keratinocytes revealed a subpopulation of growing EB3-GFP plus-ends that grew along stable Tau-Bfp2 microtubules (shown in FIG. 8D and FIG. 8E). Treatment with 22 μM paclitaxel for 96 hr further increased Tau-Bfp2 and EB3-GFP co-localization regardless of weak or strong Tau-Bfp2 expression (EB3-GFP puncta co-localized with Tau-Bfp2, weak Tau-Bfp2 expression: 85.42±1.64% of, strong Tau-Bfp2 expression: 86.13±1.22%, Tau-Bfp2+96 hr paclitaxel: 91.26±1.64%). Also the velocity of EB3-GFP plus-ends moving along these linear Tau-Bfp2 microtubules was increased with paclitaxel treatment compared to Tau-Bfp2 alone (weak Tau-Bfp2: 0.155±0.003 μm/s, high Tau-Bfp2: 0.156±0.003 μm/s vs. Tau-Bfp2+96 hr paclitaxel: 0.184±0.003 μm/s) (shown in FIG. 8F). Plotting of the X/Y positions of some microtubules revealed that EB3-GFP growth trajectories closely followed Tau-Bfp2 associated microtubule filaments (shown in FIG. 8G and FIG. 8H). Thus EB3-GFP plus-ends appeared in part to grow along pre-existing stable microtubules, which could be a means to maintain cellular organization.

### Example 8: Keratinocyte-Specific Eg5 Overexpression Promoted Cutaneous Sensory Axon Degeneration

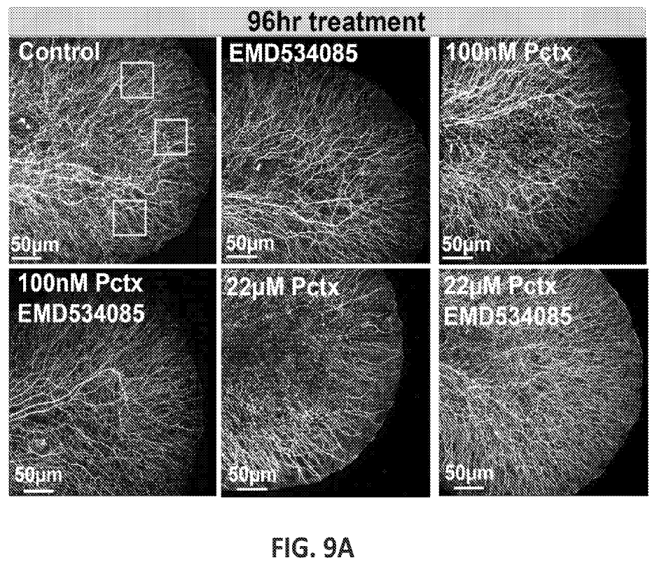

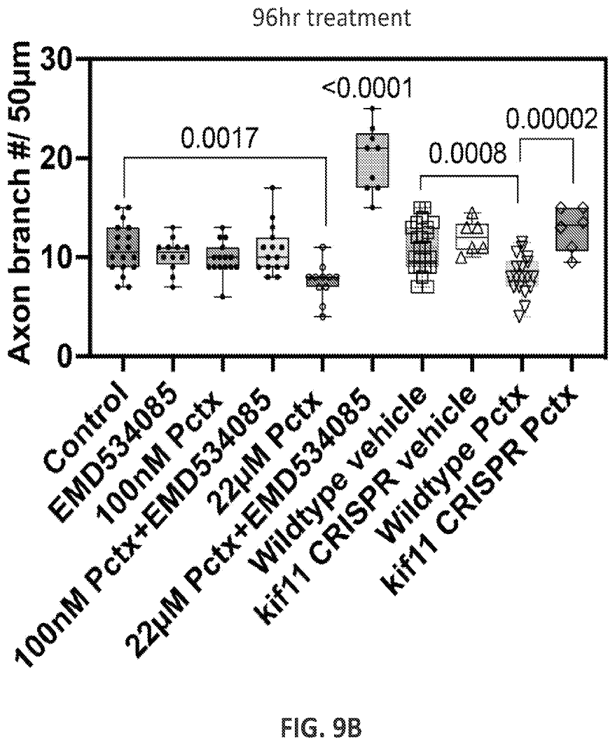

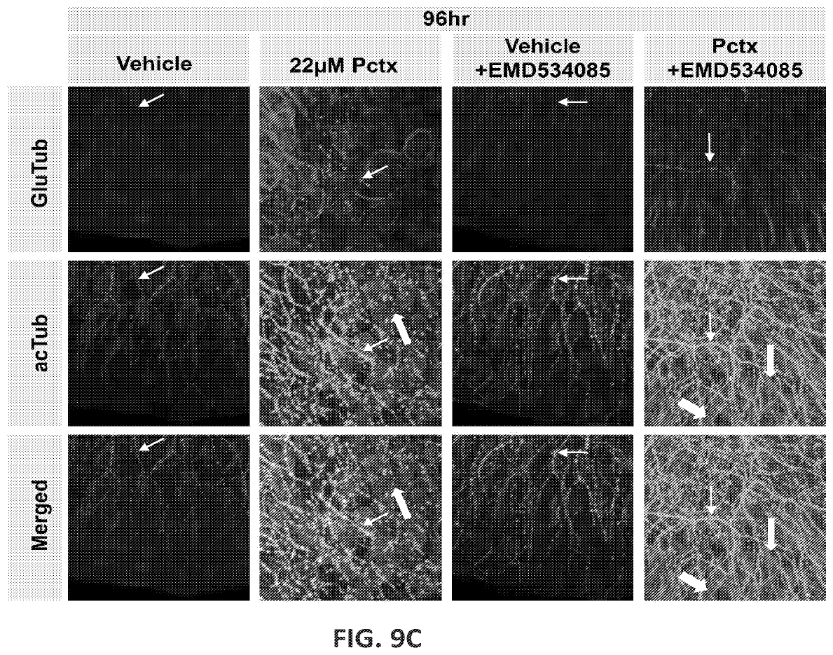

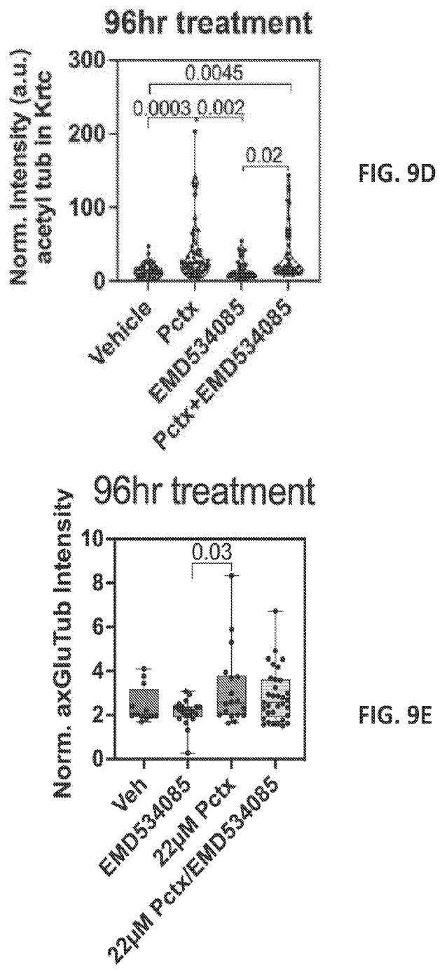

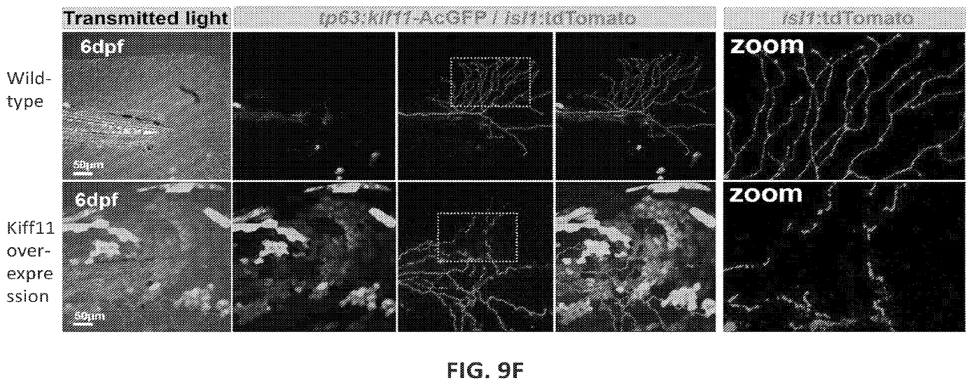

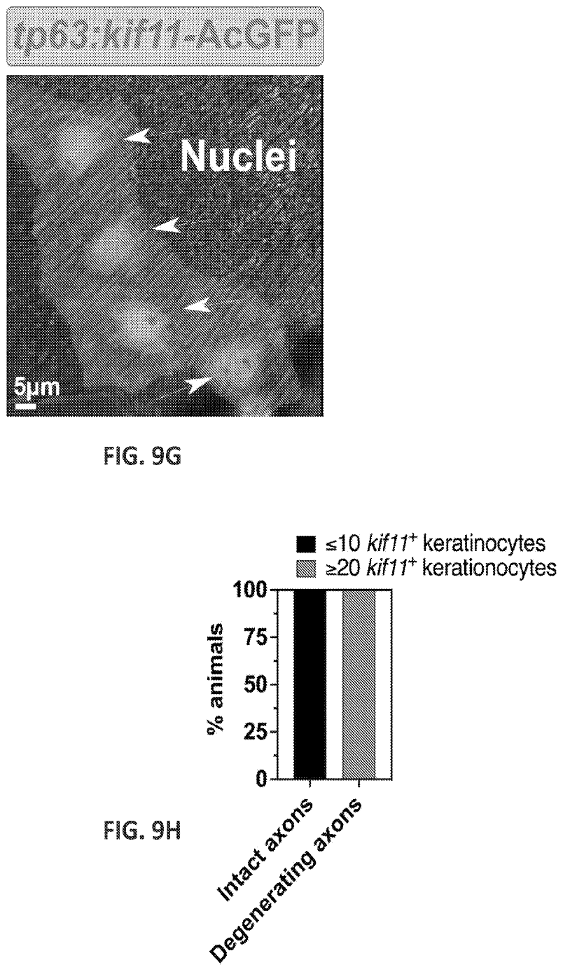

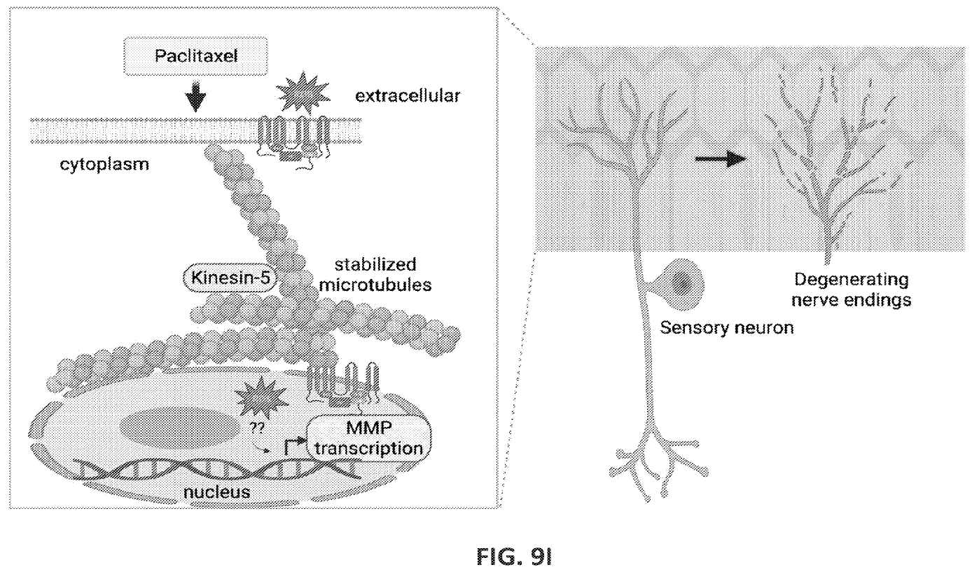

That the earliest signs of paclitaxel-induced axon degeneration are evident at the distal fin edge had been shown to be due to MMP-13 dependent ECM degradation (Lisse et al., 2016, Proceedings of the National Academy of Sciences of the United States of America 113: E2189-2198; Cirrincione et al., 2020, Sci Rep 10, 3970), consistent with the vast majority of dfMTs being present in this region (shown in FIG. 1G). To determine whether EG5-dependent dfMTs induced axon degeneration, axon degeneration in zebrafish that were treated for 96 hr with low (100 nM) and high (22 μM) paclitaxel concentrations in the presence and absence of EMD534085 was assessed. Treatment with vehicle, EMD534085 and 100 nM paclitaxel did not alter axon branch number assessed with acetylated tubulin staining. Treatment with 22 μM paclitaxel, however, reduced the axon branch number as previously shown (96 hr: vehicle: 10.83±0.58, EMD534085: 10.25±0.47, 100 nM paclitaxel: 9.86±0.43, 100 nM paclitaxel+48 hr EMD534085: 10.73±0.63, 22 μM paclitaxel: 7.54±0.56 branches/50 μm) (shown in FIG. 9A and FIG. 9B). Intriguingly, combination treatment of 22 μM paclitaxel and EMD534085 rescued axon degeneration even above control levels (19.89±1.09 branches/50 μm). These findings were consistent with observations that monastrol promotes neurite outgrowth in cultured mouse DRG neurons (Haque et al., 2004, Cell Motil Cytoskeleton 58: 10-16), suggesting a neuron-intrinsic growth effect. To further validate Eg5 as the pharmacological target of EMD534085 in axon regeneration, zebrafish injected with kif11 CRISPR oligo were also analyzed. Paclitaxel-treated kif11 CRISPR zebrafish did not show altered axon branch numbers in the caudal fin compared with vehicle-treated CRISPR and wildtype controls, although axon growth was not increased, as seen with EMD534085 (kif11-CRISPR+vehicle: 12.08±0.68, kif11-CRISPR+22 μM paclitaxel: 12.83±0.96) (shown in FIG. 9B). These findings implicated Eg5 as downstream target of paclitaxel in axon degeneration, although it was not determined why kif11 knockout modulated axon behavior differently from pharmacological Eg5 inhibition, possibly due to developmental effects of the CRISPR oligo.

Whether Eg5-dependent axon degeneration was caused by keratinocyte or neuron-specific effects (because inter alia Eg5 was also expressed neuronally (Falnikar et al., 2011, Mol Biol Cell 22: 1561-1574; Wei et al., 2022, Neurotherapeutics 19: 1401-1413) was determined as set forth herein. Possible neuron-intrinsic axon growth effects with EMD534085 were observed. Initially the extent to which axonal microtubules were stabilized was determined and whether this process was altered using EMD534085 in the presence and absence of paclitaxel. Quantification of axonal detyrosination in wildtype distal fins following 96 hr treatment with either vehicle or EMD534085 showed a punctate pattern in axons, whereas 22 μM paclitaxel but not paclitaxel/EMD534085 promoted a uniform distribution of dMTs along axon segments (shown in FIG. 9C). Quantification of the average detyrosination fluorescence intensity along individual axon segments however showed that only paclitaxel differed from EMD534085 (normalized GluTub intensity ratios, wildtype 96 hr vehicle: 2.44±0.24, EMD534085: 2.13±0.13, 22 μM paclitaxel: 3.19±0.42, 22 μM paclitaxel+EMD534085: 2.86±0.21) (shown in FIG. 9B, FIG. 9C, and FIG. 9E). Thus, paclitaxel does not appear to regulate dMT formation via Eg5 in axons.

Next a transgene encoding tp63:kif11-AcGFP for transient Eg5 overexpression in keratinocytes was injected. Plasmids isl1:tdTomato were further co-expressed in sensory neurons to label wildtype axons. Eg5 preferentially located to keratinocyte nuclei with some cytoplasmic labelling (shown in FIG. 9F and FIG. 9G), consistent with its nuclear localization in cultured interphase HeLa cells (Muretta et al., 2018, Proceedings of the National Academy of Sciences of the United States of America 115: E1779-E1788). Transient plasmid DNA injections into zebrafish typically mosaically label cells, which allowed for the observation of axons in the absence and presence of Eg5-overexpressing keratinocytes. Whereas wildtype axons in animals with few Eg5-GFP positive (≤510) keratinocytes) remained intact (shown in FIG. 9F, top panel), axons in contact with large areas of Eg5 overexpressing keratinocytes (≥20) degenerated (shown in FIG. 9F, bottom panel), FIG. 9G, and FIG. 9H). Therefore, keratinocyte-specific Eg5 overexpression mimicked paclitaxel-induced sensory axon degeneration. These findings supported a model in which Eg5 activation by paclitaxel stimulated X-ROS formation and MMP-13 dependent ECM degradation in keratinocytes, ultimately leading to axon degeneration (shown in FIG. 9I).

### Example 9: Paclitaxel Induced dfMT and Brittle Skin in Keratinocytes of Mice, which was Rescued by Eg5 Inhibitor

The data set forth herein showed that paclitaxel increased dfMT in an Eg5-dependent manner in zebra fish. To test whether this phenotype was also present in mice, 12-week old C57BL6/J mice were intraperitoneally injected with vehicle (0.5% DMSO/1×PBS), paclitaxel (24 mg/kg; one injection), and the EG5 inhibitor, EMD534085 (20 mg/kg; 3 injections: 1 before, during, and after paclitaxel), as indicated in the schematic of FIG. 11A. These mice were then sacrificed and the skin of the hind paw pad was fixed in 4% paraformaldehyde, followed by sectioning and staining with 1) anti-GluTub (to detect detyrosinated microtubules) and 2) DAPI to detect nuclei. As shown in FIG. 11B, paclitaxel treatment increased GluTub fluorescence, indicating increased dfMT. The thick white arrows in the paclitaxel image showed brittle skin in the stratum spinosum. While these cells lost their nucleus, these dark spots (empty regions) were not present in animals injected with vehicle or paclitaxel+EMD, suggesting a rescue effect. The white arrows point to cells that have increased detyrosination which was seen around the nucleus (high mag Paclitaxel image—FIG. 11B). The white arrows in the Pcxt+EMD image showed dMT; however, the high magnification image and DAPI image demonstrated that these cells were not associated with nuclei and seemed to be either dead cells or another cell type (not keratinocytes). The number of epidermal keratinocytes with dMT was quantified in FIG. 11C, which showed an increase with paclitaxel treatment. The increase was dampened when paclitaxel was injected with EMD534085.

In summary, the data showed that the phenotype observed in zebrafish was conserved in mammals, namely that paclitaxel induced dMT in epidermal keratinocytes, which can be rescued by EG5 inhibition with EMD534085.

All publications, patents, and patent applications mentioned in this specification are herein incorporated by reference to the same extent as if each individual publication, patent, and patent application was specifically and individually indicated to be incorporated by reference.

While some embodiments have been illustrated and described in detail in the appended drawings and the foregoing description, such illustration and description are to be considered illustrative and not restrictive. Other variations to the disclosed embodiments can be understood and effected in practicing the claims, from a study of the drawings the disclosure, and the appended claims. The mere fact that certain measures or features are recited in mutually different dependent claims does not indicate that the combination of these measures or features cannot be used. Any reference signs in the claims should not be construed as limiting the scope.

### REFERENCES

1. E. Gornstein, T. L. Schwarz, The paradox of paclitaxel neurotoxicity: Mechanisms and unanswered questions. Neuropharmacology 76 Pt A, 175-183 (2014).
2. A. A. Argyriou, J. Bruna, A. A. Genazzani, G. Cavaletti, Chemotherapy-induced peripheral neurotoxicity: management informed by pharmacogenetics. Nat Rev Neurol 13, 492-504 (2017).
3. A. A. Argyriou, J. Bruna, P. Marmiroli, G. Cavaletti, Chemotherapy-induced peripheral neurotoxicity (CIPN): an update. Crit Rev Oncol Hematol 82, 51-77 (2012).
4. A. A. Argyriou et al., Neurophysiological, nerve imaging and other techniques to assess chemotherapy-induced peripheral neurotoxicity in the clinical and research settings. Journal of neurology, neurosurgery, and psychiatry, (2019).
5. A. A. Argyriou, V. Zolota, O. Kyriakopoulou, H. P. Kalofonos, Toxic peripheral neuropathy associated with commonly used chemotherapeutic agents. J BUON 15, 435-446 (2010).
6. A. J. Beijers, J. L. Jongen, G. Vreugdenhil, Chemotherapy-induced neurotoxicity: the value of neuroprotective strategies. Neth J Med 70, 18-25 (2012).
7. J. A. Boyette-Davis, E. T. Walters, P. M. Dougherty, Mechanisms involved in the development of chemotherapy-induced neuropathy. Pain Manag 5, 285-296 (2015).
8. S. J. L. Flatters, P. M. Dougherty, L. A. Colvin, Clinical and preclinical perspectives on Chemotherapy-Induced Peripheral Neuropathy (CIPN): a narrative review. Br J Anaesth 119, 737-749 (2017).
9. G. J. Bennett, G. K. Liu, W. H. Xiao, H. W. Jin, C. Siau, Terminal arbor degeneration—a novel lesion produced by the antineoplastic agent paclitaxel. Eur J Neurosci 33, 1667-1676 (2011).
10. N. P. Staff et al., Pathogenesis of paclitaxel-induced peripheral neuropathy: A current review of in vitro and in vivo findings using rodent and human model systems. Experimental neurology 324, 113121 (2020).
11. M. Seretny et al., Incidence, prevalence, and predictors of chemotherapy-induced peripheral neuropathy: A systematic review and meta-analysis. Pain 155, 2461-2470 (2014).
12. S. E. Pease-Raissi et al., Paclitaxel Reduces Axonal Bclw to Initiate IP3R1-Dependent Axon Degeneration. Neuron 96, 373-386 e376 (2017).
13. I. Bobylev et al., Paclitaxel inhibits mRNA transport in axons. Neurobiol Dis 82, 321-331 (2015).
14. Y. Li et al., The Cancer Chemotherapeutic Paclitaxel Increases Human and Rodent Sensory Neuron Responses to TRPV1 by Activation of TLR4. J Neurosci 35, 13487-13500 (2015).
15. L. Brandolini et al., CXCR1/2 pathways in paclitaxel-induced neuropathic pain. Oncotarget 8, 23188-23201 (2017).
16. T. S. Lisse et al., Paclitaxel-induced epithelial damage and ectopic MMP-13 expression promotes neurotoxicity in zebrafish. Proceedings of the National Academy of Sciences of the United States of America 113, E2189-2198 (2016).
17. A. M. Cirrincione et al., Paclitaxel-induced peripheral neuropathy is caused by epidermal ROS and mitochondrial damage through conserved MMP-13 activation. Sci Rep 10, 3970 (2020).
18. G. J. Shin et al., Integrins protect sensory neurons in models of paclitaxel-induced peripheral sensory neuropathy. Proceedings of the National Academy of Sciences of the United States of America 118, (2021).
19. A. A. Hyman, S. Salser, D. N. Drechsel, N. Unwin, T. J. Mitchison, Role of GTP hydrolysis in microtubule dynamics: information from a slowly hydrolyzable analogue, GMPCPP. Mol Biol Cell 3, 1155-1167 (1992).
20. I. Arnal, R. H. Wade, How does taxol stabilize microtubules? Curr Biol 5, 900-908 (1995).
21. M. E. Hallak, J. A. Rodriguez, H. S. Barra, R. Caputto, Release of tyrosine from tyrosinated tubulin. Some common factors that affect this process and the assembly of tubulin. FEBS letters 73, 147-150 (1977).
22. J. Nieuwenhuis, T. R. Brummelkamp, The Tubulin Detyrosination Cycle: Function and Enzymes. Trends Cell Biol 29, 80-92 (2019).
23. C. Y. Chen et al., Suppression of detyrosinated microtubules improves cardiomyocyte function in human heart failure. Nature medicine 24, 1225-1233 (2018).
24. J. P. Kerr et al., Detyrosinated microtubules modulate mechanotransduction in heart and skeletal muscle. Nature communications 6, 8526 (2015).
25. A. Mialhe et al., Tubulin detyrosination is a frequent occurrence in breast cancers of poor prognosis. Cancer research 61, 5024-5027 (2001).
26. L. Peris et al., Tubulin tyrosination regulates synaptic function and is disrupted in Alzheimer's disease. Brain: a journal of neurology, (2022).
27. J. Dalous et al., Reversal of cell polarity and actin-myosin cytoskeleton reorganization under mechanical and chemical stimulation. Biophys J 94, 1063-1074 (2008).
28. M. Thery, A. Jimenez-Dalmaroni, V. Racine, M. Bornens, F. Julicher, Experimental and theoretical study of mitotic spindle orientation. Nature 447, 493-496 (2007).
29. A. J. Engler, S. Sen, H. L. Sweeney, D. E. Discher, Matrix elasticity directs stem cell lineage specification. Cell 126, 677-689 (2006).
30. K. Grote et al., Mechanical stretch enhances mRNA expression and proenzyme release of matrix metalloproteinase-2 (MMP-2) via NAD(P)H oxidase-derived reactive oxygen species. Circulation research 92, e80-86 (2003).
31. R. J. Khairallah et al., Microtubules underlie dysfunction in duchenne muscular dystrophy. Sci Signal 5, ra56 (2012).
32. B. L. Prosser, C. W. Ward, W. J. Lederer, X-ROS signaling: rapid mechano-chemo transduction in heart. Science 333, 1440-1445 (2011).
33. P. Robison et al., Detyrosinated microtubules buckle and bear load in contracting cardiomyocytes. Science 352, aaf0659 (2016).
34. C. W. Ward, B. L. Prosser, W. J. Lederer, Mechanical stretch-induced activation of ROS/RNS signaling in striated muscle. Antioxid Redox Signal 20, 929-936 (2014).
35. A. Roll-Mecak, The Tubulin Code in Microtubule Dynamics and Information Encoding. Developmental cell 54, 7-20 (2020).
36. S. Khawaja, G. G. Gundersen, J. C. Bulinski, Enhanced stability of microtubules enriched in detyrosinated tubulin is not a direct function of detyrosination level. The Journal of cell biology 106, 141-149 (1988).
37. J. Cassereau, P. Codron, B. Funalot, Inherited peripheral neuropathies due to mitochondrial disorders. Revue neurologique 170, 366-374 (2014).
38. S. J. Flatters, The contribution of mitochondria to sensory processing and pain. Prog Mol Biol Transl Sci 131, 119-146 (2015).
39. D. Pareyson, G. Piscosquito, I. Moroni, E. Salsano, M. Zeviani, Peripheral neuropathy in mitochondrial disorders. Lancet Neurol 12, 1011-1024 (2013).
40. D. S. Bilan, V. V. Belousov, In Vivo Imaging of Hydrogen Peroxide with HyPer Probes. Antioxid Redox Signal 29, 569-584 (2018).
41. D. S. Bilan et al., HyPer-3: a genetically encoded H(2)O(2) probe with improved performance for ratiometric and fluorescence lifetime imaging. ACS Chem Biol 8, 535-542 (2013).
42. A. Cirrincione, C. Reimonn, B. Harrison, S. Rieger, Longitudinal RNA sequencing of skin and DRG neurons in mice with paclitaxel-induced peripheral neuropathy. Data 7, 72 (2022).
43. A. Donkó, Z. Péterfi, A. Sum, T. Leto, M. Geiszt, Dual oxidases. Philos Trans R Soc Lond B Biol Sci 360, 2301-2308 (2005).
44. K. Bedard, K. H. Krause, The NOX family of ROS-generating NADPH oxidases: physiology and pathophysiology. Physiological reviews 87, 245-313 (2007).
45. M. Ushio-Fukai, A. M. Zafari, T. Fukui, N. Ishizaka, K. K. Griendling, p22phox is a critical component of the superoxide-generating NADH/NADPH oxidase system and regulates angiotensin II-induced hypertrophy in vascular smooth muscle cells. The Journal of biological chemistry 271, 23317-23321 (1996).
46. D. Szklarczyk et al., The STRING database in 2021: customizable protein-protein networks, and functional characterization of user-uploaded gene/measurement sets. Nucleic Acids Res 49, D605-D612 (2021).
47. S. E. Leonard, K. S. Carroll, Chemical ‘omics’ approaches for understanding protein cysteine oxidation in biology. Current opinion in chemical biology 15, 88-102 (2011).
48. J. C. Waters, R. H. Chen, A. W. Murray, E. D. Salmon, Localization of Mad2 to kinetochores depends on microtubule attachment, not tension. The Journal of cell biology 141, 1181-1191 (1998).
49. L. Jia, B. Li, H. Yu, The Bub1-Plk1 kinase complex promotes spindle checkpoint signalling through Cdc20 phosphorylation. Nature communications 7, 10818 (2016).
50. N. Hirokawa, Y. Noda, Y. Tanaka, S. Niwa, Kinesin superfamily motor proteins and intracellular transport. Nat Rev Mol Cell Biol 10, 682-696 (2009).
51. A. Leary et al., Successive Kinesin-5 Microtubule Crosslinking and Sliding Promote Fast, Irreversible Formation of a Stereotyped Bipolar Spindle. Curr Biol 29, 3825-3837.e3823 (2019).
52. I. Bobylev et al., Kinesin-5 Blocker Monastrol Protects Against Bortezomib-Induced Peripheral Neurotoxicity. Neurotox Res 32, 555-562 (2017).
53. R. P. Brandes, N. Weissmann, K. Schroder, Nox family NADPH oxidases in mechanotransduction: mechanisms and consequences. Antioxid Redox Signal 20, 887-898 (2014).
54. S. X. Ge, E. W. Son, R. Yao, iDEP: an integrated web application for differential expression and pathway analysis of RNA-Seq data. BMC Bioinformatics 19, 534 (2018).
55. J. L. Meaders, S. N. de Matos, D. R. Burgess, A Pushing Mechanism for Microtubule Aster Positioning in a Large Cell Type. Cell Rep 33, 108213 (2020).
56. A. Goulet, C. Moores, New insights into the mechanism of force generation by kinesin-5 molecular motors. Int Rev Cell Mol Biol 304, 419-466 (2013).
57. B. J. Mann, P. Wadsworth, Kinesin-5 Regulation and Function in Mitosis. Trends Cell Biol 29, 66-79 (2019).
58. M. A. Jordan, R. J. Toso, D. Thrower, L. Wilson, Mechanism of mitotic block and inhibition of cell proliferation by taxol at low concentrations. Proceedings of the National Academy of Sciences of the United States of America 90, 9552-9556 (1993).
59. L. M. Zasadil et al., Cytotoxicity of paclitaxel in breast cancer is due to chromosome missegregation on multipolar spindles. Sci Transl Med 6, 229ra243 (2014).
60. S. Zhao, Y. Tang, R. Wang, M. Najafi, Mechanisms of cancer cell death induction by paclitaxel: an updated review. Apoptosis: an international journal on programmed cell death 27, 647-667 (2022).
61. A. I. Marcus et al., Mitotic kinesin inhibitors induce mitotic arrest and cell death in Taxol-resistant and -sensitive cancer cells. The Journal of biological chemistry 280, 11569-11577 (2005).
62. X. Ren et al., Paclitaxel suppresses proliferation and induces apoptosis through regulation of ROS and the AKT/MAPK signaling pathway in canine mammary gland tumor cells. Mol Med Rep 17, 8289-8299 (2018).
63. M. A. Jordan et al., Mitotic block induced in HeLa cells by low concentrations of paclitaxel (Taxol) results in abnormal mitotic exit and apoptotic cell death. Cancer research 56, 816-825 (1996).
64. M. Distel, J. C. Hocking, K. Volkmann, R. W. Koster, The centrosome neither persistently leads migration nor determines the site of axonogenesis in migrating neurons in vivo. The Journal of cell biology 191, 875-890 (2010).
65. T. Kleele et al., An assay to image neuronal microtubule dynamics in mice. Nature communications 5, 4827 (2014).
66. H. Kadavath et al., Tau stabilizes microtubules by binding at the interface between tubulin heterodimers. Proceedings of the National Academy of Sciences of the United States of America 112, 7501-7506 (2015).
67. J. L. Ross, C. D. Santangelo, V. Makrides, D. K. Fygenson, Tau induces cooperative Taxol binding to microtubules. Proceedings of the National Academy of Sciences of the United States of America 101, 12910-12915 (2004).
68. S. A. Haque, T. P. Hasaka, A. D. Brooks, P. V. Lobanov, P. W. Baas, Monastrol, a prototype anti-cancer drug that inhibits a mitotic kinesin, induces rapid bursts of axonal outgrowth from cultured postmitotic neurons. Cell Motil Cytoskeleton 58, 10-16 (2004).
69. A. Falnikar, S. Tole, P. W. Baas, Kinesin-5, a mitotic microtubule-associated motor protein, modulates neuronal migration. Mol Biol Cell 22, 1561-1574 (2011).
70. N. Wei et al., Inhibitions and Down-Regulation of Motor Protein Eg5 Expression in Primary Sensory Neurons Reveal a Novel Therapeutic Target for Pathological Pain. Neurotherapeutics 19, 1401-1413 (2022).
71. J. M. Muretta et al., A posttranslational modification of the mitotic kinesin Eg5 that enhances its mechanochemical coupling and alters its mitotic function. Proceedings of the National Academy of Sciences of the United States of America 115, E1779-E1788 (2018).
72. T. Akera, Y. Goto, M. Sato, M. Yamamoto, Y. Watanabe, Mad1 promotes chromosome congression by anchoring a kinesin motor to the kinetochore. Nat Cell Biol 17, 1124-1133 (2015).
73. R. A. Clark, T. K. Epperson, A. J. Valente, Mechanisms of activation of NADPH oxidases. Jpn J Infect Dis 57, S22-23 (2004).
74. J. M. Cook-Mills, Hydrogen peroxide activation of endothelial cell-associated MMPs during VCAM-1-dependent leukocyte migration. Cell Mol Biol (Noisy-le-grand) 52, 8-16 (2006).
75. Y. Son et al., Mitogen-Activated Protein Kinases and Reactive Oxygen Species: How Can ROS Activate MAPK Pathways? J Signal Transduct 2011, 792639 (2011).
76. A. Dhar, M. R. Young, N. H. Colburn, The role of AP-1, NF-kappaB and ROS/NOS in skin carcinogenesis: the JB6 model is predictive. Molecular and cellular biochemistry 234-235, 185-193 (2002).
77. N. D'Ambrosi, S. Rossi, V. Gerbino, M. Cozzolino, Rac1 at the crossroad of actin dynamics and neuroinflammation in Amyotrophic Lateral Sclerosis. Front Cell Neurosci 8, 279 (2014).
78. M. A. Johnson, B. R. Henderson, The scaffolding protein IQGAP1 co-localizes with actin at the cytoplasmic face of the nuclear envelope: implications for cytoskeletal regulation. Bioarchitecture 2, 138-142 (2012).
79. D. Owen et al., The IQGAP1-Rac1 and IQGAP1-Cdc42 interactions: interfaces differ between the complexes. The Journal of biological chemistry 283, 1692-1704 (2008).
80. A. Hervera et al., Reactive oxygen species regulate axonal regeneration through the release of exosomal NADPH oxidase 2 complexes into injured axons. Nat Cell Biol 20, 307-319 (2018).
81. B. J. Benedikter et al., Redox-dependent thiol modifications: implications for the release of extracellular vesicles. Cell Mol Life Sci 75, 2321-2337 (2018).
82. A. Straube, A. Merdes, EB3 regulates microtubule dynamics at the cell cortex and is required for myoblast elongation and fusion. Curr Biol 17, 1318-1325 (2007).
83. G. T. Eisenhoffer et al., Crowding induces live cell extrusion to maintain homeostatic cell numbers in epithelia. Nature 484, 546-549 (2012).

### REFERENCES (METHODS)

1. Lisse, T. S. et al. Paclitaxel-induced epithelial damage and ectopic MMP-13 expression promotes neurotoxicity in zebrafish. Proceedings of the National Academy of Sciences of the United States of America 113, E2189-2198, doi:10.1073/pnas.1525096113 (2016).
2. O'Brien, G. S. et al. Coordinate development of skin cells and cutaneous sensory axons in zebrafish. Journal of Comparative Neurology 520, 816-831, doi:10.1002/cne.22791 (2012).
3. Meijering, E., Dzyubachyk, O. & Smal, I. Methods for cell and particle tracking. Methods Enzymol 504, 183-200, doi:10.1016/B978-0-12-391857-4.00009-4 (2012).
4. Nieuwenhuis, J. & Brummelkamp, T. R. The Tubulin Detyrosination Cycle: Function and Enzymes. Trends Cell Biol 29, 80-92, doi:10.1016/j.tcb.2018.08.003 (2019).
5. Cirrincione, A., Reimonn, C., Harrison, B. & Rieger, S. Longitudinal RNA sequencing of skin and DRG neurons in mice with paclitaxel-induced peripheral neuropathy. Data 7, 72, doi:https://doi.org/10.3390/data7060072 (2022).
6. Postma, T. J. et al. The development of an EORTC quality of life questionnaire to assess chemotherapy-induced peripheral neuropathy: the QLQ-CIPN20. Eur J Cancer 41, 1135-1139, doi:10.1016/j.ejca.2005.02.012 (2005).
7. Engelstad, J. K. et al. Epidermal nerve fibers: confidence intervals and continuous measures with nerve conduction. Neurology 79, 2187-2193, doi:10.1212/WNL.0b013e3182759608 (2012).
   De Novo Plasmids in this Disclosure:

CREST3: EB3-GFP
Sequence:
(SEQ ID NO: 25)
CCACCTAAATTGTAAGCGTTAATATTTTGTTAAAATTCGCGTTAAATTTTTGTTAAATCAGCTCATTTTT
TAACCAATAGGCCGAAATCGGCAAAATCCCTTATAAATCAAAAGAATAGACCGAGATAGGGTTGAGT
GTTGTTCCAGTTTGGAACAAGAGTCCACTATTAAAGAACGTGGACTCCAACGTCAAAGGGCGAAAAAC
CGTCTATCAGGGCGATGGCCCACGCTGGCTAAGAACTCATCAGCCTCCCCGGTCCATCTACCCACGTA
CCAATGCACCAATTGGCCACAATGACGGCTACTACATGGTGCCATTCCTTCCTCTTTATAGGAATGGAG
ACTACCTCCTGTCCAACAAGGCTCTTGGATACGAGTACGCCTACCTGTTGGACCCAGGTCATTGCACA
ACACCAGAAATGCCCTCTGATCTGCAAAAGACGTGAATATCTGTTCAGACACCCATATCCACTCTGTTC
CACACAGGTCAGAGGTTTGTCCAGGAGTTCTTGACAGAGGTGTAAAAAGTACTCAAAAATTTTACTCA
AGTGAAAGTACAAGTACTTAGGGAAAATTTTACTCAATTAAAAGTAAAAGTATCTGGCTAGAATCTTA
CTTGAGTAAAAGTAAAAAAGTACTCCATTAAAATTGTACTTGAGTATTAAGGAAGTAAAAGTAAAAGC
AAGAAAGAAAACTAGAGATTCTTGTTTAAGCTTTTAATCTCAAAAAACATTAAATGAAATGCATACAA
GGTTTTATCCTGCTTTAGAACTGTTTGTATTTAATTATCAAACTATAAGACAGACAATCTAATGCCAGT
ACACGCTACTCAAAGTTGTAAAACCTCAGATTTAACTTCAGTAGAAGCTGATTCTCAAAATTGTTAGTG
TCAAGCCTAGCTCTTTTGGGGCTGAAAAGCAATCCTGCAGTGCTGAAAAGCCTCTCACAGGCAGCCGA
TGCGGGAAGAGGTGTATTAGTCTTGATAGAGAGGCTGCAAATAGCAGGAAACGTGAGCAGAGACTCC
CTGGTGTCTGAAACACAGGCCAGATGGGCCCTCGAGCAGGAAACAGCTATGACCATGATTACGCCAA
GCTATCAACTTTGTATAGAAAAGTTGAACGAGAAACGTAAAATGATATAAATATCAATATATTAAATT
AGATTTTGCATAAAAAACAGACTACATAATACTGTAAAACACAACATATCCAGTCACTATGGTCGACC
GTATAGAAAAGTTGGTAACAGGATGTGACACGTCGTCTGCTTTAATCAGTGTTTGAGAGGGCACCTCT
ACACACTGAATGCCAAGCAAAGAAAGTCAGAAAATCTAAATAATAGCAAACATACACACACACACAT
ACACAAATATATATATCAACTCTTCATTCAGTTATTTACAAAAAAAACTGAGAATATCTGAACTAGCC
AGATGAAAAAAGAGAGCGAGAAAGGAGAAAAGAAAGAGAGAGAGAGATAAAATGAAAGAGACAGA
CAGAAACAGACAGAGGGAGAGGGAGTCAGTCTCTTGTGACCTACATGAAAGCAACGTGCCTCGCTAC
TCCTGGCTAAATGCACTCTCATATTAATATTTTAAAACGCCGTAATGTGACGGTGAAACCCTCGGAGG
ACAGATACATCAATTTGCAGGGGCCTCGGCTTCAGGAAAGAGAAACAGGGATAATTTGCCATTAGGA
GAAATTGTTCATTATGGGCAGCTTCTTAGGAACACATAAACCAAGTCGCCTCTCTCACGTGAGCTGCA
AATCCACACTTTTGAGTGAATGGAGACGTTGCCCGAGAGCTTTAATGCTCCTGGGTCATTAGTACAAA
AAGGTTTGTTGTGCTGTTAAATCTGCTGCATGTTGCGGTGATCAATCAACATGAAAGGGGCTTCCCACT
AAGACCGGATCACTGTAAATAAATCAATTAGGCCGCGAGAGTCAAGCTTTTATCAGAATTGTTGCTAC
ACACACACACACACACACACACACACAATCGGCTTATAGATTCAGTGCTGAACAGTCCGGTTCAGATT
CAGCTCAAAATAAATAAGACATCTGAGTTTGCTGATCATCGTTCAGCCAGTAAATGACACCAGCAGCA
GGCCAAGTTTCCATGAAGCTACTGTCTTCTATCGAACAAGCATGCGATATTTGCCGACTTAAAAAGCTC
AAGTGCTCCAAAGAAAAACCGAAGTGCGCCAAGTGTCTGAAGAACAACTGGGAGTGTCGCTACTCTC
CCAAAACCAAAAGGTCTCCGCTGACTAGGGCACATCTGACAGAAGTGGAATCAAGGCTAGAAAGACT
GGAACAGCTATTTCTACTGATTTTTCCTCGAGAAGACCTTGACATGATTTTGAAAATGGATTCTTTACA
GGATATAAAAGCATTGTTAACAGGATTATTTGTACAAGATAATGTGAATAAAGATGCCGTCACAGATA
GATTGGCTTCAGTGGAGACTGATATGCCTCTAACATTGAGACAGCATAGAATAAGTGCGACATCATCA
TCGGAAGAGAGTAGTAACAAAGGTCAAAGACAGTTGACTGTATCGTCGAGGTCGACCCCGGGAATTC
AGATCTCTCGAGCCGCCCCCCCGACCGATGTCAGCCTGGGGGACGAGCTCCACTTAGACGGCGAGGAC
GTGGCGATGGCGCATGCCGACGCGCTAGACGATTTCGATCTGGACATGTTGGGGGACGGGGATTCCCC
GGGTCCGGGATTTACCCCCCACGACTCCGCCCCCTACGGCGCTCTGGATATGTAGCGGATCTACTAGT
GCGGCCCCTCTCGAGCCTCTAGAACTATAGTGAGTCGTATTACGTAGATCCAGACATGATAAGATACA
TTGATGAGTTTGGACAAACCACAACTAGAATGCAGTGAAAAAAATGCTTTATTTGTGAAATTTGTGAT
GCTATTGCTTTATTTGTAACCATTATAAGCTGCAATAAACAAGTTAACAACAACAATTGCATTCATTTT
ATGTTTCAGGTTCAGGGGGAGGTGTGGGAGGTTTTTTAATTCGCGGCCATCAAGCTTAGGCCTCCAAG
GCGATCCAGACATGATAAGATACATTGATGAGTTTGGACAAACCACAACTAGAATGCAGTGAAAAAA
ATGCTTTATTTGTGAAATTTGTGATGCTATTGCTTTATTTGTAACCATTATAAGCTGCAATAAACAAGTT
AACAACAACAATTGCATTCATTTTATGTTTCAGGTTCAGGGGGAGGTGTGGGAGGTTTTTTAAAGCAA
GTAAAACCTCTACAAATGTGGTATGGCTGATTATCCGGAGTACTGTCCTCCGGGCTGGCGGAGTACTG
TCCTCCGGCAAGGTCGGAGTACTGTCCTCCGACACTAGAGGTCGGAGTACTGTCCTCCGACGCAAGGC
GGAGTACTGTCCTCCGACACTAGAGGTCGGAGTACTGTCCTCCGACACTAGAGGTCGGAGTACTGTCC
TCCGACACTAGAGGTCGGAGTACTGTCCTCCGACACTAGAGGTCGGAGTACTGTCCTCCGACACTAGA
GGTCGGAGTACTGTCCTCCGACACTAGAGGTCGGAGTACTGTCCTCCGACACTAGAGGTCGGAGTACT
GTCCTCCGACACTAGAGGTCGGAGTACTGTCCTCCGACACTAGAGGTCGGAGTACTGTCCTCCGTCCA
CTTAAGCTAGGTGGCCAGCGGTGGGATACATTGATGAGTTTGGACAAACCACAACTAGAATGCAGTGA
AAAAAATGCTTTATTTGTGAAATTTGTGATGCTATTGCTTTATTTCTAGAGGGTATATAATGGATCCCA
TCGCGTCTCAGCCTCACTTTGAGCTCCTCCACACGAATTCTTTGGCCACAAGCTGACCCTGAAGTTCAT
CTGCACCACCGGCAAGCTGCCCGTGCCCTGGCCCACCCTCGTGACCACCCTGACTACGGCGTGCAGTG
CTTCAGCATGGCCGTCAATGTGTACTCCACATCTGTGACCAGTGAAAATCTGAGTCGCCATGATATGCT
TGCATGGGTCAACGACTCCCTGCACCTCAACTATACCAAGATAGAACAGCTTTGTTCAGGGGCAGCCT
ACTGCCAGTTCATGGACATGCTCTTCCCCGGCTGTGTGCACTTGAGGAAAGTGAAGTTCCAGGCCAAA
CTAGAGCATGAATACATCCACAACTTCAAGGTGCTGCAAGCAGCTTTCAAGAAGATGGGTGTTGACAA
AATCATTCCTGTAGAGAAATTAGTGAAAGGAAAATTCCAAGATAATTTTGAGTTTATTCAGTGGTTTA
AGAAATTCTTTGACGCAAACTATGATGGAAAGGATTACAACCCTCTGCTGGCGCGGCAGGGCCAGGAC
GTAGCGCCACCTCCTAACCCAGGTGATCAGATCTTCAACAAATCCAAGAAACTCATTGGCACAGCAGT
TCCACAGAGGACGTCCCCCACAGGCCCAAAAAACATGCAGACCTCTGGCCGGCTGAGCAATGTGGCC
CCCCCCTGCATTCTCCGGAAGAATCCTCCATCAGCCCGAAATGGCGGCCATGAGACTGATGCCCAAAT
TCTTGAACTCAACCAACAGCTGGTGGACTTGAAGCTGACAGTGGATGGGCTGGAGAAGGAACGTGAC
TTCTACTTCAGCAAACTTCGTGACATCGAGCTCATCTGCCAGGAGCATGAAAGTGAAAACAGCCCTGT
TATCTCAGGCATCATTGGCATCCTCTATGCCACAGAGGAAGGATTCGCACCCCCCATGAAGCAGCACG
ACTTCTTCAAGTCCGCCATGCCCGAAGGCTACGTCCAGGAGCGCACCATCTTCTTCAAGGACGACGGC
AACTACAAGACCCGCGCCGAGGTGAAGTTCGAGGGCGACACCCTGGTGAACCGCATCGAGCTGAAGG
GCATCGACTTCAAGGAGGACGGCAACATCCTGGGGCACAAGCTGGAGTACAACTACAACAGCCACAA
CGTCTATATCATGGCCGACAAGCAGAAGAACGGCATCAAGGTGAACTTCAAGATCCGCCACAACATC
GAGGACGGCAGCGTGCAGCTCGCCGACCACTACCAGCAGAACACCCCCATCGGCGACGGCCCCGTGC
TGCTGCCCGACAACCACTACCTGAGCACCCAGTCCGCCCTGAGCAAAGACCCCAACGAGAAGCGCGA
TCACATGGTCCTGCTGGAGTTCGTGACCGCCGCCGGGATCACTCTCGGCATGGACGAGCTGTACAAGG
GCGGTGGAAGATCTGGGAATTCAAGGCCTCTCGAGCCTCTAGATTCTGCAGCCCTATAGcGCCATAGT
GACTGGATATGTTGTGTTTTACAGTATTATGTAGTCTGTTTTTTATGCAAAATCTAATTTAATATATTGA
TATTTATATCATTTTACGTTTCTCGTTCAACTTTATTATACATAGTTGATAATTCACTGGCCGTCGTTTT
ACGGTACCATCGATGATGATCCAGACATGATAAGATACATTGATGAGTTTGGACAAACCACAACTAGA
ATGCAGTGAAAAAAATGCTTTATTTGTGAAATTTGTGATGCTATTGCTTTATTTGTAACCATTATAAGC
TGCAATAAACAAGTTAACAACAACAATTGCATTCATTTTATGTTTCAGGTTCAGGGGGAGGTGTGGGA
GGTTTTTTAAAGCAAGTAAAACCTCTACAAATGTGGTATGGCTGATTATGATCCTCTAGATCAGATCTG
CGAAGATACGGCCACGGGTGCTCTTGATCCTGTGGCTGATTTTGGACTGTGCTGCTCGCAGCTGCTGAT
GAATCACATACTTCCTCCATTTTCTTCCACTGATTGACTGTTATAATTTCCCTAATTTCCAGGTCAAGGT
GCTGTGCATTGTGGTAATAGATGTGACATGACGTCACTTCCAAAGGACCAATGAACATGTCTGACCAA
TTTCATATAATGTGAAAACGATTTTCATAGGCAGAATAAATAACATTTAAATTAAACTGGGCATCAGC
GCAATTCAATTGGTTTGGTAATAGCAAGGGAAAATAGAATGAAGTGATCTCCAAAAAATAAGTACTTT
TTGACTGTAAATAAAATTGTAAGGAGTAAAAAGTACTTTTTTTTCTAAAAAAATGTAATTAAGTAAAA
GTAAAAGTATTGATTTTTAATTGTACTCAAGTAAAGTAAAAATCCCCAAAAATAATACTTAAGTACAG
TAATCAAGTAAAATTACTCAAGTACTTTACACCTCTGGTTCTTGACCCCCTACCTTCAGCAAGCCCAGC
AGATCCACTAGTTCTAGAGCGGCCGCCACCGCGGTGGAGCTCCAGCTTTTGTTCCCTTTAGTGAGGGTT
AATTGCGCGCTTGGCGTAATCATGGTCATAGCTGTTTCCTGTGTGAAATTGTTATCCGCTCACAATTCC
ACACAACATACGAGCCGGAAGCATAAAGTGTAAAGCCTGGGGTGCCTAATGAGTGAGCTAACTCACA
TTAATTGCGTTGCGCTCACTGCCCGCTTTCCAGTCGGGAAACCTGTCGTGCCAGCTGCATTAATGAATC
GGCCAACGCGCGGGGAGAGGCGGTTTGCGTATTGGGCGCTCTTCCGCTTCCTCGCTCACTGACTCGCT
GCGCTCGGTCGTTCGGCTGCGGCGAGCGGTATCAGCTCACTCAAAGGCGGTAATACGGTTATCCACAG
AATCAGGGGATAACGCAGGAAAGAACATGTGAGCAAAAGGCCAGCAAAAGGCCAGGAACCGTAAAA
AGGCCGCGTTGCTGGCGTTTTTCCATAGGCTCCGCCCCCCTGACGAGCATCACAAAAATCGACGCTCA
AGTCAGAGGTGGCGAAACCCGACAGGACTATAAAGATACCAGGCGTTTCCCCCTGGAAGCTCCCTCGT
GCGCTCTCCTGTTCCGACCCTGCCGCTTACCGGATACCTGTCCGCCTTTCTCCCTTCGGGAAGCGTGGC
GCTTTCTCATAGCTCACGCTGTAGGTATCTCAGTTCGGTGTAGGTCGTTCGCTCCAAGCTGGGCTGTGT
GCACGAACCCCCCGTTCAGCCCGACCGCTGCGCCTTATCCGGTAACTATCGTCTTGAGTCCAACCCGGT
AAGACACGACTTATCGCCACTGGCAGCAGCCACTGGTAACAGGATTAGCAGAGCGAGGTATGTAGGC
GGTGCTACAGAGTTCTTGAAGTGGTGGCCTAACTACGGCTACACTAGAAGGACAGTATTTGGTATCTG
CGCTCTGCTGAAGCCAGTTACCTTCGGAAAAAGAGTTGGTAGCTCTTGATCCGGCAAACAAACCACCG
CTGGTAGCGGTGGTTTTTTTGTTTGCAAGCAGCAGATTACGCGCAGAAAAAAAGGATCTCAAGAAGAT
CCTTTGATCTTTTCTACGGGGTCTGACGCTCAGTGGAACGAAAACTCACGTTAAGGGATTTTGGTCATG
AGATTATCAAAAAGGATCTTCACCTAGATCCTTTTAAATTAAAAATGAAGTTTTAAATCAATCTAAAGT
ATATATGAGTAAACTTGGTCTGACAGTTACCAATGCTTAATCAGTGAGGCACCTATCTCAGCGATCTGT
CTATTTCGTTCATCCATAGTTGCCTGACTCCCCGTCGTGTAGATAACTACGATACGGGAGGGCTTACCA
TCTGGCCCCAGTGCTGCAATGATACCGCGAGACCCACGCTCACCGGCTCCAGATTTATCAGCAATAAA
CCAGCCAGCCGGAAGGGCCGAGCGCAGAAGTGGTCCTGCAACTTTATCCGCCTCCATCCAGTCTATTA
ATTGTTGCCGGGAAGCTAGAGTAAGTAGTTCGCCAGTTAATAGTTTGCGCAACGTTGTTGCCATTGCTA
CAGGCATCGTGGTGTCACGCTCGTCGTTTGGTATGGCTTCATTCAGCTCCGGTTCCCAACGATCAAGGC
GAGTTACATGATCCCCCATGTTGTGCAAAAAAGCGGTTAGCTCCTTCGGTCCTCCGATCGTTGTCAGAA
GTAAGTTGGCCGCAGTGTTATCACTCATGGTTATGGCAGCACTGCATAATTCTCTTACTGTCATGCCAT
CCGTAAGATGCTTTTCTGTGACTGGTGAGTACTCAACCAAGTCATTCTGAGAATAGTGTATGCGGCGA
CCGAGTTGCTCTTGCCCGGCGTCAATACGGGATAATACCGCGCCACATAGCAGAACTTTAAAAGTGCT
CATCATTGGAAAACGTTCTTCGGGGCGAAAACTCTCAAGGATCTTACCGCTGTTGAGATCCAGTTCGA
TGTAACCCACTCGTGCACCCAACTGATCTTCAGCATCTTTTACTTTCACCAGCGTTTCTGGGTGAGCAA
AAACAGGAAGGCAAAATGCCGCAAAAAAGGGAATAAGGGCGACACGGAAATGTTGAATACTCATACT
CTTCCTTTTTCAATATTATTGAAGCATTTATCAGGGTTATTGTCTCATGAGCGGATACATATTTGAATGT
ATTTAGAAAAATAAACAAATAGGGGTTCCGCGCACATTTCCCCGAAAAGTG
tp63: EB3-GFP
Sequence:
(SEQ ID NO: 26)
CGCCATTCTGCCTGGGGACGTCGGAGCACTCGAGAGAAACCAGGCTCAGTCGGGCACGACCATTACTC
CACTTGACCAAACGTCTGCATATTTATAAATAATAATTTGCGTATTAATATGCAGATAAAGACAATTA
AAAGAAGCAACAGTAGCATTAATCATTCAAATAACAGTATACAATCAAACAACATTTTGTATATATAT
TTAAAAAAACAATACAAGGAGTAAAGACAAAGGCAATATCATTTAGTTTTTTTAATATTAACAAACCA
CAGGCTGTGTGAACCATCAGCAATTTCAAACCAGAGGGATCTGCTTTTTTGGGAACGATGTTGCAAAT
TTAATCTCTAGATTTTTTAAATCATGGATAGCCTCCCTAAAGCAAAAGCTTTTTAGGGGCTTTACTTCTC
CTGATTGCAGCCTGTGGTATAATTTGTGATTGTTAGGTGGGTCAGGTTTTGGGGTCCAGCCCTTTGTGC
ACTTTGACTATTGTCACCTTAGGCTGTCCCTTAATGCTGCCACCTCTGACCCCGGTGGGACACATAAAT
AACTTCTCCACCCGAGTTAGCATTTCAAGCCAGTGCAATAAAGAAATTAAAAATAATTGATTTATTAA
CAACTCTGTTTTATCTTTAAAAAATAAAATGTTTTATATCTAATGTTCACTCAGGTACCTGATATTGTTT
GCTGTGCAATAATGTAGGTTAGTCATTATATTGACAAAGAAAATTAAATATGATGTTGTTTATTTTTAA
TTTTACATTATAGACTAGATTAGATTTTCATTTGGAATTTAATTAGGAGTGCCTGGAGTGCATTGAGAA
CGTTAAAAAAAAAAAATCTGCTTACTCTTATAGCTTTGGAAACTATTTTCAGATTATTACTCCACTATA
GAAGCGATTCCTTTTTTTGAATAATTAAATATATATATACAGTGCTCAGCATAAATGATCATCACAGAT
CTCTATTTTAAAGTATTATTTTTCCATAGGATGCTTTACAATAATATATGTGTGTGTATACATTAGAATT
GTCAGTTACAAAATCAAAACTGCAACAAAATAACTAAACTTAAGATCTAATGAGTACACCCAAATTAA
ACCAAACATTAGTCTATGCAAAAATTAATATGTTAAGGAAAAATATTAAATACAATTTTAATATTCAG
TCATTCATTTTCTTTTTTGCTTAGTCCCTTTATTAATCTGGGGTCGCCACAGTGGAATTAACTGCCAATT
TATCCAGCATATATTTTATGCAGCGGATGCTCTTCCAGCTGCTACCCATCACTGGGAATCACCCATACA
CTCTCATTCACACACATACACTACGGACAATTTAGCCTACCCAATTCACCTATAGCGTATGTCTTTGGA
CTTGTGGGGGAAACCGGAACACACGGAGGAAACCCACGCGAACACAGGGAGATCATGCAAACTCAGA
AACGCCAACTGATCCAGCCAAGGCTCGAACCAGTGACCTTCTTGCTGTGAGGCAATAGGGCTACCCAT
TGTGTCACTGCACCGTCCACAATTTTAATAAACATGAAAAATCCCGAGAAACATAAACAATATTAAAT
GTAGTTGAAATTTTGTTGTTTGAAATTGTATTTATTAAATAAAAAAAATGTATGTATTATTATTATTTAT
TTATTTATAAAAGATGTTAGGTGGCCAAACTCTTTTTTTTAATGGATATAGTTGTTTAATAAAACAATT
TTGTTTAAATAAAACAAAATATATTGAATGAGCCATGTTGCTGTTGTTGTTTTTTACAGGCTTGATGTA
CAATATGATCTTTGTGTAGTTTTATAGTAATAAATCATTCATGTCTTGTGTATTGTACGTCATATTATCT
TAAAAGTACGCTATTAATTAAACTTGTTTATATGGTCATCTTAGACAGGACTCTCATTATAAAAGGATT
TTTGTTAATTTTATTAGTACCTTCCTGGAAAAATAAAAGATAAATAAATAAAAATGAATAAAAGTCTT
GGGCTTGTTTTCTGTACGAGTAATAAAAAAGAGTAAGCAAATATTCTGCATGACAATATTGTTCAAAG
TTTAAGGGCTGTATTTTTTATAATTGTTGGTTATTAACATGTTTTATACAAATTACTTTCAACATGGCTG
AAATAATAATTGTTATCACTATTTGAAACAGTCCTAGAATAGATTTTACTATATTTACATTTATTCCTCT
GATGACAAAGTTGAAATTCCTCTGATGACAAAGCAAAACTCTCAACTCTAGTTTTATTTTATTGTAATA
ACCATGTCAAGGGATAATGACAATGTTTTCCATTATATTTTCAATATAGAATAAAATTATAATTTATTA
ATCATTTAGTAGGGCAGCATGGTGGCACAATGGGTAGCACAATCACCTCACACCAAGAAGGTTGCTAG
TTCGAGCCCCGGCTGGGTCAGTTTGCATATCTGTGTGGAGAATGTTCTCTCTGTGTTGCCATGGGTTTC
CTCTGGGTGCTCTAGTTTCCACCACAAGTCTAAAAACAAGTGGCATCGGTGAATTGTGTAGGCTAAAT
TGTCTGTAGTGTTTGTGTGTGATTGAGTGTTTATGGATGTTTCCCAGTGATGGGTTGCAGTTGGAAGGG
CATTCTCTGCGTAAAACTATAAGTTGGCGGTTCATTCCACTGTGGTGACCCCAGATTAATAAAGGGATT
AAGCCGAAAAGAAAATGAATCATTAAATGATCATTTAGTTGATTGGAGAGCATTGCATAATGAGAATC
AAATAATGTATAGTTTTTTGTAAAATGATTGTGTATTAGAGGCACATTGTACGTAGTGTGGAATGTTTA
GGTTGTCTTTCTGATTGTTTTCATTTTGCAGAAGTTCATTTGCAGTCAGTGTAGTCAAAGCACCTCAGCT
CAATTTATAGCAGCAGCCTGCTGTGATTTAAGTGCCACAGGAGTGAAGGCTTTAACCCCTGTTGTAGA
TTGCAACCAATGTTTTTCTAAGGTAATTAGAAGCTTGTTTAGCTAACGCTACCCACCCCACCCCCCACA
AAAAAGTTGCTCTGAATAGCAGCATCTACCCCCAGGAGGGTGCAAGCTTCTGTTTTGAAGAAATTTGC
TCATGGTGTTACATAGGTGTTTAAAAATGTATGTTTTTTAATAGCATATATTTTAAAAGTGACTCATTA
ATGACATCATCTGCCATCTGCCTTTCTCAGAACTGACAAAGGACCCTTTTAAGGACCCCTATACACCAC
ATTTTTATCTATCTAATCACAAGTGGGTGATATTAGAGGGATGATTCACCTAAAAATGAATTTTCCGCA
TTTTAATTTGGGGTTCCAAACCAGTTTGAATTCTTTTGTTCTGTTGAACACAAAATAAGATGTTTTAAG
AATGTTGGAAATGTTGGAGACTTTTATAGTAGGAAAAACATATACTAATATTTGCATTCATGTTTGAAA
TGTTTAAAACATTTTTCAAAATATCTTATTTTTCAACTGTTTTGGAACAAGTCAAAAGTGAGTAAATGA
TGACTGATAAACAATTTCTTTAAATACAGATGAAAATAGCGTCTAACACATTTTGAACTAGTTCACTTT
CAACCACAGCAAGAGGTAGTCGAAAACACATTTGATCACATTTTGTTGTGGGACCGCTTGTTGTTCAG
TGTGTTTTCATTGTTGAGTTACAAACCGGTAAGTGGCATTAAAACATGTTTTGGCATCACTGATGATCA
TAAATAAATTGGTTAAAAAATTAAAAATTAGATGCTTACAGTACAGTTTGTTTAAAAACTTGCACTTTT
TGTAGTGTGAAATGACATTAGTACCTGCAGTGGGCTTTCTTTGTTCTGTATCTTCTGATGAATCTAACA
TAAAGACATTTAAATATTCACAATGGAGTGGGTTGGTTCTCATCTATCTGAAACAACATTAGTGTGTTC
AAAACAAATCTACACCAGTGATCTCTGATAGATACAAGCTGTCAACCGTAGTCCTATTGTTTCTCATGC
GACTGTCACAGGGCCATGCTAGAGGCTCTTTGGGTGGTGAGTAGAGACTCATTCTTATGCAAACAGGC
TATGGATGCTACAACTGAAGAGAATTTAGTGCTCTAAGGTCATCGCAGTTCTATAAAGCAGATTCCCT
CCAAAAAGCTAGAGAAAATTATGTGTCTGGAGGCACCATCTATCAGTGCAGTCATTCTTTTATTACTTT
TTTTTTTTGTCCATGCAATTTTTTAATCGCATTTTGAAGTATTTAAACCATTAAGAGTGTCAAATTATCA
TGTTATTATTTCAACTCATAATATCTCCTTTTAAAGATGATTCATTTGATTGTAGTTATAATAATTGTTT
TATTAATGCAAAAATTGCTTAAATGTTTGGCCTCTGAGATTTTCAGTTGACTCAATATTTTTGGAGCCT
GTTTCATGACTTTATTTAAAATATAATCTGCACTCGCAAATATTACAATTATATCATATTTTGAAAATCT
TAGATGTACATTTGTTTTGAAAATGGTTTAAAGGAAATTTAGGTCAAAATGTGTGTTTTTTTTAAATAA
TAATTCAGTCTTTTTTTTGATATAATAGGGAAAATGACAGCAATTAAAAGGCTGTTTTGGAAAGACATC
TAGGTCTGGTGCAGACTTCGTAACTTCTTAGAGTTAAATCTTGAAAACGGAGCCAGAAGACGGGCAAC
ACCTTCCTAAAAGCAGGGTGGGGTGTTGGACAGGTAGCTTCTTGGAACCTCCCCTCTTTGTCGCTGTGG
TATATATAGAAATCCCATTGAATTCCTATAGAGACCTTAAGCCCGTACCATTTACCTCTGAAGAGCTTG
ATTTAGGTGACACTATAGAATACAAGCTACTTGTTCTTTTTGCAGGATCCCATCGATTCGAATTCAAGG
CCTATGGCCGTCAATGTGTACTCCACATCTGTGACCAGTGAAAATCTGAGTCGCCATGATATGCTTGCA
TGGGTCAACGACTCCCTGCACCTCAACTATACCAAGATAGAACAGCTTTGTTCAGGGGCAGCCTACTG
CCAGTTCATGGACATGCTCTTCCCCGGCTGTGTGCACTTGAGGAAAGTGAAGTTCCAGGCCAAACTAG
AGCATGAATACATCCACAACTTCAAGGTGCTGCAAGCAGCTTTCAAGAAGATGGGTGTTGACAAAATC
ATTCCTGTAGAGAAATTAGTGAAAGGAAAATTCCAAGATAATTTTGAGTTTATTCAGTGGTTTAAGAA
ATTCTTTGACGCAAACTATGATGGAAAGGATTACAACCCTCTGCTGGCGCGGCAGGGCCAGGACGTAG
CGCCACCTCCTAACCCAGGTGATCAGATCTTCAACAAATCCAAGAAACTCATTGGCACAGCAGTTCCA
CAGAGGACGTCCCCCACAGGCCCAAAAAACATGCAGACCTCTGGCCGGCTGAGCAATGTGGCCCCCC
CCTGCATTCTCCGGAAGAATCCTCCATCAGCCCGAAATGGCGGCCATGAGACTGATGCCCAAATTCTT
GAACTCAACCAACAGCTGGTGGACTTGAAGCTGACAGTGGATGGGCTGGAGAAGGAACGTGACTTCT
ACTTCAGCAAACTTCGTGACATCGAGCTCATCTGCCAGGAGCATGAAAGTGAAAACAGCCCTGTTATC
TCAGGCATCATTGGCATCCTCTATGCCACAGAGGAAGGATTCGCACCCCCCTGAGCCTCTAGAACTAT
AGTGAGTCGTATTACGTAGATCCAGACATGATAAGATACATTGATGAGTTTGGACAAACCACAACTAG
AATGCAGTGAAAAAAATGCTTTATTTGTGAAATTTGTGATGCTATTGCTTTATTTGTAACCATTATAAG
CTGCAATAAACAAGTTAACAACAACAATTGCATTCATTTTATGTTTCAGGTTCAGGGGGAGGTGTGGG
AGGTTTTTTAATTCGCGGCCGCGGCGCCAATGCATTGGGCCCGGTACCCAGCTTTTGTTCCCTTTAGTG
AGGGTTAATTGCGCGCTTGGCGTAATCATGGTCATAGCTGTTTCCTGTGTGAAATTGTTATCCGCTCAC
AATTCCACACAACATACGAGCCGGAAGCATAAAGTGTAAAGCCTGGGGTGCCTAATGAGTGAGCTAA
CTCACATTAATTGCGTTGCGCTCACTGCCCGCTTTCCAGTCGGGAAACCTGTCGTGCCAGCTGCATTAA
TGAATCGGCCAACGCGCGGGGAGAGGCGGTTTGCGTATTGGGCGCTCTTCCGCTTCCTCGCTCACTGA
CTCGCTGCGCTCGGTCGTTCGGCTGCGGCGAGCGGTATCAGCTCACTCAAAGGCGGTAATACGGTTAT
CCACAGAATCAGGGGATAACGCAGGAAAGAACATGTGAGCAAAAGGCCAGCAAAAGGCCAGGAACC
GTAAAAAGGCCGCGTTGCTGGCGTTTTTCCATAGGCTCCGCCCCCCTGACGAGCATCACAAAAATCGA
CGCTCAAGTCAGAGGTGGCGAAACCCGACAGGACTATAAAGATACCAGGCGTTTCCCCCTGGAAGCTC
CCTCGTGCGCTCTCCTGTTCCGACCCTGCCGCTTACCGGATACCTGTCCGCCTTTCTCCCTTCGGGAAGC
GTGGCGCTTTCTCATAGCTCACGCTGTAGGTATCTCAGTTCGGTGTAGGTCGTTCGCTCCAAGCTGGGC
TGTGTGCACGAACCCCCCGTTCAGCCCGACCGCTGCGCCTTATCCGGTAACTATCGTCTTGAGTCCAAC
CCGGTAAGACACGACTTATCGCCACTGGCAGCAGCCACTGGTAACAGGATTAGCAGAGCGAGGTATG
TAGGCGGTGCTACAGAGTTCTTGAAGTGGTGGCCTAACTACGGCTACACTAGAAGGACAGTATTTGGT
ATCTGCGCTCTGCTGAAGCCAGTTACCTTCGGAAAAAGAGTTGGTAGCTCTTGATCCGGCAAACAAAC
CACCGCTGGTAGCGGTGGTTTTTTTGTTTGCAAGCAGCAGATTACGCGCAGAAAAAAAGGATCTCAAG
AAGATCCTTTGATCTTTTCTACGGGGTCTGACGCTCAGTGGAACGAAAACTCACGTTAAGGGATTTTGG
TCATGAGATTATCAAAAAGGATCTTCACCTAGATCCTTTTAAATTAAAAATGAAGTTTTAAATCAATCT
AAAGTATATATGAGTAAACTTGGTCTGACAGTTACCAATGCTTAATCAGTGAGGCACCTATCTCAGCG
ATCTGTCTATTTCGTTCATCCATAGTTGCCTGACTCCCCGTCGTGTAGATAACTACGATACGGGAGGGC
TTACCATCTGGCCCCAGTGCTGCAATGATACCGCGAGACCCACGCTCACCGGCTCCAGATTTATCAGC
AATAAACCAGCCAGCCGGAAGGGCCGAGCGCAGAAGTGGTCCTGCAACTTTATCCGCCTCCATCCAGT
CTATTAATTGTTGCCGGGAAGCTAGAGTAAGTAGTTCGCCAGTTAATAGTTTGCGCAACGTTGTTGCCA
TTGCTACAGGCATCGTGGTGTCACGCTCGTCGTTTGGTATGGCTTCATTCAGCTCCGGTTCCCAACGAT
CAAGGCGAGTTACATGATCCCCCATGTTGTGCAAAAAAGCGGTTAGCTCCTTCGGTCCTCCGATCGTTG
TCAGAAGTAAGTTGGCCGCAGTGTTATCACTCATGGTTATGGCAGCACTGCATAATTCTCTTACTGTCA
TGCCATCCGTAAGATGCTTTTCTGTGACTGGTGAGTACTCAACCAAGTCATTCTGAGAATAGTGTATGC
GGCGACCGAGTTGCTCTTGCCCGGCGTCAATACGGGATAATACCGCGCCACATAGCAGAACTTTAAAA
GTGCTCATCATTGGAAAACGTTCTTCGGGGCGAAAACTCTCAAGGATCTTACCGCTGTTGAGATCCAG
TTCGATGTAACCCACTCGTGCACCCAACTGATCTTCAGCATCTTTTACTTTCACCAGCGTTTCTGGGTG
AGCAAAAACAGGAAGGCAAAATGCCGCAAAAAAGGGAATAAGGGCGACACGGAAATGTTGAATACT
CATACTCTTCCTTTTTCAATATTATTGAAGCATTTATCAGGGTTATTGTCTCATGAGCGGATACATATTT
GAATGTATTTAGAAAAATAAACAAATAGGGGTTCCGCGCACATTTCCCCGAAAAGTGCCACCTAAATT
GTAAGCGTTAATATTTTGTTAAAATTCGCGTTAAATTTTTGTTAAATCAGCTCATTTTTTAACCAATAGG
CCGAAATCGGCAAAATCCCTTATAAATCAAAAGAATAGACCGAGATAGGGTTGAGTGTTGTTCCAGTT
TGGAACAAGAGTCCACTATTAAAGAACGTGGACTCCAACGTCAAAGGGCGAAAAACCGTCTATCAGG
GCGATGGCCCACTACGTGAACCATCACCCTAATCAAGTTTTTTGGGGTCGAGGTGCCGTAAAGCACTA
AATCGGAACCCTAAAGGGAGCCCCCGATTTAGAGCTTGACGGGGAAAGCCGGCGAACGTGGCGAGAA
AGGAAGGGAAGAAAGCGAAAGGAGCGGGCGCTAGGGCGCTGGCAAGTGTAGCGGTCACGCTGCGCG
TAACCACCACACCCGCCGCGCTTAATGCGCCGCTACAGGGCGCGTCCCATTCGCCATTCAGGCTGCGC
AACTGTTGGGAAGGGCGATCGGTGCGGGCCTCTTCGCTATTACGCCAGTCGACCATAGCCAATTCAAT
ATGGCGTATATGGACTCATGCCAATTCAATATGGTGGATCTGGACCTGTGCCAATTCAATATGGCGTAT
ATGGACTCGTGCCAATTCAATATGGTGGATCTGGACCCCAGCCAATTCAATATGGCGGACTTGGCACC
ATGCCAATTCAATATGGCGGACTTGGCACTGTGCCAACTGGGGAGGGGTCTACTTGGCACGGTGCCAA
GTTTGAGGAGGGGTCTTGGCCCTGTGCCAAGTCCGCCATATTGAATTGGCATGGTGCCAATAATGGCG
GCCATATTGGCTATATGCCAGGATCAATATATAGGCAATATCCAATATGGCCCTATGCCAATATGGCT
ATTGGCCAGGTTCAATACTATGTATTGGCCCTATGCCATATAGTATTCCATATATGGGTTTTCCTATTG
ACGTAGATAGCCCCTCCCAATGGGCGGTCCCATATACCATATATGGGGCTTCCTAATACCGCCCATAG
CCACTCCCCCATTGACGTCAATGGTCTCTATATATGGTCTTTCCTATTGACGTCATATGGGCGGTCCTAT
TGACGTATATGGCGCCTCCCCCATTGACGTCAATTACGGTAAATGGCCCGCCTGGCTCAATGCCCATTG
ACGTCAATAGGACCACCCACCATTGACGTCAATGGGATGGCTCATTGCCCATTCATATCCGTTCTCACG
CCCCCTATTGACGTCAATGACGGTAAATGGCCCACTTGGCAGTACATCAATATCTATTAATAGTAACTT
GGCAAGTACATTACTATTGGAAGGACGCCAGGGTACATTGGCAGTACTCCCATTGACGTCAATGGCGG
TAAATGGCCCGCGATGGCTGCCAAGTACATCCCCATTGACGTCAATGGGGAGGGGCAATGACGCAAA
TGGGCGTTCCATTGACGTAAATGGGCGGTAGGCGTGCCTAATGGGAGGTCTATATAAGCAATGCTCGT
TTAGGGAAC
tp63: kif11-AcGFP
Sequence:
(SEQ ID NO: 27)
CCACCTAAATTGTAAGCGTTAATATTTTGTTAAAATTCGCGTTAAATTTTTGTTAAATCAGCTCATTTTT
TAACCAATAGGCCGAAATCGGCAAAATCCCTTATAAATCAAAAGAATAGACCGAGATAGGGTTGAGT
GTTGTTCCAGTTTGGAACAAGAGTCCACTATTAAAGAACGTGGACTCCAACGTCAAAGGGCGAAAAAC
CGTCTATCAGGGCGATGGCCCACTACGTGAACCATCACCCTAATCAAGTTTTTTGGGGTCGAGGTGCC
GTAAAGCACTAAATCGGAACCCTAAAGGGAGCCCCCGATTTAGAGCTTGACGGGGAAAGCCAGAGGT
GTAAAAAGTACTCAAAAATTTTACTCAAGTGAAAGTACAAGTACTTAGGGAAAATTTTACTCAATTAA
AAGTAAAAGTATCTGGCTAGAATCTTACTTGAGTAAAAGTAAAAAAGTACTCCATTAAAATTGTACTT
GAGTATTAAGGAAGTAAAAGTAAAAGCAAGAAAGAAAACTAGAGATTCTTGTTTAAGCTTTTAATCTC
AAAAAACATTAAATGAAATGCATACAAGGTTTTATCCTGCTTTAGAACTGTTTGTATTTAATTATCAAA
CTATAAGACAGACAATCTAATGCCAGTACACGCTACTCAAAGTTGTAAAACCTCAGATTTAACTTCAG
TAGAAGCTGATTCTCAAAATTGTTAGTGTCAAGCCTAGCTCTTTTGGGGCTGAAAAGCAATCCTGCAGT
GCTGAAAAGCCTCTCACAGGCAGCCGATGCGGGAAGAGGTGTATTAGTCTTGATAGAGAGGCTGCAA
ATAGCAGGAAACGTGAGCAGAGACTCCCTGGTGTCTGAAACACAGGCCAGATCGCGTTGTAAAACGA
CGGCCAGTGAGCGCGCGTAATACGACTCACTATAGGGCGAATTGGGTACGATCCACCGGTCGCTCGAG
AGAAACCAGGCTCAGTCGGGCACGACCATTACTCCACTTGACCAAACGTCTGCATATTTATAAATAAT
AATTTGCGTATTAATATGCAGATAAAGACAATTAAAAGAAGCAACAGTAGCATTAATCATTCAAATAA
CAGTATACAATCAAACAACATTTTGTATATATATTTAAAAAAACAATACAAGGAGTAAAGACAAAGG
CAATATCATTTAGTTTTTTTAATATTAACAAACCACAGGCTGTGTGAACCATCAGCAATTTCAAACCAG
AGGGATCTGCTTTTTTGGGAACGATGTTGCAAATTTAATCTCTAGATTTTTTAAATCATGGATAGCCTC
CCTAAAGCAAAAGCTTTTTAGGGGCTTTACTTCTCCTGATTGCAGCCTGTGGTATAATTTGTGATTGTT
AGGTGGGTCAGGTTTTGGGGTCCAGCCCTTTGTGCACTTTGACTATTGTCACCTTAGGCTGTCCCTTAA
TGCTGCCACCTCTGACCCCGGTGGGACACATAAATAACTTCTCCACCCGAGTTAGCATTTCAAGCCAGT
GCAATAAAGAAATTAAAAATAATTGATTTATTAACAACTCTGTTTTATCTTTAAAAAATAAAATGTTTT
ATATCTAATGTTCACTCAGGTACCTGATATTGTTTGCTGTGCAATAATGTAGGTTAGTCATTATATTGA
CAAAGAAAATTAAATATGATGTTGTTTATTTTTAATTTTACATTATAGACTAGATTAGATTTTCATTTGG
AATTTAATTAGGAGTGCCTGGAGTGCATTGAGAACGTTAAAAAAAAAAAATCTGCTTACTCTTATAGC
TTTGGAAACTATTTTCAGATTATTACTCCACTATAGAAGCGATTCCTTTTTTTGAATAATTAAATATATA
TATACAGTGCTCAGCATAAATGATCATCACAGATCTCTATTTTAAAGTATTATTTTTCCATAGGATGCT
TTACAATAATATATGTGTGTGTATACATTAGAATTGTCAGTTACAAAATCAAAACTGCAACAAAATAA
CTAAACTTAAGATCTAATGAGTACACCCAAATTAAACCAAACATTAGTCTATGCAAAAATTAATATGT
TAAGGAAAAATATTAAATACAATTTTAATATTCAGTCATTCATTTTCTTTTTTGCTTAGTCCCTTTATTA
ATCTGGGGTCGCCACAGTGGAATTAACTGCCAATTTATCCAGCATATATTTTATGCAGCGGATGCTCTT
CCAGCTGCTACCCATCACTGGGAATCACCCATACACTCTCATTCACACACATACACTACGGACAATTTA
GCCTACCCAATTCACCTATAGCGTATGTCTTTGGACTTGTGGGGGAAACCGGAACACACGGAGGAAAC
CCACGCGAACACAGGGAGATCATGCAAACTCAGAAACGCCAACTGATCCAGCCAAGGCTCGAACCAG
TGACCTTCTTGCTGTGAGGCAATAGGGCTACCCATTGTGTCACTGCACCGTCCACAATTTTAATAAACA
TGAAAAATCCCGAGAAACATAAACAATATTAAATGTAGTTGAAATTTTGTTGTTTGAAATTGTATTTAT
TAAATAAAAAAAATGTATGTATTATTATTATTTATTTATTTATAAAAGATGTTAGGTGGCCAAACTCTT
TTTTTTAATGGATATAGTTGTTTAATAAAACAATTTTGTTTAAATAAAACAAAATATATTGAATGAGCC
ATGTTGCTGTTGTTGTTTTTTACAGGCTTGATGTACAATATGATCTTTGTGTAGTTTTATAGTAATAAAT
CATTCATGTCTTGTGTATTGTACGTCATATTATCTTAAAAGTACGCTATTAATTAAACTTGTTTATATGG
TCATCTTAGACAGGACTCTCATTATAAAAGGATTTTTGTTAATTTTATTAGTACCTTCCTGGAAAAATA
AAAGATAAATAAATAAAAATGAATAAAAGTCTTGGGCTTGTTTTCTGTACGAGTAATAAAAAAGAGTA
AGCAAATATTCTGCATGACAATATTGTTCAAAGTTTAAGGGCTGTATTTTTTATAATTGTTGGTTATTA
ACATGTTTTATACAAATTACTTTCAACATGGCTGAAATAATAATTGTTATCACTATTTGAAACAGTCCT
AGAATAGATTTTACTATATTTACATTTATTCCTCTGATGACAAAGTTGAAATTCCTCTGATGACAAAGC
AAAACTCTCAACTCTAGTTTTATTTTATTGTAATAACCATGTCAAGGGATAATGACAATGTTTTCCATT
ATATTTTCAATATAGAATAAAATTATAATTTATTAATCATTTAGTAGGGCAGCATGGTGGCACAATGG
GTAGCACAATCACCTCACACCAAGAAGGTTGCTAGTTCGAGCCCCGGCTGGGTCAGTTTGCATATCTG
TGTGGAGAATGTTCTCTCTGTGTTGCCATGGGTTTCCTCTGGGTGCTCTAGTTTCCACCACAAGTCTAA
AAACAAGTGGCATCGGTGAATTGTGTAGGCTAAATTGTCTGTAGTGTTTGTGTGTGATTGAGTGTTTAT
GGATGTTTCCCAGTGATGGGTTGCAGTTGGAAGGGCATTCTCTGCGTAAAACTATAAGTTGGCGGTTC
ATTCCACTGTGGTGACCCCAGATTAATAAAGGGATTAAGCCGAAAAGAAAATGAATCATTAAATGATC
ATTTAGTTGATTGGAGAGCATTGCATAATGAGAATCAAATAATGTATAGTTTTTTGTAAAATGATTGTG
TATTAGAGGCACATTGTACGTAGTGTGGAATGTTTAGGTTGTCTTTCTGATTGTTTTCATTTTGCAGAA
GTTCATTTGCAGTCAGTGTAGTCAAAGCACCTCAGCTCAATTTATAGCAGCAGCCTGCTGTGATTTAAG
TGCCACAGGAGTGAAGGCTTTAACCCCTGTTGTAGATTGCAACCAATGTTTTTCTAAGGTAATTAGAA
GCTTGTTTAGCTAACGCTACCCACCCCACCCCCCACAAAAAAGTTGCTCTGAATAGCAGCATCTACCCC
CAGGAGGGTGCAAGCTTCTGTTTTGAAGAAATTTGCTCATGGTGTTACATAGGTGTTTAAAAATGTATG
TTTTTTAATAGCATATATTTTAAAAGTGACTCATTAATGACATCATCTGCCATCTGCCTTTCTCAGAACT
GACAAAGGACCCTTTTAAGGACCCCTATACACCACATTTTTATCTATCTAATCACAAGTGGGTGATATT
AGAGGGATGATTCACCTAAAAATGAATTTTCCGCATTTTAATTTGGGGTTCCAAACCAGTTTGAATTCT
TTTGTTCTGTTGAACACAAAATAAGATGTTTTAAGAATGTTGGAAATGTTGGAGACTTTTATAGTAGGA
AAAACATATACTAATATTTGCATTCATGTTTGAAATGTTTAAAACATTTTTCAAAATATCTTATTTTTCA
ACTGTTTTGGAACAAGTCAAAAGTGAGTAAATGATGACTGATAAACAATTTCTTTAAATACAGATGAA
AATAGCGTCTAACACATTTTGAACTAGTTCACTTTCAACCACAGCAAGAGGTAGTCGAAAACACATTT
GATCACATTTTGTTGTGGGACCGCTTGTTGTTCAGTGTGTTTTCATTGTTGAGTTACAAACCGGTAAGT
GGCATTAAAACATGTTTTGGCATCACTGATGATCATAAATAAATTGGTTAAAAAATTAAAAATTAGAT
GCTTACAGTACAGTTTGTTTAAAAACTTGCACTTTTTGTAGTGTGAAATGACATTAGTACCTGCAGTGG
GCTTTCTTTGTTCTGTATCTTCTGATGAATCTAACATAAAGACATTTAAATATTCACAATGGAGTGGGT
TGGTTCTCATCTATCTGAAACAACATTAGTGTGTTCAAAACAAATCTACACCAGTGATCTCTGATAGAT
ACAAGCTGTCAACCGTAGTCCTATTGTTTCTCATGCGACTGTCACAGGGCCATGCTAGAGGCTCTTTGG
GTGGTGAGTAGAGACTCATTCTTATGCAAACAGGCTATGGATGCTACAACTGAAGAGAATTTAGTGCT
CTAAGGTCATCGCAGTTCTATAAAGCAGATTCCCTCCAAAAAGCTAGAGAAAATTATGTGTCTGGAGG
CACCATCTATCAGTGCAGTCATTCTTTTATTACTTTTTTTTTTTGTCCATGCAATTTTTTAATCGCATTTT
GAAGTATTTAAACCATTAAGAGTGTCAAATTATCATGTTATTATTTCAACTCATAATATCTCCTTTTAA
AGATGATTCATTTGATTGTAGTTATAATAATTGTTTTATTAATGCAAAAATTGCTTAAATGTTTGGCCTC
TGAGATTTTCAGTTGACTCAATATTTTTGGAGCCTGTTTCATGACTTTATTTAAAATATAATCTGCACTC
GCAAATATTACAATTATATCATATTTTGAAAATCTTAGATGTACATTTGTTTTGAAAATGGTTTAAAGG
AAATTTAGGTCAAAATGTGTGTTTTTTTTAAATAATAATTCAGTCTTTTTTTTGATATAATAGGGAAAA
TGACAGCAATTAAAAGGCTGTTTTGGAAAGACATCTAGGTCTGGTGCAGACTTCGTAACTTCTTAGAG
TTAAATCTTGAAAACGGAGCCAGAAGACGGGCAACACCTTCCTAAAAGCAGGGTGGGGTGTTGGACA
GGTAGCTTCTTGGAACCTCCCCTCTTTGTCGCTGTGGTATATATAGAAATCCCATTGAATTCCTATAGA
GACCTTAAGCCCGTACCATTTACCTCTGAAGCCACGGATCCCATGGTGATGGCATCATCACAAGTACC
AGCAGCCAAAAAAGATGAGAAGGGCAGAAACATACAAGTGGTTGTACGATGCAGACCCTTTAACACA
GTGGAGCGTAAATCTGGCTCTCACACTGTTGTTGAATGTGACCAGAACCGGAAAGAGGTGATTATGCG
TACTGGAGGTGCCACAGACAAAGCAGCAAGAAAAACATACACTTTTGATATGGTTTTTGGCCCTTCTG
CCAAACAAATTGAAGTTTATAGGAGTGTGGTTTGCCCCATATTAGATGAAGTTATCATGGGCTATAAC
TGCACAATCTTTGCATATGGGCAAACCGGAACAGGCAAAACCTTCACAATGGAAGGTGACCGATCACC
CAATGAGGAGTTTACTTGGGAAGAGGACCCTCTGGCTGGAATAATTCCCAGGACTCTTCATCAGATCT
TTGAAAAACTGTCTAATAATGGCACAGAGTTCTCAGTGAAGGTGTCTCTGCTGGAAATTTATAACGAG
GAACTGTTTGACCTGCTCAGCCCTGCTCCTGATGTCACAGAGAGATTACAGCTCTTTGATGATCCCAGA
AATAAAAGGGGTGTGACCATTAAAGGTCTGGAGGAAATCACCGTACACAATAAGAATGAGGTGTATC
AGATCCTGGAGAGAGGAGCAGCCAAGAGGAAGACTGCCTCCACACTCATGAATGCCTATTCCAGTCG
ATCCCACTCTGTGTTCTCTGTCACTATTCACATGAAGGAAATCACACTGGATGGGGAAGAGCTAGTTA
AGATTGGTAAACTCAACTTGGTGGATCTTGCAGGTAGTGAGAACATCGGACGATCTGGTGCTGTGGAT
AAGCGTGCACGTGAAGCTGGCAACATCAACCAGTCTCTGCTAACTCTGGGTCGTGTAATCAAGGCTTT
GGTGGAGAGAGGGCCTCACGTGCCCTACAGAGAGTCTAAACTAACCCGCATACTGCAGGACTCGCTA
GGAGGACGCACCAAAACCTCTATCATCGCCACTGTGTCTCCTGCCTCCATCAATCTGGAGGAAACTCT
GAGCACTTTAGACTATGCTAACAGAGCTAAGAGCATCATGAATAAGCCTGAGGTCAATCAAAAGCTTA
CCAAGAGAACCCTCATCAAGGAATACACAGAGGAAATTGAGAGACTGAAGAGAGATTTGGCTGCCAC
CCGTGACAAACATGGAGTGTACCTTTCCGTTGATAACTATGAGACTTTGAATGGTAAAATTGTGTCTCA
GGAAGAGCAGATTACAGAGTACACTGAGCGGATAGCTGCTATGGAGGAGGAGCTCAAAAAGATTATA
GACCTGTTCACAGATAGTAAGCAGAAATTGGAACAGTGCACCGAGGACCTGCAAGACAAAAACCAGA
GACTGGAGGAGGCTCACAAAGACCTATCGGAGACCAGGCACCGCCTCAATCAGGAGGAATTCATCAG
TACACAGCTTCAAACCAATGAGAGTCACCTCTATAACACTGCTGACCAGTTGTTAAGCACTGCTGAGG
CCAGCACACAGGATGTCGGTGGTCTCCATGCTAAGCTACAAAGGAAGAAGGATGTGGAGCTTCATAA
CAGTAAGGTTCAGGAGAGCTTCTCCCAGTGCATGGAGAACTGCTACAACAGCATGCAGACCTCACTGA
AGGAGCAGAGCCAAAAACATGCTGCTATGATTGATTATTACCGCTCCTCTGTGGGTGAGCTGCTGAAC
ACCAATGGCAAGGTGTTTAAGGAGACTTTGGGTGCTGTGTGCGAGTCTTACAGCAGTATTAAAGGAGC
TGTGGGAGAAGGTGTAGAGCGGTGTAAGGAGCAAGTGTTAAATCAGGAGAAACTCTCTCAGGATGCT
CAAAACAGCATCCTGGAAATTCTGGATGAACACAAACAGCATCTTGAGGAGGTTCTGGTTGCCCAGGC
GGTGCCAGGTATCAGGTCTGTCATGTCCATGAATGACAATTTGAAGCAAACCCTTCACAAATACCACA
ATCTGGCTGAGCAGATGCAGGGTGTGAAAGCAGATATGATGACGTTTTTTGATGCGTACACTGAATCA
CTGGCCAGTATGCGAGAGTGCGCTTTGCAGGGTTTTAACACACTGCGTGCCGAACATGACAAACTCAA
ACAACAAATCAGCCAAGCTGGAAACAGCCATCAAGTGCGTGTGGCCGAGCTGGTTCAGTGTTTGCAGA
ACCAAATGAATCTGCTGGCGGTGGACACTCAGAATGACTTTGAGGGTCTTTCACAAGCAGCATCTGCC
CAGATTCCTCCACTGGAAACATTACAGAGCTCCATAGAGAGTAAATGCACAGTGGCTGAGGAGCAGG
CTGTGTCTGTTCGTGCTCGACTGGGTTCCTCTGTTCATGGAGTGATTTCAGAGATGAATAGTGTGACTA
AGGAGGGAGAGAGGGCGCTGGAGGAGTGTGCTGGTTATTGTGGACACCTGCAGACATCGCTGGACTC
GCTGGCTGAGTCAGGACTCAAGTGGTGCGATGAAGCTAAAGGCTTGACTGAGAGTAAAGCCCAGGAG
CAACTCAAACTCATCAGACAGACAGACACGGCTGTGCAAGACCTGCTGAAGTCTGTGGAGGAAAAGG
GTGAGAAAGCTGTTCAGGACTGTGAAGCCAGATTGGGTCAAATGCAGCAGGAAATGGAAGCAACCCT
GGGTCGTGTGGAAATGCAGACCAGCAAAGATGAAGCGACATTGCAGGAGCACAGAGAGACCCTGTCC
TCCATCAACACTCAGGCACTGGACACCGTACACAACTTCATCAGCTCAGTGCTACGGCAGGATTTACC
AACAGGAACGACCCCTCAGCGCAAAGAGTACATGTACCCACGTGTGCTGAGCAGGCCGAGGAGTAGA
GAGGAGCTGGAAGAGGAGTTCAGAGCTCAGCAAGAGCAGCTTCAGTCTGAACTGAAGCCGTGTGAGA
TCGTAATGGAGGTGGAGGAGGAGAAACCTGTCGACCAGGACTCTCTGGAGGATGACGTCAGTGTTTCT
AGTGATGGAAACAACACTGAACAGTCGTGCTCTGATGAGAACCTGATATGTTATGAGAATGGAAGGA
TTCCTTTTTTCAAGAAGAAAAGCAAGAAGGAAAATGGCAGTAAATCACTGAACCGTTCAAAGGTGGA
GAATGACAGCATGTCAACACCACCCCGCTCTAAACTTCCACTCAGATGTCAGAGCAAGGGCGCCGAGC
TGTTCACCGGCATCGTGCCCATCCTGATCGAGCTGAATGGCGATGTGAATGGCCACAAGTTCAGCGTG
AGCGGCGAGGGCGAGGGCGATGCCACCTACGGCAAGCTGACCCTGAAGTTCATCTGCACCACCGGCA
AGCTGCCTGTGCCCTGGCCCACCCTGGTGACCACCCTGAGCTACGGCGTGCAGTGCTTCTCACGCTACC
CCGATCACATGAAGCAGCACGACTTCTTCAAGAGCGCCATGCCTGAGGGCTACATCCAGGAGCGCACC
ATCTTCTTCGAGGATGACGGCAACTACAAGTCGCGCGCCGAGGTGAAGTTCGAGGGCGATACCCTGGT
GAATCGCATCGAGCTGACCGGCACCGATTTCAAGGAGGATGGCAACATCCTGGGCAATAAGATGGAG
TACAACTACAACGCCCACAATGTGTACATCATGACCGACAAGGCCAAGAATGGCATCAAGGTGAACTT
CAAGATCCGCCACAACATCGAGGATGGCAGCGTGCAGCTGGCCGACCACTACCAGCAGAATACCCCC
ATCGGCGATGGCCCTGTGCTGCTGCCCGATAACCACTACCTGTCCACCCAGAGCGCCCTGTCCAAGGA
CCCCAACGAGAAGCGCGATCACATGATCTACTTCGGCTTCGTGACCGCCGCCGCCATCACCCACGGCA
TGGATGAGCTGTACAAGTGAGCGGCCGCCACCGCGGTGGAGCTCGAATTAATTCATCGATGATGATCC
AGACATGATAAGATACATTGATGAGTTTGGACAAACCACAACTAGAATGCAGTGAAAAAAATGCTTT
ATTTGTGAAATTTGTGATGCTATTGCTTTATTTGTAACCATTATAAGCTGCAATAAACAAGTTAACAAC
AACAATTGCATTCATTTTATGTTTCAGGTTCAGGGGGAGGTGTGGGAGGTTTTTTAAAGCAAGTAAAA
CCTCTACAAATGTGGTATGGCTGATTATGATCCTCTAGATCAGGCCGCCACCGCGGTGGAGCTCCAGC
TTTTGTTCCCTTTAGTGAGGGTTAATTGCGCGCTTGGCGTAATCATGGTCATAGCTGTTTCCTGTGTGGC
GAAGATACGGCCACGGGTGCTCTTGATCCTGTGGCTGATTTTGGACTGTGCTGCTCGCAGCTGCTGATG
AATCACATACTTCCTCCATTTTCTTCCACTGATTGACTGTTATAATTTCCCTAATTTCCAGGTCAAGGTG
CTGTGCATTGTGGTAATAGATGTGACATGACGTCACTTCCAAAGGACCAATGAACATGTCTGACCAAT
TTCATATAATGTGAAAACGATTTTCATAGGCAGAATAAATAACATTTAAATTAAACTGGGCATCAGCG
CAATTCAATTGGTTTGGTAATAGCAAGGGAAAATAGAATGAAGTGATCTCCAAAAAATAAGTACTTTT
TGACTGTAAATAAAATTGTAAGGAGTAAAAAGTACTTTTTTTTCTAAAAAAATGTAATTAAGTAAAAG
TAAAAGTATTGATTTTTAATTGTACTCAAGTAAAGTAAAAATCCCCAAAAATAATACTTAAGTACAGT
AATCAAGTAAAATTACTCAAGTACTTTACACCTCTGGGGGAGGTTCCCTTTAGTGAGGGTTAATTGCGC
GCTTGGCGTAATCATGGTCATAGCTGTTTCCTGTGTGAAATTGTTATCCGCTCACAATTCCACACAACA
TACGAGCCGGAAGCATAAAGTGTAAAGCCTGGTGAAGCCAGTTACCTTCGGAAAAAGAGTTGGTAGC
TCTTGATCCGGCAAACAAACCACCGCTGGTAGCGGTGGTTTTTTTGTTTGCAAGCAGCAGATTACGCGC
AGAAAAAAAGGATCTCAAGAAGATCCTTTGATCTTTTCTACGGGGTCTGACGCTCAGTGGAACGAAAA
CTCACGTTAAGGGATTTTGGTCATGAGATTATCAAAAAGGATCTTCACCTAGATCCTTTTAAATTAAAA
ATGAAGTTTTAAATCAATCTAAAGTATATATGAGTAAACTTGGTCTGACAGTTACCAATGCTTAATCAG
TGAGGCACCTATCTCAGCGATCTGTCTATTTCGTTCATCCATAGTTGCCTGACTCCCCGTCGTGTAGAT
AACTACGATACGGGAGGGCTTACCATCTGGCCCCAGTGCTGCAATGATACCGCGAGACCCACGCTCAC
CGGCTCCAGATTTATCAGCAATAAACCAGCCAGCCGGAAGGGCCGAGCGCAGAAGTGGTCCTGCAAC
TTTATCCGCCTCCATCCAGTCTATTAATTGTTGCCGGGAAGCTAGAGTAAGTAGTTCGCCAGTTAATAG
TTTGCGCAACGTTGTTGCCATTGCTACAGGCATCGTGGTGTCACGCTCGTCGTTTGGTATGGCTTCATT
CAGCTCCGGTTCCCAACGATCAAGGCGAGTTACATGATCCCCCATGTTGTGCAAAAAAGCGGTTAGCT
CCTTCGGTCCTCCGATCGTTGTCAGAAGTAAGTTGGCCGCAGTGTTATCACTCATGGTTATGGCAGCAC
TGCATAATTCTCTTACTGTCATGCCATCCGTAAGATGCTTTTCTGTGACTGGTGAGTACTCAACCAAGT
CATTCTGAGAATAGTGTATGCGGCGACCGAGTTGCTCTTGCCCGGCGTCAATACGGGATAATACCGCG
CCACATAGCAGAACTTTAAAAGTGCTCATCATTGGAAAACGTTCTTCGGGGCGAAAACTCTCAAGGAT
CTTACCGCTGTTGAGATCCAGTTCGATGTAACCCACTCGTGCACCCAACTGATCTTCAGCATCTTTTAC
TTTCACCAGCGTTTCTGGGTGAGCAAAAACAGGAAGGCAAAATGCCGCAAAAAAGGGAATAAGGGCG
ACACGGAAATGTTGAATACTCATACTCTTCCTTTTTCAATATTATTGAAGCATTTATCAGGGTTATTGTC
TCATGAGCGGATACATATTTGAATGTATTTAGAAAAATAAACAAATAGGGGTTCCGCGCACATTTCCC
CGAAAAGTG
tp63: HyPer-cyto
Sequence:
(SEQ ID NO: 28)
CCACCTAAATTGTAAGCGTTAATATTTTGTTAAAATTCGCGTTAAATTTTTGTTAAATCAGCTCATTTTT
TAACCAATAGGCCGAAATCGGCAAAATCCCTTATAAATCAAAAGAATAGACCGAGATAGGGTTGAGT
GTTGTTCCAGTTTGGAACAAGAGTCCACTATTAAAGAACGTGGACTCCAACGTCAAAGGGCGAAAAAC
CGTCTATCAGGGCGATGGCCCACTACGTGAACCATCACCCTAATCAAGTTTTTTGGGGTCGAGGTGCC
GTAAAGCACTAAATCGGAACCCTAAAGGGAGCCCCCGATTTAGAGCTTGACGGGGAAAGCCGGCGAA
CGTGGCGAGAAAGGAAGGGAAGAAAGCGAAAGGAGCGGGCGCTAGGGCGCTGGCAAGTGTAGCGGT
CACGCTGCGCGTAACCACCACACCCGCCGCGCTTAATGCGCCGCTACAGGGCGCGTCCCATTCGCCAT
TCAGGCTGCGCAACTGTTGGGAAGGGCGATCGGTGCGGGCCTCTTCGCTATTACGCCAGCTGGCGAAA
GGGGGATGTGCTGCAAGGCGATTAAGTTGGGTAACGCCAGGGTTTTCCCAGTCACGACGTTGTAAAAC
GACGGCCAGTGAGCGCGCGTAATACGACTCACTATAGGGCGAATTGGGTACCTCGAGTCGAGCAGGG
GGATCATCTAATCAAGCACAAATAAGGGGCGTGTAACACAAAAGCCAGCGACCCTTTCCAATGCAAA
TCAAACTTGCAATTCTTTGCCGTTTTTATCATTTAAGTGTCGGCTTAAGGTCCACTATCAGATGTAAAC
AGCCTTATCTAACAAAGGTATCATTACATTCTGAAATTCTCAGGCATGCAAGCTAGCTTATGACGCACT
AGGGAGTGCCACCCTTCCTTTCGCCCTAACTTCGTGATAACTCGCGCGTTTCACTCAACAGCTGCATCC
GCCCTAGTGCTACTGGGAGTTGTAGTATACAAGACGCTTACAGGCTGAATGTTCTGTCAAGACCCCGC
CTCTAGCACTTTGGGAATTCTGGACTTGATGATGTCATGGTTAATCCCCGCCCAGTAGAGGCGGCTATA
TAAAGGGTGGTTAAGGCCCGGTTCGCTCTCTTCCTCACCGGGTCTGCGGCGAGTTCTAGCTGAAGCTTC
CTGCAGGTCGACCGATCCTGAGAACTTCAGGGTGAGTTTGGGGACCCTTGATTGTTCTTTCTTTTTCGC
TATTGTAAAATTCATGTTATATGGAGGGGGCAAAGTTTTCAGGGTGTTGTTTAGAATGGGAAGATGTC
CCTTGTATCACCATGGACCCTCATGATAATTTTGTTTCTTTCACTTTCTACTCTGTTGACAACCATTGTC
TCCTCTTATTTTCTTTTCATTTTCTGTAACTTTTTCGTTAAACTTTAGCTTGCATTTGTAACGAATTTTTA
AATTCACTTTTGTTTATTTGTCAGATTGTAAGTACTTTCTCTAATCACTTTTTTTTCAAGGCAATCAGGG
TATATTATATTGTACTTCAGCACAGTTTTAGAGAACAATTGTTATAATTAAATGATAAGGTAGAATATT
TCTGCATATAAATTCTGGCTGGCGTGGAAATATTCTTATTGGTAGAAACAACTACACCCTGGTCATCAT
CCTGCCTTTCTCTTTATGGTTACAATGATATACACTGTTTGAGATGAGGATAAAATACTCTGAGTCCAA
ACCGGGCCCCTCTGCTAACCATGTTCATGCCTTCTTCTCTTTCCTACAGCTCCTGGGCAACGTGCTGGTT
GTTGTGCTGTCTCATCATTTTGGCAAAGAATTCCTCGACGGATCCACCGGTCGCCACCATGGTGAGCAA
GGGCGAGGAGCTGTTCACCGGGGTGGTGCCCATCCTGGTCGAGCTGGACGGCGACGTAAACGGCCAC
AAGTTCAGCGTGTCCGGCGAGGGCGAGGGCGATGCCACCTACGGCAAGCTGACCCTGAAGTTCATCTG
CACCACCGGCAAGCTGCCCGTGCCCTGGCCCACCCTCGTGACCACCCTGACCTACGGCGTGCAGTGCT
TCAGCCGCTACCCCGACCACATGAAGCAGCACGACTTCTTCAAGTCCGCCATGCCCGAAGGCTACGTC
CAGGAGCGCACCATCTTCTTCAAGGACGACGGCAACTACAAGACCCGCGCCGAGGTGAAGTTCGAGG
GCGACACCCTGGTGAACCGCATCGAGCTGAAGGGCATCGACTTCAAGGAGGACGGCAACATCCTGGG
GCACAAGCTGGAGTACAACTACAACAGCCACAACGTCTATATCATGGCCGACAAGCAGAAGAACGGC
ATCAAGGTGAACTTCAAGATCCGCCACAACATCGAGGACGGCAGCGTGCAGCTCGCCGACCACTACC
AGCAGAACACCCCCATCGGCGACGGCCCCGTGCTGCTGCCCGACAACCACTACCTGAGCACCCAGTCC
GCCCTGAGCAAAGACCCCAACGAGAAGCGCGATCACATGGTCCTGCTGGAGTTCGTGACCGCCGCCG
GGATCACTCTCGGCATGGACGAGCTGTACAAGTAAAGCGGCCGCCACCGCGGTGGAGCTCGAATTAAT
TCATCGATGATGATCCAGACATGATAAGATACATTGATGAGTTTGGACAAACCACAACTAGAATGCAG
TGAAAAAAATGCTTTATTTGTGAAATTTGTGATGCTATTGCTTTATTTGTAACCATTATAAGCTGCAAT
AAACAAGTTAACAACAACAATTGCATTCATTTTATGTTTCAGGTTCAGGGGGAGGTGTGGGAGGTTTT
TTAAAGCAAGTAAAACCTCTACAAATGTGGTATGGCTGATTATGATCCTCTAGATCAGATCTGTCCCG
ATCTTTCTCTTCTGTGCTGTCAATTTAGAGTAGTTTTTGAGGTAATTTGGGTGCATTCTCTGTAAAAACA
ACAAATAAATATTTATATTGTATGCAATAAATGTAACAATTACACATACACTCTCTGATTACAGCATTA
AAGGGTTAGTTCACCCAAAAATGAAAAGATGTCATTAATGACTCACCCTCATGTCGTTCCAAGCCCGT
AACACCTCCGTTCATCTTCGGAACACAGTTTAAGATATTTTAGATTTAGTCCGAGAGCTTTCTGTCCCT
CCACTGAAAATGTATGTACGGTATACTGTCCATGTCCAGAAAGGTAATAAAAACATCAAAGTAGTCCA
TGTGACATCAGTGGGTTAGGTAGAATTATTTGAAGCATCGAAAATACATTTTGGTCCAAAAATAACAA
AACCTACGACTTTATTCAGCATTGTAGCGTCACTGCGAAGTCGTGAACGCGGATTGACAACAGACCCG
GAAGAGAATACAATGCCGAATAAAGTCGTAGGTTTTGTTATTTTTGGACCAAAATGTATTCGATGCTTC
AAAAAATTCTAACTAACCCACTGATGTCACATGGACTACTTTGATGTTTTTATTACCTTTCTGGACATG
GACAGTATACCGTACATACATTCTCAATGGAGGCACAGAAAGCTCTCGGACTAAATCTAAAATATCTT
AAACTGTGTTCTGAAGATGAACGGAGGTCTTACGGGCTTGGAACGACATGAGGGCGAGTCATTAATGA
CATTATTTTCATTTTTGGGTGAACTAACCCTTTAATAAAACTGACATTTGCAGCCAATTGCACACTTTAT
AATGTTAAATACTTGTTGATTTTAGAAATACAGTAGCATTTAGTGACAATCAAACATTAATTTTATTTC
TGATTATATTGACAAAAACACAGGACTGTAAGGCTGACATGACAGGGTGAATTGATTTCATTACATTA
GTTTTGATGGTAACACTTTTACAATAAGGTTCGTTAGTTAACTACATTAGTTAACATGAACTAATAATG
AACTGCACGTATACATGTTAATTTCAACATTTACTAATACTTTATATCTTGTTAACATTAGTTAATGCAC
TGTGAACTAACATGAACAAACAATGAACATCTGTATTTCTATTAACTAACGTTAACAAAGATTAACAG
ATACAGTAACAAATGTATTGCTCATGGTTATTTAATGTTAGTTAATACATGAACTAATCAACCTCATTG
TAAAGTGTTACCGTTTTGACAATACAACATGTTTGGAAAAAAATTTCTCGCTTGGTTTTCTTGTGTTTTG
CATGAATTCAAACACAATGAGAGCACGGAATGATAATGATTATTCAAAGCTGCAATAGGCGCCAAATT
TCCCGTGGACCTAACGTTACCAATTACAAATGCATTTTAGCATTTTATTACGTGAGCCTTATATTGTCA
ATCGCATGCATTAGTGAAAATAGGCCTACTACACATATAAAAGGCGCGCCATCAACGTCAAAACATGC
ACACCCAAAAAAAAAAAAAAAAAAAAAAAAAAAAACTATCAGTAAAACAAAAGATCCACCGGTCGC
CACCATGGTGATGGAGATGGCAAGCCAGCAGGGCGAGACGATGTCCGGACCGCTGCACATTGGTTTG
ATTCCCACAGTTGGACCGTACCTGCTACCGCATATTATCCCTATGCTGCACCAGACCTTTCCAAAGCTG
GAAATGTATCTGCATGAAGCACAGACCCACCAGTTACTGGCGCAACTGGACAGCGGCAAACTCGATTG
CGTGATCCTCGCGCTGGTGAAAGAGAGCGAAGCATTCATTGAAGTGCCGTTGTTTGATGAGCCAATGT
TGCTGGCTATCTATGAAGATCACCCGTGGGCGAACCGCGAATGCGTACCGATGGCCGATCTGGCAGGG
GAAAAACTGCTGATGCTGGAAGATGGTCACTGTTTGCGCGATCAGGCAATGTCCGCCGGCTACAACAG
CGACAACGTCTATATCATGGCCGACAAGCAGAAGAACGGCATCAAGGCCAACTTCAAGATCCGCCAC
AACGTCGAGGACGGCAGCGTGCAGCTCGCCGACCACTACCAGCAGAACACCCCCATCGGCGACGGCC
CCGTGCTGCTGCCCGACAACCACTACCTGAGCTTCCAGTCCGTCCTGAGCAAAGACCCCAACGAGAAG
CGCGATCACATGGTCCTGCTGGAGTTCGTGACCGCCGCCGGGATCACTCTCGGCATGGACGAGCTGTA
CAACGTGGATGGCGGTAGCGGTGGCACCGGCAGCAAGGGCGAGGAGCTGTTCACCGGGGTGGTGCCC
ATCCTGGTCGAGCTGGACGGCGACGTAAACGGCCACAAGTTCAGCGTGTCCGGCGAGGGCGAGGGCG
ATGCCACCTACGGCAAGCTGACCCTGAAGCTGATCTGCACCACCGGCAAGCTGCCCGTGCCCTGGCCC
ACCCTCGTGACCACCCTCGGCTACGGCCTGAAGTGCTTCGCCCGCTACCCCGACCACATGAAGCAGCA
CGACTTCTTCAAGTCCGCCATGCCCGAAGGCTACGTCCAGGAGCGCACCATCTTCTTCAAGGACGACG
GCAACTACAAGACCCGCGCCGAGGTGAAGTTCGAGGGCGACACCCTGGTGAACCGCATCGAGCTGAA
GGGCATCGGCTTCAAGGAGGACGGCAACATCCTGGGGCACAAGCTGGAGTACAACGGCACCGGTTTC
TGTTTTGAAGCCGGGGCGGATGAAGATACACACTTCCGCGCGACCAGCCTGGAAACTCTGCGCAACAT
GGTGGCGGCAGGTAGCGGGATCACTTTACTGCCAGCGCTGGCTGTGCCGCCGGAGCGCAAACGCGAT
GGGGTTGTTTATCTGCCGTGCATTAAGCCGGAACCACGCCGCACTATTGGCCTGGTTTATCGTCCTGGC
TCACCGCTGCGCAGCCGCTATGAGCAGCTGGCAGAGGCCATCCGCGCAAGAATGGATGGCCATTTCGA
TAAAGTTTTAAAACAGGCGGTTTAAtgagcGGCCGCCACCGCGGTGGAGCTCGAATTAATTCATCGATGA
TGATCCAGACATGATAAGATACATTGATGAGTTTGGACAAACCACAACTAGAATGCAGTGAAAAAAA
TGCTTTATTTGTGAAATTTGTGATGCTATTGCTTTATTTGTAACCATTATAAGCTGCAATAAACAAGTTA
ACAACAACAATTGCATTCATTTTATGTTTCAGGTTCAGGGGGAGGTGTGGGAGGTTTTTTAAAGCAAG
TAAAACCTCTACAAATGTGGTATGGCTGATTATGATCCTCTAGATCAGATCTGTCCCGATCTTTCTCTT
CTGTGCTGTCAATTTAGAGTAGTTTTTGAGGTAATTTGGGTGCATTCTCTGTAAAAACAACAAATAAAT
ATTTATATTGTATGCAATAAATGTAACAATTACACATACACTCTCTGATTACAGCATTAAAGGGTTAGT
TCACCCAAAAATGAAAAGATGTCATTAATGACTCACCCTCATGTCGTTCCAAGCCCGTAACACCTCCG
TTCATCTTCGGAACACAGTTTAAGATATTTTAGATTTAGTCCGAGAGCTTTCTGTCCCTCCACTGAAAA
TGTATGTACGGTATACTGTCCATGTCCAGAAAGGTAATAAAAACATCAAAGTAGTCCATGTGACATCA
GTGGGTTAGGTAGAATTATTTGAAGCATCGAAAATACATTTTGGTCCAAAAATAACAAAACCTACGAC
TTTATTCAGCATTGTAGCGTCACTGCGAAGTCGTGAACGCGGATTGACAACAGACCCGGAAGAGAATA
CAATGCCGAATAAAGTCGTAGGTTTTGTTATTTTTGGACCAAAATGTATTCGATGCTTCAAAAAATTCT
AACTAACCCACTGATGTCACATGGACTACTTTGATGTTTTTATTACCTTTCTGGACATGGACAGTATAC
CGTACATACATTCTCAATGGAGGCACAGAAAGCTCTCGGACTAAATCTAAAATATCTTAAACTGTGTT
CTGAAGATGAACGGAGGTCTTACGGGCTTGGAACGACATGAGGGCGAGTCATTAATGACATTATTTTC
ATTTTTGGGTGAACTAACCCTTTAATAAAACTGACATTTGCAGCCAATTGCACACTTTATAATGTTAAA
TACTTGTTGATTTTAGAAATACAGTAGCATTTAGTGACAATCAAACATTAATTTTATTTCTGATTATATT
GACAAAAACACAGGACTGTAAGGCTGACATGACAGGGTGAATTGATTTCATTACATTAGTTTTGATGG
TAACACTTTTACAATAAGGTTCGTTAGTTAACTACATTAGTTAACATGAACTAATAATGAACTGCACGT
ATACATGTTAATTTCAACATTTACTAATACTTTATATCTTGTTAACATTAGTTAATGCACTGTGAACTAA
CATGAACAAACAATGAACATCTGTATTTCTATTAACTAACGTTAACAAAGATTAACAGATACAGTAAC
AAATGTATTGCTCATGGTTATTTAATGTTAGTTAATACATGAACTAATCAACCTCATTGTAAAGTGTTA
CCGTTTTGACAATACAACATGTTTGGAAAAAAATTTCTCGCTTGGTTTTCTTGTGTTTTGCATGAATTCA
AACACAATGAGAGCACGGAATGATAATGATTATTCAAAGCTGCAATAGGCGCCAAATTTCCCGTGGAC
CTAACGTTACCAATTACAAATGCATTTTAGCATTTTATTACGTGAGCCTTATATTGTCAATCGCATGCA
TTAGTGAAAATAGGCCTACTACACATATAAAAGGCGCGCCATCAACGTCAAAACATGCACACCCAAA
AAAAAAAAAAAAAAAAAAAAAAAAAACTATCAGTAAAACAAAATACTTAATGTACTTACCTCAATAT
GCTTCCTTAGGTTTGATGGCGAACTTTTGAAGGCCGATATTTCTTTATTAAGCGGGAGACAGAGGACA
CATTTCATCTTGAATGAATCTTTATTTACACCACTTAAAGAAAAGAATTCGCGAAGATACGGCCACGG
GTGCTCTTGATCCTGTGGCTGATTTTGGACTGTGCTGCTCGCAGCTGCTGATGAATCACATACTTCCTC
CATTTTCTTCCACTGATTGACTGTTATAATTTCCCTAATTTCCAGGTCAAGGTGCTGTGCATTGTGGTAA
TAGATGTGACATGACGTCACTTCCAAAGGACCAATGAACATGTCTGACCAATTTCATATAATGTGAAA
ACGATTTTCATAGGCAGAATAAATAACATTTAAATTAAACTGGGCATCAGCGCAATTCAATTGGTTTG
GTAATAGCAAGGGAAAATAGAATGAAGTGATCTCCAAAAAATAAGTACTTTTTGACTGTAAATAAAAT
TGTAAGGAGTAAAAAGTACTTTTTTTTCTAAAAAAATGTAATTAAGTAAAAGTAAAAGTATTGATTTTT
AATTGTACTCAAGTAAAGTAAAAATCCCCAAAAATAATACTTAAGTACAGTAATCAAGTAAAATTACT
CAAGTACTTTACACCTCTGGTTCTTGACCCCCTACCTTCAGCAAGCCCAGCAGATCCACTAGTTCTAGA
GCGGCCGCCACCGCGGTGGAGCTCCAGCTTTTGTTCCCTTTAGTGAGGGTTAATTGCGCGCTTGGCGTA
ATCATGGTCATAGCTGTTTCCTGTGTGAAATTGTTATCCGCTCACAATTCCACACAACATACGAGCCGG
AAGCATAAAGTGTAAAGCCTGGGGTGCCTAATGAGTGAGCTAACTCACATTAATTGCGTTGCGCTCAC
TGCCCGCTTTCCAGTCGGGAAACCTGTCGTGCCAGCTGCATTAATGAATCGGCCAACGCGCGGGGAGA
GGCGGTTTGCGTATTGGGCGCTCTTCCGCTTCCTCGCTCACTGACTCGCTGCGCTCGGTCGTTCGGCTG
CGGCGAGCGGTATCAGCTCACTCAAAGGCGGTAATACGGTTATCCACAGAATCAGGGGATAACGCAG
GAAAGAACATGTGAGCAAAAGGCCAGCAAAAGGCCAGGAACCGTAAAAAGGCCGCGTTGCTGGCGTT
TTTCCATAGGCTCCGCCCCCCTGACGAGCATCACAAAAATCGACGCTCAAGTCAGAGGTGGCGAAACC
CGACAGGACTATAAAGATACCAGGCGTTTCCCCCTGGAAGCTCCCTCGTGCGCTCTCCTGTTCCGACCC
TGCCGCTTACCGGATACCTGTCCGCCTTTCTCCCTTCGGGAAGCGTGGCGCTTTCTCATAGCTCACGCT
GTAGGTATCTCAGTTCGGTGTAGGTCGTTCGCTCCAAGCTGGGCTGTGTGCACGAACCCCCCGTTCAGC
CCGACCGCTGCGCCTTATCCGGTAACTATCGTCTTGAGTCCAACCCGGTAAGACACGACTTATCGCCAC
TGGCAGCAGCCACTGGTAACAGGATTAGCAGAGCGAGGTATGTAGGCGGTGCTACAGAGTTCTTGAA
GTGGTGGCCTAACTACGGCTACACTAGAAGGACAGTATTTGGTATCTGCGCTCTGCTGAAGCCAGTTA
CCTTCGGAAAAAGAGTTGGTAGCTCTTGATCCGGCAAACAAACCACCGCTGGTAGCGGTGGTTTTTTT
GTTTGCAAGCAGCAGATTACGCGCAGAAAAAAAGGATCTCAAGAAGATCCTTTGATCTTTTCTACGGG
GTCTGACGCTCAGTGGAACGAAAACTCACGTTAAGGGATTTTGGTCATGAGATTATCAAAAAGGATCT
TCACCTAGATCCTTTTAAATTAAAAATGAAGTTTTAAATCAATCTAAAGTATATATGAGTAAACTTGGT
CTGACAGTTACCAATGCTTAATCAGTGAGGCACCTATCTCAGCGATCTGTCTATTTCGTTCATCCATAG
TTGCCTGACTCCCCGTCGTGTAGATAACTACGATACGGGAGGGCTTACCATCTGGCCCCAGTGCTGCA
ATGATACCGCGAGACCCACGCTCACCGGCTCCAGATTTATCAGCAATAAACCAGCCAGCCGGAAGGG
CCGAGCGCAGAAGTGGTCCTGCAACTTTATCCGCCTCCATCCAGTCTATTAATTGTTGCCGGGAAGCTA
GAGTAAGTAGTTCGCCAGTTAATAGTTTGCGCAACGTTGTTGCCATTGCTACAGGCATCGTGGTGTCAC
GCTCGTCGTTTGGTATGGCTTCATTCAGCTCCGGTTCCCAACGATCAAGGCGAGTTACATGATCCCCCA
TGTTGTGCAAAAAAGCGGTTAGCTCCTTCGGTCCTCCGATCGTTGTCAGAAGTAAGTTGGCCGCAGTGT
TATCACTCATGGTTATGGCAGCACTGCATAATTCTCTTACTGTCATGCCATCCGTAAGATGCTTTTCTGT
GACTGGTGAGTACTCAACCAAGTCATTCTGAGAATAGTGTATGCGGCGACCGAGTTGCTCTTGCCCGG
CGTCAATACGGGATAATACCGCGCCACATAGCAGAACTTTAAAAGTGCTCATCATTGGAAAACGTTCT
TCGGGGCGAAAACTCTCAAGGATCTTACCGCTGTTGAGATCCAGTTCGATGTAACCCACTCGTGCACC
CAACTGATCTTCAGCATCTTTTACTTTCACCAGCGTTTCTGGGTGAGCAAAAACAGGAAGGCAAAATG
CCGCAAAAAAGGGAATAAGGGCGACACGGAAATGTTGAATACTCATACTCTTCCTTTTTCAATATTAT
TGAAGCATTTATCAGGGTTATTGTCTCATGAGCGGATACATATTTGAATGTATTTAGAAAAATAAACA
AATAGGGGTTCCGCGCACATTTCCCCGAAAAGTG
tp63: HyPer-mito
Sequence:
(SEQ ID NO: 29)
GCCACCGCGGTGGAGCTCGAATTAATTCATCGATGATGATCCAGACATGATAAGATACATTGATGAGT
TTGGACAAACCACAACTAGAATGCAGTGAAAAAAATGCTTTATTTGTGAAATTTGTGATGCTATTGCTT
TATTTGTAACCATTATAAGCTGCAATAAACAAGTTAACAACAACAATTGCATTCATTTTATGTTTCAGG
TTCAGGGGGAGGTGTGGGAGGTTTTTTAAAGCAAGTAAAACCTCTACAAATGTGGTATGGCTGATTAT
GATCCTCTAGATCAGGCCGCCACCGCGGTGGAGCTCCAGCTTTTGTTCCCTTTAGTGAGGGTTAATTGC
GCGCTTGGCGTAATCATGGTCATAGCTGTTTCCTGTGTGGCGAAGATACGGCCACGGGTGCTCTTGATC
CTGTGGCTGATTTTGGACTGTGCTGCTCGCAGCTGCTGATGAATCACATACTTCCTCCATTTTCTTCCAC
TGATTGACTGTTATAATTTCCCTAATTTCCAGGTCAAGGTGCTGTGCATTGTGGTAATAGATGTGACAT
GACGTCACTTCCAAAGGACCAATGAACATGTCTGACCAATTTCATATAATGTGAAAACGATTTTCATA
GGCAGAATAAATAACATTTAAATTAAACTGGGCATCAGCGCAATTCAATTGGTTTGGTAATAGCAAGG
GAAAATAGAATGAAGTGATCTCCAAAAAATAAGTACTTTTTGACTGTAAATAAAATTGTAAGGAGTAA
AAAGTACTTTTTTTTCTAAAAAAATGTAATTAAGTAAAAGTAAAAGTATTGATTTTTAATTGTACTCAA
GTAAAGTAAAAATCCCCAAAAATAATACTTAAGTACAGTAATCAAGTAAAATTACTCAAGTACTTTAC
ACCTCTGGGGGAGGTTCCCTTTAGTGAGGGTTAATTGCGCGCTTGGCGTAATCATGGTCATAGCTGTTT
CCTGTGTGAAATTGTTATCCGCTCACAATTCCACACAACATACGAGCCGGAAGCATAAAGTGTAAAGC
CTGGTGAAGCCAGTTACCTTCGGAAAAAGAGTTGGTAGCTCTTGATCCGGCAAACAAACCACCGCTGG
TAGCGGTGGTTTTTTTGTTTGCAAGCAGCAGATTACGCGCAGAAAAAAAGGATCTCAAGAAGATCCTT
TGATCTTTTCTACGGGGTCTGACGCTCAGTGGAACGAAAACTCACGTTAAGGGATTTTGGTCATGAGA
TTATCAAAAAGGATCTTCACCTAGATCCTTTTAAATTAAAAATGAAGTTTTAAATCAATCTAAAGTATA
TATGAGTAAACTTGGTCTGACAGTTACCAATGCTTAATCAGTGAGGCACCTATCTCAGCGATCTGTCTA
TTTCGTTCATCCATAGTTGCCTGACTCCCCGTCGTGTAGATAACTACGATACGGGAGGGCTTACCATCT
GGCCCCAGTGCTGCAATGATACCGCGAGACCCACGCTCACCGGCTCCAGATTTATCAGCAATAAACCA
GCCAGCCGGAAGGGCCGAGCGCAGAAGTGGTCCTGCAACTTTATCCGCCTCCATCCAGTCTATTAATT
GTTGCCGGGAAGCTAGAGTAAGTAGTTCGCCAGTTAATAGTTTGCGCAACGTTGTTGCCATTGCTACA
GGCATCGTGGTGTCACGCTCGTCGTTTGGTATGGCTTCATTCAGCTCCGGTTCCCAACGATCAAGGCGA
GTTACATGATCCCCCATGTTGTGCAAAAAAGCGGTTAGCTCCTTCGGTCCTCCGATCGTTGTCAGAAGT
AAGTTGGCCGCAGTGTTATCACTCATGGTTATGGCAGCACTGCATAATTCTCTTACTGTCATGCCATCC
GTAAGATGCTTTTCTGTGACTGGTGAGTACTCAACCAAGTCATTCTGAGAATAGTGTATGCGGCGACC
GAGTTGCTCTTGCCCGGCGTCAATACGGGATAATACCGCGCCACATAGCAGAACTTTAAAAGTGCTCA
TCATTGGAAAACGTTCTTCGGGGCGAAAACTCTCAAGGATCTTACCGCTGTTGAGATCCAGTTCGATGT
AACCCACTCGTGCACCCAACTGATCTTCAGCATCTTTTACTTTCACCAGCGTTTCTGGGTGAGCAAAAA
CAGGAAGGCAAAATGCCGCAAAAAAGGGAATAAGGGCGACACGGAAATGTTGAATACTCATACTCTT
CCTTTTTCAATATTATTGAAGCATTTATCAGGGTTATTGTCTCATGAGCGGATACATATTTGAATGTATT
TAGAAAAATAAACAAATAGGGGTTCCGCGCACATTTCCCCGAAAAGTGCCACCTAAATTGTAAGCGTT
AATATTTTGTTAAAATTCGCGTTAAATTTTTGTTAAATCAGCTCATTTTTTAACCAATAGGCCGAAATC
GGCAAAATCCCTTATAAATCAAAAGAATAGACCGAGATAGGGTTGAGTGTTGTTCCAGTTTGGAACAA
GAGTCCACTATTAAAGAACGTGGACTCCAACGTCAAAGGGCGAAAAACCGTCTATCAGGGCGATGGC
CCACTACGTGAACCATCACCCTAATCAAGTTTTTTGGGGTCGAGGTGCCGTAAAGCACTAAATCGGAA
CCCTAAAGGGAGCCCCCGATTTAGAGCTTGACGGGGAAAGCCAGAGGTGTAAAAAGTACTCAAAAAT
TTTACTCAAGTGAAAGTACAAGTACTTAGGGAAAATTTTACTCAATTAAAAGTAAAAGTATCTGGCTA
GAATCTTACTTGAGTAAAAGTAAAAAAGTACTCCATTAAAATTGTACTTGAGTATTAAGGAAGTAAAA
GTAAAAGCAAGAAAGAAAACTAGAGATTCTTGTTTAAGCTTTTAATCTCAAAAAACATTAAATGAAAT
GCATACAAGGTTTTATCCTGCTTTAGAACTGTTTGTATTTAATTATCAAACTATAAGACAGACAATCTA
ATGCCAGTACACGCTACTCAAAGTTGTAAAACCTCAGATTTAACTTCAGTAGAAGCTGATTCTCAAAA
TTGTTAGTGTCAAGCCTAGCTCTTTTGGGGCTGAAAAGCAATCCTGCAGTGCTGAAAAGCCTCTCACA
GGCAGCCGATGCGGGAAGAGGTGTATTAGTCTTGATAGAGAGGCTGCAAATAGCAGGAAACGTGAGC
AGAGACTCCCTGGTGTCTGAAACACAGGCCAGATCGCGTTGTAAAACGACGGCCAGTGAGCGCGCGT
AATACGACTCACTATAGGGCGAATTGGGTACGATCCACCGGTCGGCCGGCCGATTAATTAACGAGCTC
GAGAGAAACCAGGCTCAGTCGGGCACGACCATTACTCCACTTGACCAAACGTCTGCATATTTATAAAT
AATAATTTGCGTATTAATATGCAGATAAAGACAATTAAAAGAAGCAACAGTAGCATTAATCATTCAAA
TAACAGTATACAATCAAACAACATTTTGTATATATATTTAAAAAAACAATACAAGGAGTAAAGACAAA
GGCAATATCATTTAGTTTTTTTAATATTAACAAACCACAGGCTGTGTGAACCATCAGCAATTTCAAACC
AGAGGGATCTGCTTTTTTGGGAACGATGTTGCAAATTTAATCTCTAGATTTTTTAAATCATGGATAGCC
TCCCTAAAGCAAAAGCTTTTTAGGGGCTTTACTTCTCCTGATTGCAGCCTGTGGTATAATTTGTGATTG
TTAGGTGGGTCAGGTTTTGGGGTCCAGCCCTTTGTGCACTTTGACTATTGTCACCTTAGGCTGTCCCTTA
ATGCTGCCACCTCTGACCCCGGTGGGACACATAAATAACTTCTCCACCCGAGTTAGCATTTCAAGCCA
GTGCAATAAAGAAATTAAAAATAATTGATTTATTAACAACTCTGTTTTATCTTTAAAAAATAAAATGTT
TTATATCTAATGTTCACTCAGGTACCTGATATTGTTTGCTGTGCAATAATGTAGGTTAGTCATTATATTG
ACAAAGAAAATTAAATATGATGTTGTTTATTTTTAATTTTACATTATAGACTAGATTAGATTTTCATTTG
GAATTTAATTAGGAGTGCCTGGAGTGCATTGAGAACGTTAAAAAAAAAAAATCTGCTTACTCTTATAG
CTTTGGAAACTATTTTCAGATTATTACTCCACTATAGAAGCGATTCCTTTTTTTGAATAATTAAATATAT
ATATACAGTGCTCAGCATAAATGATCATCACAGATCTCTATTTTAAAGTATTATTTTTCCATAGGATGC
TTTACAATAATATATGTGTGTGTATACATTAGAATTGTCAGTTACAAAATCAAAACTGCAACAAAATA
ACTAAACTTAAGATCTAATGAGTACACCCAAATTAAACCAAACATTAGTCTATGCAAAAATTAATATG
TTAAGGAAAAATATTAAATACAATTTTAATATTCAGTCATTCATTTTCTTTTTTGCTTAGTCCCTTTATT
AATCTGGGGTCGCCACAGTGGAATTAACTGCCAATTTATCCAGCATATATTTTATGCAGCGGATGCTCT
TCCAGCTGCTACCCATCACTGGGAATCACCCATACACTCTCATTCACACACATACACTACGGACAATTT
AGCCTACCCAATTCACCTATAGCGTATGTCTTTGGACTTGTGGGGGAAACCGGAACACACGGAGGAAA
CCCACGCGAACACAGGGAGATCATGCAAACTCAGAAACGCCAACTGATCCAGCCAAGGCTCGAACCA
GTGACCTTCTTGCTGTGAGGCAATAGGGCTACCCATTGTGTCACTGCACCGTCCACAATTTTAATAAAC
ATGAAAAATCCCGAGAAACATAAACAATATTAAATGTAGTTGAAATTTTGTTGTTTGAAATTGTATTTA
TTAAATAAAAAAAATGTATGTATTATTATTATTTATTTATTTATAAAAGATGTTAGGTGGCCAAACTCT
TTTTTTTAATGGATATAGTTGTTTAATAAAACAATTTTGTTTAAATAAAACAAAATATATTGAATGAGC
CATGTTGCTGTTGTTGTTTTTTACAGGCTTGATGTACAATATGATCTTTGTGTAGTTTTATAGTAATAAA
TCATTCATGTCTTGTGTATTGTACGTCATATTATCTTAAAAGTACGCTATTAATTAAACTTGTTTATATG
GTCATCTTAGACAGGACTCTCATTATAAAAGGATTTTTGTTAATTTTATTAGTACCTTCCTGGAAAAAT
AAAAGATAAATAAATAAAAATGAATAAAAGTCTTGGGCTTGTTTTCTGTACGAGTAATAAAAAAGAGT
AAGCAAATATTCTGCATGACAATATTGTTCAAAGTTTAAGGGCTGTATTTTTTATAATTGTTGGTTATT
AACATGTTTTATACAAATTACTTTCAACATGGCTGAAATAATAATTGTTATCACTATTTGAAACAGTCC
TAGAATAGATTTTACTATATTTACATTTATTCCTCTGATGACAAAGTTGAAATTCCTCTGATGACAAAG
CAAAACTCTCAACTCTAGTTTTATTTTATTGTAATAACCATGTCAAGGGATAATGACAATGTTTTCCAT
TATATTTTCAATATAGAATAAAATTATAATTTATTAATCATTTAGTAGGGCAGCATGGTGGCACAATGG
GTAGCACAATCACCTCACACCAAGAAGGTTGCTAGTTCGAGCCCCGGCTGGGTCAGTTTGCATATCTG
TGTGGAGAATGTTCTCTCTGTGTTGCCATGGGTTTCCTCTGGGTGCTCTAGTTTCCACCACAAGTCTAA
AAACAAGTGGCATCGGTGAATTGTGTAGGCTAAATTGTCTGTAGTGTTTGTGTGTGATTGAGTGTTTAT
GGATGTTTCCCAGTGATGGGTTGCAGTTGGAAGGGCATTCTCTGCGTAAAACTATAAGTTGGCGGTTC
ATTCCACTGTGGTGACCCCAGATTAATAAAGGGATTAAGCCGAAAAGAAAATGAATCATTAAATGATC
ATTTAGTTGATTGGAGAGCATTGCATAATGAGAATCAAATAATGTATAGTTTTTTGTAAAATGATTGTG
TATTAGAGGCACATTGTACGTAGTGTGGAATGTTTAGGTTGTCTTTCTGATTGTTTTCATTTTGCAGAA
GTTCATTTGCAGTCAGTGTAGTCAAAGCACCTCAGCTCAATTTATAGCAGCAGCCTGCTGTGATTTAAG
TGCCACAGGAGTGAAGGCTTTAACCCCTGTTGTAGATTGCAACCAATGTTTTTCTAAGGTAATTAGAA
GCTTGTTTAGCTAACGCTACCCACCCCACCCCCCACAAAAAAGTTGCTCTGAATAGCAGCATCTACCCC
CAGGAGGGTGCAAGCTTCTGTTTTGAAGAAATTTGCTCATGGTGTTACATAGGTGTTTAAAAATGTATG
TTTTTTAATAGCATATATTTTAAAAGTGACTCATTAATGACATCATCTGCCATCTGCCTTTCTCAGAACT
GACAAAGGACCCTTTTAAGGACCCCTATACACCACATTTTTATCTATCTAATCACAAGTGGGTGATATT
AGAGGGATGATTCACCTAAAAATGAATTTTCCGCATTTTAATTTGGGGTTCCAAACCAGTTTGAATTCT
TTTGTTCTGTTGAACACAAAATAAGATGTTTTAAGAATGTTGGAAATGTTGGAGACTTTTATAGTAGGA
AAAACATATACTAATATTTGCATTCATGTTTGAAATGTTTAAAACATTTTTCAAAATATCTTATTTTTCA
ACTGTTTTGGAACAAGTCAAAAGTGAGTAAATGATGACTGATAAACAATTTCTTTAAATACAGATGAA
AATAGCGTCTAACACATTTTGAACTAGTTCACTTTCAACCACAGCAAGAGGTAGTCGAAAACACATTT
GATCACATTTTGTTGTGGGACCGCTTGTTGTTCAGTGTGTTTTCATTGTTGAGTTACAAACCGGTAAGT
GGCATTAAAACATGTTTTGGCATCACTGATGATCATAAATAAATTGGTTAAAAAATTAAAAATTAGAT
GCTTACAGTACAGTTTGTTTAAAAACTTGCACTTTTTGTAGTGTGAAATGACATTAGTACCTGCAGTGG
GCTTTCTTTGTTCTGTATCTTCTGATGAATCTAACATAAAGACATTTAAATATTCACAATGGAGTGGGT
TGGTTCTCATCTATCTGAAACAACATTAGTGTGTTCAAAACAAATCTACACCAGTGATCTCTGATAGAT
ACAAGCTGTCAACCGTAGTCCTATTGTTTCTCATGCGACTGTCACAGGGCCATGCTAGAGGCTCTTTGG
GTGGTGAGTAGAGACTCATTCTTATGCAAACAGGCTATGGATGCTACAACTGAAGAGAATTTAGTGCT
CTAAGGTCATCGCAGTTCTATAAAGCAGATTCCCTCCAAAAAGCTAGAGAAAATTATGTGTCTGGAGG
CACCATCTATCAGTGCAGTCATTCTTTTATTACTTTTTTTTTTTGTCCATGCAATTTTTTAATCGCATTTT
GAAGTATTTAAACCATTAAGAGTGTCAAATTATCATGTTATTATTTCAACTCATAATATCTCCTTTTAA
AGATGATTCATTTGATTGTAGTTATAATAATTGTTTTATTAATGCAAAAATTGCTTAAATGTTTGGCCTC
TGAGATTTTCAGTTGACTCAATATTTTTGGAGCCTGTTTCATGACTTTATTTAAAATATAATCTGCACTC
GCAAATATTACAATTATATCATATTTTGAAAATCTTAGATGTACATTTGTTTTGAAAATGGTTTAAAGG
AAATTTAGGTCAAAATGTGTGTTTTTTTTAAATAATAATTCAGTCTTTTTTTTGATATAATAGGGAAAA
TGACAGCAATTAAAAGGCTGTTTTGGAAAGACATCTAGGTCTGGTGCAGACTTCGTAACTTCTTAGAG
TTAAATCTTGAAAACGGAGCCAGAAGACGGGCAACACCTTCCTAAAAGCAGGGTGGGGTGTTGGACA
GGTAGCTTCTTGGAACCTCCCCTCTTTGTCGCTGTGGTATATATAGAAATCCCATTGAATTCCTATAGA
GACCTTAAGCCCGTACCATTTACCTCTGAAGCCACGGGTTTAGTGAACCGTCAGATCCGCTAGCATGTC
CGTCCTGACGCCGCTGCTGCTGCGGGGCTTGACAGGCTCGGCCCGGCGGCTCCCAGTGCCGCGCGCCA
AGATCCATTCGTTGGGGGATCTGTCCGTCCTGACGCCGCTGCTGCTGCGGGGCTTGACAGGCTCGGCC
CGGCGGCTCCCAGTGCCGCGCGCCAAGATCCATTCGTTGGGGGATCCACCGGTCGCCACCGAGATGGC
AAGCCAGCAGGGCGAGACGATGTCCGGACCGCTGCACATTGGTTTGATTCCCACAGTTGGACCGTACC
TGCTACCGCATATTATCCCTATGCTGCACCAGACCTTTCCAAAGCTGGAAATGTATCTGCATGAAGCAC
AGACCCACCAGTTACTGGCGCAACTGGACAGCGGCAAACTCGATTGCGTGATCCTCGCGCTGGTGAAA
GAGAGCGAAGCATTCATTGAAGTGCCGTTGTTTGATGAGCCAATGTTGCTGGCTATCTATGAAGATCA
CCCGTGGGCGAACCGCGAATGCGTACCGATGGCCGATCTGGCAGGGGAAAAACTGCTGATGCTGGAA
GATGGTCACTGTTTGCGCGATCAGGCAATGTCCGCCGGCTACAACAGCGACAACGTCTATATCATGGC
CGACAAGCAGAAGAACGGCATCAAGGCCAACTTCAAGATCCGCCACAACGTCGAGGACGGCAGCGTG
CAGCTCGCCGACCACTACCAGCAGAACACCCCCATCGGCGACGGCCCCGTGCTGCTGCCCGACAACCA
CTACCTGAGCTTCCAGTCCGTCCTGAGCAAAGACCCCAACGAGAAGCGCGATCACATGGTCCTGCTGG
AGTTCGTGACCGCCGCCGGGATCACTCTCGGCATGGACGAGCTGTACAACGTGGATGGCGGTAGCGGT
GGCACCGGCAGCAAGGGCGAGGAGCTGTTCACCGGGGTGGTGCCCATCCTGGTCGAGCTGGACGGCG
ACGTAAACGGCCACAAGTTCAGCGTGTCCGGCGAGGGCGAGGGCGATGCCACCTACGGCAAGCTGAC
CCTGAAGCTGATCTGCACCACCGGCAAGCTGCCCGTGCCCTGGCCCACCCTCGTGACCACCCTCGGCT
ACGGCCTGAAGTGCTTCGCCCGCTACCCCGACCACATGAAGCAGCACGACTTCTTCAAGTCCGCCATG
CCCGAAGGCTACGTCCAGGAGCGCACCATCTTCTTCAAGGACGACGGCAACTACAAGACCCGCGCCG
AGGTGAAGTTCGAGGGCGACACCCTGGTGAACCGCATCGAGCTGAAGGGCATCGGCTTCAAGGAGGA
CGGCAACATCCTGGGGCACAAGCTGGAGTACAACGGCACCGGTTTCTGTTTTGAAGCCGGGGCGGATG
AAGATACACACTTCCGCGCGACCAGCCTGGAAACTCTGCGCAACATGGTGGCGGCAGGTAGCGGGAT
CACTTTACTGCCAGCGCTGGCTGTGCCGCCGGAGCGCAAACGCGATGGGGTTGTTTATCTGCCGTGCA
TTAAGCCGGAACCACGCCGCACTATTGGCCTGGTTTATCGTCCTGGCTCACCGCTGCGCAGCCGCTATG
AGCAGCTGGCAGAGGCCATCCGCGCAAGAATGGATGGCCATTTCGATAAAGTTTTAAAACAGGCGGTT
TAAAGCGGCCGCGACTCTAGATCATAATCAGCCATACCACATTTGTAGAGGTTTTACTTGCTTTAAAAA
ACCTCCCACACCTCCCCCTGAACCTGAAACATAAAATGAATGCAATTGTTGTTGTTAACTTGTTTATTG
CAGCTTATAATGGTTACAAATAAAGCAATAGCATCACAAATTTCACAAATAAAGCATTTTTTTCACTGC
ATTCTAGTTGTGGTTTGTCCAAACTCATCAATGTATCTTA
pBSK-JC_T2_tp63_Gal4VP16_GFP-5xUAS-MCS
Sequence:
(SEQ ID NO: 30)
CTGACGCGCCCTGTAGCGGCGCATTAAGCGCGGCGGGTGTGGTGGTTACGCGCAGCGTGACCGCTACA
CTTGCCAGCGCCCTAGCGCCCGCTCCTTTCGCTTTCTTCCCTTCCTTTCTCGCCACGTTCGCCGGCTTTC
CCCGTCAAGCTCTAAATCGGGGGCTCCCTTTAGGGTTCCGATTTAGTGCTTTACGGCACCTCGACCCCA
AAAAACTTGATTAGGGTGATGGTTCACGTAGTGGGCCATCGCCCTGATAGACGGTTTTTCGCCCTTTGA
CGTTGGAGTCCACGTTCTTTAATAGTGGACTCTTGTTCCAAACTGGAACAACACTCAACCCTATCTCGG
TCTATTCTTTTGATTTATAAGGGATTTTGCCGATTTCGGCCTATTGGTTAAAAAATGAGCTGATTTAAC
AAAAATTTAACGCGAATTTTAACAAAATATTAACGCTTACAATTTGCCATTCGCCATTCAGGCTGCGCA
ACTGTTGGGAAGGGCGATCGGTGCGGGCCTCTTCGCTATTACGCCAGCTGGCGAAAGGGGGATGTGCT
GCAAGGCGATTAAGTTGGGTAACGCCAGGGTTTTCCCAGTCACGACGTTGTAAAACGACGGCCAGTGA
ATTGTAATACGACTCACTATAGGGCGACCCTGCAGAGGTGTAAAAAGTACTCAAAAATTTTACTCAAG
TGAAAGTACAAGTACTTAGGGAAAATTTTACTCAATTAAAAGTAAAAGTATCTGGCTAGAATCTTACT
TGAGTAAAAGTAAAAAAGTACTCCATTAAAATTGTACTTGAGTATTAAGGAAGTAAAAGTAAAAGCA
AGAAAGAAAACTAGAGATTCTTGTTTAAGCTTTTAATCTCAAAAAACATTAAATGAAATGCATACAAG
GTTTTATCCTGCTTTAGAACTGTTTGTATTTAATTATCAAACTATAAGACAGACAATCTAATGCCAGTA
CACGCTACTCAAAGTTGTAAAACCTCAGATTTAACTTCAGTAGAAGCTGATTCTCAAAATTGTTAGTGT
CAAGCCTAGCTCTTTTGGGGCTGAAAAGCAATCCTGCAGTGCTGAAAAGCCTCTCACAGGCAGCCGAT
GCGGGAAGAGGTGTATTAGTCTTGATAGAGAGGCTGCAAATAGCAGGAAACGTGAGCAGAGACTCCC
TGGTGTCTGAAACACAGGCCAGATGGCCCCGCGGCCGGGCCCTCGGCCGGCCGATTAATTAACGAGCT
CGAGAGAAACCAGGCTCAGTCGGGCACGACCATTACTCCACTTGACCAAACGTCTGCATATTTATAAA
TAATAATTTGCGTATTAATATGCAGATAAAGACAATTAAAAGAAGCAACAGTAGCATTAATCATTCAA
ATAACAGTATACAATCAAACAACATTTTGTATATATATTTAAAAAAACAATACAAGGAGTAAAGACAA
AGGCAATATCATTTAGTTTTTTTAATATTAACAAACCACAGGCTGTGTGAACCATCAGCAATTTCAAAC
CAGAGGGATCTGCTTTTTTGGGAACGATGTTGCAAATTTAATCTCTAGATTTTTTAAATCATGGATAGC
CTCCCTAAAGCAAAAGCTTTTTAGGGGCTTTACTTCTCCTGATTGCAGCCTGTGGTATAATTTGTGATT
GTTAGGTGGGTCAGGTTTTGGGGTCCAGCCCTTTGTGCACTTTGACTATTGTCACCTTAGGCTGTCCCTT
AATGCTGCCACCTCTGACCCCGGTGGGACACATAAATAACTTCTCCACCCGAGTTAGCATTTCAAGCC
AGTGCAATAAAGAAATTAAAAATAATTGATTTATTAACAACTCTGTTTTATCTTTAAAAAATAAAATGT
TTTATATCTAATGTTCACTCAGGTACCTGATATTGTTTGCTGTGCAATAATGTAGGTTAGTCATTATATT
GACAAAGAAAATTAAATATGATGTTGTTTATTTTTAATTTTACATTATAGACTAGATTAGATTTTCATTT
GGAATTTAATTAGGAGTGCCTGGAGTGCATTGAGAACGTTAAAAAAAAAAAATCTGCTTACTCTTATA
GCTTTGGAAACTATTTTCAGATTATTACTCCACTATAGAAGCGATTCCTTTTTTTGAATAATTAAATATA
TATATACAGTGCTCAGCATAAATGATCATCACAGATCTCTATTTTAAAGTATTATTTTTCCATAGGATG
CTTTACAATAATATATGTGTGTGTATACATTAGAATTGTCAGTTACAAAATCAAAACTGCAACAAAAT
AACTAAACTTAAGATCTAATGAGTACACCCAAATTAAACCAAACATTAGTCTATGCAAAAATTAATAT
GTTAAGGAAAAATATTAAATACAATTTTAATATTCAGTCATTCATTTTCTTTTTTGCTTAGTCCCTTTAT
TAATCTGGGGTCGCCACAGTGGAATTAACTGCCAATTTATCCAGCATATATTTTATGCAGCGGATGCTC
TTCCAGCTGCTACCCATCACTGGGAATCACCCATACACTCTCATTCACACACATACACTACGGACAATT
TAGCCTACCCAATTCACCTATAGCGTATGTCTTTGGACTTGTGGGGGAAACCGGAACACACGGAGGAA
ACCCACGCGAACACAGGGAGATCATGCAAACTCAGAAACGCCAACTGATCCAGCCAAGGCTCGAACC
AGTGACCTTCTTGCTGTGAGGCAATAGGGCTACCCATTGTGTCACTGCACCGTCCACAATTTTAATAAA
CATGAAAAATCCCGAGAAACATAAACAATATTAAATGTAGTTGAAATTTTGTTGTTTGAAATTGTATTT
ATTAAATAAAAAAAATGTATGTATTATTATTATTTATTTATTTATAAAAGATGTTAGGTGGCCAAACTC
TTTTTTTTAATGGATATAGTTGTTTAATAAAACAATTTTGTTTAAATAAAACAAAATATATTGAATGAG
CCATGTTGCTGTTGTTGTTTTTTACAGGCTTGATGTACAATATGATCTTTGTGTAGTTTTATAGTAATAA
ATCATTCATGTCTTGTGTATTGTACGTCATATTATCTTAAAAGTACGCTATTAATTAAACTTGTTTATAT
GGTCATCTTAGACAGGACTCTCATTATAAAAGGATTTTTGTTAATTTTATTAGTACCTTCCTGGAAAAA
TAAAAGATAAATAAATAAAAATGAATAAAAGTCTTGGGCTTGTTTTCTGTACGAGTAATAAAAAAGAG
TAAGCAAATATTCTGCATGACAATATTGTTCAAAGTTTAAGGGCTGTATTTTTTATAATTGTTGGTTATT
AACATGTTTTATACAAATTACTTTCAACATGGCTGAAATAATAATTGTTATCACTATTTGAAACAGTCC
TAGAATAGATTTTACTATATTTACATTTATTCCTCTGATGACAAAGTTGAAATTCCTCTGATGACAAAG
CAAAACTCTCAACTCTAGTTTTATTTTATTGTAATAACCATGTCAAGGGATAATGACAATGTTTTCCAT
TATATTTTCAATATAGAATAAAATTATAATTTATTAATCATTTAGTAGGGCAGCATGGTGGCACAATGG
GTAGCACAATCACCTCACACCAAGAAGGTTGCTAGTTCGAGCCCCGGCTGGGTCAGTTTGCATATCTG
TGTGGAGAATGTTCTCTCTGTGTTGCCATGGGTTTCCTCTGGGTGCTCTAGTTTCCACCACAAGTCTAA
AAACAAGTGGCATCGGTGAATTGTGTAGGCTAAATTGTCTGTAGTGTTTGTGTGTGATTGAGTGTTTAT
GGATGTTTCCCAGTGATGGGTTGCAGTTGGAAGGGCATTCTCTGCGTAAAACTATAAGTTGGCGGTTC
ATTCCACTGTGGTGACCCCAGATTAATAAAGGGATTAAGCCGAAAAGAAAATGAATCATTAAATGATC
ATTTAGTTGATTGGAGAGCATTGCATAATGAGAATCAAATAATGTATAGTTTTTTGTAAAATGATTGTG
TATTAGAGGCACATTGTACGTAGTGTGGAATGTTTAGGTTGTCTTTCTGATTGTTTTCATTTTGCAGAA
GTTCATTTGCAGTCAGTGTAGTCAAAGCACCTCAGCTCAATTTATAGCAGCAGCCTGCTGTGATTTAAG
TGCCACAGGAGTGAAGGCTTTAACCCCTGTTGTAGATTGCAACCAATGTTTTTCTAAGGTAATTAGAA
GCTTGTTTAGCTAACGCTACCCACCCCACCCCCCACAAAAAAGTTGCTCTGAATAGCAGCATCTACCCC
CAGGAGGGTGCAAGCTTCTGTTTTGAAGAAATTTGCTCATGGTGTTACATAGGTGTTTAAAAATGTATG
TTTTTTAATAGCATATATTTTAAAAGTGACTCATTAATGACATCATCTGCCATCTGCCTTTCTCAGAACT
GACAAAGGACCCTTTTAAGGACCCCTATACACCACATTTTTATCTATCTAATCACAAGTGGGTGATATT
AGAGGGATGATTCACCTAAAAATGAATTTTCCGCATTTTAATTTGGGGTTCCAAACCAGTTTGAATTCT
TTTGTTCTGTTGAACACAAAATAAGATGTTTTAAGAATGTTGGAAATGTTGGAGACTTTTATAGTAGGA
AAAACATATACTAATATTTGCATTCATGTTTGAAATGTTTAAAACATTTTTCAAAATATCTTATTTTTCA
ACTGTTTTGGAACAAGTCAAAAGTGAGTAAATGATGACTGATAAACAATTTCTTTAAATACAGATGAA
AATAGCGTCTAACACATTTTGAACTAGTTCACTTTCAACCACAGCAAGAGGTAGTCGAAAACACATTT
GATCACATTTTGTTGTGGGACCGCTTGTTGTTCAGTGTGTTTTCATTGTTGAGTTACAAACCGGTAAGT
GGCATTAAAACATGTTTTGGCATCACTGATGATCATAAATAAATTGGTTAAAAAATTAAAAATTAGAT
GCTTACAGTACAGTTTGTTTAAAAACTTGCACTTTTTGTAGTGTGAAATGACATTAGTACCTGCAGTGG
GCTTTCTTTGTTCTGTATCTTCTGATGAATCTAACATAAAGACATTTAAATATTCACAATGGAGTGGGT
TGGTTCTCATCTATCTGAAACAACATTAGTGTGTTCAAAACAAATCTACACCAGTGATCTCTGATAGAT
ACAAGCTGTCAACCGTAGTCCTATTGTTTCTCATGCGACTGTCACAGGGCCATGCTAGAGGCTCTTTGG
GTGGTGAGTAGAGACTCATTCTTATGCAAACAGGCTATGGATGCTACAACTGAAGAGAATTTAGTGCT
CTAAGGTCATCGCAGTTCTATAAAGCAGATTCCCTCCAAAAAGCTAGAGAAAATTATGTGTCTGGAGG
CACCATCTATCAGTGCAGTCATTCTTTTATTACTTTTTTTTTTTGTCCATGCAATTTTTTAATCGCATTTT
GAAGTATTTAAACCATTAAGAGTGTCAAATTATCATGTTATTATTTCAACTCATAATATCTCCTTTTAA
AGATGATTCATTTGATTGTAGTTATAATAATTGTTTTATTAATGCAAAAATTGCTTAAATGTTTGGCCTC
TGAGATTTTCAGTTGACTCAATATTTTTGGAGCCTGTTTCATGACTTTATTTAAAATATAATCTGCACTC
GCAAATATTACAATTATATCATATTTTGAAAATCTTAGATGTACATTTGTTTTGAAAATGGTTTAAAGG
AAATTTAGGTCAAAATGTGTGTTTTTTTTAAATAATAATTCAGTCTTTTTTTTGATATAATAGGGAAAA
TGACAGCAATTAAAAGGCTGTTTTGGAAAGACATCTAGGTCTGGTGCAGACTTCGTAACTTCTTAGAG
TTAAATCTTGAAAACGGAGCCAGAAGACGGGCAACACCTTCCTAAAAGCAGGGTGGGGTGTTGGACA
GGTAGCTTCTTGGAACCTCCCCTCTTTGTCGCTGTGGTATATATAGAAATCCCATTGAATTCCTATAGA
GACCTTAAGCCCGTACCATTTACCTCTGAAGCCTAGGATGAAGCTACTGTCTTCTATCGAACAAGCATG
CGATATTTGCCGACTTAAAAAGCTCAAGTGCTCCAAAGAAAAACCGAAGTGCGCCAAGTGTCTGAAG
AACAACTGGGAGTGTCGCTACTCTCCCAAAACCAAAAGGTCTCCGCTGACTAGGGCACATCTGACAGA
AGTGGAATCAAGGCTAGAAAGACTGGAACAGCTATTTCTACTGATTTTTCCTCGAGAAGACCTTGACA
TGATTTTGAAAATGGATTCTTTACAGGATATAAAAGCATTGTTAACAGGATTATTTGTACAAGATAATG
TGAATAAAGATGCCGTCACAGATAGATTGGCTTCAGTGGAGACTGATATGCCTCTAACATTGAGACAG
CATAGAATAAGTGCGACATCATCATCGGAAGAGAGTAGTAACAAAGGTCAAAGACAGTTGACTGTAT
CGTCGAGGTCGACCCCGGGAATTCAGATCTCTCGAGCCGCCCCCCCGACCGATGTCAGCCTGGGGGAC
GAGCTCCACTTAGACGGCGAGGACGTGGCGATGGCGCATGCCGACGCGCTAGACGATTTCGATCTGGA
CATGTTGGGGGACGGGGATTCCCCGGGTCCGGGATTTACCCCCCACGACTCCGCCCCCTACGGCGCTC
TGGATATGGCCCCCCCGACCGATGTCAGCCTGGGGGACGAGCTCCACTTAGACGGCGAGGACGTGGC
GATGGCGCATGCCGACGCGCTAGACGATTTCGATCTGGACATGTTGGGGGACGGGGATTCCCCGGGTC
CGGGATTTACCCCCCACGACTCCGCCCCCTACGGCGCTCTGGATATGGCCGACTTCGAGTTTGAGCAG
ATGTTTACCGATGCCCTTGGAATTGACGAGTACGGTGGGGGCGGCCGGCCGCGACTCTAGATCATAAT
CAGCCATACCACATTTGTAGAGGTTTTACTTGCTTTAAAAAACCTCCCACACCTCCCCCTGAACCTGAA
ACATAAAATGAATGCAATTGTTGTTGTTAACTTGTTTATTGCAGCTTATAATGGTTACAAATAAAGCAA
TAGCATCACAAATTTCACAAATAAAGCATTTTTTTCACTGCATTCTAGTTGTGGTTTGTCCAAACTCAT
CAATGTATGGCGGCCGCAGCTCGGCGCCCTTGCTCACCATGGTGGCGACCGGTCTTGTACAGCTCATC
CATGCCGTGGGTGATGGCGGCGGCGGTCACGAAGCCGAAGTAGATCATGTGATCGCGCTTCTCGTTGG
GGTCCTTGGACAGGGCGCTCTGGGTGGACAGGTAGTGGTTATCGGGCAGCAGCACAGGGCCATCGCC
GATGGGGGTATTCTGCTGGTAGTGGTCGGCCAGCTGCACGCTGCCATCCTCGATGTTGTGGCGGATCTT
GAAGTTCACCTTGATGCCATTCTTGGCCTTGTCGGTCATGATGTACACATTGTGGGCGTTGTAGTTGTA
CTCCATCTTATTGCCCAGGATGTTGCCATCCTCCTTGAAATCGGTGCCGGTCAGCTCGATGCGATTCAC
CAGGGTATCGCCCTCGAACTTCACCTCGGCGCGCGACTTGTAGTTGCCGTCATCCTCGAAGAAGATGG
TGCGCTCCTGGATGTAGCCCTCAGGCATGGCGCTCTTGAAGAAGTCGTGCTGCTTCATGTGATCGGGGT
AGCGTGAGAAGCACTGCACGCCGTAGCTCAGGGTGGTCACCAGGGTGGGCCAGGGCACAGGCAGCTT
GCCGGTGGTGCAGATGAACTTCAGGGTCAGCTTGCCGTAGGTGGCATCGCCCTCGCCCTCGCCGCTCA
CGCTGAACTTGTGGCCATTCACATCGCCATTCAGCTCGATCAGGATGGGCACGATGCCGGTGAACAGC
TCGGCGCCCTTGCTCACCATGGATCCCGTCTAGAGGGTATATAATGGATCCCATCGCGTCTCAGCCTCA
CTTTGAGCTCCTCCACACGAATTCTTTGGCCCGGAGTACTGTCCTCCGGGCTGGCGGAGTACTGTCCTC
CGGCAAGGTCGGAGTACTGTCCTCCGACACTAGAGGTCGGAGTACTGTCCTCCGACGCAAGGCGGAGT
ACTGTCCTCCGTCTAGAGGGTATATAATGGATCCCATCGCGTCTCAGCCTCACTTTGAGCTCCTCCACA
CGAATTCCCTCGACCTCGAAGACCTTAAGTTCGAATTAGGCGCGCCAGGTGATCATCATGAATCCGCT
CATTGATATCTTTTTTTTTCCTTTCTTGTACAAAGTGGGGGATCCAGACATGATAAGATACATTGATGA
GTTTGGACAAACCACAACTAGAATGCAGTGAAAAAAATGCTTTATTTGTGAAATTTGTGATGCTATTG
CTTTATTTGTAACCATTATAAGCTGCAATAAACAAGTTAACAACAACAATTGCATTCATTTTATGTTTC
AGGTTCAGGGGGAGGTGTGGGAGGTTTTTTCCAACTTTATTATACAGGTACCAAGGCCTTCGATTTTTC
GCGATTTTTTGCGAAGATACGGCCACGGGTGCTCTTGATCCTGTGGCTGATTTTGGACTGTGCTGCTCG
CAGCTGCTGATGAATCACATACTTCCTCCATTTTCTTCCACTGATTGACTGTTATAATTTCCCTAATTTC
CAGGTCAAGGTGCTGTGCATTGTGGTAATAGATGTGACATGACGTCACTTCCAAAGGACCAATGAACA
TGTCTGACCAATTTCATATAATGTGAAAACGATTTTCATAGGCAGAATAAATAACATTTAAATTAAACT
GGGCATCAGCGCAATTCAATTGGTTTGGTAATAGCAAGGGAAAATAGAATGAAGTGATCTCCAAAAA
ATAAGTACTTTTTGACTGTAAATAAAATTGTAAGGAGTAAAAAGTACTTTTTTTTCTAAAAAAATGTAA
TTAAGTAAAAGTAAAAGTATTGATTTTTAATTGTACTCAAGTAAAGTAAAAATCCCCAAAAATAATAC
TTAAGTACAGTAATCAAGTAAAATTACTCAAGTACTTTACACCTCTGGGGGAGGTTCCCTTTAGTGAG
GGTTAATTGCGCGCTTGGCGTAATCATGGTCATAGCTGTTTCCTGTGTGAAATTGTTATCCGCTCACAA
TTCCACACAACATACGAGCCGGAAGCATAAAGTGTAAAGCCTGGGGTGCCTAATGAGTGAGCTAACTC
ACATTAATTGCGTTGCGCTCACTGCCCGCTTTCCAGTCGGGAAACCTGTCGTGCCAGCTGCATTAATGA
ATCGGCCAACGCGCGGGGAGAGGCGGTTTGCGTATTGGGCGCTCTTCCGCTTCCTCGCTCACTGACTC
GCTGCGCTCGGTCGTTCGGCTGCGGCGAGCGGTATCAGCTCACTCAAAGGCGGTAATACGGTTATCCA
CAGAATCAGGGGATAACGCAGGAAAGAACATGTGAGCAAAAGGCCAGCAAAAGGCCAGGAACCGTA
AAAAGGCCGCGTTGCTGGCGTTTTTCCATAGGCTCCGCCCCCCTGACGAGCATCACAAAAATCGACGC
TCAAGTCAGAGGTGGCGAAACCCGACAGGACTATAAAGATACCAGGCGTTTCCCCCTGGAAGCTCCCT
CGTGCGCTCTCCTGTTCCGACCCTGCCGCTTACCGGATACCTGTCCGCCTTTCTCCCTTCGGGAAGCGT
GGCGCTTTCTCATAGCTCACGCTGTAGGTATCTCAGTTCGGTGTAGGTCGTTCGCTCCAAGCTGGGCTG
TGTGCACGAACCCCCCGTTCAGCCCGACCGCTGCGCCTTATCCGGTAACTATCGTCTTGAGTCCAACCC
GGTAAGACACGACTTATCGCCACTGGCAGCAGCCACTGGTAACAGGATTAGCAGAGCGAGGTATGTA
GGCGGTGCTACAGAGTTCTTGAAGTGGTGGCCTAACTACGGCTACACTAGAAGAACAGTATTTGGTAT
CTGCGCTCTGCTGAAGCCAGTTACCTTCGGAAAAAGAGTTGGTAGCTCTTGATCCGGCAAACAAACCA
CCGCTGGTAGCGGTGGTTTTTTTGTTTGCAAGCAGCAGATTACGCGCAGAAAAAAAGGATCTCAAGAA
GATCCTTTGATCTTTTCTACGGGGTCTGACGCTCAGTGGAACGAAAACTCACGTTAAGGGATTTTGGTC
ATGAGATTATCAAAAAGGATCTTCACCTAGATCCTTTTAAATTAAAAATGAAGTTTTAAATCAATCTAA
AGTATATATGAGTAAACTTGGTCTGACAGTTACCAATGCTTAATCAGTGAGGCACCTATCTCAGCGAT
CTGTCTATTTCGTTCATCCATAGTTGCCTGACTCCCCGTCGTGTAGATAACTACGATACGGGAGGGCTT
ACCATCTGGCCCCAGTGCTGCAATGATACCGCGAGACCCACGCTCACCGGCTCCAGATTTATCAGCAA
TAAACCAGCCAGCCGGAAGGGCCGAGCGCAGAAGTGGTCCTGCAACTTTATCCGCCTCCATCCAGTCT
ATTAATTGTTGCCGGGAAGCTAGAGTAAGTAGTTCGCCAGTTAATAGTTTGCGCAACGTTGTTGCCATT
GCTACAGGCATCGTGGTGTCACGCTCGTCGTTTGGTATGGCTTCATTCAGCTCCGGTTCCCAACGATCA
AGGCGAGTTACATGATCCCCCATGTTGTGCAAAAAAGCGGTTAGCTCCTTCGGTCCTCCGATCGTTGTC
AGAAGTAAGTTGGCCGCAGTGTTATCACTCATGGTTATGGCAGCACTGCATAATTCTCTTACTGTCATG
CCATCCGTAAGATGCTTTTCTGTGACTGGTGAGTACTCAACCAAGTCATTCTGAGAATAGTGTATGCGG
CGACCGAGTTGCTCTTGCCCGGCGTCAATACGGGATAATACCGCGCCACATAGCAGAACTTTAAAAGT
GCTCATCATTGGAAAACGTTCTTCGGGGCGAAAACTCTCAAGGATCTTACCGCTGTTGAGATCCAGTTC
GATGTAACCCACTCGTGCACCCAACTGATCTTCAGCATCTTTTACTTTCACCAGCGTTTCTGGGTGAGC
AAAAACAGGAAGGCAAAATGCCGCAAAAAAGGGAATAAGGGCGACACGGAAATGTTGAATACTCAT
ACTCTTCCTTTTTCAATATTATTGAAGCATTTATCAGGGTTATTGTCTCATGAGCGGATACATATTTGAA
TGTATTTAGAAAAATAAACAAATAGGGGTTCCGCGCACATTTCCCCGAAAAGTGCCAC
isl1: Gal4VP16_14xUAS-tdTomato
Sequence:
(SEQ ID NO: 31)
CCACCTAAATTGTAAGCGTTAATATTTTGTTAAAATTCGCGTTAAATTTTTGTTAAATCAGCTCATTTTT
TAACCAATAGGCCGAAATCGGCAAAATCCCTTATAAATCAAAAGAATAGACCGAGATAGGGTTGAGT
GTTGTTCCAGTTTGGAACAAGAGTCCACTATTAAAGAACGTGGACTCCAACGTCAAAGGGCGAAAAAC
CGTCTATCAGGGCGATGGCCCACGCTGGCTAAGAACTCATCAGCCTCCCCGGTCCATCTACCCACGTA
CCAATGCACCAATTGGCCACAATGACGGCTACTACATGGTGCCATTCCTTCCTCTTTATAGGAATGGAG
ACTACCTCCTGTCCAACAAGGCTCTTGGATACGAGTACGCCTACCTGTTGGACCCAGGTCATTGCACA
ACACCAGAAATGCCCTCTGATCTGCAAAAGACGTGAATATCTGTTCAGACACCCATATCCACTCTGTTC
CACACAGGTCAGAGGTTTGTCCAGGAGTTCTTGACAGAGGTGTAAAAAGTACTCAAAAATTTTACTCA
AGTGAAAGTACAAGTACTTAGGGAAAATTTTACTCAATTAAAAGTAAAAGTATCTGGCTAGAATCTTA
CTTGAGTAAAAGTAAAAAAGTACTCCATTAAAATTGTACTTGAGTATTAAGGAAGTAAAAGTAAAAGC
AAGAAAGAAAACTAGAGATTCTTGTTTAAGCTTTTAATCTCAAAAAACATTAAATGAAATGCATACAA
GGTTTTATCCTGCTTTAGAACTGTTTGTATTTAATTATCAAACTATAAGACAGACAATCTAATGCCAGT
ACACGCTACTCAAAGTTGTAAAACCTCAGATTTAACTTCAGTAGAAGCTGATTCTCAAAATTGTTAGTG
TCAAGCCTAGCTCTTTTGGGGCTGAAAAGCAATCCTGCAGTGCTGAAAAGCCTCTCACAGGCAGCCGA
TGCGGGAAGAGGTGTATTAGTCTTGATAGAGAGGCTGCAAATAGCAGGAAACGTGAGCAGAGACTCC
CTGGTGTCTGAAACACAGGCCAGATGGGCCCTCGAGCAGGAAACAGCTATGACCATGATTACGCCAA
GCTATCAACTTTGTATAGAAAAGTTGAACGAGAAACGTAAAATGATATAAATATCAATATATTAAATT
AGATTTTGCATAAAAAACAGACTACATAATACTGTAAAACACAACATATCCAGTCACTATGGTCGACC
AGAGAGGGAGAGAGGGATAGGGACAAGAGTGGCCCATCAATACAGTGACCAAACACCGGAGCTCAA
CACACACCCCTAACTCTAATCGCAAGCATCTCGATTTCGGGAAGTCCTGCAACACCTCGAACCCTCGG
CCACTTTCCCACCTTCACAGATAAAAACACGGGCACTAAGGGCCTTCTGTCCGGTTTTAAAAGTGGAC
CTAACACCGCCTTACTTTCTTGACATGGGAGACATGGGGGATCCACCGAAAAAAAAGCGTCTAATCTC
GTTGTGTGTCGGCTGTGGGAATCAAATCCATGACCAGTATATTCTGCGCGTGTCGCCGGATCTGGAGT
GGCACGCGGCGTGTTTGAAATGTGCAGAATGTAACCAGTATCTGGACGAGTCCTGTACATGTTTTGTG
CGAGACGGGAAAACTTACTGTAAACGGGACTACATCAGGTTATACGGGATCAAATGTGCAAAATGCA
ACATCGGTTTCAGCAAGAATGACTTTGTGATGAGAGCACGTTCGAAGGTTTATCATATTGAGTGTTTTA
GATGTGTGGCGTGTAGTCGGCAGCTCATCCCAGGAGATGAGTTCGCTCTGCGGGAAGACGGGCTCTTC
TGCAGGGCCGACCATGACGTGGTGGAGCGGGCAACAATGGGTGCTGGTGACCCATTAAGCCCATTAC
ATCCGGCGAGACCTTTACAAATGGCAGCAGAGCCCATTTCGGCACGTCAGCCTGCGCTTCGACCTCAT
GTGCACAAGCAACCTGAGAAAACAACCCGCGTCCGGACAGTCCTCAACGAAAAACAGCTCCATACCT
TGAGGACTTGTTACAATGCCAACCCTCGACCCGACGCCCTCATGAAAGAGCAGCTCGTTGAGATGACG
GGTCTTAGTCCGAGAGTCATCAGGGTTTGGTTTCAAAACAAGCGCTGCAAGGATAAAAAGAGGAGCA
TACTGATGAAGCAACTCCAGCAGCAGCAACCCAACGACAAAACGAACATCCAGGGGATGACAGGTAC
TCCAATGGTGGCGACCAGTCCAGAGAGACACGACGGTGGTTTGCAGGCAAACCAAGTGGAGGTGCAG
AGTTACCAACCGCCTTGGAAAGTCCTAAGTGACTTCGCACTGCAGAGTGACATCGACCAGCCTGCTTT
CCAACAACTGGTTAATTTTTCTGAAGGTGGTCCAGGCTCAAACTCCACAGGGAGCGAGGTCGCGTCAA
TGTCCTCGCAACTGCCAGATACACCAAACAGCATGGTAGCGAGTCCTATAGAGGCCTAGGCGAGTGGA
TTATCTCCTGCATCTGAAGTTTGCGAAAGGACGTGTCCGGGAACCAATCTCAGAGAGAGGCTCGTGAA
GTTTCTTCCATCCCTATTTCATTTAGGGCCGCTTCTGAATAAAAGCCCATCCTCCGACTGCACGAGGCC
TCTATATCGAAACAACCTTTGGTTGTATTTATACACTGTGATGACAATCGTGGGATTTAAACACACTCT
GACCGGGCAAGTCCAACGAGTGTTGCGAAAAGACCATTTAAAGTTGCAGACTCCAGCGAAAGTGGAC
CCTGCCGGCGCACATATTCACATACCGAACCTGACATGATCATTGCAGTGCTCCGCGGCCGAGTGGAC
CTGGTCGGCTACACTTTTGCCAAATACCCCTCCACACTTTTCCCACTTTTATGACAAATCAATGGACCT
CTACGGATATAAGACGCAAGAGAACTTAGTCTAATATATTCATTCATATATAGTCTACTTCTGTGCGCA
ACGTAAGTAGCCTATAATGAAACCATGACTGTTTATTCTGTTTCTATAAAAGATCGAGACGGACTATA
CTTGCTCACAAATAAGGGTATGGCAGCCGAGGTCTACTGCGATTACAGTTTTGTTATATGGTCTCTTTT
GAAGCCCAAGTACTAAGGCATTGCAACAAGGTATACCTCTATTTTGCCACAAGCTGTGTGGGAATTTC
ATGTGTTCGTGTCCGTCCAAGAAGTTTTTTTTCTTTCCCAAAGATGTGTATAGGTTTAATTTAAGTTAAT
ATGACTGACTAGTTCTGGAGGTTTTGTTTCGTTCCTGTATTACAAAATATTTTATTATTTATTGTCAAAA
GACATGCCACTTTTGTGTACTTTTACTTGCTGAAGAAGTAAAAAAGGAGGAAAAGAATATTGTCGACT
CCGAATGATCTATCATAAATTGACGTGTCCTGTCGTTTGATCAAAGACTGATGTAAGAACAACGCCAT
ATGTAGAGTTATCTTCAGGAAAATCTTGATCAATAAAAATCTGACGTGTACTGTAAGTCCATGAAGCT
ACTGTCTTCTATCGAACAAGCATGCGATATTTGCCGACTTAAAAAGCTCAAGTGCTCCAAAGAAAAAC
CGAAGTGCGCCAAGTGTCTGAAGAACAACTGGGAGTGTCGCTACTCTCCCAAAACCAAAAGGTCTCCG
CTGACTAGGGCACATCTGACAGAAGTGGAATCAAGGCTAGAAAGACTGGAACAGCTATTTCTACTGAT
TTTTCCTCGAGAAGACCTTGACATGATTTTGAAAATGGATTCTTTACAGGATATAAAAGCATTGTTAAC
AGGATTATTTGTACAAGATAATGTGAATAAAGATGCCGTCACAGATAGATTGGCTTCAGTGGAGACTG
ATATGCCTCTAACATTGAGACAGCATAGAATAAGTGCGACATCATCATCGGAAGAGAGTAGTAACAA
AGGTCAAAGACAGTTGACTGTATCGTCGAGGTCGACCCCGGGAATTCAGATCTCTCGAGCCGCCCCCC
CGACCGATGTCAGCCTGGGGGACGAGCTCCACTTAGACGGCGAGGACGTGGCGATGGCGCATGCCGA
CGCGCTAGACGATTTCGATCTGGACATGTTGGGGGACGGGGATTCCCCGGGTCCGGGATTTACCCCCC
ACGACTCCGCCCCCTACGGCGCTCTGGATATGTAGCGGATCTACTAGTGCGGCCCCTCTCGAGCCTCTA
GAACTATAGTGAGTCGTATTACGTAGATCCAGACATGATAAGATACATTGATGAGTTTGGACAAACCA
CAACTAGAATGCAGTGAAAAAAATGCTTTATTTGTGAAATTTGTGATGCTATTGCTTTATTTGTAACCA
TTATAAGCTGCAATAAACAAGTTAACAACAACAATTGCATTCATTTTATGTTTCAGGTTCAGGGGGAG
GTGTGGGAGGTTTTTTAATTCGCGGCCATCAAGCTTAGGCCTCCAAGGCGATCCAGACATGATAAGAT
ACATTGATGAGTTTGGACAAACCACAACTAGAATGCAGTGAAAAAAATGCTTTATTTGTGAAATTTGT
GATGCTATTGCTTTATTTGTAACCATTATAAGCTGCAATAAACAAGTTAACAACAACAATTGCATTCAT
TTTATGTTTCAGGTTCAGGGGGAGGTGTGGGAGGTTTTTTAAAGCAAGTAAAACCTCTACAAATGTGG
TATGGCTGATTATCCGGAGTACTGTCCTCCGGGCTGGCGGAGTACTGTCCTCCGGCAAGGTCGGAGTA
CTGTCCTCCGACACTAGAGGTCGGAGTACTGTCCTCCGACGCAAGGCGGAGTACTGTCCTCCGACACT
AGAGGTCGGAGTACTGTCCTCCGACACTAGAGGTCGGAGTACTGTCCTCCGACACTAGAGGTCGGAGT
ACTGTCCTCCGACACTAGAGGTCGGAGTACTGTCCTCCGACACTAGAGGTCGGAGTACTGTCCTCCGA
CACTAGAGGTCGGAGTACTGTCCTCCGACACTAGAGGTCGGAGTACTGTCCTCCGACACTAGAGGTCG
GAGTACTGTCCTCCGACACTAGAGGTCGGAGTACTGTCCTCCGTCCACTTAAGCTAGGTGGCCAGCGG
TGGGATACATTGATGAGTTTGGACAAACCACAACTAGAATGCAGTGAAAAAAATGCTTTATTTGTGAA
ATTTGTGATGCTATTGCTTTATTTCTAGAGGGTATATAATGGATCCCATCGCGTCTCAGCCTCACTTTGA
GCTCCTCCACACGAATTCTTTGGCCACAAGCTGACCCTGAAGTTCATCTGCACCACCGGCAAGCTGCCC
GTGCCCTGGCCCACCCTCGTGACCACCCTGACTACGGCGTGCAGTGCTTCAGCAATATACTCGAGTTAT
GGTGAGCAAGGGCGAGGAGGTCATCAAAGAGTTCATGCGCTTCAAGGTGCGCATGGAGGGCTCCATG
AACGGCCACGAGTTCGAGATCGAGGGCGAGGGCGAGGGCCGCCCCTACGAGGGCACCCAGACCGCCA
AGCTGAAGGTGACCAAGGGCGGCCCCCTGCCCTTCGCCTGGGACATCCTGTCCCCCCAGTTCATGTAC
GGCTCCAAGGCGTACGTGAAGCACCCCGCCGACATCCCCGATTACAAGAAGCTGTCCTTCCCCGAGGG
CTTCAAGTGGGAGCGCGTGATGAACTTCGAGGACGGCGGTCTGGTGACCGTGACCCAGGACTCCTCCC
TGCAGGACGGCACGCTGATCTACAAGGTGAAGATGCGCGGCACCAACTTCCCCCCCGACGGCCCCGTA
ATGCAGAAGAAGACCATGGGCTGGGAGGCCTCCACCGAGCGCCTGTACCCCCGCGACGGCGTGCTGA
AGGGCGAGATCCACCAGGCCCTGAAGCTGAAGGACGGCGGCCACTACCTGGTGGAGTTCAAGACCAT
CTACATGGCCAAGAAGCCCGTGCAACTGCCCGGCTACTACTACGTGGACACCAAGCTGGACATCACCT
CCCACAACGAGGACTACACCATCGTGGAACAGTACGAGCGCTCCGAGGGCCGCCACCACCTGTTCCTG
GGGCATGGCACCGGCAGCACCGGCAGCGGCAGCTCCGGCACCGCCTCCTCCGAGGACAACAACATGG
CCGTCATCAAAGAGTTCATGCGCTTCAAGGTGCGCATGGAGGGCTCCATGAACGGCCACGAGTTCGAG
ATCGAGGGCGAGGGCGAGGGCCGCCCCTACGAGGGCACCCAGACCGCCAAGCTGAAGGTGACCAAGG
GCGGCCCCCTGCCCTTCGCCTGGGACATCCTGTCCCCCCAGTTCATGTACGGCTCCAAGGCGTACGTGA
AGCACCCCGCCGACATCCCCGATTACAAGAAGCTGTCCTTCCCCGAGGGCTTCAAGTGGGAGCGCGTG
ATGAACTTCGAGGACGGCGGTCTGGTGACCGTGACCCAGGACTCCTCCCTGCAGGACGGCACGCTGAT
CTACAAGGTGAAGATGCGCGGCACCAACTTCCCCCCCGACGGCCCCGTAATGCAGAAGAAGACCATG
GGCTGGGAGGCCTCCACCGAGCGCCTGTACCCCCGCGACGGCGTGCTGAAGGGCGAGATCCACCAGG
CCCTGAAGCTGAAGGACGGCGGCCACTACCTGGTGGAGTTCAAGACCATCTACATGGCCAAGAAGCC
CGTGCAACTGCCCGGCTACTACTACGTGGACACCAAGCTGGACATCACCTCCCACAACGAGGACTACA
CCATCGTGGAACAGTACGAGCGCTCCGAGGGCCGCCACCACCTGTTCCTGTACGGCATGGACGAGCTG
TACAAGTTTATGCATATTACGCGCCATAGTGACTGGATATGTTGTGTTTTACAGTATTATGTAGTCTGT
TTTTTATGCAAAATCTAATTTAATATATTGATATTTATATCATTTTACGTTTCTCGTTCAACTTTATTATA
CATAGTTGATAATTCACTGGCCGTCGTTTTACGGTACCATCGATGATGATCCAGACATGATAAGATAC
ATTGATGAGTTTGGACAAACCACAACTAGAATGCAGTGAAAAAAATGCTTTATTTGTGAAATTTGTGA
TGCTATTGCTTTATTTGTAACCATTATAAGCTGCAATAAACAAGTTAACAACAACAATTGCATTCATTT
TATGTTTCAGGTTCAGGGGGAGGTGTGGGAGGTTTTTTAAAGCAAGTAAAACCTCTACAAATGTGGTA
TGGCTGATTATGATCCTCTAGATCAGATCTGCGAAGATACGGCCACGGGTGCTCTTGATCCTGTGGCTG
ATTTTGGACTGTGCTGCTCGCAGCTGCTGATGAATCACATACTTCCTCCATTTTCTTCCACTGATTGACT
GTTATAATTTCCCTAATTTCCAGGTCAAGGTGCTGTGCATTGTGGTAATAGATGTGACATGACGTCACT
TCCAAAGGACCAATGAACATGTCTGACCAATTTCATATAATGTGAAAACGATTTTCATAGGCAGAATA
AATAACATTTAAATTAAACTGGGCATCAGCGCAATTCAATTGGTTTGGTAATAGCAAGGGAAAATAGA
ATGAAGTGATCTCCAAAAAATAAGTACTTTTTGACTGTAAATAAAATTGTAAGGAGTAAAAAGTACTT
TTTTTTCTAAAAAAATGTAATTAAGTAAAAGTAAAAGTATTGATTTTTAATTGTACTCAAGTAAAGTAA
AAATCCCCAAAAATAATACTTAAGTACAGTAATCAAGTAAAATTACTCAAGTACTTTACACCTCTGGT
TCTTGACCCCCTACCTTCAGCAAGCCCAGCAGATCCACTAGTTCTAGAGCGGCCGCCACCGCGGTGGA
GCTCCAGCTTTTGTTCCCTTTAGTGAGGGTTAATTGCGCGCTTGGCGTAATCATGGTCATAGCTGTTTCC
TGTGTGAAATTGTTATCCGCTCACAATTCCACACAACATACGAGCCGGAAGCATAAAGTGTAAAGCCT
GGGGTGCCTAATGAGTGAGCTAACTCACATTAATTGCGTTGCGCTCACTGCCCGCTTTCCAGTCGGGA
AACCTGTCGTGCCAGCTGCATTAATGAATCGGCCAACGCGCGGGGAGAGGCGGTTTGCGTATTGGGCG
CTCTTCCGCTTCCTCGCTCACTGACTCGCTGCGCTCGGTCGTTCGGCTGCGGCGAGCGGTATCAGCTCA
CTCAAAGGCGGTAATACGGTTATCCACAGAATCAGGGGATAACGCAGGAAAGAACATGTGAGCAAAA
GGCCAGCAAAAGGCCAGGAACCGTAAAAAGGCCGCGTTGCTGGCGTTTTTCCATAGGCTCCGCCCCCC
TGACGAGCATCACAAAAATCGACGCTCAAGTCAGAGGTGGCGAAACCCGACAGGACTATAAAGATAC
CAGGCGTTTCCCCCTGGAAGCTCCCTCGTGCGCTCTCCTGTTCCGACCCTGCCGCTTACCGGATACCTG
TCCGCCTTTCTCCCTTCGGGAAGCGTGGCGCTTTCTCATAGCTCACGCTGTAGGTATCTCAGTTCGGTG
TAGGTCGTTCGCTCCAAGCTGGGCTGTGTGCACGAACCCCCCGTTCAGCCCGACCGCTGCGCCTTATCC
GGTAACTATCGTCTTGAGTCCAACCCGGTAAGACACGACTTATCGCCACTGGCAGCAGCCACTGGTAA
CAGGATTAGCAGAGCGAGGTATGTAGGCGGTGCTACAGAGTTCTTGAAGTGGTGGCCTAACTACGGCT
ACACTAGAAGGACAGTATTTGGTATCTGCGCTCTGCTGAAGCCAGTTACCTTCGGAAAAAGAGTTGGT
AGCTCTTGATCCGGCAAACAAACCACCGCTGGTAGCGGTGGTTTTTTTGTTTGCAAGCAGCAGATTAC
GCGCAGAAAAAAAGGATCTCAAGAAGATCCTTTGATCTTTTCTACGGGGTCTGACGCTCAGTGGAACG
AAAACTCACGTTAAGGGATTTTGGTCATGAGATTATCAAAAAGGATCTTCACCTAGATCCTTTTAAATT
AAAAATGAAGTTTTAAATCAATCTAAAGTATATATGAGTAAACTTGGTCTGACAGTTACCAATGCTTA
ATCAGTGAGGCACCTATCTCAGCGATCTGTCTATTTCGTTCATCCATAGTTGCCTGACTCCCCGTCGTG
TAGATAACTACGATACGGGAGGGCTTACCATCTGGCCCCAGTGCTGCAATGATACCGCGAGACCCACG
CTCACCGGCTCCAGATTTATCAGCAATAAACCAGCCAGCCGGAAGGGCCGAGCGCAGAAGTGGTCCT
GCAACTTTATCCGCCTCCATCCAGTCTATTAATTGTTGCCGGGAAGCTAGAGTAAGTAGTTCGCCAGTT
AATAGTTTGCGCAACGTTGTTGCCATTGCTACAGGCATCGTGGTGTCACGCTCGTCGTTTGGTATGGCT
TCATTCAGCTCCGGTTCCCAACGATCAAGGCGAGTTACATGATCCCCCATGTTGTGCAAAAAAGCGGT
TAGCTCCTTCGGTCCTCCGATCGTTGTCAGAAGTAAGTTGGCCGCAGTGTTATCACTCATGGTTATGGC
AGCACTGCATAATTCTCTTACTGTCATGCCATCCGTAAGATGCTTTTCTGTGACTGGTGAGTACTCAAC
CAAGTCATTCTGAGAATAGTGTATGCGGCGACCGAGTTGCTCTTGCCCGGCGTCAATACGGGATAATA
CCGCGCCACATAGCAGAACTTTAAAAGTGCTCATCATTGGAAAACGTTCTTCGGGGCGAAAACTCTCA
AGGATCTTACCGCTGTTGAGATCCAGTTCGATGTAACCCACTCGTGCACCCAACTGATCTTCAGCATCT
TTTACTTTCACCAGCGTTTCTGGGTGAGCAAAAACAGGAAGGCAAAATGCCGCAAAAAAGGGAATAA
GGGCGACACGGAAATGTTGAATACTCATACTCTTCCTTTTTCAATATTATTGAAGCATTTATCAGGGTT
ATTGTCTCATGAGCGGATACATATTTGAATGTATTTAGAAAAATAAACAAATAGGGGTTCCGCGCACA
TTTCCCCGAAAAGTG

1. A method of preventing or treating chemotherapy-induced peripheral neuropathy (CIPN) in a subject undergoing a chemotherapeutic treatment, the method comprising:
administering to the subject a therapeutic agent capable of inhibiting kinesin-5 (Eg5) function or expression.

2. The method of claim 1, wherein the therapeutic agent is an Eg5 inhibitor.

3. The method of claim 2, wherein the Eg5 inhibitor is monastrol, ispinesib, (+)-S-trityle-L-cysteine (TriC), BRD9876, filanesib (ARRY-520), litronesib (LY2523355), AZD4877, SB-743921, MK0731, 4SC-205, ARQ 621, or EMD534085.

4. The method of claim 1, wherein the therapeutic agent inhibits expression of kif/I gene encoding EG5.

5. The method of claim 4, wherein the therapeutic agent is a CRISPR-Cas9 complex comprising a single guide RNA specific for the kif11 gene.

6. The method of claim 1, wherein the chemotherapeutic treatment includes a chemotherapeutic agent capable of stabilizing microtubules in keratinocytes.

7. The method of claim 1, wherein the therapeutic agent is capable of preventing or reducing degeneration of neurons innervating epidermal keratinocytes.

8. The method of claim 6, wherein the chemotherapeutic treatment is administration of paclitaxel, docetaxel, cabazitaxel, TPI-287, albumin paclitaxel or a combination thereof.

9. The method of claim 7, wherein the therapeutic agent is delivered via intravenous administration or topical administration.

10. A pharmaceutical composition comprising a therapeutic agent capable of inhibiting Eg5 function or expression, a chemotherapeutic agent capable of stabilizing microtubules in keratinocytes, and a pharmaceutically acceptable carrier.

11. The pharmaceutical composition of claim 10, wherein the therapeutic agent is an Eg5 inhibitor.

12. The pharmaceutical composition of claim 11, wherein the Eg5 inhibitor is monastrol, ispinesib, (+)-S-trityle-L-cysteine (TriC), BRD9876, filanesib (ARRY-520), litronesib (LY2523355), AZD4877, SB-743921, MK0731, 4SC-205, ARQ 621, or EMD534085.

13. The pharmaceutical composition of claim 12, wherein the therapeutic agent inhibits expression of kif11 gene encoding Eg5.

14. The pharmaceutical composition of claim 13, wherein the therapeutic agent is a CRISPR-Cas9 complex comprising a single guide RNA specific for the kif11 gene.

15. The pharmaceutical composition of claim 10, wherein the therapeutic agent is capable of preventing or reducing degeneration of neurons innervating epidermal keratinocyte.

16. The pharmaceutical composition of claim 10, wherein the chemotherapeutic agent is paclitaxel, docetaxel, cabazitaxel, TPI-287, albumin paclitaxel, or a combination thereof.

17. The pharmaceutical composition of claim 10 for preventing chemotherapy-induced peripheral neuropathy (CIPN) by administering a therapeutically effecting amount thereof to a patient undergoing chemotherapeutic treatment with an agent that promotes degeneration of neurons innervating the skin.
